(19) United States
(12) Patent Application Publication
(10) Pub. No.: US 2025/0313903 A1
(43) Pub. Date: Oct. 9, 2025

(54) NEOANTIGEN IMMUNOTHERAPY

(71) Applicant: Gradalis, Inc., Carrollton, TX (US)

(72) Inventors: John Nemunaitis, Dallas, TX (US); Ernest Bognar, Carrollton, TX (US); Laura Nejedlik, Dallas, TX (US); Adam Walter, Carrollton, TX (US); Steven Engle, Carrollton, TX (US); David Willoughby, Carrollton, TX (US)

(21) Appl. No.: 19/240,318

(22) Filed: Jun. 17, 2025

Related U.S. Application Data
(63) Continuation of application No. PCT/US2024/057771, filed on Nov. 27, 2024.
(60) Provisional application No. 63/603,378, filed on Nov. 28, 2023.
(60) Provisional application No. 63/710,966, filed on Oct. 23, 2024.

(51) Int. Cl. C12Q 1/6886; A61K 35/13; A61P 35/00; C12Q 1/6809; C12Q 1/6844; C12Q 1/6869

(52) U.S. Cl. CPC C12Q 1/6886; A61K 35/13; A61P 35/00; C12Q 1/6809; C12Q 1/6844; C12Q 1/6869; C12Q 2600/106; C12Q 2600/156; C12Q 2600/166; C12Q 2600/172

(57) ABSTRACT

Provided herein are methods for treating cancer in a patient in need thereof by determining that the patient is homologous recombination proficient (HRP); and administering an immunotherapy (e.g., cellular or gene therapy) to the patient (e.g., a tumor-infiltrating lymphocyte (TIL) therapy). The disclosure describes mechanism of clonal vs. subclonal neoantigen targeting, evidence of preclinical and clinical benefit related to clonal neoantigens with immune therapy, and Vigil® therapy designed to expand clonal neoantigen effector cell populations.

### CROSS-REFERENCE TO RELATED APPLICATIONS

The present application is a continuation of PCT Application No. PCT/US2024/057771, filed Nov. 27, 2024, which claims benefit to U.S. Provisional Patent Application No. 63/603,378, filed on Nov. 28, 2023, and U.S. Provisional Patent Application No. 63/710,966, filed on Oct. 23, 2024, the disclosures of which are incorporated herein by reference in their entirety for all purposes.

### BACKGROUND OF THE DISCLOSURE

Clonal mutations involve the initiating molecular defects related to cellular transition of normal phenotype to malignant phenotype. Molecular assessment involving next generation and whole exome sequencing, which has advanced the field of precision therapy is now increasingly applied to biomarker determination involving targeted immune therapy.

Despite the advances in the prior art, what is needed in the art are new personalized methods for treating cancers (e.g., with immunotherapy) based on identification of properties of cancer cells, as well as the selection of patients that are most likely to respond to a particular immunotherapy. The present disclosure satisfies this need and offers other advantages as well.

### BRIEF SUMMARY OF THE DISCLOSURE

In one embodiment, the present disclosure provides a method for treating a patient having a solid tumor cancer, the method comprising:
determining that the patient is homologous recombination proficient (HRP);
determining that the patient has a clonal tumor mutation burden (cTMB) greater than a threshold; and
administering an immunotherapy to the patient.

In certain aspects, the patient has a clonal neoantigen load (cNEO) greater than a threshold.

In certain aspects, the patient has a wild-type BRCA1 gene and a wild-type BRCA2 gene.

In certain aspects, the clonal tumor mutation burden (cTMB) is determined by:
for each of a set of mutations:
determining a respective amount of tumor sequence reads that have the mutation; and
determining whether the mutation is a clonal mutation based on the respective amount, thereby determining clonal mutations; and
determining the cTMB from using the clonal mutations.

In certain aspects, the set of mutations is determined by:
performing exome sequencing of paired tumor and normal samples of a subject to obtain the tumor sequence reads and normal sequence reads; and
comparing the tumor sequence reads to the normal sequence reads to determine the set of mutations that are in the tumor sample and not in the normal sample.

In certain aspects, the method further comprises normalizing the cTMB using a size of the exome sequenced.

In certain aspects, the method further comprises:
attaching unique molecular identifiers to template nucleic acids of the paired tumor and normal samples;
amplifying the template nucleic acids prior to sequencing to generate reads;
determining a consensus read from one or more reads for each of the unique molecular identifiers, thereby generating consensus reads,
wherein the consensus reads are used for comparing the tumor sequence reads to the normal sequence reads to determine the set of mutations that are non-synonymous.

In certain aspects, the respective amount of tumor sequence reads that have the mutation is an allelic fraction.

In certain aspects, determining whether the mutation is a clonal mutation is further based on an allelic copy number and a tumor purity.

In another embodiment, the present disclosure provides a method for treating a patient having a solid tumor cancer, the method comprising:
determining that the patient is homologous recombination proficient (HRP);
determining that the patient has a clonal neoantigen load (cNEO) greater than a threshold; and
administering an immunotherapy to the patient.

In certain aspects, the clonal neoantigen load (cNEO) is determined by:
determining a set of clonal mutations that are non-synonymous;
determining an MHC haplotype of the patient using the tumor sequence reads and/or the normal sequence reads;
for each clonal mutation:
   generating a peptide corresponding to the clonal mutation at a genomic position;
   determining a likelihood of the peptide being presented on the cell surface in complex with the MHC haplotype encoded by the patient;
   identifying whether the peptide is a clonal neoantigen based on the likelihood; and
determining an amount of clonal neoantigens.

In certain aspects, determining a likelihood of the peptide being presented on the cell surface includes:
determining an MHC-1 binding affinity for the peptide; and
identifying the peptide as a clonal neoantigen when the MHC-1 binding affinity is greater than a binding threshold and is greater than a reference binding affinity of a wildtype sequence at the genomic position.

In certain aspects, the set of clonal mutations is determined by:
performing exome sequencing of paired tumor and normal samples of a subject to obtain the tumor sequence reads and normal sequence reads; and
comparing the tumor sequence reads to the normal sequence reads to determine the set of mutations that are present in the tumor samples and not in the normal samples.

In certain aspects, the method further comprises:
attaching unique molecular identifiers to template nucleic acids of the paired tumor and normal samples;
amplifying the template nucleic acids prior to sequencing to generate reads;
determining a consensus read from one or more reads for each of the unique molecular identifiers, thereby generating consensus reads, wherein the consensus reads are used for comparing the tumor sequence reads to the normal sequence reads to determine the set of mutations.

In certain aspects, determining the amount of clonal neoantigens includes counting a number of peptides identified as a clonal neoantigen.

In certain aspects, determining the amount of clonal neoantigens includes counting a number of clonal mutations resulting in at least one clonal neoantigen.

In certain aspects, the set of clonal mutations is determined by:
for each of a set of mutations:
determining a respective amount of tumor sequence reads that have the mutation; and
determining whether the mutation is a clonal mutation based on the respective amount, thereby determining clonal mutations.

In certain aspects, the respective amount of tumor sequence reads that have the mutation is an allelic fraction.

In certain aspects, determining whether the mutation is a clonal mutation is further based on an allelic copy number and a tumor purity.

In certain aspects, the method further comprises normalizing the amount of clonal neoantigens using a size of the exome sequenced.

In certain aspects, the threshold is determined using training samples having a known responder classification to an immunotherapy treatment, wherein the immunotherapy treatment induces the immune system to attack cells carrying the neoantigens.

In certain aspects, the patient has a clonal tumor mutation burden (cTMB) greater than a threshold.

In certain aspects, the patient has a wild-type BRCA1 gene and a wild-type BRCA2 gene.

In certain aspects, the immunotherapy comprises a therapeutically effective population of tumor infiltrating lymphocytes (TILs).

In certain aspects, the TILs specifically target the clonal neoantigen.

In another embodiment, the present disclosure provides a method for treating cancer in a patient in need thereof, the method comprising:
determining that the patient is homologous recombination proficient (HRP); and
administering an immunotherapy to the patient.

In certain aspects, the patient has a clonal tumor mutation burden (cTMB) greater than a threshold.

In certain aspects, the patient has a clonal neoantigen load (cNEO) greater than a threshold.

In certain aspects, the patient has a wild-type BRCA1 gene and a wild-type BRCA2 gene.

In certain aspects, the method further comprises administering to the patient:
(i) a neoantigen that is predicted to be presented by an HLA molecule encoded by an HLA allele which has been determined not to have been lost in a tumor;
(ii) an immune cell which recognizes a neoantigen that is predicted to be presented by an HLA molecule encoded by an HLA allele which has been determined not to have been lost in a tumor; or
(iii) an antibody which recognizes a neoantigen that is predicted to be presented by an HLA molecule encoded by an HLA allele which has been determined not to have been lost in a tumor.

In certain aspects, determination of whether said HLA allele has been lost comprises the steps of:
aligning HLA allele sequence information from a tumor sample from said subject with an HLA allele reference sequence which is based on said subject's HLA type; and
determining the specific copy number of said HLA allele in said tumor.

In certain aspects, the cancer is selected from the group consisting of a solid tumor cancer, ovarian cancer, adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, prostate cancer, sarcoma, stomach cancer, uterine cancer, thyroid cancer, and a hematological cancer.

In certain aspects, the method further comprises administering to the individual at least one dose of an additional therapeutic agent.

In certain aspects, the additional therapeutic agent is a checkpoint inhibitor or an angiogenesis inhibitor.

In certain aspects, the additional therapeutic agent is a checkpoint inhibitor.

In certain aspects, the immunotherapy comprises a therapeutically effective population of engineered cells selected from the group consisting of tumor infiltrating lymphocytes (TILs), T cell receptor (TCR) cells, chimeric antigen receptor (CAR) T cells, and natural killer (NK) cells.

In certain aspects, the therapeutically effective population of engineered cells is tumor infiltrating lymphocytes (TILs).

In certain aspects, the therapeutically effective population of engineered cells is chimeric antigen receptor (CAR) T cells.

In certain aspects, the therapeutically effective population of engineered cells is natural killer (NK) cells.

In one embodiment, the present disclosure provides a method for identifying a patient being a responder to a tumor infiltrating lymphocytes (TIL) therapy for treating cancer, the method comprising:
administering an immunotherapy;
assessing the effectiveness of the immunotherapy for treating cancer; and
determining that the patient is homologous recombination proficient (HRP) if the immunotherapy is effective.

In certain aspects, the method further comprises, before the administering step, determining that the patient has a clonal tumor mutation burden (cTMB) greater than a threshold.

In certain aspects, the method further comprises, before the administering step, determining that the patient has a clonal neoantigen load (cNEO) greater than a threshold.

In certain aspects, the method further comprises, before the administering step, determining that the patient has a wild-type BRCA1 gene and a wild-type BRCA2 gene.

In certain aspects, the immunotherapy comprises a therapeutically effective population of TILs.

In yet another embodiment, the present disclosure provides an engineered tumor cell construct comprising an exogenous gene to express an exogenous protein.

In yet another embodiment, the present disclosure provides a method of treating cancer in a patient in need thereof, the method comprising:
selecting a responder by first selecting a patient who is HRP; and
administering the engineered tumor cell construct to the patient who is HRP.

In certain aspects, the method further comprises, before the administering step, determining that the responder has a clonal tumor mutation burden (cTMB) greater than a threshold.

In certain aspects, the method further comprises, before the administering step, determining that the responder has a clonal neoantigen load (cNEO) greater than a threshold.

In certain aspects, the method further comprises, before the administering step, determining that the responder has a wild-type BRCA1 gene and a wild-type BRCA2 gene.

In one embodiment, the present disclosure provides a method of determining a treatment for a subject having a particular type of cancer, the method comprising:
performing exome sequencing of paired tumor and normal samples of a subject to obtain tumor sequence reads and normal sequence reads;
comparing the tumor sequence reads to the normal sequence reads to determine a set of mutations that are in the tumor sample and not in the normal sample;
determining a clonal tumor mutation burden (cTMB) by:
for each of the set of mutations:
determining a respective amount of the tumor sequence reads that have the mutation; and
determining whether the mutation is a clonal mutation based on the respective amount, thereby determining clonal mutations; and
determining the cTMB from using the clonal mutations;
comparing cTMB to a reference value, wherein the reference value is determined using training samples of training subjects that have the particular type of cancer and that have a known responder classification to an immunotherapy treatment, wherein the immunotherapy treatment that includes genetically modified tumor cells of the subject; and
determining whether the subject is a responder to the immunotherapy treatment based on the comparison.

In certain aspects, the method further comprises:
determining that the subject is homologous recombination proficient (HRP); and
determining whether the subject is the responder to the immunotherapy treatment further based on the subject being homologous recombination proficient (HRP).

In certain aspects, the subject not being homologous recombination proficient (HRP) is determined to not respond to the immunotherapy treatment.

In certain aspects, the training samples are of training subjects that are homologous recombination proficient (HRP).

In certain aspects, where the cTMB is determined from only the non-synonymous clonal mutations.

In one embodiment, the present disclosure provides a method of determining a treatment for a subject having cancer, the method comprising:
performing exome sequencing of paired tumor and normal samples of a subject to obtain tumor sequence reads and normal sequence reads;
comparing the tumor sequence reads to the normal sequence reads to identify mutations that are present in only the tumor samples but not the normal samples;
determining a set of non-synonymous clonal mutations;
determining an MHC haplotype of the subject using the tumor sequence reads and/or the normal sequence reads;
for each nonsynonymous clonal mutation:
   generating a peptide corresponding to the mutation at a genomic position;
   determining a likelihood of the peptide being presented on the cell surface in complex with the MHC haplotype encoded by the subject;
   identifying whether the peptide is a clonal neoantigen based on the likelihood
determining an amount of clonal neoantigens;
comparing the amount of clonal neoantigens to a reference value, wherein the reference value is determined using training samples having a known responder classification to an immunotherapy treatment, wherein the immunotherapy treatment induces the immune system to attack cells carrying the clonal neoantigens; and
determining whether the subject is a responder to the immunotherapy treatment based on the comparison.

In certain aspects, the subject is a responder to the immunotherapy treatment, the method further comprising:
administering the immunotherapy treatment to the subject.

In certain aspects, determining a clonal mutation of the set of clonal mutations uses a chromosomal position of the clonal mutation, a sequencing read depth for reads that match the reference sequence at the chromosomal position, a sequencing read depth for reads that match the variant sequence, allelic copy number for a major allele and a minor allele at the chromosomal position, and an estimated percentage tumor DNA in the tumor genomic DNA sample.

In certain aspects, determining the amount of clonal neoantigens includes determining a count of all peptides having a specified length range and being identified as a clonal neoantigen.

In certain aspects, the specified length range is 8-11 amino acids.

In certain aspects, determining the count includes:
generating of possible peptides within the specified length range that contain one or more non-consensus amino acids derived from the clonal mutation; and
determining a count of the possible peptides that are identified as clonal neoantigens.

In certain aspects, the amount is normalized by a length of an exome sequenced.

In certain aspects, determining the amount of clonal neoantigens includes:
determining a number of the set of non-synonymous clonal mutations that have a peptide as being a clonal neoantigen.

In one embodiment, the present disclosure provides a computer product comprising a non-transitory computer readable medium storing a plurality of instructions that, when executed, cause a computer system to perform the method of any one of the preceding claims.

In one embodiment, the present disclosure provides a system comprising:
the computer product; and
one or more processors configured to execute instructions stored on the computer readable medium.

These and other objects, aspects and embodiments will become more apparent when read with the detailed descriptions and drawings that follow.

### BRIEF DESCRIPTION OF THE DRAWINGS

FIG. 1A illustrates data from a Phase 2b clinical trial (VITAL) that demonstrated statistically significant RFS and OS results in HRP patients treated with Vigil®, which is an autologous cancer immunotherapy, compared to placebo; FIG. 1B shows robust differences maintained with further follow up.

FIG. 2 shows that Vigil® improved overall survival following frontline chemotherapy as maintenance therapy in newly diagnosed Stage IIIb-IV HRP ovarian cancer patients. Comparison of Vigil® vs. placebo OS at 2 year and 3 year follow up as maintenance in newly diagnosed Stage IIIb-IV ovarian cancer patients.

FIG. 3 illustrates a schematic workflow for generation of CTMB and CCNA data.

FIG. 4 illustrates a schematic workflow for generation of CTMB and CCNA data including key software tools.

FIG. 5 illustrates that T cell receptor (TCR) is a complex of integrated membrane proteins that participate in activation of T cells in response to clonal neoantigens. Stimulation of TCR is triggered by major histocompatibility complex molecules on tumor cells displaying the clonal neoantigen.

FIGS. 6A-6C illustrate that A) expansion of T cell TCR targeting clonal neoantigen via dendritic cell response; B) improved activity of T cell activity against high clonal neoantigens (low subclonal); C) limited T cell activity against high subclonal (low clonal neoantigens).

FIG. 7. Flow diagram for clonal neoantigen detection bioinformatics pipeline. Output data from a step that is used as input for a downstream step is represented by arrows between steps. Red dotted line arrows indicate BAM files from the non-UMI alignment module flowing to the subsequent steps. Green arrows represent additional data fields from the output of variant calling and annotation and allelic copy number determination included in the PyClone variant level report.

FIGS. 8A and 8B. Percentage of variants detected by the range of CCF values in the truth set for (A) S2R1 and (B) S2R3 simulated tumor genomes. The PPA is plotted for four different samples by CCF split into four bins as labeled on the x-axis. Varying shades of gray to blue represent simulated sequencing data containing 25%, 50%, 75%, or 100% simulated tumor genome sequence content, with the balance from simulated normal genome.

FIG. 9. Correlation between cTMB values generated by the bioinformatics pipeline as compared to those calculated from supplementary data tables of Riaz et al. Red and green filled circles represent patients with a complete/partial response or progressive disease, respectively, in response to Nivolumab treatment.

FIG. 10. Comparison of PR/CR and PD groups for cNEO metric. Raw sequencing data from Riaz et al for seven patients with partial or complete response to Nivolumab (green bars) or stable disease (red bars) were analyzed using the complete bioinformatics pipeline to generate cNEO values. Filtering of high-scoring potential peptides to select likely peptide noeantigens and calculation of the cNEO metric was conducted as described in Methods. Dotted line illustrates hypothetical threshold defining potential correlation to clinical response with cNEO. P-value shown was calculated by application of a two tailed unpaired T-test on log-transformed cNEO values.

FIGS. 11A-11C. Plots of tumor vs. Vigil levels, for 9 ovarian cancer patients selected from the Vigil Phase IIB trial, for cTMB (A), cNEO (B) and ITH (C). Patient 103_0009 in which no clonal variants were detected, is represented by red shaded circle.

FIG. 12. cTMB level of HRP patients entered into VITAL trial randomized to gemogenovatucel-T or placebo. Median value distinguishes high vs. low cTMB. There was no difference between the two groups based on the Welch Two Sample t-test (p=0.1333).

FIGS. 13A-13D. (A) High cNEO subpopulation of HRP patients demonstrates suggested OS advantage of gemogenovatucel-T vs. placebo. (B) Low ITH subpopulation of HRP patients demonstrates OS advantage of gemogenovatucel-T vs. placebo. (C) High cTMB in the ITT population did not demonstrate OS advantage. (D) High cTMB in the nonHRP population (BRCA mutant, HRD) did not demonstrate OS advantage.

FIG. 14. Long term survival analysis of gemogenovatucel-T vs. placebo HRP/cTMB high.

FIGS. 15A-15D. (A) High cNEO subpopulation of HRP patients entered into VITAL trial demonstrates OS advantage of gemogenovatucel-T treated patients vs. standard of care placebo treated patients (HR 0.39; 0.12,1.26, 95% CI p=0.051). (B) Low ITH subpopulation of HRP patients demonstrates OS advantage of gemogenovatucel-T treated patients vs. placebo treated patients (HR 0.30 (0.08,1.10, 95% CI p=0.028). High cTMB in the ITT population did not demonstrate (C) OS (HR 0.69; 0.35,1.38, 95% CI, p=0.147). (D) High cTMB in the nonHRP population (BRCA mutant/HRD) [HR 0.99 (0.39, 2.47, 95% CI) p=0.488].

FIG. 16A shows an example report for a first subject that includes cTMB and cNEO. FIG. 16B shows an example report for a second subject that includes cTMB and cNEO.

FIG. 17 is a flowchart of a method for using cTMB to identify responders to an immunotherapy treatment according to embodiments of the present disclosure.

FIG. 18 is a flowchart of a method for using cNEO to identify responders to an immunotherapy treatment according to embodiments of the present disclosure.

FIG. 19 illustrates a measurement system according to an embodiment of the present invention.

FIG. 20 shows a block diagram of an example computer system usable with systems and methods according to embodiments of the present invention.

### DETAILED DESCRIPTION OF THE DISCLOSURE

### I. Definitions

Unless defined otherwise, all technical and scientific terms used herein have the same meaning as is commonly understood by one of skill in the art to which the claimed subject matter belongs.

As used herein, ranges and amounts can be expressed as “about” a particular value or range. About also includes the exact amount. Hence “about 5 μg” means “about 5 μg” and also “5 μg.” Generally, the term “about” includes an amount that would be expected to be within experimental error. In some embodiments, “about” refers to the number or value recited, “+” or “−” 20%, 10%, or 5% of the number or value.

The terms “effective amount” or “therapeutically effective amount,” as used herein, refer to a sufficient amount of an agent or a compound being administered which will relieve to some extent one or more of the symptoms of the disease or condition being treated or prevent the onset or recurrence of the one or more symptoms of the disease or condition being treated. In some embodiments, the result is reduction and/or alleviation of the signs, symptoms, or causes of a disease, or any other desired alteration of a biological system. For example, an “effective amount” for therapeutic uses is the amount of the immunotherapy (e.g., cellular or gene therapy) required to provide a clinically significant decrease in disease symptoms without undue adverse side effects. In another example, an “effective amount” for therapeutic uses is the amount of the immunotherapy (e.g., cellular or gene therapy) as disclosed herein required to prevent a recurrence of disease symptoms without undue adverse side effects. An appropriate “effective amount” in any individual case may be determined using techniques, such as a dose escalation study. The term “therapeutically effective amount” includes, for example, a prophylactically effective amount. An “effective amount” of a compound disclosed herein, is an amount effective to achieve a desired effect or therapeutic improvement without undue adverse side effects. It is understood that, in some embodiments, “an effective amount” or “a therapeutically effective amount” varies from subject to subject, due to variation in metabolism of the immunotherapy (e.g., cellular or gene therapy), age, weight, general condition of the subject, the condition being treated, the severity of the condition being treated, and the judgment of the prescribing physician.

A “reference genome” or “reference sequence” may be an entire genome sequence of a reference organism, one or more portions of a reference genome that may or may not be contiguous, a consensus sequence of many reference organisms, a compilation sequence based on different components of different organisms, or any other appropriate reference sequence. As examples, a reference genome/sequence can at least 1,000, 10,000, 50,000, 100,000, 500,000, 1,000,000, 5,000,000, 10,000,000, 50,000,000, 100,000,000, 500,000,000, one billions, or 3 billion nucleotides long, e.g., a full human genome or a repeat masked human genome. A reference may also include information regarding variations of the reference known to be found in a population of organisms.

The term “fragment” (e.g., a DNA or an RNA fragment), as used herein, can refer to a portion of a polynucleotide or polypeptide sequence that comprises at least 3 consecutive nucleotides. A nucleic acid fragment can be double-stranded or single-stranded, methylated or unmethylated, intact or nicked, complexed or not complexed with other macromolecules, e.g. lipid particles, proteins. A nucleic acid fragment can be a linear fragment or a circular fragment. As part of an analysis of a biological sample, a statistically significant number of fragments can be analyzed, e.g., at least 1,000 fragments can be analyzed. As other examples, at least 5,000, 10,000 or 50,000 or 100,000 or 500,000 or 1,000,000 or 5,000,000 fragments, or more, can be analyzed, and such fragments can be randomly selected or selected according to one or more criteria. Such fragments can be sequenced.

A “sequence read” refers to a string of nucleotides obtained from any part or all of a nucleic acid molecule (fragment). For example, a sequence read may be a short string of nucleotides (e.g., 20-150 nucleotides) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes as may be used in microarrays, or amplification techniques, such as the polymerase chain reaction (PCR) or linear amplification using a single primer or isothermal amplification. Example sequencing techniques include massively parallel sequencing, targeted sequencing, Sanger sequencing, sequencing by ligation, ion semiconductor sequencing, and single molecule sequencing (e.g., using a nanopore, or single-molecule real-time sequencing (e.g., from Pacific Biosciences)). Such sequencing can be random sequencing or targeted sequencing (e.g., by using capture probes hybridizing to specific regions or by amplifying certain region, both of which enrich such regions). Example probe-based techniques include real-time PCR and digital PCR (e.g., droplet digital PCR). As part of an analysis of a biological sample, a statistically significant number of sequence reads can be analyzed, e.g., at least 1,000 sequence reads can be analyzed. As other examples, at least 5,000, 10,000, 50,000, 100,000, 500,000, 1,000,000, or 5,000,000 sequence reads, or more, can be analyzed. Additionally, amounts of sequence reads determined for embodiments of the present disclosure can be at least 1,000, 5,000, 10,000, 50,000, 100,000, 500,000, 1,000,000, or 5,000,000.

The term “mapping” or “aligning” refers to a process that relates a sequence to a location or coordinate (e.g., a genomic coordinate) in a reference (e.g., a reference genome) having a known reference sequence, where the sequence is similar to the known reference sequence at the location in the reference. The degree of similarity can be measured or reported in terms of a “mapping quality.” In one example of a mapping quality used herein, a mapping quality of X for a sequence with respect to a reported location or coordinate in a reference indicates that the probability of the sequence mapping to a different location is no greater than 10\hat{ }(−X/10). For instance, a mapping quality of 30 indicates a less than 0.1% probability of the sequence mapping to an alternate location.

The term “classification” as used herein refers to any number(s) or other characters(s) that are associated with a particular property of a sample. For example, a “+” symbol (or the word “positive”) could signify that a sample is classified as having deletions or amplifications. The classification can be binary (e.g., positive or negative) or have more levels of classification (e.g., a scale from 1 to 10 or 0 to 1), including probabilities. Different techniques for determining a classification can be combined to obtain a final classification from the initial or intermediate classification for each of the different techniques, e.g., by majority vote or a requirement that all initial/intermediate classifications are the same (e.g., positive).

The terms “cutoff” and “threshold” refer to predetermined numbers used in an operation. For example, a cutoff size can refer to a size above which fragments are excluded. As another example, a threshold value may be a value above or below which a particular classification applies. Either of these terms can be used in either of these contexts. A cutoff or threshold may be “a reference value” or derived from a reference value that is representative of a particular classification or discriminates between two or more classifications. A cutoff may be predetermined with or without reference to the characteristics of the sample or the subject. For example, cutoffs may be chosen based on the age or sex of the tested subject. A cutoff may be chosen after and based on output of the test data. For example, certain cutoffs may be used when the sequencing of a sample reaches a certain depth. A reference value can be selected as representative of one classification (e.g., a mean) or a value that is between two clusters of the metrics (e.g., chosen to obtain a desired sensitivity and specificity). As another example, a reference value can be determined based on statistical simulations of samples. Any of these terms can be used in any of these contexts. Such a reference value can be determined in various ways, as will be appreciated by the skilled person. For example, metrics can be determined for two different cohorts of subjects with different known classifications, and a reference value can be selected as representative of one classification (e.g., a mean) or a value that is between two clusters of the metrics (e.g., chosen to obtain a desired sensitivity and specificity). As another example, a reference value can be determined based on statistical simulations of samples. A particular value for a cutoff, threshold, reference, etc. can be determined based on a desired accuracy (e.g., a sensitivity and specificity).

The term “cTMB” refers to clonal tumor mutation burden. The total tumor mutation burden (TMB) is calculated as the sum of the cluster-specific TMBs for a sample and the cTMB is defined as the TMB for the cluster with the highest cancer cell fraction (CCF) that is a minimum of 0.9. Tumor samples with no associated clusters having a CCF >=0.9 do not have a primary clone and therefore their cTMB is reported as 0. PyClone assigns each variant detected in a tumor to a cluster, each with an estimated CCF. The cluster-specific TMB is calculated as the number of non-synonymous polymorphisms (including missense, nonsense, nonstop, in-frame insertions, in-frame deletions, frameshifts, or changes in the start codon) assigned to the cluster by PyClone divided by the length of the exome in Mb.

In certain instances, the threshold for cTMB is the median value of a statistically significant cohort of patients with a specific cancer. A cTMB value above the cohort median is a high cTMB and a cTMB value below the cohort median is a low cTMB. A person of ordinary skill in the art will recognize that in certain instances, the mean can be used for categorizing high and low cTMB values.

The term “cNEO” refers to clonal neoantigen load. A cNEO score is the number of predicted high binding affinity peptides that span a clonal non-synonymous variant and have higher affinity than the corresponding reference-derived peptide sequence, per Mb of exome.

In certain instances, the threshold for cNEO is a statistically significant value in a cohort of patients with a specific cancer, in which the value of a cNEO is the dividing line between for therapy-responsive patients (CR/PR) compared to the cNEO of non-responsive patients (PD).

As used herein, the terms “subject,” “individual,” and “patient” are used interchangeably. None of the terms are to be interpreted as requiring the supervision of a medical professional (e.g., a doctor, nurse, physician's assistant, orderly, hospice worker). As used herein, the subject is any animal, including mammals (e.g., a human or non-human animal) and non-mammals. In one embodiment of the methods and immunotherapy (e.g., cellular or gene therapy) provided herein, the mammal is a human.

As used herein, the terms “treat,” “treating,” or “treatment,” and other grammatical equivalents, including, but not limited to, alleviating, abating, or ameliorating one or more symptoms of a disease or condition, ameliorating, preventing or reducing the appearance, severity, or frequency of one or more additional symptoms of a disease or condition, ameliorating or preventing the underlying metabolic causes of one or more symptoms of a disease or condition, inhibiting the disease or condition, such as, for example, arresting the development of the disease or condition, relieving the disease or condition, causing regression of the disease or condition, relieving a condition caused by the disease or condition, preventing recurrence or prophylactically treating recurrence of the disease or condition, or inhibiting the symptoms of the disease or condition either prophylactically and/or therapeutically. In a non-limiting example, for prophylactic benefit, an immunotherapy (e.g., cellular or gene therapy) composition disclosed herein is administered to an individual at risk of developing a particular disease or condition, predisposed to developing a particular disease or condition, or to an individual previously suffering from and treated for the disease or condition. In some embodiments, the disease or condition is ovarian cancer.

As used herein, the term “prevention” means a prophylactic treatment performed before the subject suffers from a disease or the disease previously diagnosed is deteriorated, thereby enabling the subject to avoid, prevent or reduce the likelihood of the symptoms or related diseases of the disease. The subject may be a subject with an increased risk of developing a disease or a disease previously diagnosed to be deteriorated.

As used herein, the term “intradermal injection” refers to direct injection of a substance into dermis, wherein the substance is an aqueous solution consisting of water-soluble components and is prepared in an injectable volume (i.e., not to exceed 1 ml). The dermis includes the layer of the skin beneath the epidermis. In some embodiments, volumes formulated for dermal injection include, but are not limited to, about 0.1 ml, about 0.2 ml, about 0.3 ml, about 0.4 ml, about 0.5 ml, about 0.6 ml, about 0.7 ml, about 0.8 ml, about 0.9 ml, or about 1.0 ml. Intradermal injection may occur via, e.g., micro-needles, syringe, pre-filled syringe, needleless injector, etc.

As used herein, the term “transfection” refers to the introduction of foreign DNA into eukaryotic cells. In some embodiments, transfection is accomplished by any suitable means, such as for example, calcium phosphate-DNA co-precipitation, DEAE-dextran-mediated transfection, polybrene-mediated transfection, electroporation, microinjection, liposome fusion, lipofection, protoplast fusion, retroviral infection, or biolistics.

As used herein the term “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. In some embodiments, nucleic acid molecules are composed of monomers that are naturally-occurring nucleotides (such as DNA and RNA), or analogs of naturally-occurring nucleotides (e.g., α-enantiomeric forms of naturally-occurring nucleotides), or a combination of both. In some embodiments, modified nucleotides have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, in some embodiments, the entire sugar moiety is replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. In some embodiments, nucleic acid monomers are linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. In some embodiments, the term “nucleic acid” or “nucleic acid molecule” also includes so-called “peptide nucleic acids,” which comprise naturally-occurring or modified nucleic acid bases attached to a polyamide backbone. In some embodiments, nucleic acids are single stranded or double stranded.

As used herein, the term “expression vector” refers to nucleic acid molecules encoding a gene that is expressed in a host cell. In some embodiments, an expression vector comprises a transcription promoter, a gene, and a transcription terminator. In some embodiments, gene expression is placed under the control of a promoter, and such a gene is said to be “operably linked to” the promoter. In some embodiments, a regulatory element and a core promoter are operably linked if the regulatory element modulates the activity of the core promoter. As used herein, the term “promoter” refers to any DNA sequence which, when associated with a structural gene in a host yeast cell, increases, for that structural gene, one or more of 1) transcription, 2) translation or 3) mRNA stability, compared to transcription, translation or mRNA stability (longer half-life of mRNA) in the absence of the promoter sequence, under appropriate growth conditions.

As used herein the term “bi-functional” refers to a shRNA having two mechanistic pathways of action, that of the siRNA and that of the miRNA. The term “traditional” shRNA refers to a DNA transcription derived RNA acting by the siRNA mechanism of action. The term “doublet” shRNA refers to two shRNAs, each acting against the expression of two different genes but in the “traditional” siRNA mode.

As used herein, the term “homologous recombination (HR)” refers to a mechanism cells employ to repair double-stranded DNA breaks using a homologous template. HR deficiency may affect DNA repair. However, when only HR is deficient, the activities of other DNA repair mechanisms can prohibit the accumulation of excessive DNA damage and apoptosis. As used herein, the term “homologous recombination deficiency-positive,” “HRD-positive,” and “HRD” are used interchangeably, and they refer to the status that HR is deficient. Conversely, the term “homologous recombination deficiency negative,” “HRD-negative,” “homologous recombination proficient,” and “HRP” are used interchangeably, and they refer to the status that HR is not deficient.

In some embodiments, the HRD can be evaluated by screening germline or somatic mutations of genes related to HR repair. For example, DNA from blood or other tissues can be analyzed by next generation sequencing.

In some embodiments, to characterize whether an individual is HRD-positive or HRD-negative (or HRP), an HRD score can be determined. In some embodiments, an HRD score can be calculated based on scores for the loss of heterozygosity (LOH), telomeric allelic imbalance (TAI), and large-scale state transitions (LSTs). In some embodiments, the LOH is indicated by the presence of a single allele. In some embodiments, the LOH is defined as the number of chromosomal loss of heterozygosity regions longer than 15 Mb. In some embodiments, the TAI is indicated by a discrepancy in the 1 to 1 allele ratio at the end of the chromosome. In some embodiments, the LSTs are indicated by transition points between regions of abnormal and normal DNA or between two different regions of abnormality. In some embodiments, the LSTs are defined as the number of break points between regions longer than 10 Mb after filtering out regions shorter than 3 Mb. In certain embodiments, the HRD score is calculated as the sum of the LOH, TAI, and LST scores. Methods of determining an HRD score is available in the art, e.g., as described in Takaya et al., Sci Rep. 10 (1): 2757, 2020, Telli et al., Clin Cancer Res 22 (15): 3764-73, 2016, and Marchetti and McNeish, Cancer Breaking News 5 (1): 15-20, 2017. Further, commercial services for HRD score determination are also available, for example, services provided by Ambry Genetics, Caris Life Sciences, Counsylgenetic, Foundation Medicine, GeneDX, Integrated Genetics, Invitac, Myriad Genetics, and Neogenomics.

In methods described herein, an individual has a wild-type BRCA1 gene, a wild-type BRCA2 gene, or a combination thereof. In some embodiments, an individual having a wild-type BRCA1 gene, a wild-type BRCA2 gene, or a combination thereof can be HRD-negative or HRD-positive. In other embodiments, a mutation in the BRCA1/2 gene can lead to HRD. In other words, a mutation in the BRCA1/2 gene can lead to an individual having a HRD-positive status. In particular embodiments, an individual identified as having an HRD-positive status has an HRD score of 42 or greater (e.g., 42, 43, 44, 45, 46, 47, 48, 49, 50, 51, 52, 53, 54, 55, 56, 57, 58, 59, 60, or greater). Other mechanisms, such as germline and somatic mutations in other homologous recombination genes and epigenetic modifications, may also be implicated in homologous recombination.

In some embodiments, the method further comprises determining a status (i.e., wild type or mutant) of the BRCA1 gene, a BRCA2 gene, or the combination thereof in the individual. In some embodiments, the determining comprises sequencing of the BRCA1 gene, BRCA2 gene, or a combination thereof. In some embodiments, the sequencing comprises Sanger sequencing or next generation sequencing. In some embodiments, the next generation sequencing comprises massively parallel sequencing. In some embodiments, the determining comprises hybridization of nucleic acid extracted from the individual to an array. In some embodiments, the array is a microarray. In some embodiments, the determining comprises array comparative genomic hybridization of nucleic acid extracted from the individual.

U.S. Pat. No. 11,400,170 incorporated herein by reference, discloses methods of treating a cancer in an individual in need thereof by administering to the individual an expression vector (Vigil®) comprising: a) a first insert comprising a nucleic acid sequence encoding a Granulocyte Macrophage Colony Stimulating Factor (GM-CSF) sequence; and b) a second insert comprising a sequence according to SEQ ID NO:1, in which the individual has a wild-type BRCA1 gene, a wild-type BRCA2 gene, or a combination thereof, and is identified as homologous recombination deficiency (HRD)-negative i.e., HRP.

| SEQ ID | | |
|---|---|---|
| NO | Name | Sequence |
| 1 | bi-shRNA^{furin} | GAUCCUGCUGUUGACAGUGAGC |
| | | GCGGAGAAAGGAGUGAAACCU |
| | | UAGUGAAGCCACAGAUGUAAG |
| | | GUUUCACUCCUUUCUCCUUGC |
| | | CUACUGCCUCGGAAGCAGCUC |
| | | ACUACAUUACUCAGCUGUUGA |
| | | CAGUGAGCGCGGAGAAAGAUA |
| | | UGAAACCUUAGUGAAGCCACA |
| | | GAUGUAAGGUUUCACUCCUUU |
| | | CUCCUUGCCUACUGCCUCGGA |
| | | AGCUUAAUAAAGGAUCUUUUA |
| | | UUUUCAUUGGAUC |
| 2 | Passenger | GGAGAAAGGAGUGAAACCUUA |
| | strand in first | |
| | stem-loop | |
| | structure in | |
| | bi-shRNA^{furin} | |
| 3 | Guide strand | UAAGGUUUCACUCCUUUCUCC |
| | in both first | |
| | and second | |
| | stem-loop | |
| | structures in | |
| | bi-shRNA^{furin} | |
| 4 | Passenger | GGAGAAAGAUAUGAAACCUUA |
| | strand in | |
| | second stem- | |
| | loop structure | |
| | in bi- | |
| | shRNA ^{furin} | |
| 5 | miR-30a loop | GUGAAGCCACAGAUG |

Vigil® is an autologous cancer immunotherapy in which tumor cells are harvested from patients at the time of debulking surgery or other procedure and transfected via electroporation extracorporeally with a plasmid encoding for the GMCSF gene, an immune-stimulatory cytokine, and a bifunctional, short hairpin RNA (bi-shRNA) which specifically knocks down the expression of furin, the critical convertase responsible for activation of two TGFβ isoforms TGFβ-1 and TGFβ-2 (cancer immune effector suppressors). By downregulating TGFβ expression, cancer cells have a reduced ability to evade host immune responses. Vigil® is a personalized “neoantigen educating” immunotherapy which has been administered safely at doses up to 2.5×10e7 cells/injection and has shown evidence of benefit in Phase 2 testing of cancer patients including ovarian cancer (OC). Improved expression of clonal tumor neoantigen and reduced tumor suppressor effect of TGFβ is hypothesized to synergistically enhance activity of checkpoint inhibitor treatment. Moreover, preclinical evidence supports that prior administration of neoantigen educating therapy prior to cycle 1 (C1) will enhance immunotherapeutic anticancer activity of C1.

### II. Embodiments

The disclosure herein provides methods for treating cancer in a patient in need thereof. The methods involve a step of determining the homologous recombination status of the patient. As discussed herein, good DNA repair function in homologous recombination proficient (HRP) patients could be a potential mechanism that serves to increase the patient's immune response attributed to improved clonal neoantigen visualization. We have demonstrated that there was clinical benefit in the patient population of BRCA-wt, HRP treated with Vigil®, compared to similar placebo treated patients (Rocconi, et al., Lancet Oncol. 2020; 21:1661-1672; Rocconi et al., Gynecol Oncol. 2021; 161:676-680; Walter et al., Gynecol Oncol. 2021; 163:459-464).

The methods for treating cancer in a patient in need thereof include the first step of determining that the patient is HRP; and then administering an immunotherapy (e.g., cellular, gene, vaccine, or checkpoint inhibitor therapy) to the patient. The immunotherapy can include a therapeutically effective population of engineered cells selected from the group consisting of tumor infiltrating lymphocytes (TILs), T cell receptor (TCR) cells, chimeric antigen receptor (CAR) T cells, and natural killer (NK) cells. In other embodiments, the immunotherapy can include autologous tumor or dendritic cells (i.e. cell-based vaccines). Vigil therapy is a personalized cancer immunotherapy that works by taking a patient's own tumor cells, genetically modifying them to enhance their visibility to the immune system, and then re-injecting them back into the patient, “training” the immune system to recognize and attack the cancer cells more effectively; this modification involves inhibiting furin, which reduces the production of TGFB1 and TGFB2 thereby helping cancer cells to evade immune detection, while simultaneously stimulating the production of granulocyte macrophage colony-stimulating factor (GM-CSF) which activates immune cells to better recognize the cancer cells as foreign.

In some embodiments, the immunotherapy can include cell-based vaccines such as autologous tumor or dendritic cells genetically modified to promote T-cells (or other immune cell) response to the tumor. In some embodiments, autologous tumor or dendritic cells are engineered to present clonal neoantigens in complex with MHC-I or MHC-II on their cell surface. RNA or DNA vectors are designed to efficiently express a pro-peptide containing proteolytic cleavage site sequences flanking the mature clonal neoantigen peptide. The RNA or DNA expression vectors encoding the pro-peptide are introduced into autologous tumor or dendritic cells in culture, and after a period of incubation in vitro the cells are transferred to the human subject. In some embodiments the pro-peptide is encoded by a viral vector such as an engineered retrovirus, vaccinia virus, or adenovirus. In other embodiments pre-proteins or mature neoantigen peptides can be directly incubated with autologous tumor or dendritic cells in culture under conditions that promote uptake and processing of the pro-protein or peptides for presentation on the cell surface in complex with MHC-1 or MHC-2 molecules. After a period of incubation of the peptide with the cells in vitro, the cells are introduced into the human subject. The autologous tumor or dendritic cells engineered to present the clonal neoantigen peptides in complex with MHC-1 or MHC-2 may also be engineered with additional genetic elements (e.g. an expression vector encoding GM-CSF) designed to promote a response of T-cells or other immune cells to tumor cells carrying the neoantigen, and to suppress cellular pathways which allow for escape of immune surveillance. In some cases the autologous tumor cells are irradiated or treated with other agents to block further cell division prior to introduction of the cells into the human subject.

In some embodiments the immunotherapy is comprised of a non-cell-based vaccine consisting of one or more clonal neoantigen peptides or engineered RNA, DNA, or viruses encoding the pro-peptide as described above for cell-based vaccines. Engineered viral, RNA, or DNA vectors, or pro-peptides complexed with adjuvant can be delivered by intradermal, intraperitoneal, or intravenous injection or injected directly into the tumor of the patient. Once the engineered RNA, DNA or viruses are taken up by cells in vivo clonal neoantigen peptide(s) are presented on the cell surface in complex with MHC-1 or MHC-2 promoting eventual destruction of any cell horing the clonal neoantigen peptide. Many more embodiments of non-cell based vaccines carrying or producing clonal neoantigen peptides can be contemplated by one with knowledge and skill in the art.

In other aspects, personalized cancer vaccines can be prepared from a variety of antigen sources, e.g. from resected tumors, RNA or DNA obtained from autologous tumor cells and autologous tumor neoantigens in the form of synthetic peptides or proteins. Autologous dendritic cells can also be used to create personalized vaccines. In some embodiments the immunotherapy comprises a checkpoint inhibitor (CPI) as described in detail below.

In some embodiments, the therapeutically effective population of engineered cells is tumor infiltrating lymphocytes (TILs). In particular embodiments, the therapeutically effective population of engineered cells is chimeric antigen receptor (CAR) T cells. In particular embodiments, the therapeutically effective population of engineered cells is natural killer (NK) cells.

The disclosure also provides methods for treating patients with a solid tumor cancer. As described herein, a high level of cTMB in the patient has been associated with patients responding well to an immunotherapy. The method comprises determining that the patient with the solid tumor cancer is HRP and also determining that the patient has a higher than threshold level of cTMB. In some embodiments, the patient also has a low level of intra-tumor homogeneity (ITH). The method further administers an immunotherapy to the patient. In some embodiments, the solid tumor cancer is ovarian cancer. In some embodiments, the patient has a wild-type BRCA1 gene and a wild-type BRCA2 gene. The immunotherapy can be selected from one or more of the immunotherapies described above for treatment of patients selected based on HRP status.

The disclosure also provides methods for treating patients with a solid tumor cancer. As described herein, a high level of clonal neoantigen load (cNEO) in the patient has been associated with patients responding well to an immunotherapy. The method comprises determining that the patient with the solid tumor cancer is HRP and also determining that the patient has a higher than threshold level of cNEO. In some embodiments, the patient also has a low level of intra-tumor homogeneity (ITH). The method further administers an immunotherapy to the patient. The immunotherapy can be selected from one or more of the immunotherapies described above for treatment of patients selected based on HRP status. In some embodiments, the solid tumor cancer is ovarian cancer. In some embodiments, the patient has a wild-type BRCA1 gene and a wild-type BRCA2 gene.

In some embodiments, the immunotherapy (e.g., cellular or gene therapy) includes a therapeutically effective population of tumor infiltrating lymphocytes (TILs). TILs include T cells and B cells of the immune system. In some embodiments, TILs can be isolated from a cancer patient or collected from resected tumor material of the patient and enhanced and expanded ex-vivo, and delivered back to the patient as the therapeutic agent. Immunotherapies also include chimeric antigen receptor T-cell therapies in diverse indications.

The TILs can be used to target tumor neoantigens. Tumors often exhibit unique alterations in their DNA such as single nucleotide changes, insertions, and deletions that accumulate in the tumors and lead to frameshift and structural variants. Neoantigens are the protein products of the altered genes that are processed and presented on MHC molecules that are capable of eliciting T cell responses. Generation of neoantigen-specific T cells is a stochastic event. Not all random mutations affect protein-coding regions and not all mutated proteins can be recognized by T cells. The more somatic mutations a given tumor has, the greater the likelihood of that tumor possessing neoantigens and responding to agents that stimulate endogenous anti-tumor immunity. In some embodiments, TILs enriched with neoantigen-specific T cells can be used to treat tumors with a low mutational burden.

In some embodiments of the method, the patient has a clonal neoantigen load (cNEO) greater than a threshold. The term “clonal neoantigen” refers to a neoantigen that is present on all tumor cells of a tumor type. Clonal neoantigens as well as neoantigens in general may elicit T cell immunoreactivity and sensitivity to immune checkpoint blockage. One contributing factor in antitumor immunity is the repertoire of neoantigens created by genetic mutations within tumor cells. Like the corresponding mutations, these neoantigens show intratumoral heterogeneity. Some neoantigens are present in all tumor cells (clonal), and others are present in only a fraction of cells (subclonal). If the neoantigens are clonal they are counted as part of the total clonal neoantigen load (cNEO). The T-cell immunoreactivity generated against a clonal neoantigen will be more effective in targeting all tumor cells for destruction by the immune system since all tumor cells will present the same target antigen and therefore are marked for elimination by immune effector cells recognizing that antigen.

In certain cases, the method further comprises administering to the patient a neoantigen that is predicted to be presented by an HLA molecule encoded by an HLA allele which has been determined not to have been lost in a tumor. In some embodiments, comparative analysis of next generation sequencing (NGS) exome sequencing data of tumor and healthy tissue can be used for identification of an HLA allele and the protein it encodes. In some embodiments, the method further comprises administering to the patient an immune cell that recognizes a neoantigen that is predicted to be presented by an HLA molecule encoded by an HLA allele which has been determined not to have been lost in a tumor. After using NGS to identify the HLA allele and the protein it encodes, expression of the protein can be measured by RNAseq of tumor tissue. Later, putative T cell epitopes can be identified, synthesized, and then evaluated for recognition by an immune cell, such as a TIL. Immune cells (e.g., TILs) that can recognized the protein encoded by the HLA allele can be administered to the patient. In some embodiments of the method, immune cells (e.g., TILs) administered to the patient specifically target a clonal neoantigen of the patient. The neoantigen is produced and loaded on the MHC-1 molecule to be presented to the T-cells.

In certain cases, the method further comprises administering to the patient an antibody which recognizes a neoantigen that is predicted to be presented by an HLA molecule encoded by an HLA allele which has been determined not to have been lost in a tumor. To determine whether the HLA allele has been lost in a tumor sample, a number of approaches can be used. In certain embodiments, the sequence information of an HLA allele from a tumor sample of a patient can be determined first. The sequence of the HLA allele from the tumor sample can be aligned with an HLA allele reference sequence which is based on the patient's HLA type. The alignment of the two sequences can then be used to determine the specific copy number of the HLA allele in the tumor of the patient. Various methods of HLA typing are known in the art and are described in, e.g., Yi et al., Brief Bioinform. 22 (3): bbaa 143, 2021; Anzar et al., HLA 99 (4): 313-327, 2022; and Creary et al., Hum Immunol. 80 (7): 449-460, 2019.

In some embodiments of the method, the patient has a clonal tumor mutation burden (cTMB) greater than a threshold. The term “cTMB” refers to clonal tumor mutation burden. The total tumor mutation burden (TMB) is calculated as the sum of the cluster-specific TMBs for a sample and the cTMB is defined as the TMB for the cluster with the highest cancer cell fraction (CCF). Tumor samples with no associated clusters do not have a primary clone and therefore their cTMB is reported as 0. The term “clonal tumor mutation burden (cTMB)” refers to the total number of clonal mutations, e.g., somatic mutations detectable in 100% of tumor cells, present in a sequenced portion of a tumor genome. TMB can refer to the number of coding, base substitution, and indel mutations per megabase of a tumor genome being examined, and cTMB refers to the subset of those mutations that are expected to be present in all cells making up a tumor.

TMB and cTMB can be indicative for detecting, evaluating, calculating, or predicting the sensitivity and/or resistance to a cancer therapeutic agent or drug, e.g., immune cell therapy (e.g., TILs), immune checkpoint inhibitors, and/or antibodies. Tumors that have higher levels of cTMB may express more clonal neoantigens and may allow for a more robust immune response and therefore a more durable response to immunotherapy. The immune system relies on a sufficient number of neoantigens in order to appropriately respond, the number of somatic mutations may be acting as a proxy for determining the number of neoantigens in a tumor. cTMB may be used to deduce robustness of an immune response to a drug treatment and efficacy of a drug treatment in a subject. Germline and somatic variants can be bioinformatically distinguished to identify antigenic somatic variants, such as described in PCT/US2018/52087, incorporated by reference herein.

The method described herein can be used to treat a number of cancers, for example, a solid tumor cancer, ovarian cancer, adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, prostate cancer, sarcoma, stomach cancer, uterine cancer, thyroid cancer, and a hematological cancer.

In another aspect, the disclosure also features a method for treating a patient having a solid tumor cancer (e.g., an ovarian cancer), the method comprising: determining that the patient is homologous recombination proficient (HRP); determining that the patient has a clonal tumor mutation burden (cTMB) greater than a threshold; and administering an immunotherapy to the patient.

In addition to the immunotherapy (e.g., cellular or gene therapy) administered to the patient, in some embodiments, the method further comprises administering to the patient at least one dose (e.g., at least two, three, four, five, six, seven, eight, nine, or ten doses) of an additional therapeutic agent to treat the cancer. In some embodiments, the additional therapeutic agent can be dosed from 100 mg to 3000 mg, e.g., from 200 mg to 3000 mg, from 300 mg to 3000 mg, from 400 mg to 3000 mg, from 500 mg to 3000 mg, from 600 mg to 3000 mg, from 700 mg to 3000 mg, from 800 mg to 3000 mg, from 900 mg to 3000 mg, from 1000 mg to 3000 mg, from 1200 mg to 3000 mg, from 1400 mg to 3000 mg, from 1600 mg to 3000 mg, from 1800 mg to 3000 mg, from 2000 mg to 3000 mg, from 2200 mg to 3000 mg, from 2400 mg to 3000 mg, from 2600 mg to 3000 mg, from 2800 mg to 3000 mg, from 100 mg to 2800 mg, from 100 mg to 2600 mg, from 100 mg to 2400 mg, from 100 mg to 2200 mg, from 100 mg to 2000 mg, from 100 mg to 1800 mg, from 100 mg to 1600 mg, from 100 mg to 1400 mg, from 100 mg to 1200 mg, from 100 mg to 1000 mg, from 100 mg to 900 mg, from 100 mg to 800 mg, from 100 mg to 700 mg, from 100 mg to 600 mg, from 100 mg to 500 mg, from 100 mg to 400 mg, from 100 mg to 300 mg, or from 100 mg to 200 mg.

The additional therapeutic agent can be an anticancer agent, which refers to a natural or synthetic agent that is used in combination with an immunotherapy (e.g., cellular or gene therapy) described herein in methods of treating cancer. In some embodiments, an anticancer agent may be a cancer antigen. A cancer antigen is an antigen that is expressed preferentially by cancer cells (i.e., it is expressed at higher levels in cancer cells than on non-cancer cells) and in some instances it is expressed solely by cancer cells. The cancer antigen may be expressed within a cancer cell or on the surface of the cancer cell. A cancer antigen may be associated with a specific type of tumor, such as a solid tumor, a lymphoma, a carcinoma, a sarcoma, or a melanoma. A cancer antigen may elicit immune responses against the cancer (i.e., a cancerous tumor). In some embodiments, an anticancer agent may be an antibody or a fragment thereof such as a fragment antigen-binding (Fab) domain, which recognizes and binds to antigens (e.g., cancer antigens). In some embodiments, an anticancer agent may be an agent that inhibits and/or down regulates the activity of a protein that prevents immune cell activation (i.e., checkpoint inhibitors). Examples of proteins that prevent immune cell activation include, but are not limited to, PD-1 (programmed cell death protein 1), PD-L1 (programmed cell death-ligand 1), CTLA-4 (cytotoxic T-lymphocyte-associated protein 4), and LAG3 (lymphocyte-activation protein 3). An anticancer agent may be checkpoint inhibitor, such as an anti-PD-1 antibody, an anti-PD-L1 antibody, an anti-CTLA-4 antibody, or an anti-LAG3 antibody. Some examples of checkpoint inhibitors include, but are not limited to, anti-PD1 inhibitors such as nivolumab and pembrolizumab, anti-PD-L1 inhibitors such as atezolizumab, avelumab, and durvalumab, and anti-CTLA-4 inhibitors such as ipilimumab. Additional checkpoint inhibitors are described in, e.g., Li et al., Curr Med Chem 26 (17): 3009-3025, 2019; and Haanen, Prog Tumor Res 42:55-66, 2015. In yet other embodiments, the additional therapeutic agent can be an angiogenesis inhibitor. In particular embodiments, the additional therapeutic agent is a checkpoint inhibitor.

In some embodiments, the individual received an initial therapy. In some embodiments, administration of an initial therapy results in a clinical completely response of the cancer to the therapy. In some embodiments, the initial therapy comprises debulking, administration of a chemotherapy, administration of a therapeutic agent, or the combination thereof. In some embodiments, the chemotherapy comprises a platinum-based drug, a taxane, or a combination thereof. In some embodiments, the platinum-based drug comprises cisplatin, carboplatin, oxaliplatin, nedaplatin, triplatin tetranitrate, phenanthriplatin, picoplatin, satraplatin, or a combination thereof. In some embodiments, the platinum-based drug comprises carboplatin. In some embodiments, the taxane comprises paclitaxel, docetaxel, cabazitaxel, or a combination thereof. In some embodiments, the taxane comprises paclitaxel. In some embodiments, the therapeutic agent comprises an angiogenesis inhibitor, a checkpoint inhibitor, or a combination thereof. In some embodiments, the angiogenesis inhibitor comprises a vascular endothelial growth factor (VEGF) inhibitor. In some embodiments, the VEGF inhibitor comprises sorafenib, sunitinib, bevacizumab, pazopanib, axitinib, cabozantinib, levatinib, or a combination thereof. In some embodiments, the VEGF inhibitor is bevacizumab. In some embodiments, the checkpoint inhibitor comprises a PD-1 inhibitor, a PD-L1 inhibitor, a CTLA-4 inhibitor, or a combination thereof. In some embodiments, the checkpoint inhibitor comprises pembrolizumab, nivolumab, cemiplimab, atezolizumab, avelumab, durvalumab, ipilimumab, or a combination thereof. In some embodiments, the ovarian cancer is resistant or refractory to the chemotherapy or the therapeutic agent.

In certain aspects, the immunotherapy (e.g., cellular or gene therapy) is administered to the patient with an additional therapeutic agent. In some embodiments, at least one first dose of the immunotherapy (e.g., cellular or gene therapy) is administered to the individual in the absence of the additional therapeutic agent and at least one second dose of the immunotherapy (e.g., cellular or gene therapy) is administered to the individual in combination with at least one dose of the additional therapeutic agent. In some embodiments, as used herein, “in combination with” refers to administration of a dose of the additional therapeutic agent within 1 day, 2 days, 3 days, 4 days, 5 days, 6 days, 1 week, 2 weeks, 3 weeks, 4 weeks, or more of the dose of an immunotherapy (e.g., cellular or gene therapy) or on the same day as the dose of the immunotherapy (e.g., cellular or gene therapy). The additional therapeutic agent can be administered before, concurrently or after the immunotherapy (e.g., cellular or gene therapy).

In one illustrative example, an individual receives two doses of the immunotherapy (e.g., cellular or gene therapy) spaced one month apart via intradermal injection, and starting from the third month receives both: (i) an additional 10 doses of the immunotherapy (e.g., cellular or gene therapy) each spaced one month apart, and (ii) 12 doses of atezolizumb each spaced 3 weeks apart via intravenous infusion.

In another illustrative example, an individual receives two doses of the immunotherapy (e.g., cellular or gene therapy) spaced one month apart via intradermal injection, and starting from the third month receives both: (i) an additional 10 doses of the therapy each spaced one month apart, and (ii) 10 doses of atezolizumb administered via intravenous infusion on the same day as the additional 10 doses of the immunotherapy (e.g., cellular or gene therapy).

In some embodiments, administration of the immunotherapy (e.g., cellular or gene therapy) to the individual first followed by administration of a combination of the immunotherapy (e.g., cellular or gene therapy) and additional therapeutic agent results in a reduced toxicity relative to administration of the additional therapeutic agent alone. In some embodiments, administration of the immunotherapy (e.g., cellular or gene therapy) to the individual first followed by administration of a combination of the immunotherapy (e.g., cellular or gene therapy) and a checkpoint inhibitor results in a reduced toxicity relative to administration of the checkpoint alone.

In some embodiments, at least 1, 2, 3, 4, 5, 6, 7, 8, 9, 10, 11, or 12 doses of the immunotherapy (e.g., cellular or gene therapy) is administered to the individual prior to administration of the immunotherapy (e.g., cellular or gene therapy) in combination with the additional therapeutic agent. In some embodiments, at least two doses of the immunotherapy (e.g., cellular or gene therapy) are administered to the individual prior to administration of the combination of the immunotherapy (e.g., cellular or gene therapy) and additional therapeutic agent.

In some embodiments, the additional therapeutic agent comprises an angiogenesis inhibitor, a checkpoint inhibitor, or a combination thereof. In some embodiments, the angiogenesis inhibitor comprises a vascular endothelial growth factor (VEGF) inhibitor. In some embodiments, the VEGF inhibitor comprises sorafenib, sunitinib, bevacizumab, pazopanib, axitinib, cabozantinib, levatinib, or a combination thereof. In some embodiments, the VEGF inhibitor is bevacizumab. In some embodiments, the checkpoint inhibitor comprises a PD-1 inhibitor, a PD-L1 inhibitor, a CTLA-4 inhibitor, or a combination thereof. In some embodiments, the checkpoint inhibitor comprises pembrolizumab, nivolumab, cemiplimab, atezolizumab, avelumab, durvalumab, ipilimumab, or a combination thereof. In some embodiments, the additional therapeutic agent is a checkpoint inhibitor. In some embodiments, the checkpoint inhibitor is atezolizumab. In some embodiments, the additional therapeutic agent is a VEGF inhibitor. In some embodiments, the VEGF inhibitor is bevacizumab. In some embodiments, the additional therapeutic agent is administered by intravenous infusion.

In some embodiments, the additional therapeutic agent comprises a therapeutically effective dose of atezolizumab. In some embodiments, the therapeutically effective dose of atezolizumab is from about 900 mg to about 1500 mg or from about 1100 mg to 1300 mg. In some embodiments, the therapeutically effective dose of atezolizumab is about 900 mg, 1000 mg, 1100 mg, 1200 mg, 1300 mg, 1400 mg, or 1500 mg. In some embodiments, the therapeutically effective dose of atezolizumab is about 1200 mg. In some embodiments, the atezolizumab is administered by intravenous infusion.

In some embodiments, the additional therapeutic agent comprises a therapeutically effective dose of γIFN (gamma interferon). In some embodiments, the therapeutically effective dose of γIFN is from about 50 μg/m² to about 100 μg/m². In some embodiments, the therapeutically effective dose of γIFN is about 50 μg/m², about 60 μg/m², about 70 μg/m², about 80 μg/m², about 90 μg/m², or about 100 μg/m².

In some embodiments, the expression vector or the immunotherapy (e.g., cellular or gene therapy) is administered with an additional therapeutic agent. In some embodiments, the additional therapeutic agent comprises a therapeutically effective dose of γIFN (gamma interferon). In some embodiments, the therapeutically effective dose of γIFN is from about 50 μg/m² to about 100 μg/m². In some embodiments, the therapeutically effective dose of γIFN is about 50 μg/m², about 60 μg/m², about 70 μg/m², about 80 μg/m², about 90 μg/m², or about 100 μg/m². In some embodiments, the additional therapeutic agent comprises an angiogenesis inhibitor, a checkpoint inhibitor, or a combination thereof. In some embodiments, the angiogenesis inhibitor comprises a vascular endothelial growth factor (VEGF) inhibitor. In some embodiments, the VEGF inhibitor comprises sorafenib, sunitinib, bevacizumab, pazopanib, axitinib, cabozantinib, levatinib, or a combination thereof. In some embodiments, the VEGF inhibitor is bevacizumab. In some embodiments, the checkpoint inhibitor comprises a PD-1 inhibitor, a PD-L1 inhibitor, a CTLA-4 inhibitor, or a combination thereof. In some embodiments, the checkpoint inhibitor comprises pembrolizumab, nivolumab, cemiplimab, atezolizumab, avelumab, durvalumab, ipilimumab, or a combination thereof.

In some embodiments, the additional therapeutic agent is administered in addition to the immunotherapy (e.g., cellular or gene therapy) as a separate entity, i.e., the additional therapeutic agent is administered together (i.e., substantially simultaneously) with the immunotherapy (e.g., cellular or gene therapy) or administered separately from the immunotherapy (e.g., cellular or gene therapy) in methods of treating cancer. In some embodiments, an additional therapeutic agent may be capable of treating cancer or inhibiting the further development and/or progression of cancer. In some embodiments, an additional therapeutic agent may inhibit the proliferation of and/or kill cancer cells. An additional therapeutic agent may be a small molecule, a peptide, or a protein.

As discussed herein, good DNA repair function in HRP patients could be a potential mechanism that serves to increase the patient's immune response attributed to improved clonal neoantigen visualization. The disclosure also provides methods for identifying a patient being a responder to a tumor infiltrating lymphocytes (TIL) therapy for treating cancer. The method includes the first step of administering a immunotherapy (e.g., cellular or gene therapy) (e.g., TIL therapy), then assessing the effectiveness of the immunotherapy (e.g., cellular or gene therapy) for treating cancer. Once a population of patients for whom the immunotherapy (e.g., cellular or gene therapy) is effective is identified, one can then determine whether a patient in the population HRP.

### III. Other Embodiments

Anticancer activity induced by immunotherapy requires a series of steps, which include cancer identification and ability to separate cancer from non-cancer. In essence, the cancer needs to be recognized as foreign. Moreover, neoantigens which are the immune targets of cancer recognition, ideally will need to be involved and recognized on all cancer cells. Clonal neoantigens are the neoantigens present on every cancer cell, whereas subclonal neoantigens are present on only subsets of the total tumor population. Over time, as cancer cells evolve, particularly under therapeutic pressure, resistance will develop through molecular selection and subpopulations with resistant molecular profiles prevail (Turke, et al, 2010; 17:77-88; Diaz et al., 2012; 486:537-540; Wilmott et al, 2012; 11:2704-2708; Chen et al., 2012; 17:978-985). Many examples of induced molecular resistance have been demonstrated (Engelman et al., 2007; 316:1039-1043; Katayama et al., 2012; 4: 120ra117; Hata et al., 2016; 22:262-269; Russo et al, et al. 2019; 366:1473-1480). Tumor DNA repair efficiency regulates mutation rates. For example, increased DNA deficiency such as observed in BRCA1/2 mutant and/or homologous recombination deficient (HRD) cancer signaling is associated with defective DNA repair and higher mutational rate, whereas homologous recombination proficient (HRP) cancer signaling profile is associated with stable DNA repair and lower mutation rate. Russo et al recently demonstrated downregulation of DNA repair machinery as a key driver of resistance mutations in colorectal cancer (Russo et al., 2019; 366:1473-1480).

Immune checkpoint inhibitor (CPI) therapy, which requires recognition of tumor neoantigens for effect, has dramatically changed cancer management with demonstration of clinical benefit across multiple solid tumors (Larkin, et al., 2015; 373:23-34; Motzer, et al., 2015; 373:1803-1813; Borghaci, et al., 2015; 373:1627-1639). However, in many cancers, CPIs have therapeutic efficacy limited to smaller subpopulations. It has been well shown that neoantigen specific effector cells (CD8+ T cells) play a major role (Lennerz, et al., 2005; 102:16013-16018; Matsushita, et al., 2012; 482:400-404; DuPage et al., 2012; 482:405-409; Robbins et al., 2013; 19:747-752; Gubin, et al., 2014; 515:577-581) in engaging tumor cells following initiation of CPI therapy (Mellman et al., 2011; 480:480-489). Although other components of the immune response participate in the process involving CPI activity (CD4+ T cells, NK cells, dendritic cells and myeloid cells) and modulate the CD8+ T cell directed attack (Alspach, et al., 2019; 574:696-701), the most effective anticancer response to CPI therapy, is dependent on presence of neoantigen specific CD8+ T cell targeting effector cells which are able to induce direct anti-cancer activity (Ceglia, et al., 2021; 12:735584; Joshi, et al., 2018; 8) by targeting specific neoantigens. For complete immune system control and elimination of all the cancer cells there must be targeting of neoantigens contained on all cancer cells, the clonal neoantigens (Litchfield, et al., 2021; 184:596-614 e514; McGranahan, et al., 2016; 351:1463-1469). Clonal neoantigen targets have limited to no advantage as a biomarker to chemotherapy or other non-immune targeted therapies. Although clonal mutations demonstrating resistance to targeted therapy may be of novel relevance to subsequent clonal neoantigen determination, work by Wu et al. (Front Immunol 2020, 11, 1366) demonstrated response to CPI (nivolumab) in an advanced metastatic non-small cell lung cancer (NSCLC) patient who demonstrated progressive disease following acquired resistance to epidermal growth factor receptor-tyrosine kinase inhibitor (EGFR-TKI) at the development of an EGFR exon 19 deletion mutation. T cell response was shown to clonal neoantigens encoded by the EGFR 19 deletion. This patient achieved durable partial response now ongoing greater than 1 year after treatment initiation with nivolumab.

We discuss herein the role of clonal neoantigens in targeted immune response treatment, particularly addressing clonal neoantigen role in CPI effect. We will also focus on clinical management of advanced cancer and novel approaches such as Vigil® in clinical development that may enhance anticancer attack via induction of effector cell response targeting clonal neoantigens.

### A. Relevance of Clonal Mutation Assessment

DNA repair pathways are a critical determinant of cancer mutational activity, which lead to neoantigen expression (Riaz, et al., 2016; 28:411-419). For cancer to evolve, driver clonal mutations are required as inciting events within a normal cell to transition to a cancer cell phenotype. These clonal mutations are a small proportion of the tumor mutational burden (TMB) at onset of cancer and they most commonly involve signal pathways associated with cancer growth, spread and survival (Hanahan, 2022; 12:31-46; Dupont, et al., 2021; 288:6142-6158). Generally, cancer growth is initiated in the body many years before discovery and most events are controlled and eliminated by innate immune response. However, once the minimum cancer survival support pathways are met subsequent divergent mutational events occur following further cell divisions. These involve subclonal mutations which are generated at later stages of cancer evolution (McGranahan, et al., 2016; 351:1463-1469; Al Bakir, et al., 2023; 616:534-542; Gerlinger, et al., 2012; 366:883-892). Subclonal mutations however are less effective (compared to clonal) at inducing T cell populations with capacity to bind and react to all cancer cells. Subclonal neoantigens are not contained on all cancer cells. Moreover, subclonal neoantigens that present on tumor cell subpopulations are thought to facilitate tumor escape through the outgrowth of antigen-deficient cells leading to an ineffective dendritic response (Wu Y, Biswas D, Swanton C. 2022; 84:89-102; Gonzalez P A, Carreno L J, Coombs D, et al. 2005; 102:4824-4829; Valitutti S, 1995; 375:148-151; Davis M M, Boniface J J, Reich Z, et al. 1998; 16:523-544.)

The immune system's ability to detect and eliminate clonal neoantigen-bearing cells depends on the clonal neoantigen fraction within the tumor (Gejman, et al., 2018; 7) as well as quality and quantity of clonal neoantigen targeting effector cells. Attempting to achieve immune control across all metastatic disease sites by targeting subclonal antigens is not effective as the same subclonal neoantigens are generally not contained on all cancer cells at all metastatic sites. Preclinical support involving a murine model of controlled intratumor heterogeneity, which mixed multiple immune susceptible clones (i.e., of different subclonal neoantigens) actually resulted in a polyclonal, immune-resistant tumor. This work highlights the resilience of cancer resistant clones (Wolf et al., 2019; 179:219-235 e221).

Effective immune therapy involves effector cells that contain “trained” TCR regions to bind and react to neoantigens on all cancer cells. As described, successful anticancer activity requires immune recognition of all cancer cells, not just subpopulations. Measurement of the clonal tumor mutation burden (cTMB) prior to CPI therapy is thus a logical approach. Evidence of support via targeting clonal neoantigen expression suggested by McGranahan et al (McGranahan, et al., 2016; 351:1463-1469) was performed assessing response of melanoma and lung cancer patients to CPI therapy. Most mutation-driven neoantigens are derived from passenger mutations, which are divergent subclonal mutations acquired following the clonal driver mutation. Subclonal mutations can actively drive cancer progression and therefore involve subpopulations to provide a selective advantage to various subpopulation growth and/or spread. Despite attractive reasons for targeting clonal neoantigens that are derived from functionally important proteins, there is still evidence of cancer resistance occurrence. Thus, thoughtful monitoring of molecular biomarkers and possibly circulating cancer derived DNA at a molecular level will continue to be needed. For example, in one closely monitored case, following initial regression of 7 of 7 colorectal cancer metastases with KRAS-G12D-directed adaptive cell therapy, one metastasis recurred many months later. Following resection, this solitary lesion was found to have a unique strategic mutation involving HLA haplotype loss. As a result, this subpopulation of cancer cells developed resistance to the KRAS-G12D adaptive cell therapy, revealing a directly related mechanism of tumor immune evasion (Anderson, et al., 2004; 64:5456-5460). In another study, the metastasis of a primary tumor bearing an immunogenic BRAF clonal neoantigen (V599E) induced a HLA-restricted cytotoxic T cell response to the clonal BRAF neoantigen in the metastatic lesion as the inciting event. However, at recurrence there was no trace of the inciting oncogenic target mutation on follow up sequencing analyses (Andersen M H, Fensterle J, Ugurel S, et al. 2004; 64:5456-5460). These are examples of subsequent “strategic” mutations enabling immune resistance following targeted therapeutic intervention. Such reports exemplify limits of single agent targeted therapies even when directed to a clonal neoantigen. Future work regardless of neoantigen clonality should still involve a strategy towards combinatorial or sequential approaches which target multiple clonal neoantigens, if not all clonal neoantigens. To address shortcoming of selecting limited targets, an autologous cellular therapy (as opposed to allogenic) with demonstration of broad clonal neoantigen expression is a logical consideration. As previously discussed, increasing the number of expressed subclonal neoantigens hinders optimal cognate peptide-MHC complex affinity (Wu, et al., 2022; 84:89-102) and thus limits the effectiveness of the immune response. An increasing proportion of subclonal neoantigens adversely affects immune response to clonal neoantigen s limiting the therapeutic effect (Wu, et al., 2022; 84:89-102).

Poor tumor DNA repair is associated with higher mutation rate, high TMB, lower clonal neoantigen fraction, and higher subclonal mutation and subclonal neoantigen rate (Hocijmakers, 2001; 411:366-374). Additionally, as previously mentioned preclinical induction of subclonal neoantigen burden by ultraviolet light mediated DNA damage (UVB) induced to increased subclonal TMB in correlation with reduction in anticancer immune response to CPI (Wolf et al., 2019; 179:219-235 e221). UVB metagenesis involving syngeneic mice is a routine model for carcinogenesis induction (Bowman et al., 2021; 4.) related to DNA disruption and associated DNA repair deterioration. Wolf et al also showed that a reduction of subclonal neoantigens correlated with response improvement in correlation with decreased tumor growth (Wolf et al., 2019; 179:219-235 e221).

Similarly, in NSCLC, exposure to tobacco increases the mutational load of tumors and, more specifically, mutations that result in transversions (Govindan, et al., 2012; 150:1121-1134; “Cancer Genome Atlas Research N. Comprehensive molecular profiling of lung adenocarcinoma. Nature.,” 2014; 511:543-550). Defects in DNA-repair pathways could also lead to further increases in the number of mutations and subsequent subclonal neoantigens through a hypermutator phenotype (Le, et al., 2015; 372:2509-2520; Rizvi, et al., 2015; 348:124-128). Retrospective clinical data also show cancer patients receiving CPI with high tumor subclonal neoantigen content had associated decrease in OS compared to similar patients with low subclonal neoantigen content and high clonal neoantigen (McGranahan, et al., 2016; 351:1463-1469; Rizvi, et al., 2015; 348:124-128.).

Recently, we suggested good DNA repair function in HRP, BRCA-wt tumor molecular profile ovarian cancer patients as a potential mechanism to explain the increased immune response attributed to improved clonal neoantigen visualization (Nemunaitis, et al., “Rationale of Homologous Recombination Proficient Molecular Profile as Biomarker for Therapeutic Targeting in Ovarian Cancer,” n.d.). Clinical benefit (shown in next section) was shown in this population (BRCA-wt, HRP) with Vigil® compared to similar placebo treated patients (Rocconi, et al., 2020; 21:1661-1672; Rocconi et al., 2021; 161:676-680; Walter et al., 2021; 163:459-464).

### B. TCR Interaction

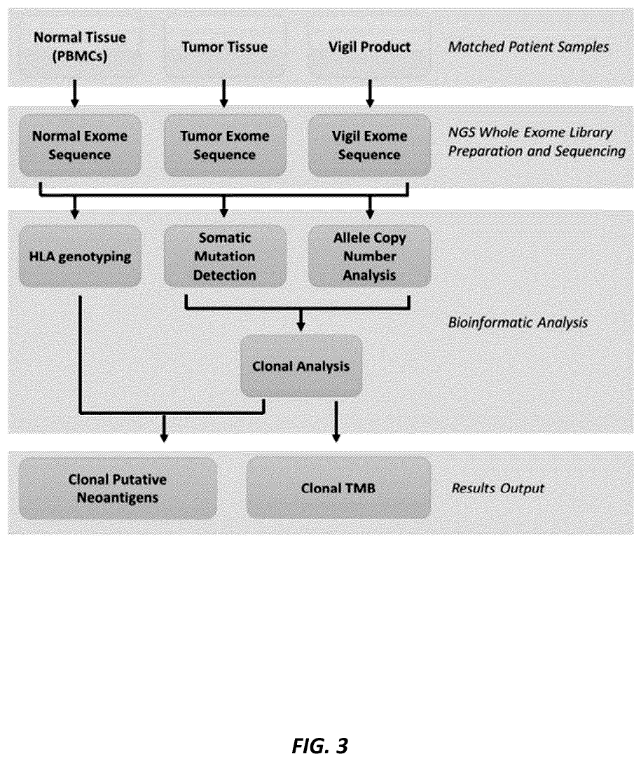

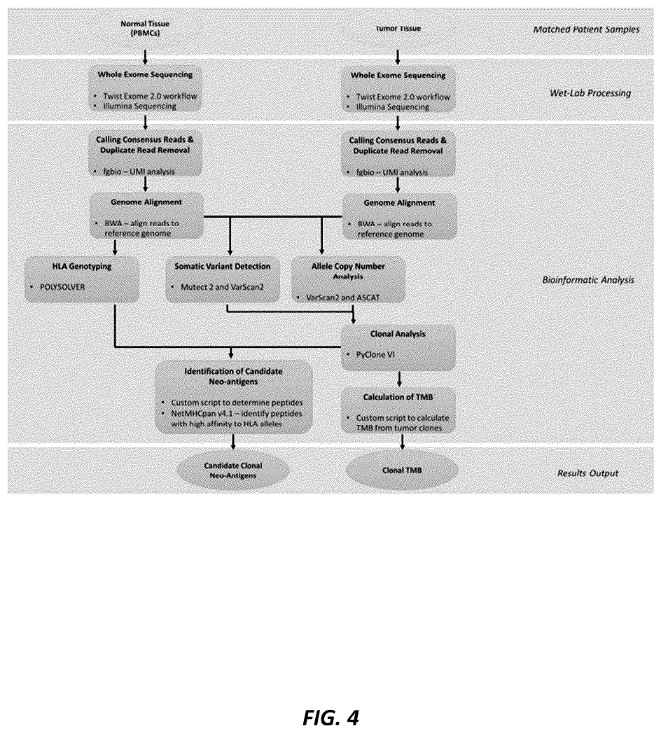

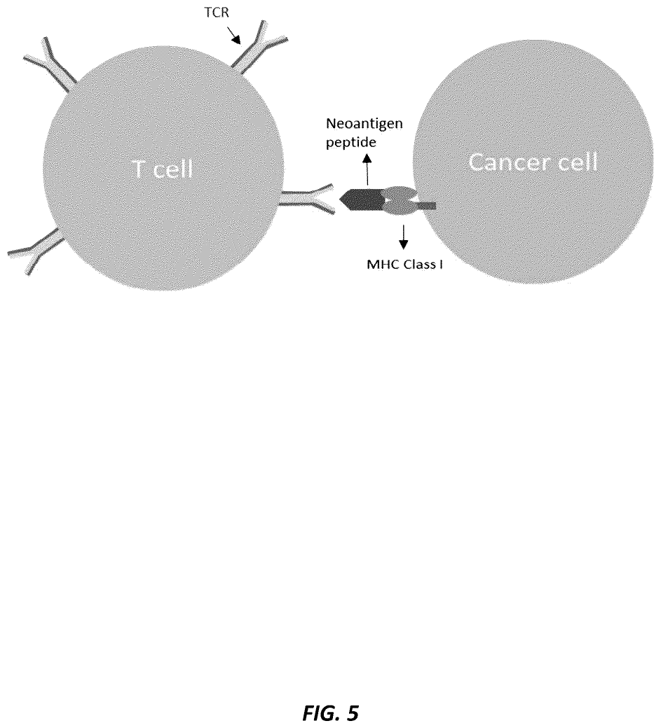

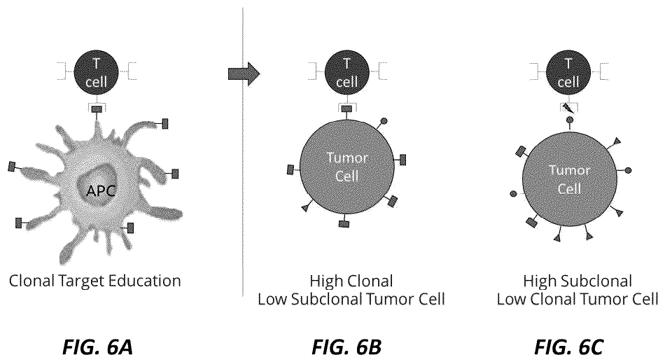

T cells are activated through specific T cell receptor (TCR)-antigen interactions. V(D)J recombination can generate a massive diversity of T cell clonotypes via thymus interaction followed by positive and negative selection processes to yield as many as 1010 circulating T cell clonotypes (Lythe et al., 2016; 389:214-224). Each clonotype TCR can bind individual T cell antigens, thereby defining T cell specificity. T cell antigens are presented on two types of major histocompatibility complex (MHC) molecules, termed human leukocyte antigens (HLAs). MHC class I (MHC-I) molecules are expressed by all nucleated cells, whereas MHC class II (MHC-II) molecules are expressed by antigen-presenting cells (APCs), epithelial cells and some tumors (Wosen, et al., 2018; 9:2144). TCR binding and relationship to relevant effector cell is shown in FIG. 5. Antitumor T cell immune response is antigen-specific and antigen selective (Coulie, et al., 2014; 14:135-146). Clinical response activity of TCR-engineered adoptive cell transfer treatment approaches are obvious examples of these assertions (Anderson, et al., 2004; 64:5456-5460; Tran, et al., 2014; 344:641-645). Lifileucel (LN44), in particular, has demonstrated robust evidence of clinical activity in advanced melanoma including in those who failed CPI therapy (Sarnaik et al., 2021; 39:2656-2666).

Mutation-derived neoantigens are aberrant proteins derived from cancer-distinct sequences encoded by somatic point mutations, frameshifts or chromosomal abnormalities. Non-synonymous mutated proteins can lead to tumor specific antigen generation if their degradation results in neopeptide HLA-binding (Vogelstein, et al., 2013; 339:1546-1558). These amino acid changes may alter the immunogenicity of an HLA-binding peptide (Peri, et al., 2021; 131) or, if they occur in anchor positions, can turn a non-binding sequence into an HLA-binding one (Duan, et al., 2014; 211:2231-2248). Additionally, a mutated amino acid could also give rise to a new proteasomal cleavage site, thus allowing peptide processing and HLA loading (Spierings, et al. 2003; 102:621-629). Evidence of the role neoantigens play in immune-mediated tumor regression is supported by the association between tumor mutational burden (TMB), in particular clonal TMB, and immunotherapy response (Litchfield et al., 2021; 184:596-614 e514). However, for response related to neoantigen exposure, T cell infiltration to the tumor environment needs to take place in order to generate T cell response. T cell priming is achieved through serial binding of multiple MHC-TCR binding complexes. Subclonal neoantigens result in suboptimal T cell priming and activation since the subclonal neoantigens are not located on all cancer cells compared to clonal neoantigens which are located on all cancer cells. Clonal neoantigens in essence result in a more durable T cell response and activation. Thus, T cell binding via TCR region interaction with clonal neoantigens induces a more effective immune effector response and release of cancer cytotoxic cytokines leading to optimal anticancer activity.

T cell response involves three stages of activation to generate anticancer activity. First is activation as related to foreign antigen stimulation which clonal neoantigen provides optimal signal. Stage 2 involves differentiation which requires complex immune effector cell interaction against the expanded cancer identifying neoantigens. Stage 3 involves a memory cell development and establishes a longer lasting almost “preventative” stage attempting to provide durability to the anticancer response (Pennock et al. Adv Physiol Educ 2013, 37, 273-283; Kumar et al., Immunity 2018, 48, 202-213). It is important to note however, that the T cell infiltration process itself (hot tumor microenvironment) has been characterized as a prognostic indication of good response and has been identified as a biomarker of CPI response benefit in some patient populations (Chyuan et al., Cancers (Basel) 2021, 13; Petitprez et al., Front Immunol 2020, 11, 784). Sufficient infiltration into the tumor microenvironment along with successful activation of effector T lymphocytes against tumor cells has been identified as predictive for response (Zhang et al., Immunotherapy 2019, 11, 201-213; Melero et al., Cancer Discov 2014, 4, 522-526; Lanitis et al., Ann Oncol 2017, 28) to immunotherapies such as Vigil (Rocconi et al., Lancet Oncol 2020, 21, 1661-1672; Walter et al., Gynecol Oncol 2021, 163, 459-464) which is designed to increase clonal neoantigen targeting T effector cells and CPIs which release inhibitory components of cancer protection to enhance access to tumor cells, even those contained within fibrotic matrix.

Optimal TCR peptide-MHC interactions can then take place under conditions that maximize serial engagement of clonal effector cells of a suitable affinity so that signal effectivity and dissociation can readily take place. Minimum affinity is required for productive signaling at each TCR (McKeithan, et al, 1995; 92:5042-5046; Grakoui, et al., 1999; 285:221-227; Savage, et al., 1999; 10:485-492), high affinity interactions may also impair overall T cell activation through prolonged dwell times and reduced serial TCR binding (Wu, et al., 2022; 84:89-102; Kalergis., 2001; 2:229-234). Increased peptide-MHC density has been shown to overcome the impairment of T cell activation by high affinity interactions as the abundance of peptide-MHC complexes compensates for reduced serial TCR engagement due to longer dwell times (Gonzalez, et al., 2005; 102:4824-4829). Clonal neoantigens, by virtue of being expressed on all tumor cells, appear to provide the requisite peptide-MHC density to engage more TCRs than subclonal neoantigens, thereby overcoming suboptimal binding affinity. As such, clonal neoantigens for these additional reasons demonstrate more effective capacity to active T cells compared to subclonal neoantigens. Engagement of multiple TCRs is required for efficient T cell activation which further supporting rationale of autologous tissue which contains all clonal neoantigens as a source for “TCR training”. Cognate peptide-MHC complexes with optimal affinity (i.e., high enough to signal, low enough to dissociate and engage further TCRs) are able to serially engage TCRs for effective T cell signaling. T cell activation is hindered by sub-optimal cognate peptide-MHC complex affinity. Subclonal neoantigens are especially handicapped by reduced serial TCR engagement due to relatively reduced peptide-MHC density. Increased peptide-MHC density of clonal neoantigens (present on all cancer cells) can overcome limitations of non-optimal TCR affinity as there is less reliance on serial engagement of TCRs (Wu, Biswas et al. 2022). Clinical evidence related to natural immune surveillance independent of combination response to immunotherapy also demonstrates OS and PFS of untreated non small cell lung cancer (NSCLC) can be predicted by defining clonal versus subclonal neoantigen concentration or TMB in association. In addition to evidence supporting clonal neoantigen targeting, some have suggested also that persistent mutations that are not clonal may also affect cancer response (Niknafs, Balan, et al. 2023) as these mutations likely occurred early in the tumor evolution (before the copy number gain event).

Several have also studied response to CPI treatment in patients with high clonal TMB or neoantigen and have also demonstrated improved OS, PFS in comparison to patients with low clonal TMB or high subclonal TMB or neoantigen expression (McGranahan, et al, 2016; 351:1463-1469; Wu, et al., 2022; 84:89-102; Rizvi, et al., 2015; 348:124-128; Snyder, et al., 2014; 371:2189-2199). A limit of clonal antigen assessment however, involves the complexity of the molecular sequencing combined with the algorithm based thresholds to determine definition of clonal vs. subclonal populations. Such assessment is not a routine process of application as of yet for clinical management.

### C. Methods of Clonal Neoantigen Identification and Analysis

Several biomarkers have demonstrated the ability to predict response to CPI including most notably PD-L1 expression, although varying therapeutic product indication thresholds have been defined ranging from 1% to 50% expression levels. Other companion diagnostic indicators such as MSI high levels and TMB also have levels of inconsistency with patient response. There are numerous examples of benefit and lack of benefit above and below thresholds set for biomarker predictability. Inconsistency demonstrated may be related to clonal vs. subclonal TMB expression which was not tracked. Additionally, inconsistency in some response correlation could be related to varied TMB levels depending on malignancy biopsy site (Rosenthal, et al., 2019; 567:479-485).

Less than 2% of the screened mutations fulfill clonal neoantigen status and are recognized by T cells. As mentioned, clonal neoantigens are neoantigens derived from the initiating signal mutations which lead to the transition evolution of normal cell phenotype to malignant cell phenotype. Such mutations typically involve molecular signal pathways involving a combination of the ten hallmark pathways of cancer survival (Hanahan, et al., 2022; 12:31-46). Mutation associated neoantigens are also typically individually unique to each patient (private neoantigens), which narrows their applicability to a “one signal fits all” therapeutic development strategy (Leko, et al., 2020; 38:454-472). In contrast, recurrent or public neoantigens derived from both point mutations and larger genetic aberrations have also been identified (Peri, et al., 2021; 131; Clark, et al., 2001; 98:2887-2893; Vanderlee, et al., 2019; 129:774-785) in rare subset populations and may be opportunity for allogencic approaches.

The advent of next-generation sequencing (NGS) has allowed the systematic, unbiased survey of mutations from individual tumors (Segal, et al., 2008; 68:889-892). These data, in turn, have been used to guide antigen discovery (Garcia-Garijo, Fajardo, Gros, 2019; 10:1392; Kalaora, et al., 2019:203-214; Castle, et al., 2012; 72:1081-1091; Bassani-Sternberg, et al., 2016; 41:9-17) either through T cell-based assays or with HLA peptidomics (Leko, et al., 2020; 38:454-472; Garcia-Garijo, Fajardo, Gros, 2019; 10:1392; Schumacher, et al., 2019; 37:173-200).

But assay development for routine clonal neoantigen assessment is complex, costly and not yet a readily available opportunity for routine clinical management. However, the field of molecular and bioinformatic technology is rapidly advancing. Data generated from whole-exome sequencing-based screening has also been used to show that tumor-infiltrating lymphocyte (TIL) reactivities against mutation-derived neoantigens actually exist in the majority of cancers, not just in tumor types known to be amenable to immunotherapy (i.e., melanoma) (Leko, Rosenberg, 2020; 38:454-472). One of the first attempts at using whole exome sequencing data to characterize neoantigens in breast and colon cancer used binding to HLA-A02: 01 to predict mutated peptides (Segal, et al., 2008; 68:889-892). Later the technique was optimized with use of patient specific HLA allele characterization (Riaz, et al., 2016; 28:411-419).

The advancement in bioinformatic pipeline developments have also now enabled the analysis of combined NGS and whole exome sequencing data as well as transcriptome data for biomarker discovery, particularly the identification of clonal neoantigens and clonal TMB.

To determine clonal neoantigens, first both tumor and normal DNA must be extracted and verification of quality and yield performed. Sequencing libraries using commercially available kits are then generated. It is important to ensure the kit chosen covers MHC-1 and MHC-2 loci for HLA typing. Generated libraries are again assessed for quality and yield before sequencing. Sequencing is performed on an Illumina sequencer with sufficient depth for variant calling. Typically, normal tissue is sequenced to a depth of 50-100× and tumor tissue between 400-500×. Sequencing reads are then processed through three different bioinformatic pipelines 1) HLA germline genotyping, 2) somatic mutation detection 3) copy number variation analysis. Somatic variant calls annotated with copy number variation data for each patient is inputted into PyClone-VI or other software for clonal deconvolution, which uses a Bayesian statistical method to determine clonal populations 70.

The variants that are associated with a primary clone based on the Pyclone output can be further analyzed to calculate clonal TMB and for the identification of a short-list of clonal neoantigens. Clonal TMB is calculated as the count of non-synonymous mutations associated with the primary clone per megabase of DNA covered by the exome panel used for sequencing. An initial broad set of candidate clonal neoantigens is generated computationally as the set of all possible 9-11-mer or 8-11 mer amino acid sequences that contain one or more mutated amino acids that are predicted to arise from one of the primary clonal variants in the DNA. The list of candidate neoantigens peptides for each patient is narrowed by utilization of NetMHCpan and NetMHCIIpan software to predict the affinity of binding of the peptides to the HLA-I and HLA-II molecules, respectively, using the predicted HLA haplotypes and list of candidate peptides as input. Peptides with a predicted IC50 of <500 nM for binding to the HLA molecule presentation sites are retained for further analysis. The candidate clonal neoantigen list can be further narrowed based on examination of the predicted cleavage sites and transport binding sites in the peptide or parent protein sequence. The narrowed candidate list of peptides can then be tested to determine the likelihood that each has been previously presented and encountered by T cells in the patient. This can be accomplished by culturing the patient's PBMCs in the presence of candidate neoantigen peptides and wild-type sequence controls with periodic replenishment with a media containing IL-2, IL-7, and IL-15, and then measuring the level of T cell activation using an IFN-γ ELISpot assay.

Immunopeptidomics can also be utilized to determine clonal neoantigens. In this approach, tumor cells are lysed and immunoprecipitation is used to identify MHC-I ligands. These peptides are then analyzed by LC-MS/MS (Kote, et al., 2020; 12:535). Numerous examples of clonal and subclonal determination in preclinical testing and retrospective clinical assessment in comparison to clinical benefit parameters have been discussed. Establishment of prospective studies to validate relationship of tumor clonal neoantigen or clonal TMB level to clinical benefit are thus justified.

### D. Response to CPI

Prior to the advent of targeted immune therapy led by development involving checkpoint inhibitors (CTL-4, PD-1, PD-L1 inhibitors), precision therapy or driver gene targeted therapy was initiated. Precision therapy is defined as the selection of a specific molecular therapy for management of cancer treatment based on molecular understanding of that disease. In simple terms, it is a “targeted therapy that targets the etiologic cancer target” which likely involves clonal mutated genes. Such molecular based targeting requires understanding of the role of the signal to driver gene function that generates cancer cell survival advantage. Knowing the gate keeping function of cancer survival thus provides the justification for therapeutically disrupting that signal. Early signal targets involve the hallmark pathway of cancer survival (i.e., Her2, EGFR, mTor, ALK, BRAF, RAS, CDK4/6, BRCA1, NTRK).

Precision therapy products have demonstrated significant clinical benefit involving OS, RFS, PFS and duration of response (Haslem, et al., 2017; 13: e108-e119; Radovich, et al., 2016; 7:56491-56500; Jardim, et al., 2015; 107; Schwaederle, et al., 2016; 2:1452-1459; Schwaederle, et al., 2015; 33:3817-3825; Dhir, et al., 2017; 6:195-206). Such benefit has led to lower hospitalization rate and ER visits. Moreover, fewer treatment related toxic deaths and payer cost advantage when precision therapy is utilized early in cancer management has also been commonly observed in comparison to standard of care chemotherapy (Chawla, et al., 2018; 2; Handorf, et al., 2012; 8:267-274; Pennell, et al., 2019; 3:1-9). As such, commercial experience now justifies implementation of comprehensive molecular profiling assessment early in patient disease diagnosis (Barlesi, et al., 2016; 387:1415-1426; Signorovitvh, et al. 2017; 35:6599-6599). Benefit related to “targeting the correct target” is an important point in understanding precision immunotherapy (i.e., CPI's) particularly as our awareness of the role of clonal neoantigens expands. The lower toxicity profile of precision therapy also further justifies utilization of combination treatment with other signal pathway precision therapeutics and/or with precision immunotherapy combinations. Moreover, improved clinical benefits involving PFS and OS have been well demonstrated with combination precision therapy as opposed to single agent (Sicklick, et al., 2019; 25:744-750). Improved clinical benefit has also been suggested with combination immunotherapy (i.e., CTL-4/PD-1/PD-L1 inhibitor combination).

Targeting and interruption of receptor-ligand interactions using CPI, release the inhibitory “brakes”, and enhance immune anti-tumor T cell reactivity, thus promoting immune control over the cancerous cells. As of now CPI agents are classified based on the target interactions they disrupt: anti-CTLA-4, anti-PD-1 and anti-PD-L1 agents. Clinical response to CPI varies based on tumor histology, line of therapy, treatment regimen (CPI monotherapy vs. combination of two CPI agents), and expression of tumor molecular markers such as PD-L1, TMB, MSI and MMR status, or BRAF mutation. Several observations however, have pointed to significant responses to CPI therapy, irrespective of the aforementioned markers, highlighting the difference in sensitivity of CPI to subclonal (“passenger” mutations) vs. clonal neoantigen expression (McGranahan, et al., 2016; 351:1463-1469; Wu, et al., Wolf et al., 2019; 179:219-235 e221; Rosenthal, et al., 2019; 567:479-485). The currently defined TMB pools together, and does not distinguish between clonal/driver neoantigen expression and subclonal neoantigens, possibly explaining the less-than-expected responses to CPI in tumors, otherwise expected to be highly neo-antigenic secondary to significant carcinogen exposure, such as small-cell lung cancer for example.

Ipilimumab is the first, and only FDA approved CTLA-4 inhibitor, which was initially approved in 2011 based on the pivotal Phase 3 MDX010-020 trial in patients with unresectable, previously treated, late-stage melanoma, revealing OS advantage for ipilimumab at a dose of 3 mg/kg every 3 weeks (alone or in combination with gp100) compared to gp100 alone (Hodi, et al., 2010; 363:711-72). In CheckMate-069, ipilimumab in combination with nivolumab in untreated melanoma unresectable/metastatic melanoma patients, provided superior objective response rates (ORR) and PFS advantage compared to ipilimumab alone (median PFS: not reached vs. 4.4 months; HR: 0.40; 95% CI: 0.23-0.68; p<0.001), granting the combination FDA approval for BRAF V600 wild-type tumors (Postow, et al., 2015; 372:2006-2017). CheckMate-067 trial data provided expanded approval of the combination, regardless of BRAF mutation, with significant OS advantage (Wolchok, et al., 2017; 377:1345-1356). The nivolumab/ipilimumab combination has also shown significant response rates (42% vs. 27%), OS (median not reached vs. 26 months) and PFS advantage (11.6 vs. 8.4 months) compared to standard-of-care sunitinib in treatment-naïve, advanced, intermediate-poor risk clear cell renal cell carcinoma, irrespective of PD-L1 status (CheckMate-214) (Motzer et al., 2018; 378:1277-1290). Last, patients with untreated, metastatic colorectal cancer with MSI-high/dMMR tumors treated with combination of nivolumab and ipilimumab demonstrated robust and durable clinical benefit with 69% response rate, 84% disease control rate, and 13% complete response (CheckMate-142) (Lenz, et al., 2022; 40:161-170).

Response to PD-1 CPI: In 2014, the FDA approved antibodies targeting PD-1-pembrolizumab and nivolumab—for the treatment of metastatic melanoma. Both are fully humanized IgG4 antibodies targeting PD-1 that selectively block the interaction of the receptor with PD-L1 and PD-L2.

Nivolumab achieved response rates of 32% in patients with metastatic melanoma whose tumors progressed after anti-CTLA-4 therapy (CheckMate-037) 93 and 40% in previously untreated patients who had metastatic melanoma with wild-type BRAF (CheckMate-067 and CheckMate-069) (Larkin, et al., 2015; 373:23-34; Hodi, et al., 2010; 363:711-723; Robert, et. Al., 2015; 372:320-330). The FDA further expanded the indication for nivolumab monotherapy to the adjuvant setting in patients with lymph-node positive or metastatic melanoma after complete resection, based on significantly longer recurrence-free survival (RFS) rates and lower toxicity, compared to adjuvant ipilimumab (12-month RFS 70.5% vs. 60.8%, respectively; p<0.001) (CheckMate-238) (Robert, et al., 2015; 372:320-330). In 2015, nivolumab was approved for patients with non-small cell lung cancer (NSCLC) who progressed after platinum-doublet chemotherapy, based on the result of the CheckMate-017 and CheckMate-057 trials, which revealed higher response rates (ORR: 19-20% vs. 9-12%) and survival benefit (median OS and PFS: 9.2-12.2 months and 2.3-3.5 months, vs. 6.0-9.4 and 2.8-4.2 months, respectively; p<0.001), regardless of PD-L1 expression level (Borghaci, et al., 2015; 373:1627-1639; Brahmer, et al., 2015; 373:123-135). In patients with small-cell lung cancer (SCLC) who progressed on platinum therapy and at least one other line of therapy, addition of nivolumab conferred increased response rate (11.6%) and OS (24-months OS rates: 17.9% and median OS: 4.7 months) (CheckMate-032) (Ready, et al., 2020; 15:426-435). Nivolumab also increased OS in patients with advanced renal cell cancer compared to everolimus (25.0 vs. 19.6 months; p=0.002) (CheckMate-025) (Motzer, et al., 2015; 373:1803-1813). In 2016, nivolumab obtained FDA indication for relapsed/refractory metastatic squamous cell cancer of head and neck, in second-line setting based on OS benefit (7.5 months) compared to standard therapy (5.1 months; p=0.01) (CheckMate-141) (Ferris, et al., 2016; 375:1856-1867).

Pembrolizumab was first approved in patients with BRAF^{V600E} mutated metastatic melanoma, refractory to CTLA-4 therapy and BRAF inhibitor, based on objective response rates of 24% in a Phase 1 trial (Robert, et al., 2014; 384:1109-1117). Based on Keynote-006 Phase 3 trial, the indication was expanded to include untreated patients with advanced melanoma, regardless of BRAF status, with proven OS (1-year OS: 74.1% vs. 58.2%; p=0.0005) and PFS (47.3% vs. 26.5%; p<0.001) advantage compared to ipilimumab (Robert, et al., 2015; 372:2521-2532). Pembrolizumab is also approved in the adjuvant setting based on the Phase 3 Keynote-054 trial which reveals prolonged RFS (65.33% vs. 49.4%; p<0.0001) compared to placebo (Eggermont, et al., 2021; 22:643-654). In 2015, pembrolizumab was FDA approved for use in NSCLC, after OS benefit (10.4 vs. 8.5 months; p=0.0008) was demonstrated compared to docetaxel in Keynote-010 trial for patients with PD-L1 positive tumors and irrespective of EGFR or ALK status (Borghaci, et al., 2015; 373:1627-1639; Herbst, et al., 2016; 387:1540-1550). Keynote-024 Phase 3 trial in patients with untreated NSCLC and PD-L1 ≥50%, without sensitizing EGFR or ALK mutations, revealed significantly longer OS (6-months OS: 80.2% vs. 72.4%; p=0.005), PFS (10.3 vs. 6.0 months; p<0.001), and response rates (44.8% vs. 27.8%) compared to chemotherapy (Reck, et al., 2016; 375:1823-1833). Indication of pembrolizumab was further expanded to non-squamous NSCLC patients without sensitizing mutations (in combination with chemotherapy), in the first-line setting and irrespective of PD-L1 expression, based on data from Keynote-021, with improved response rates, and PFS advantage (19 vs. 8.9 months) (Borghaci, et al., 2017; 12: S1791). Data from Keynote-189 (Ghandi, et al., 2018; 378:2078-2092) and Keynote-407 (Paz-Ares, et al., 2018; 379:2040-2051) revealed consistent response rate, PFS and OS advantage for pembrolizumab (vs. chemotherapy), irrespective of PD-L1 status. Pembrolizumab is also approved for recurrent or metastatic head and neck squamous cell cancer in the second-line (ORR: 16%; Keynote-012 trial) and first-line settings (as a monotherapy for combined positive score (CPS) ≥1% or in combination with chemotherapy; OS 12.3 vs. 10.3 months; p=0.0086) (Sciwert, et al., 2016; 17:956-965; Burtness, et al., 2019; 394:1915-1928).

In patients with refractory classical Hodgkin's lymphoma, treatment with pembrolizumab showed a response rate of 69% with 22% complete response and a median duration of response of 11 months (Keynote-087) (Chen et al., 2017; 35:2125-2132). In patients with refractory/relapse primary mediastinal large B-cell lymphoma, pembrolizumab showed improved response rates (45%; 13% complete response rates; Keynote-170), securing FDA approval (Armand et al., 2019; 37:3291-3299).

In patients with unresectable or metastatic urothelial cancer who progressed on first-line platinum therapy, pembrolizumab treatment conferred 3 months OS benefit compared to chemotherapy (10.3 vs. 7.4 months; p=0.002) based on data from Keynote-045 trial (Bellmunt et al., 2017; 376:1015-1026). In the first-line setting, and in patients with CPS≥10% who are not eligible for cisplatin-based therapy, pembrolizumab increased ORR significantly (38%), securing further FDA-expanded indication (Keynote-052) (Balar et al, 2017; 18:1483-1492).

Pembrolizumab has also gained tumor-agnostic FDA approval based on TMB levels and MSI/MMR status in solid tumors. In patients with MSI-H and dMMR solid tumors, pembrolizumab revealed overall response rates, nearing 40% based on multiple clinical trials (Keynote-012, 016, 028, 158 and 164). Similarly, the FDA granted approval to pembrolizumab for the treatment of patients with unresectable/metastatic solid malignancies, with a TMB-H status (>10 mutations/megabase). This was based on a prospectively-planned retrospective analysis of 10 cohorts of patients with various previously treated unresectable or metastatic TMB-H solid tumors enrolled in Keynote-158. Treatment with pembrolizumab revealed response rates of 29% in TMB-H patients compared to 6% in TMB-low patients (Marabelle et al., 2020; 21:1353-1365).

Cemiplimab is also a human monoclonal antibody against PD-1 and was approved by the FDA in 2018 to treatment locally advanced, metastatic squamous cell carcinoma patients who are not candidates for curative surgery or radiation. This was based on data from the Phase 1 expansion cohorts were consistent with an ORR of 50%, with response rates of 47 in the Phase 2 metastatic-disease cohort (Migden et al., 2018; 379:341-351).

Response to PD-L1 CPI: Avelumab, durvalumab and atezolizumab are all PD-L1 blocking antibodies. Despite a slightly different mechanism of action, they target and disrupt the PD-1/PD-L1 interaction. In a meta-analysis comparing PD-1 and PD-L1 monoclonal antibodies in terms of efficacy and toxicity in NSCLC, there was no difference in response rates between both agents (19% and 18.6%), and the overall incidence of adverse events (64% and 66%) (Pillai et al., 2018; 124:271-277). Avelumab is approved for use in patients with metastatic Merkel cell carcinoma, locally advanced/metastatic urothelial carcinoma (second-line setting), and in combination with Axitinib for first-line treatment of patients with advanced renal cell carcinoma (Kaufman et al., 2016; 17:1374-1385; Keilholz et al, 2019; 7:12; Motzer et al., 2019; 380:1103-1115).

Durvalumab is also approved in patients with advanced/metastatic urothelial carcinoma who had disease progression within 12 months of receiving platinum-based chemotherapy. In this group of patients, receipt of durvalumab results in response rates of 31% in all patients, 46.4% in PD-L1 positive tumors, and none in the PD-L1 negative subgroup (Massard et al., 2016; 34:3119-3125). Durvalumab is also approved for use in stage III NSCLC patients after receipt of chemo-radiation, demonstrating significant PFS (16.8 vs. 5.6 months; p<0.001) and ORR (28.4% vs. 16%; p<0.001) benefit compared to placebo (PACIFIC trial) (Antonia et al., 2017; 377:1919-1929).

Atezolizumab is approved for use in patients with metastatic urothelial carcinoma, who either progressed or were not candidate to receive platinum-based chemotherapy, regardless of PD-L1 expression (IMvigor210 trial), providing ORR of 23%, median PFS of 2.7 months and median OS of 15.9 months (Balar et al., 2017; 389:67-76). In the OAK and POPLAR trials, atezolizumab provided OS benefit to patients with metastatic NSCLC who progressed on platinum-based chemotherapy, regardless of PD-L1 expression or histology (Rittmeyer, et al., 2017; 389:255-265; Fehrenbacher et al., 2016; 387:1837-1846.). In patients with extensive-stage SCLC, addition of atezolizumab to carboplatin and etoposide chemotherapy in the first-line setting, resulted in OS (12.3 vs. 10.3 months; p=0.007) and PFS (5.2 vs. 4.3 months; p=0.02) benefit compared to placebo (IMpower133) (Horn, et al., 2018; 379:2220-2229).

The data discussed above, reveals not only a wide variability in terms of response rates across tumor histologies, but also highlights a lack of a consistently predictive biomarker of response to CPI. For example, response of PFS and OS specifically to CPI treatment has been shown to be strongly correlated with high TMB in melanoma (Johnson et al., 2019; 7:1755-1759), NSCLC (Ricciuti et al., 2022; 8:1160-1168) and urothelial carcinoma (Balar et al., 2017; 389:67-76). This however is not the case in tumors such as esophageal, gastric, urinary tract or small cell carcinomas, which are considered to be “hyper-mutated” given the tight relationship between tumorigenesis and significant carcinogen exposure. In a study by Rousseau and colleagues, these tumor types were classified as “TMB-insensitive” tumors whereby TMB-H status did not necessarily correlate with better response to CPI (HR: 0.84; 95% CI: 0.63-1.11), compared to “TMB-sensitive” tumors (NSCLC, melanoma; HR: 0.52; 95% CI: 0.41-0.64)¹²⁷. These observations thus warrant exploring other predictive biomarkers of CPI response and clonal neoantigen may be the key signal to understanding the actual predictive power of TMB.

Table 3 summarizes the treatment responses to FDA-approved checkpoint inhibitors (CPIs) and respective indications. Overall survival (OS); progression free survival (PFS); recurrent free survival (RFS); overall response rate (ORR); microsatellite instability (MSI); mismatch repair deficient (dMMR); duration of response (DoR).

TABLE 3

| CPI (Target) | Indication/population | Study (arms) | Response | Ref. |
|---|---|---|---|---|
| Pembrolizumab | Unresectable or metastatic | NCT01295827 | ORR: 24% | Robert et al., |
| (anti-PD-1) | BRAF^{V600E} mutated | (Pembrolizumab) | | Lancet 2014, |
| | metastatic melanoma, | | | 384, 1109-1117 |
| | refractory to CTLA-4 | | | |
| | therapy and BRAF inhibitor | | | |
| | Unresectable or metastatic, | Keynote-006 | 1-year OS: 74.1% | Robert et al., |
| | untreated melanoma | (Pembrolizumab vs. | (vs. 58.2%; | N Engl J Med 2015, |
| | regardless or BRAF status | Ipilimumab) | p = 0.0005); 1-year | 372, 2521-2532 |
| | | | PFS: 47.3% (vs. | Carlino et al., |
| | | | 26.5%; p < 0.001); | Eur J Cancer 2018, |
| | | | PD-L1 ≥1%: 24- | 101, 236-243 |
| | | | months OS and | |
| | | | PFS: 58% and 33% | |
| | | | vs. 45% and 13%, | |
| | | | respectively | |
| | Stage III melanoma/adjuvant | Keynote-054 | RFS: 65.33% vs. | Eggermont et al., |
| | | (pembrolizumab vs. | 49.4%; p < 0.0001 | Lancet Oncol 2021, |
| | | placebo) | | 22, 643-654 |
| | Previously treated, PD-L1 | Keynote-010 | OS: 10.4 vs. 8.5 | Borghaei et al., |
| | positive, advanced NSCLC | (pembrolizumab vs. | months; p = 0.0008 | Engl J Med 2015, |
| | | docetaxel) | | 373, 1627-1639; |
| | | | | Herbst et al., |
| | | | | Lancet 2016, |
| | | | | 387, 1540-1550 |
| | Untreated, PD-L1 ≥50 | Keynote-024 | 6-months OS: | Reck et al., |
| | advanced NSCLC | (pembrolizumab vs. | 80.2% vs. 72.4%; | N Engl J Med |
| | | chemotherapy) | p = 0.005 | 2016, 375, |
| | | | Median PFS: 10.3 | 1823-1833 |
| | | | vs. 6.0 months; | |
| | | | p < 0.001 | |
| | | | ORR: 44.8% vs. 27.8% | |
| | Untreated, non-squamous | Keynote-021 | Median PFS: 19.0 | Borghaei et al., |
| | NSCLC without sensitizing | (pembrolizumab plus | vs. 8.9 months | Journal of Thoracic |
| | mutations, regardless of | chemotherapy vs. | (HR: 0.53; p = 0.0049) | Oncology 2017, 12, |
| | PD-L1 level | chemotherapy | ORR: 56.7% vs. | S1791; |
| | | | 26.4%; p = 0.0016 | Gandhi et al., |
| | | | Note: data from | N Engl J Med 2018, |
| | | | Keynote-189 and | 378, 2078-2092; |
| | | | 407 revealed | Paz-Ares et al., |
| | | | consistent PFS and | N Engl J Med 2018, |
| | | | OS advantage for | 379, 2040-2051 |
| | | | pembrolizumab | |
| | Recurrent/metastatic head | Keynote-012 | ORR: 16% | Seiwert et al., |
| | and neck squamous cancer; | (pembrolizumab) | | Lancet Oncol 2016, |
| | 2^{nd} line or 1^{st} line in CPS ≥1 | | | 17, 956-965; |
| | | | | Burtness et al., |
| | | | | Lancet 2019, 394, |
| | | | | 1915-1928 |
| | Unresectable/metastatic | Keynote-045 | Median OS: 10.3 | Bellmunt et al., |
| | urothelial cancer; 2^{nd} line | (pembrolizumab) | vs. 7.4 months | N Engl J Med 2017, |
| | | | (p = 0.002) | 376, 1015-1026 |
| | Unresectable/metastatic | Keynote-052 | ORR: 38% | Balar et al., |
| | urothelial cancer, cisplatin- | (pembrolizumab) | | Lancet Oncol 2017, |
| | ineligible; 1^{st} line in CPS ≥10 | | | 18, 1483-1492 |
| | MSI-H, dMMR or TMB-H | Keynote-012, 016, | MSI-H or dMMR | Chung et al., |
| | solid tumors | 028, 158* and 164 | studies: ORRs ~40% | J Clin Oncol 2019, |
| | | (pembrolizumab) | TMB-H: ORR: | 37, 1470-1478; |
| | | | 29% (vs. 6% TMB-L) | O'Malley et al., |
| | | | Cervical cancer | J Clin Oncol 2022, |
| | | | cohort/MSI-H or | 40, 752-761; |
| | | | dMMR: ORR: 12.2% | Marabelle et al., |
| | | | Endometrial cancer | Lancet Oncol 2020, |
| | | | cohort/MSI-H or | 21, 1353-1365 |
| | | | dMMR: ORR: 48% | |
| | Persistent/recurrent or | Keynote-826 | Median OS: not | Colombo et al., |
| | metastatic cervical cancer | (pembrolizumab vs. | reached vs. 16.3 | Annals of Oncology |
| | with PD-L1 CPS ≥1 | placebo + | months (p = 0.0001) | 2021, 32, S1307-S1308 |
| | | paclitaxel/platinum +/− | Median PFS: 10.4 | |
| | | bevacizumab | vs. 8.2 months | |
| | | | (p < 0.0001) | |
| | | | ORR: 68% vs. | |
| | | | 50%; median DOR: | |
| | | | 18 vs. 10.4 months | |
| Nivolumab | Persistent/recurrent | NRG-GY002 | 6-months PFS: 16%; | Santin et al., |
| (anti-PD-1) | cervical cancer | (nivolumab) | 6-months OS: 78% | Gynecol Oncol 2020, |
| | | | Median duration of | 157, 161-166 |
| | | | SD: 5.7 months | |
| | Persistent/recurrent | CheckMate-358 | ORR: 26.3% | Naumann et al.., |
| | cervical cancer | (nivolumab) | Median OS: 21.9 | J Clin Oncol 2019, |
| | | | months | 37, 2825-2834 |
| Cemiplimab | Recurrent or metastatic | NCT03257267 | ORR: 16.4% vs. 6.3% | Tewari et al., |
| (anti-PD-1) | cervical cancer after | (cemiplimab vs. | Median OS: 12.0 | N Engl J Med 2022, |
| | platinum-based chemotherapy, | investigator-choice | vs. 8.5 months | 386, 544-555 |
| | regardless of PD-L1 | chemotherapy) | (p < 0.001) | |
| | | | Median PFS: HR | |
| | | | 0.75, p < 0.001 | |
| Dostarlimab-gxly | Primary advanced or | RUBY (dostarlimab-gxly | Median PFS: 30.3 | Mirza et al., |
| (anti-PD-1) | recurrent dMMR or MSI-H | vs. placebo + carboplatin- | vs. 7.7 months | N Engl J Med 2023, |
| | endometrial cancer | paclitaxel, followed by | (p < 0.0001) | 388, 2145-2158 |
| | | dostarlimab or placebo | | |
| Avelumab | Chemo-refractory, metastatic | NCT02155647 | ORR: 31.8% | Kaufman et al., |
| (anti-PD-L1) | Merkel cell carcinoma, | (Avelumab) | (28/88); CR: 8/88 | Lancet Oncol 2016, |
| | regardless of PD-L1 | | and PR: 20/88 | 17, 1374-1385 |
| | Advanced urothelial | JAVELIN Solid Tumor | ORR: 16.5%; CR: | Keilholz et al., |
| | carcinoma, 2^{nd} line setting | (Avelumab) | 4.1%; PR: 12.4% | J Immunother Cancer |
| | | | Median DoR: 20.5 | 2019, 7, 12; |
| | | | months | Apolo et al., |
| | | | Median PFS: 1.6 | J Immunother Cancer |
| | | | months | 2020, 8 |
| | | | Median OS: 7.0 | |
| | | | months; 24-month | |
| | | | OS: 20.1% | |
| | Advanced renal cell | JAVELIN Renal | PD-L1+: median | Motzer et al., |
| | carcinoma (1^{st} line in | (Avelumab + Axitinib | PFS: 13.8 vs. 7.2 | N Engl J Med 2019, |
| | combination with Axitinib | vs. sunitinib) | months (p < 0.001); | 380, 1103-1115 |
| | | | ORR: 55.2% (vs. 25.5%) | |
| | | | Overall population: | |
| | | | median PFS: 13.8 | |
| | | | vs. 8.4 (p < 0.001) | |
| Durvalumab | Advanced/metastatic | NCT01693562 | ORR: 31% (all | Massard et al., |
| (anti-PD-L1) | urothelial carcinoma, | (Durvalumab) | patients), 46.4% | J Clin Oncol 2016, |
| | platinum-refractory | | (PD-L1 positive), | 34, 3119-3125 |
| | | | 0% (PD-L1 negative) | |
| | Stage III NSCLC, | PACIFIC Trial | Median PFS: 16.8 | Antonia et al., |
| | maintenance therapy, | (Durvalumab vs. | vs. 5.6 months | N Engl J Med 2017, |
| | post-chemo-radiation | placebo) | (p < 0.001) | 377, 1919-1929 |
| | | | ORR: 28.4% vs. | |
| | | | 16% (p < 0.001) | |
| Atezolizumab | Metastatic urothelial | IMvigor210 | ORR: 23% | Balar et al, |
| (anti-PD-L1) | carcinoma, platinum | (Atezolizumab) | Median PFS: 2.7 | Lancet 2017, |
| | ineligible or refractory, | | months | 389, 67-76 |
| | regardless of PD-L1 | | Median OS: 15.9 | |
| | | | months | |
| | Metastatic NSCLC, 2^{nd} line | OAK/POPLAR | Median OS: 12.6- | Rittmeyer et al., |
| | post platinum-based | (Atezolizumab vs. | 13.8 vs. 9.6-.7 | Lancet 2017, 389, |
| | chemotherapy, regardless | docetaxel) | months (p0.05) | 255-265; |
| | of PD-L1 | | | Fehrenbacher et al., |
| | | | | Lancet 2016, 387, |
| | | | | 1837-1846 |
| | Extensive stage SCLC, | IMpower 133 | Median OS: 12.3 | Horn et al., |
| | 1^{st} line setting | (Chemo/atezolizumab | vs. 10.3 months | N Engl J Med 2018, |
| | | vs. chemotherapy/ | (p = 0.007) | 379, 2220-2229 |
| | | placebo) | Median PFS: 5.2 | |
| | | | vs. 4.3 months | |
| | | | (p = 0.02) | |

*Included 98 patients with advanced cervical cancer (ORR: 12.2%; 12/98) and 90 patients with advanced endometrial cancer (ORR: 48%; 43/90).

### E. Biomarkers of CPI Response

Curiously, multiple biomarkers have been utilized to define CPI sensitive populations. There are currently 3 FDA approved biomarkers of CPI sensitivity: PD-L1 expression, dMMR/MSI-H, and TMB-H. None of these biomarkers are perfectly predict nor preclude response to CPI.

PD-L1 is the most extensively studied predictive biomarker of CPI and is typically measured by immunohistochemistry. It is quantified in a categorical manner with variable cutoffs of “positivity” (<1 vs. ≥1, <5 vs. ≥5, <10 vs. ≥10, and <50 vs. ≥50). Similarly, differences exist in the source of PD-L1 quantification, whether accounting for PD-L1 on tumor cells only (tumor proportion score-TPS) or its expression on tumor cells, lymphocytes and macrophages from the tumor microenvironment (CPS: combined positive score) (De Marchi P, Leal L F, Duval da Silva V, da Silva E C A, Cordeiro de Lima V C, Reis R M. 2021; 74:735-740; Ito T, Okamoto I, Tokashiki K, et al. 2022; 42:1547-1554). Different FDA approvals for CPI were contingent on PD-L1 status, but at different cut points. For example, pembrolizumab is approved as a monotherapy in metastatic NSCLC patients with PD-L1 >50% (TPS), while investigators in Impassion 130 for example, used PD-L1 (TPS) ≥1% as a cut-off that showed OS benefit to atezolizumab treatment of triple-negative breast cancer (Planchard et al., 2018; 29: iv 192-iv237; Schmid et al., 2018; 379:2108-2121). Keynote-048 on the other hand used CPS score ≥1% as predictive of benefit from pembrolizumab in patients with head and heck squamous carcinoma (Burtness et al., 2019; 394:1915-1928). Last, and although PD-L1 positivity in metastatic NSCLC had a higher response rates to pembrolizumab (45%), 15% of PD-L1 negative tumors still responded (Garon, et al. 2015; 372:2018-2028). Taken altogether, PD-L1 remains an imprecise marker for CPI response, due to special and temporal heterogeneity within a specific tumor sample and across metastatic sites (Saito, et al., 2019; 11:4982-4991; Li et al., 2018; 18:4), lack of standardization in terms of quantification methods and selection of a threshold to define positivity, and variation between assays and antibody clones (Hirsch et al., 2017; 12:208-222).

MSI-H/dMMR is the first tissue agnostic biomarker that predicts response to anti-PD-1 in solid tumors (Le et al., 2017; 357:409-413), with high rates of durable response in tumors with somatic dMMR regardless of tissue of origin (ORR: 40% in metastatic colorectal cancer and ORR: 71% in non-colorectal cancers) (Le D T, Uram J N, Wang H, et al. 2015; 372: 2509-2520). Similar supportive data exist for single agent nivolumab and combination of nivolumab and ipilimumab (ORR: 31% and 55%, respectively) (Let et al., 2017; 357:409-413; Overman et al., 2018; 36:773-779). In a pan-cancer study of 27 advanced non-colorectal dMMR tumors, pembrolizumab resulted in an ORR of 34% across tumor types, with a complete response achieved in 10% of patients (Marabelle et al., 2020; 38:1-10). Deficient MMR/MSI-H status has been proposed as a surrogate marker of the presence of neoantigen, defining higher immunogenicity, and thus higher response rates to CPI. Unlike TMB which reflects the actual total tumor mutation burden, a deficient mismatch repair status does not precisely predict the level of neoantigen, but rather act as a predisposing mechanism for the accumulation of mutations and neoantigens. Similarly, dMMR/MSI-H status does not distinguish between passenger (subclonal) or clonal antigens.

An early evidence of tumor mutation burden and outcome to CPI treatment was made by Snyder et al examining a series of outlier responses in melanoma patients who received anti-CTLA-4 therapy (Snyder, et al., 2014; 371:2189-2199). Later TMB was related to anti PD-1 therapy (Let et al., 2015; 372:2509-2520; Rizvi et al., 2015; 348:124-128; Hugo et al., 2016; 165:35-44; Choucair et al., 2020; 27:841-853). Unfortunately, despite evidence of significance in subset cancer populations, responses have been inconsistent with evidence of lack of response in subpopulations who fulfill biomarker sensitivity parameters and further evidence of response is occasionally seen in populations fulfilling poor biomarker sensitivity parameters (Rizvi et al., 2015; 348:124-128; Brahmer et al., 2015; 373:123-135; Garon et al., 2015; 372:2018-2028; Herbst et al., 2014; 515:563-567). This inconsistency provides difficulties in clinical management decision making. The “sensitive” biomarkers (Let et al., 2015; 372:2509-2520; Le et al., 2017; 357:409-413; Marabelle et al., 2020; 38:1-10; Vilar, 2010; 7:153-162; Yamamoto, 2019; 46:261-270; Germano et al., 2017; 552:116-120; Mardis, 2019; 11:71) generally apply to about a 30% of the cancer population (microsatellite high (MSI-H), mismatch repair deficient (dMMR), LOH-H, HRD/BRCA-m, TMB ≥10 mu/mb DNA). DNA mismatch repair (MMR) high tumors demonstrate a particular unique subpopulation for sensitivity to CPI treatment. Patients with this molecular profile also tend to have high TMB (Let et al., 2015; 372:2509-2520; Alexandrov et al., 2020; 578:94-101; Lawrence et al., 2013; 499:214-218).

Recent evidence by Litchfield et al suggest that clonal TMB and its relationship to clonal neoantigen may be the key signal to understanding tumor mutation burden effect (Litchfield et al., 2021; 184:596-614 e514; McGranahan et al., 2016; 351:1463-1469; Rizvi et al., 2015; 348:124-128; Riaz et al., 934-949 e916; Miao et al., 2018; 50:1271-1281). Litchfield's meta-analysis involved 1008 patients and 12 independent trials and demonstrated very effectively marked relationship of clonal TMB impact on OS involving patients receiving FDA approved CPIs (p=0.000000029). However, subclonal TMB measured in the same patient population was not a significant correlating factor with OS. Results support fairly conclusively that clonal TMB is the exclusive biomarker indicating optimal OS response to CPI therapy (Litchfield, et al., 2021; 184:596-614 e514). Thus, clonal TMB appears to be an independent biomarker of improved outcome to clinical response involving CPI treatment that warrants further prospective clinical study assessment. From a biomarker standpoint in assessing clinical response to CPI therapy, work by Litchfield and others also reveal reduced to no capacity of benefit to subclonal TMB. Subclonal neoantigens have in fact been shown to be fairly ineffective at inducing robust induction of activated T cell effector populations capable of demonstrating significant antitumor reactivity to whole tumors (Litchfield, et al., 2021; 184:596-614 e514; McGranahan et al., 2016; 351:1463-1469; Hanahan, 2022; 12:31-46; Wu, et al, 2022; 84:89-102; Wolf, et al., 2019; 179:219-235 e221; Rizvi et al, 2015; 348:124-128; Snyder et al., 2014; 371:2189-2199; Johnson et al., 2014; 343:189-193). These results suggest high subclonal TMB is unlikely to provide an optimal target population for CPI therapy and likely would be expected to be a poor target for CAR-T, tumor infiltrating lymphocyte therapy or vaccine approaches.

Another observation supporting the role of clonal TMB emerges from Merkel cell cancer and Hodgkin lymphoma, both with low TMB, but yet with high response rates to CPI. In fact, both malignancies harbor clonal Merkel cell polyomavirus and Epstein-Barr virus genomes (Anagnostopoulos et al., 1989; 74:810-816); Sastre-Garau et al., 2009; 218:48-56), and these “public” clonal viral neoantigens, when targeted demonstrate high response rates to CPI therapy (Ansell et al., 2015; 372:311-319; Nghiem, et al., 2016; 374:2542-2552; D'Angelo et al., 2018; 4: e180077) thereby supporting additional evidence that clonal neoantigen is of greater priority over TMB. Likewise, despite a relatively high TMB, melanomas with higher number of subclonal mutations have been reported to have lower response rates to CPI therapy (Wolf et al., 2019; 179:219-235 e221). Results suggest clinical management needs for routine measure of clonal neoantigen or TMB. CPI therapy response would likely benefit from assessment of clonal and subclonal molecular profiles prior to treatment. This would more than likely expand CPI treatment population. Two recent studies involving mice support evidence that clonal neoantigens are superior at engaging effective tumor immune surveillance. Gejman et al demonstrated that a minimum fraction of tumor cells must express a neoantigen before adaptive immune rejection and also that there is a dosage threshold which varies between the different neoantigens (Gejman, et al., 2018; 7). In the second study, Wolf et al showed again in mice clonal neoantigen tumors were more readily rejected by the immune system compared with heterogenous tumors, independent of total mutational burden (Wolf, et al., 2019; 179:219-235 e221). Also, the TRACERx study revealed that tumors with high clonal neoantigen burdens were more densely infiltrated by T cells (AbdulJabbar et al., 2020; 26:1054-1062). Ovarian cancer has shown very little responsiveness to CPI therapy. Despite this, most OC tumors demonstrate presence of tumor infiltrating lymphocytes (TILs) at the degree of TIL infiltration that is strongly and reproducibly correlated with survival advantage (Li J, Wang J, Chen R, Bai Y, Lu X. 2017; 8:15621-15631; Hamanishi J, Mandai M, Iwasaki M, et al. 2007; 104:3360-3365). A meta-analysis including 21 studies and almost 3000 ovarian cancer patients confirmed that high levels of intra-epithelial CD3+ or CD8+ T-cells were most significantly associated with both improved progression free and overall survival (Hwang et al., 2012; 124:192-198). This positive correlation suggests that using ICIs, such as anti-PD-L1/PD-1 therapies, could be effective if appropriate targeting is directed to subpopulations with high clonal neoantigens expression. We hypothesize that there may be a threshold of minimum number of clonal neoantigen effector cells available, active and containing clonal TCR sensitivity to engage malignant cells with high clonal and low subclonal neoantigen burden. Technology with capacity to increase the population of clonal neoantigen burden would thus be an obvious course of action and likely attractive combination therapy with CPI's. There is evidence that Vigil® may provide such activity (Rocconi et al., 2020; 21:1661-1672; Rocconi et al., 2021; 161:676-680; Rocconi et al., 2022; 29:369-382; Brave et al., 2022; 16:11795549221110501).

Sensitive biomarkers attempt to convey evidence of a “hot” tumor microenvironment, one filled with inflammatory cells generating evidence of anticancer activity. Interestingly, these biomarkers also portray a molecular profile of reduced DNA repair capacity. At least four, partly overlapping damage repair pathways operate exist-nucleotide-excision repair (NER), homologous recombination, base-excision repair (BER) and end joining. Numerous hereditary syndromes of defective genome maintenance related to the above mechanism exist. All are associated with cancer development predisposition (Table 1).

Table 1. Human syndromes with defective genome maintenance

TABLE 1

| | Affected maintenance | Main type of | Major cancer |
|---|---|---|---|
| Syndrome | mechanism | genome instability | predisposition |
| Xeroderma | NER (±Transcription | Point mutations | UV-induced skin |
| pigmentosum | coupled repairCR) | | cancer |
| Cockayne syndrome | Transcription coupled | Point mutations | None* |
| | repairCR | | |
| Trichothiodystrophy | NER/Transcription | Point mutations | None* |
| | coupled repairCR | | |
| Ataxia telangiectasia | DSB response/repair | Chromosome | Lymphomas |
| (AT) | | aberrations | |
| AT-like disorder | DSB response/repair | Chromosome | Lymphomas |
| | | aberrations | |
| Nijmegen breakage | DSB response/repair | Chromosome | Lymphomas |
| syndrome | | aberrations | |
| BRCA 1/BRCA 2 | Homologous | Chromosome | Breast (ovarian) |
| | recombinationR | aberrations | cancer |
| Werner syndrome | Homologous | Chromosome | Various cancers |
| | recombinationR?/TLS? | aberrations | |
| Bloom syndrome | Homologous | Chromosome | Leukemia, |
| | recombinationR? | aberrations (SCE↑) | lymphoma, others |
| Rothmund-Thomson | Homologous | Chromosome | Osteosarcoma |
| syndrome | recombinationR? | aberrations | |
| Ligase IV | EJ | Recombination | Leukemia(?) |
| deficiency† | | fidelity | |
| HNPCC | MMR | Point mutations | Colorectal cancer |
| Xeroderma | TLS‡ | Point mutations | UV-induced skin |
| pigmentosum variant | | | cancer |
| ERCC6L2 | NER | Point mutations | hematologic |
| deficiency | | | |
| Constitutional | MMR | Point mutations and | Hematologic, brain |
| mismatch repair | | insertion/deletions | and intestinal tract |
| disorder | | | |
| Fanconi Anemia | FA | Chromosomal | SCC, AML, MDS |
| | | aberrations | |

*Defect in transcription-coupled repair triggers apoptosis, which may protect against UV-induced cancer.
†One patient with leukemia and radiosensitivity described with active-site mutation in ligase IV.
‡Specific defect in relatively error-free bypass replication of UV-induced cyclobutene pyrimidine dimers.
Abbreviations: base-excision repair (BER); double-strand break (DSB); hereditary non-polyposis colorectal cancer (HNPCC); mismatch repair (MMR); nucleotide-excision repair (NER); sister-chromatid exchange (SCE); translesion synthesis (TLS); mismatch repair (MMR); nucleotide excision repair (NER); end joining (EJ); squamous cell carcinoma (SCC); acute myeloid leukemia (AML); myelodysplastic syndrome (MDS); Fanconi anemia (FA).

It appears CPI therapy is less effective in cancer populations with good DNA repair capacity, those with microsatellite instability stable (MSI-S), mismatch repair proficient (pMMR), loss of heterozygosity low (LOH-L), HRP/BRCA-wt, and TMB <10 mu/mb DNA profiles. Surprisingly, these populations would be expected to contain a high clonal TMB or clonal neoantigen load (Hocijmakers J H. 2001; 411:366-374). If altered to a state of immune activation in essence going from “cold” to “hot”. This would be an ideal management prior to CPI treatment likely enabling greater CPI sensitivity and responsiveness. No FDA approved therapies have been designed specifically to impact altering of clonal or subclonal TMB/neoantigen expression and enhance activation of immune responsiveness to CPI treatment. Recognition of this direction in anticancer therapy development would appear warranted.

### F. CPI Difficulty in Ovarian Cancer

Two Phase 3 studies evaluating CPI in ovarian cancer have failed to show evidence of clinical benefit (Moore et al., 2021; 39:1842-1855; Ledermann et al., 2020; 159:13-14.). As a result, there is concern regarding further immunotherapeutic development in ovarian cancer.

In the first trial, Javelin 100 evaluated single agent avelumab (n=332) or in combination with chemotherapy (n=331) vs. control of chemotherapy alone (n=335) in stage III-IV ovarian cancer patients frontline maintenance following surgical debulking and platinum doublet chemotherapy. The primary endpoint of the trial was PFS. The trial was stopped by the data monitoring committee for futility as the PFS endpoint involving avelumab performed worse (single agent; HR 1.43 95% CI 1.05-1.95 p=0.99 and combination HR 1.14 95% CI 0.83-1.56 p=0.79). Median PFS in the avelumab only group was 16.8 months (95% CI 13.4-not estimable (NE), 18.1 months (95% 14.8-NE) in the combination and NE (95% 18.2-NE) in the control group (Monk et al., 2021; 22:1275-1289). In subsequent biomarker subset analysis, PD-L1, BRCA1/2 mutation status and CD8 positivity did not predict response to avelumab as single agent or in combination (Ledermann et al., 2020; 30: A1-A1). The authors concluded that new biomarkers are needed to select appropriate candidates.

Next, in the IMAGYN050 trial, atezolizumab was added to frontline chemotherapy and bevacizumab following either primary cytoreductive surgery or interval cytoreduction in patients with newly diagnosed stage III/IV ovarian cancer. The primary endpoint was PFS. Patients were assigned to either receive atezolizumab with carboplatin plus paclitaxel (CP) and bevacizumab or placebo with CP and bevacizumab. The median PFS was 19.5 months in the atezolizumab containing arm vs. 18.4 months in the placebo arm (HR 0.92 95% CI, 0.79 to 1.07 p=0.28). In a planned exploratory analysis of PD-L1+ patients there was still no difference in PFS, 20.8 months vs. 18.5 months, (HR 0.80 95% CI, 0.65 to 0.99 p=0.038) (Moore et al., 2017; 28: V350-V351). OS data was immature at the time of publication. In either trial, single agent CPI or in combination with chemotherapy and bevacizumab, there were no new safety signals identified.

Of note, less than 25% of patients demonstrated >5% PD-L1+immune cells (Moore et al., 2017; 28: V350-V351). In contrast, in tumors known to be immune responsive, such as non-small cell lung cancer, PD-L1 expression ranges from 24% to 60% (Yu et al., 2016; 11:964-975). The low response rate to PD-L1 inhibition in OC could be in part explained by the low expression level of PD-L1 on tumor cells. Additionally, while some studies have demonstrated clinical response in tumors with high TMB, it is notable that ovarian cancer tumors typically demonstrate low TMB (Rizvi, et al. 2015; 348:124-128; Goodman, et al., 2017; 16:2598-2608; Riviere, et al., 2020; 19:2139-2145; Strickler, et al, 2021; 27:1236-1241; Martin et al., 2016; 11: e0155189; Maleki et al., 2018; 6:157; Fan et al., 2020; 89:107126).

Proportion of ovarian cancer tumors containing HRP versus BRCA-m/HRD tumor molecular profile involves nearly 50% of the ovarian cancer population. Clonal TMB or neoantigen would be expected to be independent of low >5% PD-L1+expressive immune cells (Moore et al., 2021; 39:1842-1855) and low TMB (Strickler et al., 2021; 27:1236-1241) as we previously described the clonal TMB is only 2% of the total TMB. The proportion of HRP profile in ovarian cancer is among the lowest of all solid tumor malignancies (under 50%) (Hecke et al., 2018; 2018). Over 80% of bladder cancer, cervical cancer and prostate cancer are somatic HRP profile. Data with Vigil® support increased clinical benefit in newly diagnosed IIIB-IV BRCA-wt HRP ovarian cancer with Vigil® as maintenance following surgical debulking induction platinum/taxane chemotherapy (Rocconi, et al., 2020; 21:1661-1672; Rocconi et al., 2021; 161:676-680; Walter et al., 2021; 163:459-464).

### G. Vigil®

Vigil® is a novel late stage immunotherapy (Rocconi, et al., 2020; 21:1661-1672; Rocconi et al., 2021; 161:676-680; Walter et al., 2021; 163:459-464) utilizing GMCSF-wt-bi-shRNA-furin plasmid transfected autologous tumor tissue designed to deliver clonal neoantigen targets thereby expanding clonal neoantigen targeting and expanding CD8+ T cell clonal neoantigen targeting effector cells within the tumor microenvironment. In essence, Vigil® is designed to turn “cold” to “hot” within the tumor microenvironment. By introducing GMCSF and reducing TGFβ1 and B2 at the intradermal site Vigil® inoculation enhances dendritic and T cell response to the inoculation site with the purpose of providing a natural full display of clonal neoantigens. Most, if not all autologous tumor tissues, contain robust clonal neoantigen expression activity at initial presentation in correlation with effective TCR responding effector cells to the clonal neoantigens. Specifically, attraction of dendritic cells to the clonal neoantigen expressed on the Vigil tissue following therapeutic intradermal injection enables direct dendritic cell interaction (Wculek et al., Nat Rev Immunol 2020, 20, 7-24). Dendritic cells are a group of specialized antigen presenting cells with a key role in the initiation and regulation of innate and adaptive immune response. This interaction is facilitated by Vigil utilizing the exposed clonal neoantigens from the autologous tissue used to construct Vigil. Vigil plasmid induces enhancement of local immune function through expression of GMCSF and immune suppression by reduction of TGFβ via activity of bi-shRNA-furin local knockdown. Following the dendritic cell activation targeting the personal clonal neoantigen targets the dendritic cells move to lymph node tissue to interact with T and B cells and induce a systemic adaptive immune response thereby expanding immune effector cells now able to target the same clonal neoantigen creating anticancer activity against systemic cancer sites. Evidence of this is supported by positive ELISpot assay induction against clonal neoantigen peptides and/or clonal neoantigen expressive tumor cells, as shown in multiple trials with Vigil® (Senzer, et al., 2012; 20:679-686; Senzer et al., 2013; 4:209; Oh et al., 2016; 143:504-510). Further work in determining threshold clonal TMB and tumor specific clonal neoantigen profiles of patients treated with Vigil® is underway. Phase 1 testing revealed correlation of ELISpot response using autologous tumor as the response induction signal against circulating autologous mononuclear cells harvested before and after Vigil® was induced by Vigil® and associated with improved OS compared to similar patients treated by Vigil® who did not demonstrate ELISpot response (HR 0.23, p=0.038) (Senzer, et al., 2012; 20:679-686). ELISpot response appeared to have been tempered in activity in some patients in whom extensive chemotherapy was administered leading to less or no ELISpot response. Follow up Phase 2 testing in patients who had newly diagnosed ovarian cancer and minimal prior chemotherapy to induce immune suppression revealed more impressive ELISpot response. In newly diagnosed ovarian cancer 31 of 31 patients who received Vigil® had positive ELISpot response compared to 0/8 control patients who did not receive Vigil®. ELISpot response at baseline in virtually all patients prior to receiving Vigil® was negative. Results are correlated with improved RFS of Vigil® treated patients compared to similar concurrent control patients who did not receive Vigil® and in whom RFS was not improved (p=0.0165) (Oh et al., 2016; 143:504-510). Clinical benefit was also shown to be durable in long term follow up beyond 3 years (Oh et al., 2020; 34:100648). Moreover, in long term follow up, it was shown in control patients who achieved relapse and received Vigil® after follow up relapse and that all turned positive with ELISpot response, although the overall level of ELISpot response activity was lower. Whether benefit was observed in late Vigil® treatment could not be conclusively determined, although the longest surviving control patients who crossed over to Vigil® after recurrent relapse were the longest survivors in the control group (Oh et al., 2020; 34:100648).

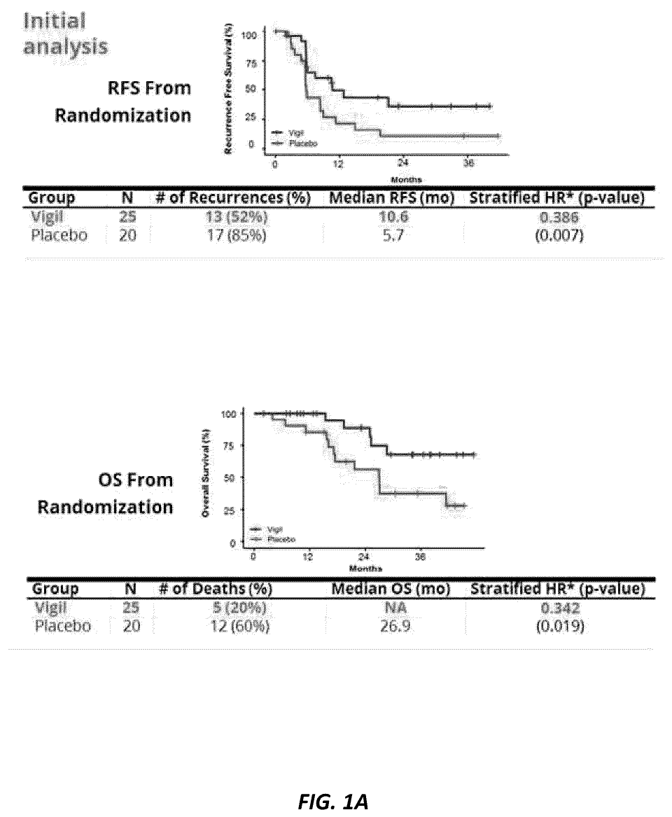

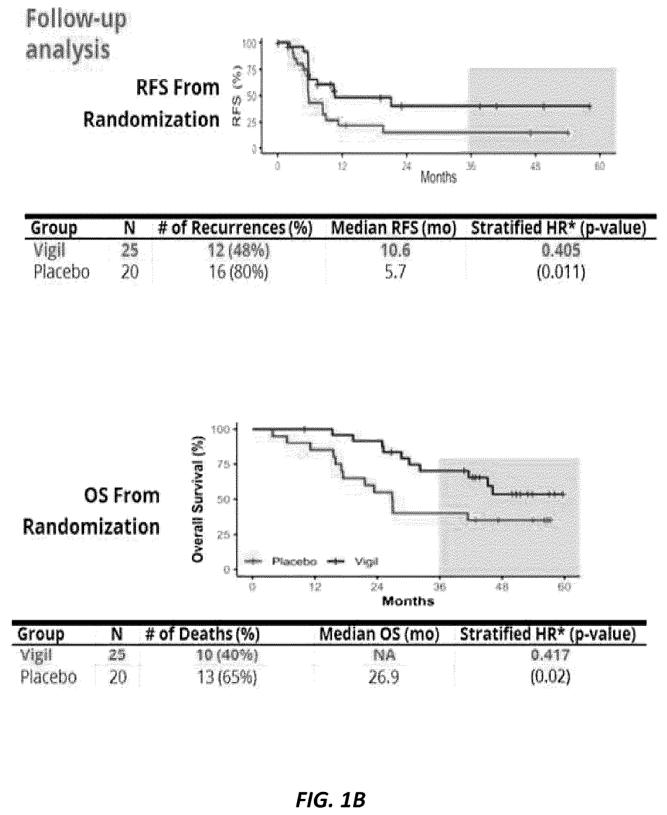

Later in a double blind Phase 2b trial (VITAL) involving 91 newly diagnosed ovarian cancer patients with Stage IIIb/IV resectable disease results revealed a trend to benefit in all patients (HR 0.688, p=0.078) but breakdown of patients with good tumor DNA repair (BRCA-wt, HRP) versus poor DNA repair (BRCA-m, HRD) revealed significant benefit related to Vigil® in both BRCA-wt patients (RFS HR 0.54, p=0.020; OS HR 0.493, p=0.049) and HRP patients (RFS HR 0.386, p=0.007 and OS HR 0.342, p=0.019) (Rocconi et al, 2020; 21:1661-1672; Rocconi, et al., 2021; 161:676-680; Walter et al., 2021; 163:459-464). Kaplan Meier results are shown in FIGS. 1A and 1B. Follow up also revealed that proportion of patients who were HRP positive profile who received Vigil® also demonstrated continuation of durable benefit in RFS, OS beyond 3 years (FIG. 2). These results support prior assessment that optimal Vigil® induced response involves the HRP positive population, which relates to good DNA repair and importantly likely higher proportion of clonal neoantigen expressive tumors (Nemunaitis, et al., “Rationale of Homologous Recombination Proficient Molecular Profile as Biomarker for Therapeutic Targeting in Ovarian Cancer”).

### H. Conclusion

Durable clinical benefit has been correlated with identification of CD8+ T cells recognizing clonal neoantigen and not subclonal neoantigen (McGranahan, et al., 2016; 351:1463-1469; Hanahan, 2022; 12:31-46; Rizvi, et al., 2015; 348:124-128; Snyder, et al., 2014; 371:2189-2199; Johnson, et al., 2014; 343:189-193). Results presented in this review suggest that clonal tumor mutation burden and clonal neoantigen targets are potentially the most ideal clinical direction for biomarker development to enhance more consistent and broad CPI utilization. There is a strong suggestion that all solid tumor cancer may be a target opportunity for CPI treatment pending assessment of clonal neoantigen. It is also likely that ovarian cancer is not an immune refractory malignancy, but the ovarian cancer paradigm must be aligned to subpopulations of patients who have presence of high clonal TMB or which clonal neoantigen with accompanying clonal targeting effector cells. Also, methods to increase presence of clonal neoantigen targeting effector cells (i.e., Vigil®) should be explored. BRCA-m and HRD tumors have DNA repair defects which lead to increased mutational load and proportion of subclonal over clonal cancer cells in relationship to HRP, BRCA-wt tumors (McGranahan, et al. 2016; 351:1463-1469; Germano, et al., 2017; 552:116-120; Strickland, et al., 2016; 7:13587-13598; Birkbak, et al., 2013; 8: e80023). Increased presence of subclonal neoantigens will decrease efficiency of clonal effector cell targeting and will also limit CPI response. As described, tumor clonal TMB and clonal neoantigen high load correlates with OS and PFS improvement to immune therapy, most notably CPI therapy (McGranahan, et al., 2016; 351:1463-1469; Rizvi, et al. 2015; 348:124-128; Snyder, et al., 2014; 371:2189-2199). Increased subclonal neoantigen heterogeneity in HRD tumors may indicate less effective immunoediting and/or lower expansion of antigen specific CD8+ T cells responses to clonal neoantigens which involve all the tumor cells. In this scenario, a high clonal neoantigen fraction and clonal TMB in the HRP group being induced to reactivity by Vigil® would create a “hot” microenvironment more supportive of a CPI sensitive tumor microenvironment. In fact, p53 mutant DNA (Sliheet, et al., 2022; 29:993-1000) and ENTPD1 high RNA expression (Rocconi, et al., 2022; 2:106) have been identified as relevant immune response biomarkers to Vigil® sensitivity and may further contribute to induction of “hot” tumor microenvironment and greater immune responsiveness to the clonal neoantigens who contain the appropriate TCR region to bind specific clonal neoantigen epitopes enabling appropriate anticancer immune response. As quoted by Riaz et al (Raiz, 2016; 28:411-419), “even with the brakes off (CPI treatment), the adaptive immune system still must recognize some portion (or ideally all) of cancer as foreign in order to facilitate selective elimination of the cancer.” Lack of a sufficient presence of clonal neoantigen targeting effector cells will not provide sufficient targeting components as the immune response to enable CPI anticancer effect. Exploration of clonal TMB or neoantigen as a surrogate to clonal neoantigen targeting effector cells may enhance CPI activity in ovarian cancer and in other solid tumor populations not as responsive to CPI therapy. Other approaches attempting to impact antitumor effect dependent targeted immune response (i.e., tumor infiltrating lymphocyte therapy, CAR-T, neoantigen vaccines) may also benefit from involving clonal neoantigen targeting and expression assessment.

### IV. Examples

### Example 1. Identification of Clonal Neoantigens and Assessment of Clonal TMB in Patients from a Phase 2b Vigil® Clinical Trial

The project goals are to determine clonal tumor mutational burden (CTMB) and candidate clonal neo-antigens (CCNA) in tumors from 91 patients who participated in a phase 2b Vigil (CL-PTL-119/126) clinical trial. The project steps include assembly of a bioinformatics processing pipeline for whole exome sequencing data, qualification of the performance of the pipeline with test sequencing data, proof of concept of the method using genomic DNA from matched PBMC, tumor, and Vigil (or FFPE biopsy) samples from 9 patients, sequencing of whole exomes from existing PBMC and tumor genomic DNA samples from an additional 82 patients, data processing, and report generation.

The samples to be used for the study include matched archived tumor and PBMC genomic DNA (gDNA) extracted in 2019 and 2020 from 91 patients who participated in the phase 2b Vigil study (see Table 2).

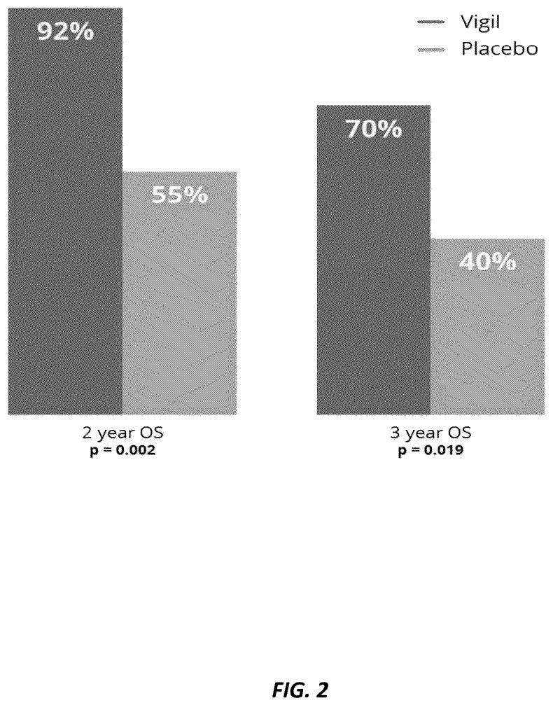

The proposed technical approach to generating CTMB and CCNA data begins with extraction of genomic DNA (gDNA) and verification of gDNA quality and yield for the Vigil vials of nine selected patients and any additional FFPE tissue or PBMC samples required for the complete set of matched samples for the 91 patients. Sequencing libraries can be prepared using the Twist Biosciences Exome 2.0 workflow starting from genomic DNA of matched tumor and PBMC samples and from Vigil samples of the 9 select patients. The libraries can be hybridized to the Exome 2.0 capture probe mix covering 36 Mb of exon sequence with superior coverage of major genetic databases (RefSeq, CCDS, GenCode, Clinvar, ACMG73 and more); importantly the Twist 2.0 Exome fully covers the MHC-1 and MHC-2 loci. Following hybridization, the target-specific PBMC libraries can be sequenced to a mean depth of 100×, while the tumor and Vigil product libraries can be sequenced to a mean depth of at least 650× (940× for the pilot). The sequence reads will then be processed through three separate bioinformatic pipelines, one for HLA germline genotyping, one for somatic mutation detection, and one for copy number analysis (FIG. 3 and FIG. 4).

For each patient, tables of somatic variant calls annotated with allelic copy number variation for the tumor and Vigil product samples can be inputted into PyClone VI software for clonal analysis. Non-synonymous variant calls, annotated with designated clonal or subclonal associations can be used to calculate the tumor mutational burden (TMB) (Vanderwalde et al. Cancer medicine vol. 7, 3:746-756, 2018) for each clone and subclone. The tables of non-synonymous variants associated with the primary clone for each sample, together with the HLA genotyping data will be used to identify putative clonal neo-antigens by first generating potential 9-11-mer peptide sequences and then predicting binding affinity to HLA germline alleles using netMHCpan-4.1 software.

### Bioinformatics Pipeline Development

Pipelines can generate the CTMB and CCNA data from source FASTQ files. The key aspects of the primary pipeline will include: (1) calling consensus sequencing reads from Unique Molecular Identifiers (UMIs), (2) aligning the consensus reads to the reference genome, (3) calculating coverage and on-target statistics, (4) quality score recalibration and local realignment, (5) cropping BAM files based on the target exome, (6) calling germline variants using Haplotype Caller, (7) calling somatic variants using Mutect 2 and/or VarScan 2 using a paired tumor/normal approach, (8) variant quality filtering, (9) determination of allele specific copy number using VarScan2 and ASCAT, (9) processing variant and copy number data into the appropriate input format for PyClone VI (10), and processing the variant and copy number data through PyClone VI to annotate variants with clone and subclone associations (FIG. 4).

Additional scripts can process the output from PyClone VI into usable predictions of putative clonal neo-antigens and to calculate TMB values for each subclone and clone of each sample. A set of scripts can automate the calling of HLA germline alleles from the PBMC sequencing data using either Polysolver or Optitype software. The script for predicting putative clonal neo-antigens will use the non-synonymous mutations for the primary clone from each sample to generate a set of all possible 9-11-mer peptide sequences that include each identified clonal non-synonymous somatic mutation and then use netMHCpan-4.1 software together with the predicted HLA haplotypes, from the PBMC sequencing data, to identify the set of peptides with high affinity for the determined MHC alleles.

### Initial Qualification

A qualification, including both wet lab and dry lab components, can confirm adequate performance of the laboratory process and the bioinformatics pipeline. A qualification plan can describe the samples and source data to be utilized and the metrics to be assessed related to assay performance.

The bioinformatics pipeline through the PyClone VI step will first be tested in a dry lab experiment using downloaded simulated tumor sequencing data available from the Somatic Mutation Calling Tumor Heterogenity Challenge (SMC-Het). SMC-Het provides specific challenges with benchmarks for accuracy of subclonal reconstruction from sequencing data. The data generated from the pipeline can be compared with the expected outcomes to confirm integrity of the pipeline.

Additional preliminary validation of the bioinformatics pipeline can be performed on previously published work by Rizvi et al. (Science 348 (6230): 124-8, 2015). The raw sequencing data is publicly available through the Cancer Genome Atlas (TCGA) cBio portal and database. The Rizvi cohort data has been analyzed in studies investigating clonal neoantigens and response to checkpoint inhibitor therapy. One such study by McGranahan et al. determined a high proportion of clonal neoantigens provided clinical benefit to patients receiving checkpoint inhibitor therapy in NSCLC and melanoma. Data from this study can be found at the database of Genotypes and Phenotypes (dbGAP (accession no. phs000980.v1.p1). Verification will be done using data from 6 patients to confirm that the clonal neoantigens generated from the pipeline match the published data.

For the wet lab portion of the qualification a set of three reference genomic DNA samples derived from distinct tumors can be diluted at various ratios into a healthy control genomic DNA sample in order to create contrived controls. Genomic DNA can be extracted and purified from Vigil product sample of nine patients from the phase 2b Vigil study (Table 2, bolded and underlined patient IDs), evaluated for yield and purity by UV spectrophotometry and for size distribution by 0.8% agarose gel electrophoresis. Quality metrics for the genomic DNA can be assembled into a quality control report, which will be delivered with the final data analysis report. A set of 27 samples comprised of PBMC, tumor, and Vigil samples from nine patients (Table 2, bolded and underlined IDs) and the four contrived controls can be processed starting from genomic DNA through sequencing following the Twist Biosciences Exome 2.0 workflow, targeting a mean coverage depth of 940× for tumor, Vigil, and contrived control samples, and 100× for PBMC samples. Raw sequencing reads can be processed through the bioinformatic pipeline through PyClone VI clone/subclone assignment and through TMB determination and putative clonal antigen prediction. The output from PyClone VI for the reference genomic DNA mixtures can be evaluated to confirm that the detected subclones and their expected allele fractions match the expected composition based on physical mixing. The sample data can be evaluated to confirm that the system can identify a primary clone in at least some of the patients and that calculated TMB values differs between subclones/clones for at least a portion of the patients evaluated.

Table 2. Amount of extracted DNA from normal (PBMC) and tumor tissue and availability of Vigil sample vials of patients that participated in the phase 2b Vigil clinical trial.

TABLE 2

| | PBMC | PBMC | Tumor | Tumor | No. Vigil |
|---|---|---|---|---|---|
| | Conc. | Amount | Conc. | Amount | Vials |
| Subject ID | (ng/uL) | Left (ng) | (ng/uL) | Left (ng) | Remaining |
| 100_0025 | 82.4 | 7740 | 10.304 | 612.664 | 6 |
| 100_0035 | 9.35 | 516.3 | 14.05 | 1986.67 | 29* |
| 100_0042 | 75.75 | 7075 | 6.15 | 858.386 | 9 |
| 100_0046 | 19.65 | 1465 | 7.9 | 839.77 | 9* |
| 100_0057 | 9.05 | 486.9 | 9.35 | 1526.343 | 11 |
| 100_0060 | 6.5 | 337 | 12.95 | 944.316 | 10* |
| 100_0065 | 14.55 | 955 | TA | TA | 10 |
| 100_{—}0067 | 14.4 | 940 | 21.7 | 1801.664 | 21* |
| 101_0001 | 10.35 | 614.3 | 14.85 | 1130.084 | 3 |
| 101_0003 | 36.05 | 3105 | 15.35 | 1179.625 | 20* |
| 102_{—}0003 | 14.9 | 1060.2 | 45.1 | 3693.69 | 3 |
| 102_0007 | 19.35 | 1435 | 25.15 | 1614.63 | 6 |
| 103_0004 | 11.5 | 727 | 30.05 | 2619.967 | 1 |
| 103_{—}0009 | 8.8 | 462.4 | 11 | 753.1635 | 29* |
| 103_0010 | 8.55 | 437.9 | 33.3 | 2938.502 | 2 |
| 103_0012 | 7.35 | 320.3 | 30.25 | 2241.525 | 5 |
| 103_0013 | 18.25 | 1325 | 7.25 | 385.5 | 9* |
| 103_0016 | 5.6 | 248.8 | 8.3 | 488.2794 | 10* |
| 103_0017 | 7.05 | 290.9 | 21.458 | 1734.968 | 11 |
| 103_0020 | 8 | 400 | 19.25 | 1561.697 | 3 |
| 103_0022 | 39.25 | 3446.5 | 11.65 | 415.905 | 6 |
| 103_0031 | 9.9 | 570.2 | 9.2 | 576.1206 | 6 |
| 110_0002 | 12.2 | 795.6 | 14.9 | 1135.227 | 12 |
| 110_0003 | 10.35 | 614.3 | 15.4 | 1183.888 | 10* |
| 112_0001 | 19.65 | 1525.7 | 15.249 | 1138.904 | 11 |
| 112_0006 | 11 | 678 | 13.75 | 624.25 | 10 |
| 112_0007 | 13.5 | 850 | 35.4 | 3144.336 | 1 |
| 112_0010 | 6.6 | 260 | 23.55 | 1982.798 | 2 |
| 112_0012 | 17.75 | 1339.5 | 25.786 | 2150.456 | 3 |
| 129_0004 | 27.3 | 2230 | 17.9 | 1429.234 | 12 |
| 129_0005 | 9.6 | 540.8 | 30.35 | 2649.188 | 12 |
| 129_0007 | 17.45 | 1310.1 | 38.9 | 3487.113 | 12 |
| 136_0002 | 13.65 | 937.7 | 14.1 | 1056.459 | 43* |
| 136_0003 | 27 | 2246 | 4.24 | 186.56 | 4 |
| 136_0008 | 51.35 | 4632.3 | 22.4 | 1870.289 | 6 |
| 136_0013 | 33.8 | 2912.4 | 7.104 | 356.984 | 8 |
| 136_{—}0015 | 12.95 | 795 | 31.35 | 2590.397 | 5 |
| 136_0021 | 18.9 | 1390 | 20.3 | 1562.9 | 6 |
| 136_0022 | 33.95 | 2927.1 | 41.55 | 3539.034 | 9* |
| 136_0024 | 47.25 | 4230.5 | 24.75 | 2100.411 | 21* |
| 136_0027 | 24.95 | 1995 | 24.35 | 2061.308 | 6 |
| 137_0006 | 53.1 | 4803.8 | 10.5 | 704.3423 | 12 |
| 139_{—}0010 | 26.3 | 2130 | 14.7 | 1115.926 | 18* |
| 144_0002 | 69.9 | 6490 | 33.3 | 2537.46 | 26* |
| 144_0004 | 47.55 | 4259.9 | 15.25 | 1169.71 | 11 |
| 144_0005 | 55.15 | 5015 | 21.65 | 1796.709 | 4 |
| 144_0007 | 67 | 6166 | 9.75 | 630.4384 | 12 |
| 144_0008 | 76.35 | 7082.3 | 12.75 | 924.7663 | 10* |
| 144_0009 | 16.85 | 1351.3 | 14.805 | 1096.28 | 11 |
| 144_0011 | 26.85 | 2331.3 | 16.3 | 1190.814 | 12 |
| 144_0012 | 6.75 | 361.5 | 26.8 | 2301.564 | 1 |
| 153_0003 | 53 | 4800 | 7.8 | 439.4256 | 9* |
| 153_0010 | 14.2 | 1091.6 | 13.65 | 1012.348 | 26 |
| 156_0001 | 11.5 | 827 | 13.95 | 1041.985 | 9* |
| 163_0002 | 51.7 | 4670 | 21.6 | 1791.615 | 9* |
| 163_0009 | 6.65 | 351.7 | 15.25 | 1169.172 | 9* |
| 163_0010 | 9.85 | 665.3 | 2.44 | 80.56 | 10 |
| 163_0014 | 7.65 | 449.7 | 18.35 | 1473.485 | 9* |
| 163_0015 | 8.35 | 518.3 | 22.9 | 1919.331 | 12 |
| 163_0026 | 6.4 | 327.2 | 51.7 | 4342.8 | 6 |
| 163_0027 | 5.45 | 234.1 | 17.25 | 1365.291 | 12 |
| 163_0039 | 9.1 | 591.8 | 10.231 | 657.176 | 6 |
| 173_0006 | 14.45 | 1116.1 | 12.725 | 896.6 | 4 |
| 173_0009 | 34.15 | 2915 | 27.65 | 2384.707 | 2 |
| 173_0011 | 35.2 | 3020 | 17 | 1340.929 | 12 |
| 173_0020 | 5.65 | 253.7 | 36.05 | 3208.022 | 1 |
| 173_0028 | 8.2 | 503.6 | 19.7 | 1605.549 | 6 |
| 178_{—}0007 | 5.65 | 399.7786 | 11.25 | 777.6954 | 9* |
| 178_0008 | 47.1 | 4210 | 27.25 | 2345.647 | 12 |
| 178_{—}0011 | 3.65 | 225.6099 | 21.15 | 1747.795 | 1* |
| 178_0014 | 3.8 | 186.0171 | 18.05 | 1443.944 | 9* |
| 178_0015 | 4.2 | 253.808 | 25.9 | 2213.084 | 9 |
| 180_{—}0002 | 5.9 | 403.7934 | 19.5 | 1586.113 | 9* |
| 180_0008 | 10.2 | 814.9905 | 17.55 | 1395.15 | 8 |
| 185_0002 | 4.05 | 267.2356 | 6.628 | 312.448 | 6 |
| 197_{—}0001 | 4.4 | 282.5284 | 5.3 | 251.22 | 8 |
| 206_0005 | 12.25 | 1020.923 | 16.3 | 1272.559 | 2 |
| 206_0011 | 1.1 | 47.63677 | 18.65 | 1502.7 | 10* |
| 206_0015 | 5.35 | 385.5116 | 20.15 | 1649.541 | 9* |
| 206_0024 | 5.5 | 380.7865 | 16.7 | 1228.399 | 8 |
| 206_0027 | 6.55 | 486.1934 | 18 | 1349.067 | 12 |
| 206_0029 | 8.2 | 625.556 | 12.246 | 789.386 | 6 |
| 206_0030 | 80.05 | 7505 | 16.1 | 1252.676 | 6 |
| 206_0031 | 40.05 | 3505 | 9.2 | 576.7521 | 10 |
| 206_0032 | 39.4 | 3440 | 15.1 | 1154.893 | 12 |
| 206_0034 | 38.1 | 3310 | 8.05 | 423.3514 | 6 |
| 206_0036 | 51.95 | 4695 | 16.55 | 1296.731 | 6 |
| 235_0001 | 34.7 | 2970 | 30.3 | 2644.565 | 5 |
| 242_0002 | 9.65 | 777.3487 | 20.862 | 1677.752 | 12 |
| 242_0010 | 4.6 | 316.2397 | 6.035 | 273.31 | 9 |
| 242_0013 | 8.7 | 689.7075 | 26.75 | 2296.388 | 5 |

Patients in bold and underlined are included in the first 9 analyzed
TA = tissue available
*D2 cryovial

The tumor sequencing read files can be subsampled to confirm that acceptable data can be obtained when the mean coverage depth is reduced to ˜600×.

### Sample Analysis

Following approval of the qualification report, matched tumor and PBMC genomic DNA from an additional 82 patients consisting of the remaining patients from the phase 2b Vigil study will be processed according to the established standard method and analyzed using the established bioinformatics pipeline in order to determine clonal TMB and to generate a list of candidate clonal neoantigens.

Genomic DNA from each PBMC and tumor sample for each patient can be processed alongside two reference standard genomic DNAs per batch, through preparation of sequencing libraries and hybridization-based capture using the Twist Biosciences Exome 2.0 workflow.

Pooled libraries can be sequenced on the NovaSeq 6000 instrument to mean depth of 650× and 100× for tumor and germline samples, respectively.

The raw sequence data can be processed through the bioinformatics pipeline described above.

Tabular lists of annotates somatic polymorphisms can be generated for each patient for each primary clone and subclone in the tumor. Tables of TMB scores for each patient for each clone and subclone will also be prepared. Lists of candidate neoantigens for each tumor with an identified primary clone can be produced. Sequencing quality metrics including coverage depth and on-target percentage can be provided for each batch of sample processed.

### Example 2. Robust Anti-Tumor Activity in Homologous Recombination Proficient Cells

For HRD and HRP cell lines, ovarian cancer cell lines UWB1.289, UWB1.289 BRCA1, can be used as cellular models. Cells are grown according to the supplier's instructions with cellular survival measured after treatment with the immunotherapy (e.g., cellular or gene therapy). The cancer cell lines can be seeded in 96 well-plates and incubated 24 hours at 37° C. before the addition of therapy. Following exposure, to the immunotherapy (e.g., cellular or gene therapy), cell survival is measured using the XTT assay (Thermo, Cat #: X12223).

Briefly, the XTT solution is added directly to each well containing cell culture and the cells incubated for 4 hours at 37° C. before reading the absorbance at 485 nm using a microplate reader (VICTOR Nivo Plate Reader, Perkinelmer). Cell survival is calculated as the ratio of living treated cells to living mock-treated cells. The IC50 (which represents the dose at which 50% of the cells are viable) is calculated by a non-linear regression model using GraphPad Prism software (version 5.04) by plotting the percentage viability against the Log of the drug concentration on each cell line.

The cells treated with cellular, or gene therapy will show significant benefits in cancer patients with homologous recombination proficiency (HRP) over the cells which are homologous recombination deficient (HRD) induced by BRCA mutations.

### Example 3. Exome Sequencing Shows Same Pattern of Clonal Tumor Mutational Burden, Intratumor Heterogenicity and Clonal Neoantigen between Autologous Tumor and Vigil Product

### Overview

Extensive retrospective data supports a beneficial relationship of overall survival (OS) to high clonal tumor mutation burden (cTMB), high clonal neoantigen load (cNEO) and low intratumor heterogeneity (ITH) in cancer patients who receive immunotherapy. In order to explore this relationship prospectively with Vigil, a triple function targeted immunotherapy involving ovarian cancer patients in long term follow up of Phase 2b VITAL trial, developed a lab exome sequencing procedure was developed and associated bioinformatics pipeline to determine clonal signal patterns.

DNA libraries containing exome sequences tagged with unique molecular identifiers (UMI) were prepared from paired tumor and normal samples and sequenced on Illumina sequencers to high coverage depths of ˜930× and ˜ 130× for tumor and normal, respectively. Raw sequence reads were processed into optimized binary alignment map (BAM) files, using the UMI information. The BAM files were inputted into modules for calling MHC-I alleles, calling and annotating single nucleotide variants (SNVs) and small insertions/deletions (InDels), and for determination of allelic copy number. The outputs were used to predict the sequence of peptide neoantigens, and to perform clonality analysis in order to assign each SNV and InDel in a patient tumor sample to a primary clone or subclone.

The functionality of the bioinformatics pipeline was assessed using whole exome Illumina sequencing data from three previously published studies. Evaluation of the pipeline using synthetic sequencing data from a sub-clonal deconvolution tool benchmarking study, showed good correspondence of the cancer cell fraction (CCF) called by our system versus the benchmark data, as well as a positive predictive value (PPV) and positive percent agreement (PPA) of >97.5% and >96.5%, respectively, for SNV and InDel detection with minimum requirements for variant density and allele fraction. Haplotype calls from the bioinformatic pipeline MHC-I/MHC-II typing module matched a published benchmark for 91.5% of the calls in a sample of 99 patients. Analysis of exome sequencing data from 14 patients with advanced melanoma revealed strong correlation with cTMB values calculated from the published data (R²=0.99). Furthermore, clinical response to nivolumab was associated with higher levels of cNEO as determined by our pipeline, consistent with the previously published results.

The entire wet lab process and bioinformatic system was applied to a set of matched normal (peripheral blood mononuclear cells), tumor, and Vigil product samples from 9 (n=27 samples) ovarian cancer subjects entered into the VITAL (CL-PTL-119) trial. Results demonstrated marked correlation (R²=0.98) of cTMB between tumor used to construct Vigil and Vigil product. Correlation between tumor and Vigil for the cNEO and ITH metrics were also strong, showing R2 values of 0.95 and 0.87, respectively.

The consistency of the bioinformatic pipeline results with previously published data as well as the agreement between results for tumor and Vigil for the entire system provide a strong basis of support for utilization of this system for prospective determination of cTMB, cNEO, and ITH values in clinical tumor tissue in order to explore possible correlative relationships with clinical response parameters.

### Introduction

Higher levels of clonal tumor mutational burden (cTMB) and clonal neoantigen load (cNEO) along with lower levels of intra-tumor homogeneity (ITH) in tumor samples in a wide variety of malignancies have been associated with significantly improved overall survival following treatment with immune checkpoint inhibitors [1-6]. Mutations in the genomic DNA that occur early in the development of a tumor and are carried forward in all tumor cells are considered to be clonal. These clonal mutations are responsible for the biochemical transition from a normal cell to a cell that is associated with the development of the malignant state. Sub-clonal mutations arise later in the development of the tumor and are carried forward in daughter cells that make up only a sub-portion of the tumor [7]. Tumor mutational burden (TMB) is defined as the number of non-synonymous mutations per million base pairs (Mb) of genomic DNA [8,9]. cTMB is calculated in the same manner as TMB but includes only clonal mutations [1,2]. ITH is the estimated fraction of non-synonymous mutations in a population of tumor cells that are sub-clonal versus clonal [3]. Non-synonymous mutations in tumor cells may give rise to mutated proteins which are either ubiquitinated and processed into peptides that are presented on the cell surface in complex with MHC-1 molecules or they may be endocytosed by antigen-presenting cells and processed through the lysosomal pathway into peptides to be presented on the antigen-presenting cell surface also in complex with MHC-I [10]. Novel peptides derived from mutated proteins that are presented on the tumor cell surface in complex with MHC-I are termed neoantigens. Methods that generate a list of potential peptides derived from variant protein sequences and estimate the binding affinity of those peptides to the patient's own MHC-1 molecules derived from the HLA-A, HLA-B, and HLA-C loci, can be used to predict with some level of accuracy which peptides will have a high affinity for their specific MHC molecules and therefore are likely to be presented on the cell surface [11,12]. The set of peptides that are derived from tumor-associated clonal mutations and are predicted to have high affinity for the patient's MHC molecules, but are not encoded by the germline genome are termed clonal neoantigens. The cNEO is the count of these peptide neoantigens per Mb of genomic DNA.

Vigil is a clinically tested precision immunotherapy that is prepared by harvesting autologous tumor cells. These cells are transfected with a dual plasmid expressing granulocyte macrophage colony stimulating factor (GM-CSF) as well as a bi-functional short hairpin (bi-sh) RNA that blocks the expression of furin, an enzyme responsible for TGFβ1 and TGFβ2 supply. Following irradiation and satisfaction of product release criteria, Vigil is injected intradermally into the patient from whom the tumor tissue used to construct Vigil was obtained [13]. The resulting down-regulation of TGFβ1 and B2 expression by furin knockdown and the expression of GM-CSF by the injected tumor cells as we have previously shown generate a dendritic cell response in the host that results in the expansion of effector cells that can target tumor neoantigens demonstrated effectively by ELISpot assay [7]. Vigil has been shown in multiple studies to elicit an effective dendritic cell response resulting in relapse free survival (RFS) and OS clinical benefit [14]. We thus hypothesized that levels of cTMB, cNEO and ITH will classify benefit and resistance of patients undergoing therapy with Vigil and will define clinical responders or non-responders. Moreover, we predicted that there would be a strong correlation across patients in cTMB, cNEO and ITH levels between autologous tumor used to construct Vigil and Vigil product. Consequently, our team constructed a bioinformatics computational pipeline and process algorithm to determine quantitative cTMB, cNEO, and ITH levels and to compare clonal signal expression between the tumor used to construct Vigil and Vigil product.

### Results

### Bioinformatics Pipeline Development

We developed a complete bioinformatics pipeline that takes whole exome sequencing data from matched tumor and normal genomic DNA samples as input and outputs values for cTMB and ITH, and identifies a list of potential clonal neoantigens peptides expected to bind to the patient's MHC-1 molecules with high affinity. By analyzing the allelic frequency and allele-specific copy number of tumor-specific variants using PyClone 6.1 it has been shown to be possible to identify non-synonymous “clonal” variants that are likely to be present in virtually all cells of the tumor [15,16]. The quantity of non-synonymous clonal variants per Mb of exome is the determined cTMB, whereas the fraction of non-clonal variants relative to total variants across the exome, again considering only non-synonymous variants, is the ITH. A list of potential clonal neoantigens is arrived at by in silico synthesis of 8-11-mer peptides associated with each non-synonymous clonal variant and examining their expected binding affinity to the patients predicted MHC Class I molecules [12,17]. A cNEO score which is the number of predicted high binding affinity peptides that span a clonal non-synonymous variant and have higher affinity than the corresponding reference-derived peptide sequence, per Mb of exome is then calculated.

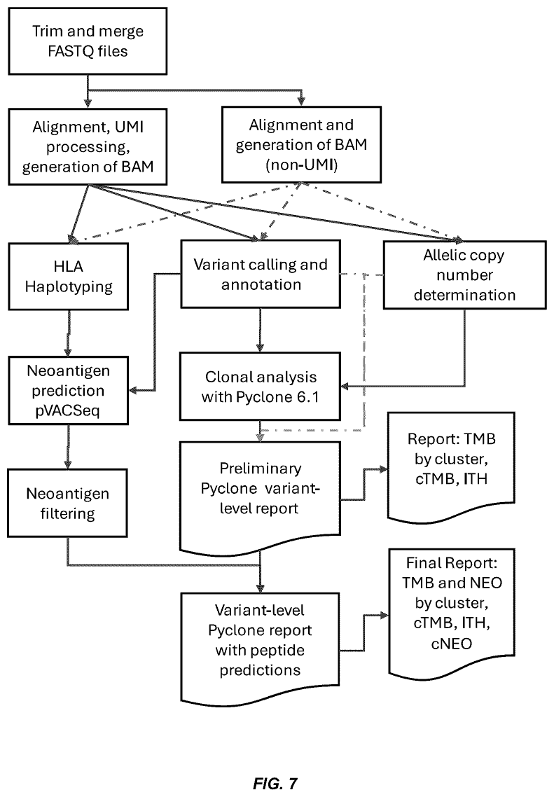

The procedural steps required for the pipeline were based on previously published work [2,3,18], and are outlined in FIG. 7. The pipeline includes initial steps to process raw sequencing reads to optimized BAM files either with or without UMIs. These steps include a module for calling MHC-I and MHC-II haplotypes using HLA*LA [19], a module for calling and annotating SNVs and InDels using GATK Mutect2 and Funcotator, and a module for determining allelic copy number using FACETS [20]. The outputs of the variant calling along with the MHC-1 haplotype calls are used as input for peptide neoantigen prediction using pVACSeq [21]. The output of variant calling and allelic copy number determination are used as input to clonal analysis using PyClone 6.1 which produced a tabular report with tumor-associated variants assigned to individual clusters, each having an associated CCF. Proprietary custom scripts are used to add information about predicted peptide neoantigens to the PyClone report, to filter the report to include only non-synonymous variants, and to summarize and calculate the key metrics for each patient tumor sample and write them into a TMB report. Additional information regarding the bioinformatics pipeline framework and processing steps as well as metric calculations are provided in Methods below.

### Overview of Pipeline Performance Verification

The bioinformatics pipeline was qualified by using input Illumina-type whole exome NGS data from three different published studies and comparing the results generated from our pipeline or modules thereof to the published results. The developed bioinformatics pipeline contains modules for both UMI-based and non-UMI based alignment and production of BAM files. The UMI-based approach uses a combination of processing steps available in the fgbio software package and alignment with bwa-mem2 to identify multiple sequence reads derived from the same original genomic DNA molecule and combine them into a consensus sequence prior to additional alignment with bwa-mem2. This approach is expected to reduce the measured sequencing error rate and improve the accuracy of estimation of allelic frequency [22,23]. Although it would be desirable to verify the performance of both the UMI and non-UMI modules for BAM file generation, it was not possible to identify publicly available tumor-derived whole exome sequencing data generated using UMIs, and therefore in the pre-wet lab bioinformatics pipeline verification only the non-UMI module was used.

### Evaluation of Core Bioinformatic Pipeline Steps Using Simulated Sequencing Data.

The performance of the core portion of the pipeline, which included read trimming, BAM file generation, variant calling, allelic copy number determination and clonal analysis with PyClone version 6.1 was evaluated using simulated tumor exome sequencing data generated as part of a clonal deconvolution benchmarking study [18]. The authors of the study generated simulated sequencing reads from 9 different simulated tumor genomes and mixed each with simulated reads from a matching normal healthy genome in order to generate samples having 100%, 75%, 50%, or 25% tumor DNA. The tumor genomes were designed to simulate a tumor undergoing evolution with three to four generations of subclonal differentiation; each of these subclones have specific variants associated with them, with variants associated with subclones arising later in the evolution having lower allelic frequency. Simulated sequencing data from three of the tumor genomes, S1R1 containing a frequency of 1×10⁶ and 2×10⁻⁷ SNVs and InDels, respectively, and S2R1, and S2R3 that contained a 10-fold higher frequency of these mutation types, were analyzed in paired format with the simulated reads from their corresponding matching normal healthy genomes. The coverage depth analyzed was 250× for tumor and 60× for normal simulated sequencing reads. The PyClone variant-level preliminary report for each of the 12 simulated tumor DNA sequence samples from the Clonal Neoantigen pipeline was compared to a truth set from the Tanner et al publication with respect to the variants detected versus those known to be in the truth set, the expected versus determined tumor purity, and the overall copy number for the chromosomal region spanning a variant versus the determined total copy number for that region (Table 4).

TABLE 4
Comparison of PyClone variant-level report data to Tanner et al., truth set.

| | | | | | | Correlation (R) | Mean |
|---|---|---|---|---|---|---|---|
| | Determined | Truth Set | | Variants | | Determined | absolute |
| | Tumor | Tumor | Variants | Found in | PPV (TP/ | CCF vs. Truth | difference |
| Sample ID | Purity | Purity | Detected | Truth Set | (TP + FP) | Set CCF | in ploidy |
| S1R1_N-S1R1_A | 99.6% | 100% | 49 | 46 | 93.9% | 95.4% | 0.118 |
| S1R1_N-S1R1_B | 74.8% | 75% | 44 | 41 | 93.2% | 97.1% | 0.103 |
| S1R1_N-S1R1_E | 60.4% | 50% | 41 | 38 | 92.7% | 97.4% | 0.112 |
| S1R1_N-S1R1_F | 55.2% | 25% | 26 | 22 | 84.6% | 82.6% | 0.149 |
| S2R1_N-S2R1_A | 99.6% | 100% | 444 | 438 | 98.6% | 92.4% | 0.319 |
| S2R1_N-S2R1_B | 74.0% | 75% | 408 | 403 | 98.8% | 92.9% | 0.312 |
| S2R1_N-S2R1_E | 68.0% | 50% | 348 | 342 | 98.3% | 92.1% | 0.265 |
| S2R1_N-S2R1_F | 45.3% | 25% | 239 | 235 | 98.3% | 84.9% | 0.188 |
| S2R3_N-S2R3_A | 99.1% | 100% | 431 | 424 | 98.4% | 95.9% | 0.219 |
| S2R3_N-S2R3_B | 73.5% | 75% | 391 | 385 | 98.5% | 96.9% | 0.213 |
| S2R3_N-S2R3_E | 48.4% | 50% | 329 | 321 | 97.6% | 95.3% | 0.214 |
| S2R3_N-S2R3_F | 31.0% | 25% | 236 | 231 | 97.9% | 83.5% | 0.187 |

### Analytical Specificity of Variant Detection

The positive predictive value (PPV), calculated as the number of true positive variants detected divided by the total variants detected (TP/(TP+FP)) was >97.5% for synthetic sequencing data derived from synthetic tumor genomes S2R1 and S2R3, regardless of tumor purity which ranged from 25% to 100% (Table 4), indicating that less than 2.5% of the variants in the final list were false positives for the simulated samples that had an average of 10 SNVs and 2 InDels per megabase of exome DNA. The system was less accurate for the S1R1 simulated tumor genome tumor which had a 10-fold lower density of SNVs and InDels as compared to the other two simulated tumor genomes tested. However, it is not surprising that across the 50,446,305 nucleotide exome that 5-10 false positives would arise in the result set, and since the expected number of variants, combining SNVs and InDels, for tumor genome S1R1 was only 60, these few false positives had a disproportionate impact on the PPV. In fact, the maximum number of false positives for any of the samples was 6, yielding a worst case false positive rate of 1.19*10⁻⁷ when considering all nucleotides.

### Accuracy of Prediction of CCF Values

The accuracy of prediction of the CCF of the true positive detected variants was assessed by calculating the correlation coefficient to the known CCF values in the truth set. Excluding the 25% purity simulated tumor genomes, the correlation between the truth set CCF values and the determined CCF values was >92% for all simulated tumor sequencing data samples (mean 95.0%). In the Tanner simulated tumor genomes, 40% of the total variants are represented at 100% CCF, 15% at 50% CCF, and the remaining 45% of variants range from 1%-20% CCF (Supplemental Table I). Since virtually all of the variants are heterozygous, the allelic frequency is half of the CCF, so in the synthetic data from 25% tumor purity samples, 45% of the variants must have an effective allele fraction of 2.5% or less (20%*0.5*0.25). The expected minimum allelic frequency (AF) detectable at 250× coverage at 99% efficiency is 5% AF [24], and therefore 45% of the variants in the 25% tumor samples would be below the typical limit of detection, leading to greater error in CCF estimation.

### Allelic Copy Number Determination Accuracy

FACETS estimates the total and minor allele copy number for chromosomal regions, and proprietary code in the bioinformatics pipeline converts these values to major and minor allele copy number and provides it as input to PyClone vs. 6.1 (see Methods). The sum of the major and minor allele copy number taken from the PyClone results files was compared to the total copy number (ploidy) from the truth set for the chromosomal region spanning each variant. The mean absolute difference across all variants detected in a sample was 0.2 copies, with the poorest performing sample showing a mean difference of 0.32 copies (Table 4). This compared favorably to the mean absolute copy number difference from the truth set of 0.591, reported by Tanner et al in Supplemental Table IV of their publication [18], for 75% and 100% purity samples. These results show that the FACETS application is running properly as a component of the pipeline and the output is faithfully utilized and reported in the PyClone results files.

### Sensitivity of Variant Detection

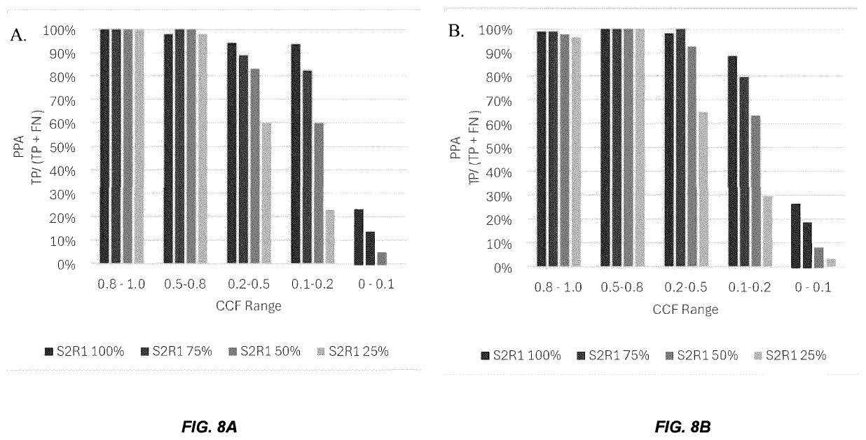

To examine the sensitivity of detection of variants by the bioinformatic pipeline, the variants in the Tanner data sets for were binned by expected CCF value and the positive percent agreement (PPA) was calculated for each bin as TP/(TP+FN). This analysis was performed only for the S2R1 and S2R3 synthetic genomes due to their 10-fold higher variant density relative to S1R1. As expected, the ability to detect specific expected variants in the simulated genomes decreased with the known CCF. When the expected CCF was greater than or equal to 0.5 the positive percent agreement (PPA) was >96.5% and this was consistent down to 25% tumor purity (FIGS. 8A and 8B). The PPA values lower than 100% for variants with CCF ≥0.5 were due to a small number (0-3) variants that were not detected in each sample. The reason for the lack of detection of these variants was investigated and in each case it was due to removal in the FilterMutect2Calls quality filtering step based on one or more of the error modes comprising clustered events, orientation bias, or presence in the panel of normals (not shown). Thus, at CCF >0.5 the PPA for variant detection was 100% prior to quality filtering. As the expected CCF range drops, a greater percentage of variants become undetectable and this is exasperated as the percentage of tumor in the simulated tumor sample genome drops from 100% to 25% (FIGS. 8A and 8B).

Evaluation of the Reliability of the Entire Clonal Neoantigen Pipeline Using Published Data from Riaz et al [4].

The complete non-UMI version of the bioinformatics pipeline was qualified using available raw data from a Riaz et al publication [4] examining the relationship between the fraction of mutations that were clonal, the mutational load, the clonal mutational load, and levels of peptide neoantigens, among other markers, to the patient response to nivolumab treatment in advanced melanoma [4]. There were a total of 68 patients treated with nivolumab in the study but most had been previously treated with ipilimumab which impacted the make-up of predicted peptide neoantigens in the patient tumors. Therefore, patients that had not previously received ipilimumab therapy were selected for analysis; this included all 7 non-pre-treated patients that showed a partial or complete response (PR/CR) to nivolumab and the first 7 patients who did not respond to nivolumab (progressive disease, PD). Riaz et al., noted a consistently high fraction of clonal mutations (i.e. low ITH) in the PR/CR patients relative to the PD patients, and a significant survival advantage for patients with a high clonal mutation load (i.e. high cTMB).

A strong correlation between the Riaz data and our tabulated results was observed for both ITH (R2=0.758) and cTMB (R2=0.990) (Table 5). However, ITH values from the Riaz data were an average of 37.1% lower as compared to our bioinformatic pipeline, and the Riaz cTMB scores averaged 63.7% higher than the values for the same samples from our pipeline (Table 5). The higher ITH and lower cTMB achieved with our bioinformatic pipeline resulted from a trend towards higher CCF scores in the Riaz data and not to a lower rate of detection of non-synonymous variants (Table 5).

TABLE 5
Comparison of cTMB and ITH level for the same raw sequencing
data analyzed by Riaz versus internal pipeline

| | | | Riaz | | | | | | | |
|---|---|---|---|---|---|---|---|---|---|---|
| | | | Clonal | | | | Internal | | | |
| | | | Muts | Riaz | | | Pipeline | Internal | Internal | Internal |
| Patient | | | (>0.85 | Total | | | Total | Pipeline | Pipeline | Pipeline |
| ID | Sex | Group | CCF) | Muts | Riaz ITH | Riaz cTMB | Muts | ITH | cTMB | cNEO |
| Pt18 | Male | CR | 212 | 212 | 0.000 | 5.470 | 240 | 0.079 | 4.381 | 8.841 |
| Pt3 | Male | CR | 166 | 176 | 0.057 | 4.283 | 207 | 0.208 | 3.251 | 4.698 |
| Pt30 | Female | CR | 35 | 65 | 0.462 | 0.903 | 93 | 0.634 | 0.674 | 0.714 |
| Pt44 | Male | CR | 350 | 358 | 0.022 | 9.031 | 374 | 0.214 | 5.828 | 12.726 |
| Pt7 | Male | CR | 1463 | 1480 | 0.011 | 37.749 | 1354 | 0.185 | 21.865 | 36.177 |
| Pt72 | Female | CR | 387 | 407 | 0.049 | 9.986 | 413 | 0.380 | 5.075 | 13.321 |
| Pt94 | Male | CR | 395 | 395 | 0.000 | 10.192 | 280 | 0.139 | 4.777 | 11.616 |
| Pt11 | Female | PD | 83 | 95 | 0.126 | 2.142 | 133 | 0.323 | 1.784 | 3.548 |
| Pt25 | Male | PD | 72 | 93 | 0.226 | 1.858 | 111 | 0.505 | 1.090 | 2.458 |
| Pt28 | Female | PD | 14 | 42 | 0.667 | 0.361 | 72 | 0.681 | 0.456 | 1.051 |
| Pt5 | Female | PD | 22 | 49 | 0.551 | 0.568 | 144 | 0.764 | 0.674 | 1.289 |
| Pt74 | Male | PD | 37 | 42 | 0.119 | 0.955 | 44 | 0.182 | 0.714 | 1.586 |
| Pt8 | Male | PD | 76 | 86 | 0.116 | 1.961 | 87 | 0.126 | 1.507 | 2.617 |
| Pt84 | Female | PD | 3 | 10 | 0.700 | 0.077 | 19 | 0.526 | 0.178 | 0.456 |

| | | | | | | | | |
|---|---|---|---|---|---|---|---|---|
| Mean | 236.8 | 250.7 | 0.222 | 6.110 | 255.1 | 0.353 | 3.732 | 7.221 |

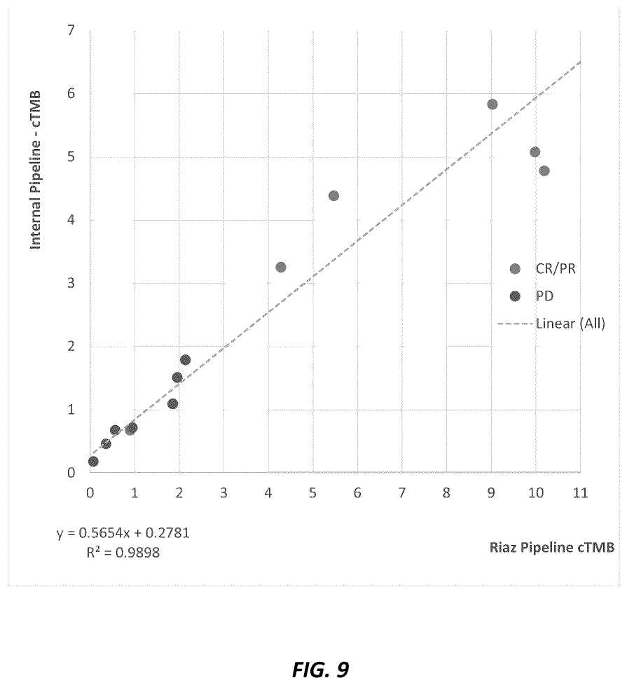

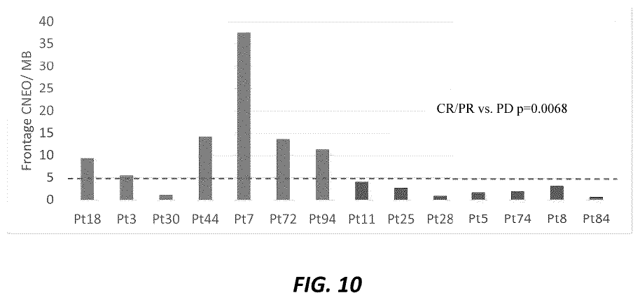

Importantly, the analysis of the Riaz raw data using the bioinformatics pipeline developed in this study reached a similar conclusion to the published results. The cTMB values were generally higher in patients likely to respond to nivolumab as compared to non-responders, as evidenced by the separation (p=0.0068) between CR/PR and PD patients on the plot of Riaz's versus our cTMB values (FIG. 9). The current bioinformatic pipeline predicts a set of peptides that are likely to bind to each patient's predicted MHC I molecules and calculates a cNEO value based on the number of high-scoring peptides that are derived from variants associated with the primary clone. The cNEO values generated by the pipeline are reported in Table 5 for each of the 14 patients selected from the Riaz study for use in the qualification. As was observed for the relationship between patient response and cTMB values, cNEO levels were significantly (p=0.0068) higher in therapy-responsive patients (CR/PR) (patients 18, 3, 30, 44, 7, 72, 94) as compared to the non-responsive patients (PD) (patients 11, 25, 28, 5, 74, 8, 84) (FIG. 10).

### Evaluation of the HLA Haplotyping Module of the Bioinformatics Pipeline

Publicly available exome sequencing data for 99 subjects that participated in the 1000 genomes project was utilized for evaluation of the HLA haplotyping module of the bioinformatics pipeline. The patients were selected from among 820 total patients used to evaluate several different sequencing-based HLA haplotyping tools [25]. CRAM-format sequencing data as well as a truth set containing the expected MHC-I and MHC-II haplotypes for each of the 99 patients were downloaded from the authors Github repository [25]. The sequencing data files were pre-processed as described in methods to generate BAM files which were used as input to the HLA haplotyping module. The available truth set was originally developed as part of a previous study, by Abi-Rached et al, using the same 1000 genomes input exome sequencing data and PolyPheMe software [26]. HLA*LA generates G-type or 3-field haplotype calls that are based on both the protein and underlying nucleotide sequences in the antigen recognition domain (ARD). The Abi-Rached truth set was available in 2-field resolution which is based only on the predicted amino acid sequence of the ARD [25]; thus HLA*LA output data was truncated to two fields for purposes of comparison. The HLA haplotyping module for the bioinformatics pipeline showed an initial percentage match to the Abi-Rached truth set of 86.9%-94.9% depending on the locus (Table 6). Because it was noted in Thuesen, et al., that some of the haplotype calls in the Abi-Rached truth set were proven to be incorrect, the calls that did not match Abi-Rached were also compared to the published calls from Thuesen et al [25], using the same HLA*LA software. From 92.9%-100% of the haplotype calls matched either the Abi-Rached truth set or those published by Thuesen. All 7 of the remaining unmatched calls in HLA-C were shown to be caused by a difference of a single allele which was called as C*02:10 by our bioinformatics module versus C*02:02 in the Abi-Rached truth set and the Thuesen HLA*LA data. The match rate to the truth set obtained by Thuesen for all 820 patients is shown for reference (Table 6).

TABLE 6
Evaluation of HLA Haplotyping Bioinformatics Module

| | | | | | Remaining | Thuesen % |
|---|---|---|---|---|---|---|
| | | Gradalis vs. | Additional | % Match to | non-matching | Correct (820 |
| | Gradalis vs. | Abi-Rached | Matches to | Abi-Rached | haplotype | samples - |
| Locus | Abi-Rached | % | Thuesen | or Thuesen | calls | adjusted |
| HLA-A | 88 | 88.9% | 8 | 97.0% | 3 | 92.80% |
| HLA-B | 92 | 92.9% | 6 | 99.0% | 1 | 97.80% |
| HLA-C | 86 | 86.9% | 6 | 92.9% | 7 | 96.60% |
| HLA-DQB | 94 | 94.9% | 3 | 98.0% | 2 | 97.20% |
| HLA-DRB | 93 | 93.9% | 6 | 100.0% | 0 | 94.00% |

### Implementation of Twist Exome 2.0 and UMI-Based Processing to Improve Clonal Variant Identification in Whole Exome Sequencing

In order to improve the accuracy of clonal variant detection, this study utilized the Exome 2.0 panel recently developed by Twist Biosciences, incorporated UMI oligonucleotides into the library preparation process, and utilized UMI information in the bioinformatics pipeline. Twist exome capture technology was found to provide broader and more uniform coverage of the exome as compared to industry standard Agilent exome technology [27]. Incorporating UMIs in library preparation and utilizing the incorporated UMI tags to combine duplicate reads into a consensus sequence improves the accuracy of low frequency variant calling [28].

A set of 27 cryopreserved samples from 9 stage III/IV ovarian cancer patients selected from the Phase IIB trial of Vigil, comprised of a tumor, a Vigil product, and a normal PBMC sample from each were processed through genomic DNA extraction, library preparation, exome sequencing, and bioinformatic analysis incorporating the UMI processing. Genomic DNA samples from ATCC comprising a matched set of a non-small cell lung cancer line (NCI-H2126) and a B lymphoblast cell line (NCI-BL2126) from the same patient were processed alongside the clinical trial samples. Prior to exome selection, DNA libraries were mixed at a 1:9.4 ratio of normal versus tumor and Vigil in order to target a yield of sequence reads that was 9.4× higher in tumor or Vigil as compared to PBMC. An average of 558 million, 544 million, and 65 million sequencing reads were obtained from Tumor, Vigil, and PBMC/normal control samples, respectively (Table 7).

TABLE 7
Coverage statistics for exome sequencing experiment.

| Patient/Sample ID | Normal | Tumor | Vigil |
|---|---|---|---|
| Total read pairs (millions) | 64.67 | 558.13 | 544.29 |
| Aligned read pairs (millions) | 63.71 | 548.35 | 533.76 |
| On target bases (millions, | 6,233 | 53,178 | 51,925 |
| before UMI) | | | |
| On target bases (millions, | 4,480 | 34,039 | 34,038 |
| after UMI) | | | |
| Percentage duplicate reads | 28% | 36% | 34% |
| Mean target coverage before | 171 | 1,459 | 1,424 |
| UMI processing | | | |
| Mean target coverage after | 123 | 934 | 934 |
| UMI processing | | | |
| Percentage loss in coverage | 28.1% | 36.2% | 34.4% |
| % Target bases at 50X (pre-UMI) | 98.4% | 98.8% | 98.8% |
| % Target bases at 100X (pre-UMI) | 93.6% | 98.7% | 98.7% |
| % Target bases at 250X (pre-UMI) | 6.8% | 98.5% | 98.5% |
| % Target bases at 500X (pre-UMI) | 0.1% | 97.3% | 97.1% |
| % Target bases at 50X (post-UMI) | 98.0% | 98.7% | 98.7% |
| % Target bases at 100X (post-UMI) | 74.9% | 98.6% | 98.6% |
| % Target bases at 250X (post-UMI) | 0.5% | 97.9% | 98.2% |
| % Target bases at 500X (post-UMI) | 0.0% | 88.6% | 90.5% |

Coverage metrics were collected prior to and after de-duplication as a result of UMI processing. Approximately 36%, 34%, and 28% of on-target bases were eliminated as duplicates during UMI processing from tumor, Vigil, and PBMC/normal sample data, respectively; these duplicates were collapsed into consensus reads improving the accuracy of the base calls. The coverage uniformity appeared excellent with an average of over 98% of nucleotide positions in the target for PBMC/normal samples having coverage of at least 50×. Tumor and Vigil samples had coverage of at least 500× for an average of 88.6% and 90.5% of nucleotide positions in the target, and ˜ 98% of bases covered at 250× or higher (Table 7, supplemental Excel file 1). The total TMB for NCI-H2126, calculated by summing the TMB for each cluster of variants in the tumor, was 22.5 which was nearly identical to the published value of ˜22.0 [9], supporting the conclusion that the alignment, UMI processing, variant calling modules, and proprietary reporting scripts of the pipeline were performing as expected.

Comparison of key metrics between tumor and Vigil samples

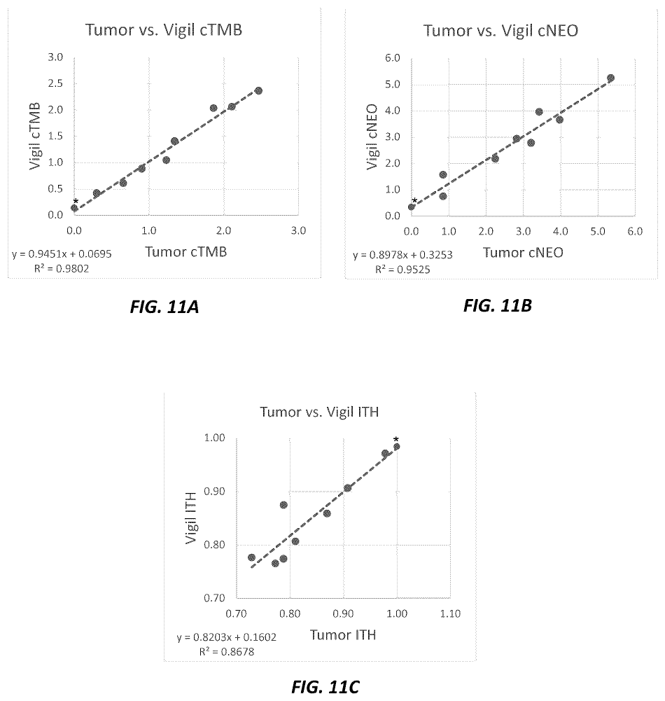

cTMB, ITH, and cNEO scores were calculated for the tumor and Vigil samples from the 9 Phase IIB trial patients as well as the control NCI-H2126 non-small cell lung carcinoma cell line. Values of ITH, cTMB, and cNEO showed strong correlations (R²) between tumor and Vigil of 0.87, 0.98, and 0.95, respectively (FIGS. 11A-11C). For all samples except for one a primary clone was identified in each tumor and Vigil sample. No primary clone was identified for the tumor sample from patient 103_009 (FIGS. 11A-11C, circle with asterisk). A primary clone corresponds to a cluster of variants that have been assigned a CCF >0.9 by PyClone 6.1, making it likely that the variants are present in nearly 100% of the cells making up the tumor. The highest CCF value for any cluster in the tumor for patient 103_009 was 0.879. The Vigil sample for the same patient did have a cluster with a CCF >0.9 but it included only 5 variants, versus an average of 44.2 variants for all clinical trial tumor and Vigil samples.

### Consistency in Detection of Specific Clonal Variants Between Tumor and Vigil

The similarity in cTMB, cNEO and ITH scores between tumor and Vigil for each patient suggested that the exome DNA sequence was highly similar between tumor and Vigil samples of the same patient, and that the entire system, including the wet-lab and bioinformatic steps were reproducible. However, it was important to confirm that the specific set of non-synonymous variants detected were similar between tumor and Vigil within patients and that the top scoring peptides derived from each of these variants were the same. For the purposes of this analysis, we focused on the non-synonymous clonal variants since these are the set of variants that are used to calculate the cTMB score and for the predictions of peptides used to calculate the cNEO score. Additionally, the clonal variants should have the highest raw allele fraction in the sequencing data and therefore should be detected with the highest reproducibility.

For each patient the clonal variants that were detected in the tumor were compared to those found in the Vigil sample. Except for the two samples with ITH >0.95 which had a low number of clonal variants, 82.2% or more of the clonal variants were also detected and classified as clonal in the paired Vigil sample. The search for the tumor clonal variants was expanded to include both clonal and non-clonal variants in Vigil. Regardless of ITH score, a minimum of 88.9% of the clonal tumor variants were detected in Vigil as clonal or non-clonal. In five out of 9 patients 100% of the clonal variants detected in tumor were found in the Vigil sample as either clonal or non-clonal (Table 8, see Supplemental Excel file 2-Clonal-variants-tables to view individual clonal variants detected).

TABLE 8
Clonal variants agreement between tumor and Vigil by percentage

| | | | % Clonal | % Clonal | | |
|---|---|---|---|---|---|---|
| | | | tumor | Vigil Variants | | |
| | % Clonal | % Clonal | variants | found in | | |
| | tumor | Vigil variants | found in Vigil | tumor (as | | |
| | variants | found in | (as clonal or | clonal and | | |
| Patient ID | found in Vigil | tumor | non-clonal) | non-clonal) | Mean ITH | Mean cTMB |
| 100_0067 | 97.0% | 100.0% | 100.0% | 100.0% | 0.864 | 0.891 |
| 102_0003 | 92.6% | 85.1% | 100.0% | 100.0% | 0.808 | 1.947 |
| 103_0009 | NA | 0.0% | NA | 100.0% | 0.992 | 0.069 |
| 136_0015 | 96.1% | 98.7% | 98.7% | 98.7% | 0.753 | 2.085 |
| 139_0010 | 83.3% | 90.9% | 95.8% | 100.0% | 0.907 | 0.631 |
| 178_0007 | 100.0% | 96.1% | 100.0% | 98.0% | 0.769 | 1.371 |
| 178_0011 | 82.2% | 97.4% | 88.9% | 100.0% | 0.831 | 1.138 |
| 180_0002 | 63.6% | 46.7% | 100.0% | 100.0% | 0.975 | 0.357 |
| 197_0001 | 93.3% | 97.7% | 100.0% | 98.8% | 0.781 | 2.414 |

Bolded cells indicate lower versus higher agreement in variants detected between the tumor and Vigil sample types for each patient. Mean cTMB and ITH scores, calculated by averaging the values for tumor and Vigil from a patient, are underlined for patients with ITH >0.95 where assignment of variant to the primary clone was less reliable on a percentage basis. Note that no clonal variants were detected in patient 103_009 and therefore they could not be matched to those detected in Vigil. Supporting tables and primary data are provided in Supplemental Excel File 2.

The results were even better in the reverse comparison, where the clonal variants detected in Vigil were compared to those detected in tumor for each patient. Again except for the two patients with ITH >0.95, a minimum of 88.9% of the Vigil clonal variants were detected and classified as clonal in the matching tumor sample from the matching patient. Ninety-eight percent or higher of the Vigil clonal variants were detected and classified as either clonal or non-clonal in the tumor sample, and in most patients there was a 100% match (Table 8).

The results indicate that the detection of the variants by the wet lab and bioinformatics system was very reliable but that the assignment of variants to the primary clone or a subclone by PyClone 6.1 was subject to a greater level of variability. Detailed supporting data and the final PyClone reports which contain the primary data used for all analyses can be reviewed in Supplemental Excel File 2.

### Prediction of Peptide Neoantigen Sequences

The pVAC-SEQ component of the pipeline takes the variant list generated by Mutect2 and generates the protein sequence spanning each variants, and then generates the peptide sequences of varying lengths (default 8-11-mers) that span the variant amino acid sequence [21]. The peptides are then scored based on their binding affinity (IC50) to the patient's own MHC molecules for which the haplotypes were predicted by HLA*LA from the sequencing data, and the binding affinity for the variant peptide sequence is compared to that for the wild-type peptide sequence derived from the same genomic coordinates. Best peptides are selected and reported in the final primary data as the variant-derived peptide with the highest binding affinity (lowest IC50) for the patient's predicted MHC-I molecules where the binding-affinity of the variant-derived peptide is stronger than the wild-type binding affinity. These high scoring peptides for the method qualification experiment are reported for each patient in the ‘Best-peptides-table’ of Supplemental Excel File 2.

Since the matching normal sample was used to determine the HLA haplotype for both the tumor and Vigil samples of a patient, it would be expected that the top variant for a given peptide sequence would be consistent regardless of sample origin. As expected, for all clonal variants detected in both sample types, the predicted best peptides were identical between the tumor and Vigil samples of the same patient. The result confirms that the pVAC-Seq portion of the pipeline as well as the reporting of the results are reliable.

### Sub-Sampling Analysis

Excluding the tumor sample for patient 102_0003 that had only a 535× mean coverage, the minimum mean target coverage depth in the sequencing run described above was 853× for tumor and Vigil samples and 110× for normal (i.e. PBMC and non-tumor control) samples after UMI processing (Supplemental Excel file 1). However, in a large study, due to variability in pooling and sample quality it is expected that some tumor and normal samples would have as low as 650× and 80× mean target coverage depth. Re-analysis of sub-sampled FASTQs from the original sequencing run was therefore undertaken in order to establish that the reduction in coverage depth to these limits would not impact the reliability of determination of cTMB, cNEO and ITH values.

Reads were randomly subsampled to a mean target depth, before UMI processing, of 940× and 100× for tumor/Vigil and PBMC/normal samples, respectively in order to achieve coverage depth after UMI processing and duplicate removal of around 650× for the tumor and Vigil samples and 80× for the PBMC and normal samples. The actual coverage depth achieved after UMI processing and duplicate removal ranged from 665-727× (mean 686×) for the tumor (except 1 outlier) and Vigil samples and 78-83× (mean 81×) for the PBMC and normal samples (Supplemental Excel file 3).

The cTMB, cNEO and ITH scores were compared between the processed results of the original and subsampled data files (Supplemental Excel file 4). Tumor samples from all 9 patients plus the tumor control and 7 Vigil samples showed differences in cTMB, cNEO and ITH of 20% or less between the original analysis and the subsampled data (Table 9), demonstrating that a coverage depth of greater than or equal to 665× for tumor samples and 78× for PBMC or normal samples is sufficient to deliver accurate metrics for the tumor samples. However, the two Vigil samples with the highest mean ITH and the lowest mean cTMB showed large percentage differences in cTMB and cNEO between the original and sub-sampled data (Table 9). The results suggest that when the ITH is very high (e.g. >0.95) the reproducibility of the measurements are more sensitive to reduction in coverage depth.

TABLE 9
Comparison of cTMB, cNEO and ITH scores between original and sub-sampled data

| | ITH | | | | cTMB | | | | cNEO | | | |
|---|---|---|---|---|---|---|---|---|---|---|---|---|
| Patient/ | Tumor | | Vigil | | Tumor | | Vigil | | Tumor | | Vigil | |
| Sample ID | 934X | 686X | 934X | 686X | 934X | 686X | 934X | 686X | 934X | 686X | 934X | 686X |
| 100_0067 | 0.87 | 0.86 | 0.86 | 0.85 | 0.91 | 0.91 | 0.88 | 0.82 | 2.25 | 2.25 | 2.17 | 2.14 |
| 102_0003 | 0.81 | 0.80 | 0.81 | 0.79 | 1.87 | 1.95 | 2.03 | 1.89 | 3.43 | 3.51 | 3.95 | 3.46 |
| 103_0009 | 1.00 | 1.00 | 0.98 | 0.95 | 0.00 | 0.00 | 0.14 | 0.33 | 0.00 | 0.00 | 0.33 | 0.77 |
| 136_0015 | 0.73 | 0.70 | 0.78 | 0.74 | 2.11 | 2.08 | 2.06 | 2.06 | 5.35 | 5.35 | 5.24 | 5.24 |
| 139_0010 | 0.91 | 0.88 | 0.91 | 0.90 | 0.66 | 0.63 | 0.60 | 0.55 | 0.85 | 0.77 | 0.74 | 0.69 |
| 178_0007 | 0.77 | 0.76 | 0.77 | 0.77 | 1.34 | 1.34 | 1.40 | 1.29 | 2.83 | 2.83 | 2.93 | 2.77 |
| 178_0011 | 0.79 | 0.76 | 0.87 | 0.88 | 1.23 | 1.23 | 1.04 | 0.99 | 3.21 | 3.21 | 2.77 | 2.66 |
| 180_0002 | 0.98 | 0.98 | 0.97 | 1.00 | 0.30 | 0.25 | 0.41 | 0.03 | 0.85 | 0.71 | 1.56 | 0.08 |
| 197_0001 | 0.79 | 0.76 | 0.77 | 0.77 | 2.47 | 2.52 | 2.36 | 2.30 | 3.98 | 4.03 | 3.65 | 3.57 |
| NCI-H2126 | 0.25 | 0.24 | | | 16.81 | 16.65 | | | 37.52 | 37.00 | | |

Bolded and underlined cells show samples where there is more than a 20% difference between the original and subsampled data.

### Discussion

A complete laboratory and bioinformatics workflow has been developed in order to rapidly and accurately identify tumor-associated variants within coding regions of the genome, determine the allelic copy number for chromosomal regions spanning the exome, and to sort the variants into clusters based on common CCF. The bioinformatics pipeline also further incorporates functions to perform MHC-1 HLA typing and prediction of potential peptide sequences spanning tumor-associated variant sequences and uses this information to generate a list of potential neoantigen peptides based on predicted binding affinity of the patient's MHC-1 molecules to the variant-associated peptides. Together, this information can be used to generate detailed variant-level reports for each tumor sample that provide information about CCF, protein-impacted, the type of mutation, allelic frequency, and associated likely peptide neoantigens for each variant. A summary report is also generated which contains the calculated cTMB, cNEO and ITH scores for each tumor sample represented in the sequencing run.

Application of this workflow revealed impressive similarity of clonal signal involving all parameters (cTMB, cNEO, ITH) including most notably cTMB between Vigil product and actual autologous tumor tissue used to construct Vigil. Generation of results for both wet-lab and bioinformatics pipeline, for matched tumor, Vigil, and PBMC (normal) samples from 9 ovarian cancer patients that participated in the VITAL trial of Vigil (CL-PTL-119) [31], demonstrated the robustness of the platform as evidenced by correlation coefficients (R²) between Vigil product and autologous tumor sample used to construct Vigil of 0.9802, 0.9525, and 0.8678 for cTMB, cNEO, and ITH, respectively. The high percentages of the tumor exome covered at a minimum of 250× or 500× and PBMC/normal exome covered at 50× suggested that the lab and bioinformatic processing procedures were suitable to achieve a clinical diagnostic goal of being able to consistently detect variants present at a CCF of 25% or higher in biopsy samples containing 20% tumor content. The high-level of cross correspondence of clonal non-synonymous variants detected in the same patient between tumor and Vigil samples further demonstrated the platform reliability for clinical application.

The laboratory and bioinformatic process steps implemented followed closely the steps originally elucidated by McGranahan and colleagues [2,3,18]. However, several modifications were made which were expected to improve the accuracy and throughput of the method. The utilization of the Twist Biosciences exome capture technology is expected to provide better exome coverage uniformity than the widely used Agilent exome capture technology [27], allowing for more comprehensive detection of variants. The use of oligonucleotide adapters containing UMIs during the library preparation and the utilization of the UMI information to collapse duplicate reads into consensus reads is expected to improve the reliability of the base calls and the accuracy of the determined allele fraction for variants [28]. However, at the current sequencing depth, only about 35% of the reads were removed as duplicates during UMI processing using the fgbio software suite, suggesting that a maximum of 35% of the collapsed consensus sequencing reads contain information from more than one parent read. Greater advantages of using UMIs can be achieved as gross sequencing depth increases.

Besides the introduction of UMI processing, several changes, relative to the McGranahan study, were made to the bioinformatic pipeline that are expected to improve the reliability of the cTMB, cNEO and ITH scores. Prior to utilization for variant calling and copy number determination, base quality scores were re-calibrated and the BAM file was filtered to only retain sequence reads overlapping the exome target. For variant calling, we used the combination of GATK Mutect2 and FilterMutectCalls, versus the McGranahan study which used the intersection of variants called by Mutect version 1.1.4 plus Varscan2 to select true variants. The sensitivity of GATK Mutect2 compared favorably to several alternative variant callers including VarScan2 [18,29], and the combination of GATK Mutect2 and FilterMutectCalls allowed for precise identification and removal of germline variants and variants found in a panel of normal as well as false-positive variants resulting from sequencing artifacts [30]. FACETS was utilized in the bioinformatic pipeline for allelic copy number analysis because it was shown to be one of the best performing allele-specific copy number analysis tools specifically designed for sequencing data as compared to ASCAT, the tool that was used by McGranahan and colleagues, which was originally designed for microarray data [18,20]. PyClone 6.1 also replaced the original PyClone version 0.12.7 used in the earlier study, with the main advantage being processing speed and a built-in capability to produce reliable results for studies with only a single tumor sample per patients [15]. Similar substitutions in tools to characterize the clonal architecture of tumor cells have been made by other researchers in recent studies. For example, a combination of GATK Mutect2 and Strelka v 2.9.10 for variant calling, an improved HLA haplotyping tool (Optitype), and an improved MHC-I peptide binding affinity prediction tool (MHC Flurry 2.0.4) were used to identify a correlation between higher cTMB and higher cNEO and responsiveness of urothelial cancer to atezolizumab therapy [1].

Our approach to identification of potential peptide neoantigens was to use the patient's own MHC-I haplotype calls generated by HLA*LA software, which was demonstrated to be one of the most accurate and efficient software tools of its class [25], combined with variant calls from Mutect2/FilterMutect2Calls as input data for pVACSeq version 4.0 which is able to generate the sequences for all non-synonymous variants associated peptides of length 8-11 amino acids and has the built-in capability to utilize 8 different MHC Class I algorithms for peptide-MHC-1 binding affinity predictions [21]. The count of high scoring peptides for each variant as well as the top scoring peptide sequences were then associated, based on the variant nucleotide change and genomic location, to the matching variants in the PyClone report in order to stratify the data as clonal/non-clonal enabling the automatic calculation of a cNEO score for each tumor sample. The suitability of this approach was demonstrated by the significant difference in cNEO scores between advanced melanoma patients who responded and failed to respond to Ipilimumab treatment.

This bioinformatics pipeline was benchmarked using three different published datasets. Variant calling, determination of allelic copy number, and prediction of CCF for each variant were verified using synthetic NGS data generated and analyzed by the authors of a subclonal deconvolution benchmarking study [18]. The accuracy of MHC-I and MHC-II typing was determined using whole exome NGS data from 99 patient whole blood samples that were generated as part of the 1000 genomes project and compared to a truth set compiled by the authors of an HLA typing benchmarking study [25]. Finally, publicly available whole exome NGS data was used to verify the integrity of the entire non-UMI version of the pipeline by comparing cTMB and ITH values generated to those published by the authors of a study that identified a significant association in response to nivolumab in melanoma patients to cTMB and ITH values [4].

The analysis of HLA typing results for 99 patients and comparison of the MHC-I and MHC-II haplotype calls to the published truth set showed an overall 91.5% match between the implemented HLA haplotyping module and a truth set [25,26], and a 97.4% match to either the truth set or the results from a previously published comparison of HLA haplotype calling tools [25].

Analysis of the simulated tumor sequencing data from Tanner et al showed that the PPV [calculated as TP/(TP+FP)] of variant calls by reference to the published truth set, was >97.5% at an SNV and InDel density in the exome of 1×10⁻⁵ and 2×10⁻⁶, respectively. The PPA which was calculated as TP/(TP+FN) and is equivalent to the analytical sensitivity, was at least 96.5% for all samples when considering variants with a known CCF of at least 0.5, and this PPA value could be adjusted to 100% if variants that were filtered out of the final data table by Mutect2 Filter were removed from consideration as true positives. The performance of the allelic copy number determination and CCF estimation by FACETS and PyClone 6.1, respectively, as part of the pipeline, was also deemed acceptable. Based on tumor sequencing depth of 250× in the Tanner data and the established relationship between coverage depth and variant detection, similar PPV and PPA metrics should be able to be achieved for lab sequencing data with a tumor genome coverage depth of 500× or higher for variants with known CCFs as low as 0.25 in samples containing as low as 20% tumor content.

Processing of publicly banked raw sequencing data, from a 2015 study of the relationship between genomic biomarkers and responsiveness of melanoma tumors to nivolumab treatment, through the entire non-UMI version of the bioinformatics pipeline generated cTMB and ITH values that were highly correlated to the published values [4,18,25]. Also consistent with the publication's conclusion, patients with partial response or a complete response to nivolumab, as compared to patients with stable disease had significantly higher values of cTMB and cNEO scores calculated by our pipeline.

The current results, particularly with respect to the re-analysis of the Riaz et al Ipilimumab-response correlation to cTMB and cNEO levels, reinforce the potential utility of cTMB and cNEO as separate possibly related markers for likely responsiveness of a wide variety of cancers to immunotherapy [1-4,32]. Immune checkpoint inhibitors and Vigil share a common biological mechanism of promoting the destruction of tumor cells. However, immune checkpoint inhibitors lack a key component of inducing an optimal anticancer immune response that Vigil provides. As we now demonstrate with preservation of clonal signals between the growing tumor of the patient and the Vigil product, Vigil provides clonal signals of cTMB and cNEO, which have the capacity to induce expansion of circulating clonal neoantigen-specific cytotoxic T-cells [7,33-35]. We hypothesize that Ovarian cancer patients with higher cTMB and/or cNEO levels may show greater responsiveness to Vigil particularly within the HRP subpopulation in which stable DNA repair activity will minimize expansion of subclonal neoantigen profiles. High cTMB and/or cNEO can be optimized with low subclonal neoantigen expression (low ITH) [7,36-39]. The current results also demonstrate that the wet lab and bioinformatics pipeline for generation of cTMB, cNEO, and ITH scores can be reliably used to investigate the relationship between overall survival and in particular cTMB levels for patients involved in the CL-PTL-119 VITAL trial who have been prospectively followed since randomization to Vigil or placebo as well as additional assessment of patients undergoing checkpoint inhibitor therapy and/or other immune therapies. It is also of interest to understand if targeting on clonal neoantigen targeting of various neoantigen based vaccines and/or CAR-T approaches can be improved via use of bioinformatic pipeline assessment to determine cTMB, cNEO and ITH.

### Methods

### Sample Selection and DNA Extraction

A set of 9 patients were selected from among 91 patients that participated in the CL-PTL-119 VITAL trial of Vigil in stage III/IV ovarian cancer patients [31]. Patients were selected to adequately represent the previously studied molecular profiles of all patients in the study, including representation from BRCA wildtype, BRCA mutant, homologous recombination proficient (HRP) and homologous recombination deficient (HRD) patients.

Genomic DNA was previously isolated, in 2019 and 2020, from cryopreserved tumor cell suspensions and matched normal peripheral blood mononuclear cells (PBMCs) samples of the 9 patients using the Qiagen MagAttract HMW DNA Kit. Genomic DNA was also extracted from cryopreserved Vigil preparations for each of the 9 patients using the Perkin Elmer Chemagic360 instrument according to the manufacturer's instructions. After extraction, the gDNA was quantified by UV spectrophotometry and checked for integrity by electrophoresis on 0.8% agarose gels. DNA was stored at −80C except when removed on ice to be used in library preparation procedures.

Matched genomic DNA samples isolated from a non-small cell lung cancer cell line (NCI-H2126) and a B lymphoblast cell line (NCI-BL2126) derived from the same patient were obtained from the American Tissue Culture Collection (ATCC) The TMB for NCI-H2126 had been previously characterized [9] and could be compared to the value obtained from our method.

### Whole Exome Sequencing

DNA sequencing libraries for the 27 patient and 2 control samples were prepared from 50 nanograms of gDNA using the Twist Exome 2.0 kit following the kit instructions except that Twist UMI adapters were used instead of Twist Universal Adapters. Prior to hybridization-based enrichment of exome-containing DNA fragments, libraries were pooled in batches of 6-8 such that the concentration of each DNA library derived from PBMC samples was 10.64% of the concentration of tumor or Vigil DNA libraries in the same pool. The hybridization utilized the Twist exome v2.0.2 baits which covers a 36.46 Mb human exome. DNA library pools were shipped to Discovery Life Sciences for sequencing on the Illumina NovaSeq X instrument to obtain 552+/−35 million reads (mean +/−standard deviation) for each tumor or Vigil sample and 65+/−5 million reads for each PBMC or normal control sample. The read format was paired-end, 150 nucleotides each.

### Bioinformatics Analysis

### Bioinformatics Pipeline Framework

The key elements of the pipeline framework include a primary python script, a config file, and sample information file. The config file contains settings for the desired bioinformatics steps to be performed in the run, as well as the settings, and paths to annotation files needed for each step. The sample information table is a tab-delimited list of the sequencing data identifiers that links each identifier to a specific patient ID and its tumor status (Tumor or NAT). The primary python script generates tables from the configuration file and sample information file, identifies the bioinformatic steps to be performed and passes the configuration and sample information to secondary python scripts that manage each major bioinformatic processing step. The secondary scripts read the sample information and config file, write linux shell scripts for each sample or tumor/normal pair, and launch the shell scripts as SLURM jobs on compute nodes of a cluster. Each submitted shell script launches a Docker environment that contains the appropriate executables and dependencies for each step.

### Sequencing Read Preparation

Sequencing reads were trimmed with Trimmomatic version 0.39 to remove Illumina adapter sequences and low quality bases (Q<25) from 3′-ends. Additionally, non-template encoded nucleotides were removed from 5′-end of sequencing reads obtained from public non-UMI containing sequencing data used for pipeline verification studies but not for the UMI-containing reads generated during this study.

Generation of BAM Files from Non-UMI-Containing Sequencing Data

Trimmed sequence reads were aligned to the hg38 genome with bwa-mem and then sorted using Samtools sort. Duplicate reads were marked with Picard MarkDuplicates. Coverage metrics were generated with Picard HsMetrics. Read group notation was then added to the BAM files using GATK AddOrReplaceReadGroups, and the base quality scores were recalibrated using GATK BaseRecalibrator and ApplyBQSR. Following base score recalibration, the BAM files were cropped to contain only sequences covered by the exome using SamTools View and the corresponding bed file for the exome. Finally, the previously marked duplicate reads were removed using Picard Markduplicates.

Generation of BAM Files from UMI-Containing Sequencing Data

Trimmed sequencing reads contain UMIs that were processed using fgbio software according to the recommended Best Practices to generate consensus reads, remove duplicates, and align the consensus reads to the genome. The process steps comprised in order:
(1) creating BAM files containing unaligned reads with the UMI sequences removed and stored in tags using fgbio FastqToBam given the read structure “5M2S+T 5M2S+T”,
(2) aligning reads to the hg38 reference genome using bwa-mem2,
(3) sorting the reads using Samtool sort and then recovering headers and tags for unmapped reads using fgbio ZipperBams,
(4) generating initial coverage metrics using picard CollectHsMetrics,
(5) Grouping of reads based on identical UMIs sequences using fgbio GroupReadsbyUMI,
(6) calling consensus reads based on UMI sequence and mapped position with fgbio CallMolecularConsensusReads, based on a minimum of 1 read and a minimum base quality of 20,
(7) aligning these consensus reads to the hg38 reference genome using bwa-mem2,
(8) sorting the alignments generated from the consensus reads using Samtools sort,
(9) recovering headers and UMI tags with fgbio ZipperBams,
(10) filtering mapped consensus reads to select reads based on at least 1 consensus reads having a minimum base quality of 20 and maximum base error rate of 0.2 using fgbio FilterConsensusReads, and finally,
(11) generating an additional set of coverage metrics for the mapped consensus reads.

Following completion of the fgbio best practices processing steps, the alignment files containing the consensus reads were further processed through base quality score recalibration and cropping to retain only exome sequences as described above for the non-UMI-containing sequencing data. Note that during the alignment steps using bwa-mem2, alignments were piped directly to Samtools view and compressed to binary format using that tool in order to save disk space.

### Preparation of BAM Files for HLA Haplotype Calling

Trimmed raw unaligned sequence reads were converted to BAM files using fgbio FastqToBam. Samtools merge was used to combine these unmapped reads with the BAM files containing aligned, base quality score recalibrated, and exome-cropped alignments. In the case of non-UMI containing reads the BAM files containing aligned reads had duplicates marked but not removed. In the case of UMI-containing reads, FastqToBam was given the read structure “5M2S+T 5M2S+T”, and the BAM files containing aligned reads used were the final output of the processing sequence described above for UMI-containing sequence reads. Merged BAM files were indexed with Samtools index. Note that the sequencing data from normal samples, and not tumor samples is utilized for HLA haplotype calling.

### HLA Haplotyping

The bioconda package for HLA*LA along with the data package PRG_MHC_GRCh38_withIMGT were downloaded and installed per the ReadMe.md file at https://github.com/DiltheyLab/HLA-LA/. Merged BAM files, generated as described above were used as input for HLA*LA in order to generate haplotypes for each allele of the HLA Class I (MHC-I) loci A through G and the seven different HLA Class II (MHC-II) loci.

### Variant Calling

GATK Mutect2 was used to call tumor-specific SNVs and InDels in paired tumor/normal mode. Paths to the 1000 genomes hg38 panel of normals database and the AF-only gnomad hg38 germline resource were provided to Mutect2 in order to exclude variants that occur with some frequency in the healthy human population. GATK LearnReadOrientationModel was used to generate tables of orientation bias artifacts using the flr2.tar.gz files generated by Mutect2 during the variant calling. GATK FilterMutect2Calls was used to add the codes “PASS” or various error-mode codes to the INFO column of the VCF files generated by Mutect2 given an additional sample-specific input table of orientation bias generated by LearnReadOrientationModel. The filtered VCF files were annotated with GATK Funcotator.

### Allelic Copy Number Determination

Allele specific copy number was estimated for chromosomal segments comprising the majority of the non-telomeric and -centromeric long arm and short arm of each chromosome using FACETs v0.5.14 (Shen & Seshan, 2016). FACETs estimates and reports the start and stop position, the total copy number, and the minor allele copy number for each of those segments and estimates the overall tumor purity for the tumor sample (i.e. determines the level of contamination from normal tissue). The Docker image available for installation from https://github.com/vanallenlab/facets was used for this step according to the provided instructions. Briefly, single nucleotide polymorphism (snp)-pileup was used to prepare input files for FACETS using a table of the hg38 reference genome, a table of known snps (00-common_all_prefix.dict-hg38minimal.sort.vcf) and the tumor and normal BAM files as input using the options minimum map quality 15, minimum base quality 20, and pseudo-snps 100. The r_script.R script included in the Docker package was then run using the facets input built by snp-pileup using settings initial seed 1234, cval 250, min_nhet 15, specifying the hg38 genome build.

### Clonal Analysis Using PyClone Version 6.1

A proprietary code was developed to handle extraction and formatting of data from filtered Mutect2 VCF files and FACETs tabular output in order to build PyClone input files for each tumor sample. The information required for each variant was the mutation_id which contains the chromosomal position and sequence change, the allelic depth for the reference and variant sequence, the normal copy number, the major allele copy number, the minor allele copy number, and the overall tumor purity estimate for the sample. PyClone 6.1 [15], with settings of a maximum of 20 clusters and a total of 50 restarts was then used to generates output which includes the assigned cluster and the estimated CCF for each mutation_id. Additional proprietary code was developed to add additional information from the Mutect2 VCF and FACETS output tables to the PyClone 6.1 output tables to facilitate downstream steps and interpretation; key information added includes the variant annotation information derived from Funcotator, the coverage depth for each allele in both the tumor and normal sample, and the input information from FACETs. The finished table was termed the “preliminary PyClone variant-level report” (FIG. 7).

### Determination of Total TMB, Clonal TMB, and Intra Tumor Heterogeneity

PyClone 6.1 assigns each variant detected in a tumor to a cluster, each with an estimated CCF. The cluster-specific tumor mutational burden (TMB) was calculated as the number of non-synonymous polymorphisms (including missense, nonsense, nonstop, in-frame insertions, in-frame deletions, frameshifts, or changes in the start codon) assigned to the cluster by PyClone 6.1 divided by the length of the exome in Mb. The total TMB was calculated as the sum of the cluster-specific TMBs for a sample and the cTMB was defined as the TMB for the cluster with the highest CCF that is a minimum of 0.9. Tumor samples with no associated clusters having a CCF >=0.9 do not have a primary clone and therefore their cTMB is reported as 0. Intratumor heterogeneity (ITH), was calculated as total TMB-cTMB. A proprietary script was developed to calculate each of the above metrics and assemble them into a tab-delimited text format “clonal TMB report”.

### Prediction of Peptide Neoantigens

Unannotated, quality-filtered VCF files generated as described above using GATK Mutect2 and GATK FilterMutect2Calls were annotated using Ensembl VEP software using a hg38.fa file as reference. VEP software was implemented using the Docker ensemblorg/ensembl-vep available from Dockerhub according to the documentation provided by Ensembl, using the plugins Frameshift and Wildtype. Note that the INSTALL.pl script was used to pre-download and install the data cache and FASTA file for human GRCh38 along with the required plugins.

pVACSeq software was used to generate a list of peptide antigens along with estimated MHC-1 molecule binding affinity and fold-difference in binding affinity between mutant and wild-type peptides for each non-synonymous tumor-associated variant identified by Mutect2 and annotated with VEP. pVACSeq was installed by pulling the Docker image griffithlab/pvactools from DockerHub. pVACSeq was executed using the VEP-annotated VCF and a string containing the patient's predicted MHC-1 A, B, and C haplotypes, from HLA*LA software, as input using the following parameters: peptide length “8,9,10,11”, number of threads “10”, minimum fold change “1”, only process VCF rows with PASS in info, prediction algorithm “MHCflurry” [41]. pVAC-Seq generated all possible 8-11-mer peptide sequences that overlap with at least one variant amino acid associated with a non-synonymous variant as well as the corresponding peptides for the reference genome sequence and generated a predicted MHC-1 binding affinity (IC50) using the MHCflurry algorithm, the fold-change in binding affinity for reference versus mutant peptide, and a percentile rank for each peptide among all predicted peptides for a sample.

The pVacSeq filter command was used to filter the peptide list to select peptides with IC50<500 nM, a fold-change difference in IC50 for reference versus variant peptide of >1, and finally to select peptides falling among the lowest 5% of total peptides in Kb value (selected experiments only). A proprietary script was used to update the preliminary PyClone variant-level report to include the quantity of unique peptides predicted for each non-synonymous variant that were retained in the filtered list from pVacSeq Filter, and also the sequence of the mutant peptide with the lowest IC50 along with its' IC50 score and the score of the corresponding peptide derived from the reference sequence. This final variant-level PyClone report was filtered to include only nonsynonymous variants.

### Creation of Final Report Clonal Neoantigen Summary Report

A proprietary script was developed to update the original “clonal TMB report” produced as described above, to include summary metrics for the count of predicted neantigen peptides that met the filter metrics. A NEO score for each cluster was calculated by summing the filtered peptide count for all non-synonymous variants associated with the cluster. The cNEO value for the tumor/normal sample pair was then determined by dividing NEO for the primary clone by the size of the exome in Mb; a tumor with no primary clone will have a cNEO of zero.

Versions of software used in the pipeline: Trimmomatic 0.39, fgbio 2.1.0, bwa 0.7.17, bwa2 2.2.1, Samtools 1.18, picard 2.27.5, bedtools 2.31.1, gatk 4.3.0.0, HLA*LA 1.0.3, VEP 111.0, pVACseq 4.0.4, FACETS 0.5.14-2, PyClone 0.13.1.

### Pipeline Verification Using Published Data Sets

### Verification of HLA Haplotyping Module

Whole exome sequencing data from the 1000 Genomes Project was utilized for verifying the performance of the HLA Haplotyping module of the bioinformatics pipeline. Data from the first 99 individuals from a list of 820 patients that were analyzed previously with several HLA haplotyping tools were downloaded from the European Bioinformatics Repository, in CRAM format, using the links provided in Thuesen's GitHub repository (https://github.com/nikolasthuesen/hla-typing-benchmark/blob/main/reference_data/gold_standard_url_list.txt). The sequence files were converted from CRAM to FASTQ, aligned to the human genome using bwa-mem, and the output BAM files were further processed according to the flow chart provided in the supplementary materials of the Thuesen paper. The pre-processed BAM files, which contained both aligned and unaligned reads, were used as input to the bioinformatics module that utilizes the HLA*LA software. The output haplotypes for the HLA-A, HLA-B, HLA-C, HLA-DQB, and HLA-DRB genes were compared to haplotypes generated for the same 1000 Genomes patients using the same input exome sequencing data and PolyPheMe software [26]. The final Abi-Rached set of HLA-1 and HLA-2 haplotype calls for 2693 individuals in the 1000 Genomes Project were downloaded from Thuesen's GitHub archive

(https://github.com/nikolasthuesen/hla-typing-benchmark/blob/main/reference_data/2018_1129_HLA_types_full_1000_Genomes_Project_panel.txt). For the purposes of the comparison, if the truth set contained more than one potential haplotype for one or both alleles of an HLA locus, the data from the bioinformatics pipeline was considered correct if each of the two haplotypes identified were represented among the multiple options in the truth set.

Verification of Core Bioinformatics Pipeline Performance with Synthetic Sequencing Data.

The performance of the core portion of the bioinformatics pipeline, which included the overall pipeline framework, processing of raw sequencing data files (FASTQ) to optimized BAM files, detection of tumor-related variants (SNPs and InDels), allelic copy number estimation, and estimation of CCF and clonal assignment for each variant utilized to generate TMB, cTMB, and ITH scores, was evaluated using simulated tumor exome sequencing data [18]. Simulated sequencing reads from genomes S1R1, S2R1, and S2R3 that had been mixed in silico with normal healthy genomic DNA to generate mixtures having 100%, 75%, 50%, and 25% tumor DNA, a total of 12 simulated tumor sequencing data samples, were chosen for analysis. The mean target coverage depth was 250× for the tumor and 60× for the normal sample sequencing reads. The density of expected SNVs and InDels in simulated tumor genome SIRI was 1×10-6 and 2×10-7 respectively, while the density of SNVs and InDels was 10-fold higher in samples S2R1 and S2R3. The synthetic sequence reads were trimmed as described in the methods for the Tanner et al. publication, then processed as described above for non-UMI containing sequence data to generate base quality recalibrated, de-duplicated, and exome cropped BAM files. BAM files were then processed through variant calling, allelic copy number determination, clonal analysis and preparation of the clonal TMB report.

The PyClone report for each of the 12 simulated tumor DNA sequence samples from the Clonal Neoantigen pipeline run (described in section 5.4.6 above) was compared to a truth set available on Georgette Tanner's GitHub (https://github.com/GeorgetteTanner/benchmarking/) with respect to the expected versus determined tumor purity, the variants detected versus those known to be in the truth set, and the overall copy number for the chromosomal region spanning a variant versus the determined total copy number for that region. For the purposes of matching variants between the Clonal Neoantigen pipeline output and the truth set, variants in the truth set that were present in low coverage region of the genome, as represented by normal sample coverage of <30× were not considered as true positives. A +/−3 nt genomic starting position window was used to match the identified variants to the truth set because of difference in encoding of the genomic start position for some insertions and deletions between mutect2 and the truth set.

Evaluation of Complete Bioinformatics Pipeline Performance Using Published Data from Advanced Melanoma Patients

Evaluation of the complete bioinformatics pipeline was performed using publicly available sequencing data from advanced melanoma patients [4]. Illumina 75 nucleotide paired-end sequencing data, collected using the Agilent version 2 exome-kit, was downloaded from the NIH sequence repository (SRA: SRP095809; BioProject: PRJNA359359). Fourteen patients that had not previously received ipilimumab therapy were selected for analysis including all 7 non-pre-treated patients that showed a PR or CR to nivolumab and the first 7 patients which did not respond to nivolumab (progressive disease, PD). FASTQ files from both tumor and normal were processed through the non-UMI module for generation of quality-score recalibrated, de-duplicated, and exome-cropped BAM files, followed by all subsequent processing steps as described above under Methods. The mean coverage depth for the sequencing data was 150× for both tumor and whole blood genomic DNA.

cTMB and ITH values from the bioinformatics pipeline described in this publication were compared to values calculated from Riaz et al supplementary data. cTMB and ITH values for comparison were computed from the list of non-synonymous mutations detected and their cellular prevalence values (equivalent to CCF) reported on column 5 in the PyClone reports that were archived in a GitHub project (https://www.github.com/riazn/bms038_analysis) described in the publication. For the purposes of the comparison of values between Riaz et al. and internal results, variants with a CCF of >0.85 as reported in the Riaz PyClone data were considered primary clonal variants. Note that the current study utilized PyClone 6.1 versus the original release of PyClone used by Riaz et al. In each case, only non-synonymous variants were considered in the calculations. A two-tailed unpaired t test was applied to log-transformed cNEO values to compare PR/CR and PD groups.

### References for Example 3

(1) Boll L M, Perera-Bel J, Rodriguez-Vida A, et al. The impact of mutational clonality in predicting the response to immune checkpoint inhibitors in advanced urothelial cancer. Sci Rep. 2023 Sep. 15; 13 (1): 15287.
(2) Litchfield K, Reading J L, Puttick C, et al. Meta-analysis of tumor- and T cell-intrinsic mechanisms of sensitization to checkpoint inhibition. Cell. 2021 Feb. 4; 184 (3): 596-614 e14.
(3) McGranahan N, Furness A J, Rosenthal R, et al. Clonal neoantigens elicit T cell immunoreactivity and sensitivity to immune checkpoint blockade. Science. 2016 Mar. 25; 351 (6280): 1463-9.
(4) Riaz N, Havel J J, Makarov V, et al. Tumor and Microenvironment Evolution during Immunotherapy with Nivolumab. Cell. 2017 Nov. 2; 171 (4): 934-949 c16.
(5) Snyder A, Makarov V, Merghoub T, et al. Genetic basis for clinical response to CTLA-4 blockade in melanoma. N Engl J Med. 2014 Dec. 4; 371 (23): 2189-2199.
(6) Van Allen E M, Miao D, Schilling B, et al. Genomic correlates of response to CTLA-4 blockade in metastatic melanoma. Science. 2015 Oct. 9; 350 (6257): 207-211.
(7) Nemunaitis J, Stanbery L, Willoughby D, et al. Clonal Neoantigen: Emerging “Mechanism-based” Biomarker of Immunotherapy Response. Cancers (Basel). 2023 Nov. 28; 15 (23): 5616.
(8) Chalmers Z R, Connelly C F, Fabrizio D, et al. Analysis of 100,000 human cancer genomes reveals the landscape of tumor mutational burden. Genome Med. 2017 Apr. 19; 9 (1): 34.
(9) Vega D M, Yee L M, McShane L M, et al. Aligning tumor mutational burden (TMB) quantification across diagnostic platforms: phase II of the Friends of Cancer Research TMB Harmonization Project. Ann Oncol. 2021 December; 32 (12): 1626-1636.
(10) Leone P, Shin E C, Perosa F, et al. MHC class I antigen processing and presenting machinery: organization, function, and defects in tumor cells. J Natl Cancer Inst. 2013 Aug. 21; 105 (16): 1172-87.
(11) Wells D K, van Buuren M M, Dang K K, et al. Key Parameters of Tumor Epitope Immunogenicity Revealed Through a Consortium Approach Improve Neoantigen Prediction. Cell. 2020 Oct. 29; 183 (3): 818-834 e13.
(12) Hundal J, Carreno B M, Petti A A, et al. pVAC-Seq: A genome-guided in silico approach to identifying tumor neoantigens. Genome Med. 2016 Jan. 29; 8 (1): 11.
(13) Senzer N, Barve M, Kuhn J, et al. Phase I trial of “bi-shRNAi (furin)/GMCSF DNA/autologous tumor cell” vaccine (FANG) in advanced cancer. Mol Ther. 2012 March; 20 (3): 679-86.
(14) Nemunaitis J, Stanbery L, Walter A, et al. Gemogenovatucel-T (Vigil): bi-shRNA plasmid-based targeted immunotherapy. Future Oncol. 2024 Aug. 5:1-16.
(15) Gillis S, Roth A. PyClone-VI: scalable inference of clonal population structures using whole genome data. BMC Bioinformatics. 2020 Dec. 10; 21 (1): 571.
(16) Roth A, Khattra J, Yap D, et al. PyClone: statistical inference of clonal population structure in cancer. Nat Methods. 2014 April; 11 (4): 396-8.
(17) Creaney J, Ma S, Sneddon S A, et al. Strong spontaneous tumor neoantigen responses induced by a natural human carcinogen. Oncoimmunology. 2015 July; 4 (7): e 1011492.
(18) Tanner G, Westhead D R, Droop A, Stead L F. Benchmarking pipelines for subclonal deconvolution of bulk tumour sequencing data. Nat Commun. 2021 Nov. 4; 12 (1): 6396.
(19) Dilthey A T, Mentzer A J, Carapito R, et al. HLA*LA-HLA typing from linearly projected graph alignments. Bioinformatics. 2019 Nov. 1; 35 (21): 4394-4396.
(20) Shen R, Seshan V E. FACETS: allele-specific copy number and clonal heterogeneity analysis tool for high-throughput DNA sequencing. Nucleic Acids Res. 2016 Sep. 19; 44 (16): c 131.
(21) Hundal J, Kiwala S, McMichael J, et al. pVACtools: A Computational Toolkit to Identify and Visualize Cancer Neoantigens. Cancer Immunol Res. 2020 March; 8 (3): 409-420.
(22) Frank M S, Fuss J, Steiert T A, et al. Quantifying sequencing error and effective sequencing depth of liquid biopsy NGS with UMI error correction. Biotechniques. 2021 April; 70 (4): 226-232.
(23) Osterlund T, Filges S, Johansson G, Stahlberg A. UMIErrorCorrect and UMIAnalyzer: Software for Consensus Read Generation, Error Correction, and Visualization Using Unique Molecular Identifiers. Clin Chem. 2022 Nov. 3; 68 (11): 1425-1435.
(24) Petrackova A, Vasinck M, Sedlarikova L, et al. Standardization of Sequencing Coverage Depth in NGS: Recommendation for Detection of Clonal and Subclonal Mutations in Cancer Diagnostics. Front Oncol. 2019; 9:851.
(25) Thuesen N H, Klausen M S, Gopalakrishnan S, et al. Benchmarking freely available HLA typing algorithms across varying genes, coverages and typing resolutions. Front Immunol. 2022; 13:987655.
(26) Abi-Rached L, Gouret P, Yeh J H, et al. Immune diversity sheds light on missing variation in worldwide genetic diversity panels. PLOS One. 2018; 13 (10): c0206512.
(27) Yaldiz B, Kucuk E, Hampstead J, et al. Twist exome capture allows for lower average sequence coverage in clinical exome sequencing. Hum Genomics. 2023 May 3; 17 (1): 39.
(28) Kou R, Lam H, Duan H, et al. Benefits and Challenges with Applying Unique Molecular Identifiers in Next Generation Sequencing to Detect Low Frequency Mutations. PLOS One. 2016; 11 (1): c0146638.
(29) Maruzani R, Brierley L, Jorgensen A, Fowler A. Benchmarking UMI-aware and standard variant callers for low frequency ctDNA variant detection. BMC Genomics. 2024 Sep. 3; 25 (1): 827.
(30) Benjamin D, Sato T, Cibulskis K, et al. Calling Somatic SNVs and Indels with Mutect2. bioRxiv. 2019:861054.
(31) Rocconi R P, Grosen E A, Ghamande S A, et al. Gemogenovatucel-T (Vigil) immunotherapy as maintenance in frontline stage III/IV ovarian cancer (VITAL): a randomised, double-blind, placebo-controlled, phase 2b trial. Lancet Oncol. 2020 December; 21 (12): 1661-1672.
(32) Rizvi N A, Hellmann M D, Snyder A, et al. Cancer immunology. Mutational landscape determines sensitivity to PD-1 blockade in non-small cell lung cancer. Science. 2015 Apr. 3; 348 (6230): 124-8.
(33) Goleva E, Lyubchenko T, Krachenbuchl L, et al. Our current understanding of checkpoint inhibitor therapy in cancer immunotherapy. Ann Allergy Asthma Immunol. 2021 June; 126 (6): 630-638.
(34) Nemunaitis J, Senzer N, Plato L. Tumor vaccines and cellular immunotherapies. Ann Transl Med. 2016 October; 4 (Suppl 1): S24.
(35) Rocconi R P, Stanbery L, Madeira da Silva L, et al. Long-Term Follow-Up of Gemogenovatucel-T (Vigil) Survival and Molecular Signals of Immune Response in Recurrent Ovarian Cancer. Vaccines (Basel). 2021 Aug. 12; 9 (8): 894.
(36) Wolf Y, Bartok O, Patkar S, et al. UVB-Induced Tumor Heterogencity Diminishes Immune Response in Melanoma. Cell. 2019 Sep. 19; 179 (1): 219-235 c21.
(37) Birkbak N J, Kochupurakkal B, Izarzugaza J M, et al. Tumor mutation burden forecasts outcome in ovarian cancer with BRCA1 or BRCA2 mutations. PLOS One. 2013; 8 (11): c80023.
(38) Strickland K C, Howitt B E, Shukla S A, et al. Association and prognostic significance of BRCA1/2-mutation status with neoantigen load, number of tumor-infiltrating lymphocytes and expression of PD-1/PD-L1 in high grade serous ovarian cancer. Oncotarget. 2016 Mar. 22; 7 (12): 13587-98.
(39) Germano G, Lamba S, Rospo G, et al. Inactivation of DNA repair triggers neoantigen generation and impairs tumour growth. Nature. 2017 Dec. 7; 552 (7683): 116-120.
(40) McLaren W, Gil L, Hunt S E, et al. The Ensembl Variant Effect Predictor. Genome Biol. 2016 Jun. 6; 17 (1): 122.
(41) O'Donnell T J, Rubinsteyn A, Laserson U. MHCflurry 2.0: Improved Pan-Allele Prediction of MHC Class I-Presented Peptides by Incorporating Antigen Processing. Cell Syst. 2020 Oct. 21; 11 (4): 418-419.
(42) Gourraud P A, Khankhanian P, Cereb N, et al. HLA diversity in the 1000 genomes dataset. PLOS One. 2014; 9 (7): e97282.

### Example 4. Gemogenovatucel-T Maintenance Immunotherapy Demonstrates Overall Survival Advantage in Frontline Stage III/IV Homologous Recombination Proficient Ovarian Cancer with High Clonal Tumor Mutation Burden

### Overview

A subset of patients with newly diagnosed Stage IIIb/IV ovarian cancer and homologous recombinant proficient (HRP) molecular profile do not have therapeutic options that affect overall survival (OS) and are considered an unmet medical need population. Gemogenovatucel-T is a triple function targeted cellular immunotherapy. Previously, we demonstrated proof of principle clinical benefit of maintenance treatment with gemogenovatucel-T compared to placebo in an ad hoc assessment of the HRP subset of patients following debulking surgery and adjuvant/neoadjuvant platinum-based chemotherapy in the double blind randomized VITAL trial. Retrospective evidence supports the relationship of clonal tumor mutation burden (cTMB), which is the density of mutation present in all cells of a tumor, to OS advantage in non-ovarian cancer patients receiving immunotherapy. We hypothesized that patients with stable DNA repair, which is associated with the HRP phenotype, would contain clonal mutations that were stably maintained in all tumor cells and furthermore that the subpopulation of HRP patients with high cTMB would show greater responsiveness to gemogenovatucel-T and a corresponding OS advantage. We employed whole exome sequencing and analysis with a proprietary, clinically adapted, bioinformatic pipeline to analyze the DNA of 91 patients entered into VITAL trial undergoing continued long term follow up in order to determine cTMB levels and examine the association of cTMB with OS in the HRP subpopulation. We now demonstrate through prospective evaluation, for the first time, that a subpopulation of HRP patients with high cTMB receive a robust OS benefit from gemogenovatucel-T. The primary objective was achieved as evidenced by an OS of 68 (30.2, not achieved, 95% CI) months with gemogenovatucel-T vs. only 19 (15.6, not achieved, 95% CI) months with placebo from time of randomization in the high cTMB/HRP subpopulation (HR=0.23, p=0.008). Preplanned restricted mean survival time estimate of OS was 63 (48-78) months with gemogenovatucel-T vs. 33 (18-47) months with placebo, p=0.007 and analysis from procurement showed OS of 75 (38.6, NA, 95% CI) months with gemogenovatucel-T vs. 26 (21.3, NA, 95% CI) months with placebo from procurement (HR=0.23, p=0.008). No Grade 3 treatment related toxicity was observed. In conclusion, results support a role for gemogenovatucel-T as maintenance therapy in newly diagnosed HRP/cTMB profile Stage IIIb/IV ovarian cancer patients who are of unmet medical need.

### Introduction

Standard of care for newly diagnosed ovarian cancer (Stage IIIb/IV) involves debulking surgery and adjuvant chemotherapy with paclitaxel and carboplatin [1, 2]. Although the majority of patients achieve complete remission, up to 75% will relapse within 2 years and 5-year survival is poor at 11 to 25% [3]. A number of studies have attempted to improve outcome in frontline treated advanced stage ovarian cancer by administering maintenance therapy after patients achieve complete response despite benefit in progression free survival (PFS), none have demonstrated significant advantage in overall survival (OS) [4-7].

PARPi's have offered clinicians a novel platform for frontline maintenance, but activity is predominantly seen in patients with BRCA-1,-2 germline and/or somatic mutations and or/a homologous recombinant deficient (HRD) profile [4]. Niraparib is the only PARPi approved for patients with Stage IIIb/IV newly diagnosed disease during maintenance therapy regardless of BRCA-1,-2 status; however, the magnitude of benefit for niraparib is greatest in patients with tumors containing a BRCA-mutation (BRCA-m) or evidence of a HRD molecular profile [5]. Patients receiving niraparib for maintenance with homologous recombinant proficient (HRP) type tumors demonstrate no to minimal clinical benefit and show no OS advantage (overall HR=1.01; HRD HR-0.95, HRP HR=0.93) with follow up at 74 months [6, 8]. Interestingly, patients with HRP tumors compared to HRD or BRCA-m tumors demonstrate a worse prognosis “across the board” when treated with standard of care regimens involving only chemotherapy, angiogenesis inhibitors and/or PARPi's [7, 9]. Prospective study by Ni et al. also revealed, poor response by the HRP profile subset compared to non-HRP. Also, results from the GOG-0218 study retrospectively evaluating impact on OS of 1873 patients receiving bevacizumab, demonstrated nearly 20 months lower survival in patients with HRP tumors (43.4 months) when compared to the patient subtype with BRCA-m and HRD pattern disease (62.6 months), supporting the need to improve therapies in HRP ovarian cancer patients receiving bevacizumab and highlighting evidence of unmet medical need in the HRP population [7].

Limits of use of bevacizumab and/or niraparib maintenance therapy also relate to significant drug related toxicity [5, 10]. Specifically, over 65% of patients who are treated with niraparib or bevacizumab develop Grade 3/4 product related adverse events, necessitating dose modification or discontinuation.

Extensive trials with checkpoint inhibitor therapy and most recently combination checkpoint inhibitor therapy with PARPi therapy also show no clinical benefit in this subpopulation. This is highlighted by the recent results of the ATHENA COMBO trial which investigated combined maintenance with both nivolumab and the PARP inhibitor rucaparib in newly diagnosed advanced high-grade ovarian cancer patients. The combination failed to demonstrate PFS advantage compared to rucaparib alone (HR=1.3, 15.0 vs. 20.2 months). The subpopulation of BRCA-wild type (BRCA-wt)/loss of heterozygosity (LOH) low patients demonstrated worse PFS (HR=1.3, 11.0 vs. 12.1 months) [8]. Additionally, OS was found to be unchanged with rucaparib when combined with nivolumab (HR=1.29) compared to rucaparib alone [8]. Similarly, in the IMagyn050 trial of atezolizumab, paclitaxel, carboplatin and bevacizumab versus placebo, paclitaxel, carboplatin, and bevacizumab, no PFS advantage was demonstrated in the overall population or HRP subgroup (HR=0.82) and the trial was stopped early [11]. Moreover, no evidence of OS benefit was observed [8, 11].

Optimal immune response relates to generation and preservation of systemic populations of activated immune effector cells with the capacity to target unique tumor signals present on all cancer cells and not evident on normal cells. Vast preclinical and retrospective clinical evidence supports immune targeting of clonal mutations. Presentation of peptides derived from clonal mutations in the tumor on the cell surface in complex with MHC-I has the potential to promote and generate optimal immune response [12-19]. Clonal mutations are the progenitor mutations that first occur during the transition of cells from “normal to cancer” [20, 21] and are stably maintained in all cancer cells. The density of these clonal mutations per Mb of DNA is the clonal tumor mutational burden (cTMB) [22]. These mutations are patient specific and are identified on the initial cancer cell as well as all subsequent metastatic lesions [23]. Retrospective studies have demonstrated high clonal TMB (cTMB-H) correlation with OS advantage in early stage cancer patients and advanced cancer patients receiving immunotherapy [12-15, 18, 22, 24]. Assessment by Litchfield of 1008 cancer patients involving multiple cancer histologies receiving immunotherapy revealed correlation of high clonal TMB with marked OS advantage (p=0.00000013).

In comparison within the same analysis high subclonal TMB demonstrated no correlation at all to OS [22]. Preclinical assessment by Wolf et al [25], support importance of clonal signals and actually lack of benefit to subclonal signals. They demonstrated correlation of decreased OS and immune response in murine models induced to higher subclonal neoantigen expression (discussed in his work as high intratumoral heterogeneity, ITH) compared to lower subclonal neoantigen expression [25]. Studies have also been done to induce T cell targeting against subclonal neoantigens and results have been lacking for significance of clinical benefit [14-18, 22, 25, 26].

Previously, we published benefit with gemogenovatucel-T in Stage III/IV ovarian cancer as maintenance therapy in the BRCA-1/2-wt and HRP subgroups [27-29]. Functionally, BRCA-1/2-wt with HRP profile provide stable cancer cell DNA repair function. Conversely, in ovarian cancer patients, a BRCA-1/2 mutant genotype and/or an HRD molecular profile is associated with poor DNA repair capacity, which has been associated with an increase in subclonal neoantigens. Evidence supports that a high subclonal fraction created with a poor DNA repair environment (HRD, BRCA-1/2 mutation) will diminish immune editing capability, dendritic cell response and will reduce relevant reactive antigen specific T cell activity, thereby enhancing antitumor blockade and T cell exhaustion [18, 19, 25, 30-32].

Gemogenovatucel-T [33-35] is an intradermally administered DNA engineered immunotherapy utilizing autologous tumor cells as a source of the full matrix of a patient's tumor-related clonal signals. Construction of gemogenovatucel-T involves surgical harvest of autologous tumor tissue followed by transfection with a DNA plasmid containing two genetic therapeutic modifications in order to promote a “triad” of molecular functionality in the transfected tumor cells. Specifically, treatment with gemogenovatucel-T promotes i) patient tumor-specific clonal neoantigen presentation, ii) dendritic cell activation (via plasmid expression of GMCSF), and iii) tolerance escape (via plasmid generated interference of TGF-β1, -β2 activation consequent to reduction of expression of the furin cleavage enzyme) [36, 37]. To construct gemogenovatucel-T, autologous cancer cells are harvested as part of standard of care. These cancer cells contain stably maintained cancer initiating mutations involved in the transformation of normal to malignant cell phenotype (clonal mutations). The density of these clonal mutations per million base pairs (Mb) of genomic DNA is defined as cTMB and can be determined by bioinformatics pipeline analysis using whole exome next generation sequencing data [12, 13, 15, 18, 19, 22, 38, 39].

Recently, we verified that final gemogenovatucel-T product cTMB signal is comprised of the same clonal DNA mutations utilized to turn on the systemic effector cell response is the same as the harvested autologous tumor used to construct gemogenovatucel-T [39]. Correlation coefficient of 98% (R2-0.9802) was observed of cTMB signals between harvested tumor used to construct gemogenovatucel-T and the gemogenovatucel-T product [39]. High correlation coefficient of 95% (R2-0.9525) was also observed between harvested tumor clonal neoantigen (cNEO) signals and gemogenovatucel-T cNEO signals. These results verify that gemogenovatucel-T product preserves cTMB signal and provides accurate clonal neoantigen display of the individual patient's tumor [39].

We thus provided a statistical analysis plan to FDA to define prospectively the impact of high cTMB and gemogenovatucel-T treatment on OS of HRP profile in reanalysis of patients undergoing long term follow up within the continued ongoing VITAL trial. Results are presented below.

### Methods

### Patient Population

Gemogenovatucel-T or placebo was administered in the VITAL trial (CL-PTL-119, NCT02346747) as maintenance therapy in frontline Stage IIIb/IV ovarian cancer patients (high grade serous, endometroid) in clinical complete response after debulking surgery and adjuvant or neoadjuvant chemotherapy using carboplatin/paclitaxel as previously published in a multicenter, double-blind, 1:1 randomized trial. 91 patients received gemogenovatucel-T (n=47) or placebo (n=44) and were previously analyzed for safety and efficacy [27].

Patients were monitored for long term follow up for disease recurrence through third party assessment by CT scan of the chest, abdomen and pelvis every 3 months in years 1-3, every 6 months through year 5, and then every year through year 10. Patients remained on study beyond recurrence and followed for OS. All raw data was verified by PharPoint Research (Durham, NC) for statistical calculations. Original analysis was performed Mar. 6, 2020 (Stat Beyond Consulting, Irvine, CA) and independently verified Dec. 9, 2020 (PharPoint Research, Durham, NC). Updated follow up analysis was done Apr. 7, 2021 (Stat Beyond Consulting, Irvine, CA). Trial evaluation with collection of clinical data (e.g., adverse events, recurrence, post therapies) and OS was continued and uninterrupted until clinical database lock 10April2024 prior to clonal signal results determination. On Sep. 17, 2024 CTMB, cNEO and ITH molecular determination was completed and transferred to Stat Beyond Consulting (Irvine, CA) for analysis.

### Hypothesis

Gemogenovatucel-T is designed to carry the full set of personal cancer clonal neoantigens on its cell surface and generates a dendritic cell response that induces expansion of effector cell populations that target the patient's own clonal neoantigens [19]. We hypothesize, in HRP profile patients with intact DNA repair, OS will correlate with high cTMB. If significant, we will also explore these factors effect against recurrence free survival (RFS).

### Endpoints

### Primary Endpoint

The primary endpoint of this study is comparison of gemogenovatucel-T vs. placebo from randomization for cTMB high subjects in the HRP population.

OS from randomization is the duration from the randomization date until the date of death. All subjects alive or without death date will be censored as alive at their last date known alive.

### Secondary Endpoint

The secondary endpoint of this study is RFS from randomization for cTMB high subjects in the HRP population.

RFS from randomization is the time from the randomization date to either the date the subject is first recorded as having disease recurrence (even if the subject went off treatment because of toxicity), or the date of death if the subject dies due to any causes before recurrence. These are the same definitions previously published with initial trial assessment of all ovarian cancer patients [27].

All study endpoints were tested using the hierarchical testing method according to the pre-specified order listed below. For each test, the one-sided stratified (by the randomization stratification factors) log rank test at the alpha=0.05 level of significance were utilized to compare if there was any treatment difference between the gemogenovatucel-T and placebo groups.
1. OS from randomization for cTMB high subjects in the HRP population
2. RFS from randomization for cTMB high subjects in the HRP population

The samples used for study include matched archived tumor and PBMC genomic DNA (gDNA) extracted in 2019 and 2020 for patients (n=91). The clinical database of OS and RFS was locked on 10APR2024.

### Statistical Methodology

Demographics and baseline characteristics including age, race, ethnicity, ECOG, FIGO stage, frontline chemotherapy, number of chemotherapy cycles, frontline surgery residual disease status, histology, and days from last chemo given to first dose of gemogenovatucel-T/placebo were determined for the cTMB/HRP subgroup as per prior publication [27].

Number of patients who received subsequent anti-cancer therapy and types of subsequent anti-cancer therapy including chemotherapy, immunotherapy, PARP inhibitor, VEGF inhibitor was provided for the cTMB/HRP determined subgroup.

The median cTMB value from the HRP population was identified and utilized to classify each subject as cTMB high (above or equal to median) or cTMB low (below median).

OS and RFS distributions for all populations analyzed were summarized using the Kaplan-Meier method and treatment groups were compared by using the stratified log-rank test for all populations. HR was estimated using the Cox proportional hazards model and stratified by the randomization stratification factors. OS/RFS distributions in the gemogenovatucel-T vs. placebo arms were compared using a one-sided stratified (by the randomization stratification factors) log rank test at the alpha=0.05 level of significance. P-value was provided according to the pre-specified hierarchical testing method. Kaplan-Meier estimate was used to estimate OS at 1, 2, 3 and 5 years and RFS at 6, 12 and 24 months.

Restricted mean survival time (RMST) analysis was used per statistical analysis plan to analyze primary and secondary endpoints. RMST difference analysis was performed using a truncation point equal to the minimum of the longest follow-up time of each group without covariate adjustment. The one-sided p-value for the RMST difference was provided.

### Blinding and Unblinding

Sample codes were used to blind physical test articles and to obscure whether a subject received active product or placebo. Samples submitted to Frontage Laboratories (Deerfield Beach, FL) are identified by blinded code or neutral identifiers (Subject ID, lot number). Test provider (Frontage Laboratories, Deerfield Beach, FL) generated the blinded test data (cTMB, cNEO, ITH), identified only by code or neutral identifier. QA received the blinded data from Frontage Laboratories (Deerfield Beach, FL), and added the clinical data by matching identifiers, and generated the unblinded information for analysis. QA then sent the full unblinded data set to the Statistician. The statistician provided the unblinded analysis and summary of results.

The clinical database was scrubbed and locked on 10APR2024. Meanwhile the project steps including: assembly of a bioinformatics processing pipeline, verification of the performance of the pipeline, and proof of concept of the method using genomic DNA from matched PBMC, tumor, and product samples from a subset of patients was confirmed. Upon confirmation of suitability of the platform, available samples from all subjects in the intent to treat population were placed into the processing pipeline. Frontage Laboratories (Deerfield Beach, FL) evaluated the samples and generated the data. The summary data of cTMB, ITH and cNEO were ingested into the clinical database. The ingested data was verified against the summary and raw datasets provided by Frontage Laboratories (Deerfield Beach, FL) to ensure cleanliness of migration. The clinical dataset including RFS, OS, cTMB, ITH, and cNEO data were presented to the statistician for analysis against findings of molecular testing according to the Statistical Analysis Plan for Neoantigen Related Data Analysis.

### Sample Analysis

Whole exome DNA libraries were generated from fifty (50) nanograms of genomic DNA per sample isolated from cryopreserved tumor cell and PBMC cell suspensions as part of a previously published study using the Twist Exome 2.0 kit according to the manufacturer's recommendations, except that Twist unique molecular identifier (UMI) adapters were utilized instead of Twist Universal Adapters for ligation to double-stranded genomic DNA fragments. Batches of DNA libraries representing the tumor and PBMC samples from 3-4 patients were pooled together at a DNA concentration ratio of 9.4:1 tumor to PBMC for each patient and hybridized to the Twist Exome 2.0 probe set and enriched library fragments were recovered according to the manufacturer's instructions. DNA library pools were sequenced on the NovaSeq X instrument at the Discovery Life Sciences genomic center in Huntsville, Alabama to a depth sufficient to achieve a mean coverage depth of post-UMI processed consensus reads of at least 665× and 78× for each tumor and PBMC sample, respectively.

### Bioinformatics

Sequencing data was processed through a bioinformatics pipeline as previously described in detail in order to calculate levels of cTMB, clonal neo-antigen load (cNEO) and intra-tumor heterogeneity (ITH) (LNS 981558, LNS 964942). Data processing steps in the pipeline were based on the framework established by McGranahan et al [18, 22, 40] but utilized state-of-the-art applications for each step based on recent benchmarking studies [40-43]. Optimized BAM files were generated from raw reads by first removing adapters and low quality trailing nucleotides with Trimmomatic, processing sequencing data according to fgbio best practices (https://github.com/fulcrumgenomics/fgbio/blob/main/docs/best-practice-consensus-pipeline.md) to generate BAM files containing consensus reads, and then performing base score recalibration and exome cropping using GATK applications and SamTools View, respectively. The optimized BAM files were used as input (1) to call tumor associated single nucleotide variants (SNVs) and small insertions/deletions (InDels) using GATK Mutect2 in paired tumor/normal mode, (2) to calculate allele-specific copy number using FACETS [44], and (3) along with unaligned read input, to call MHC-I alleles using HLA*LA [45]. VCF files from GATK Mutect2 were filtered and annotated using GATK FilterMutect2Calls and GATK Funcotator, respectively. Output VCF files along with allele-specific copy number and total tumor purity data from FACETS were used as input to Pyclone 6.1 in order to group SNVs and InDels into defined clusters each with an estimated cancer cell fraction (CCF) [46]. To identify potential peptide neoantigens, unannotated, filtered VCFs from GATK Mutect2 were annotated with Ensembl VEP software and then processed through pVACSeq v 4.0.4 [41, 48] along with each patients' specific MHC-I A, B, and C predicted haplotypes from HLA*LA; potential peptides of 8-11 amino acids of length that spanned a non-synonymous variant were considered and binding affinities (IC50) of potential peptides were calculated using the MHCflurry algorithm built in to pVACSeq v 4.0.4 [42]. The peptide list was filtered using the built-in filtering module of pVACSeq to select only peptides with an IC50<500 nM and where IC50 for the mutant peptide was lower than that for the comparable peptide derived from the human genome reference sequences. All sequence processing steps used version hg38 human reference sequences.

Clonal variants were defined as non-synonymous SNPs and Indels belonging to the cluster with the highest CCF that was 0.9 or higher. ITH was calculated as the percentage of non-synonymous SNPs and Indels that were not clonal. Tumor mutational burden (TMB) was determined as the count of non-synonymous SNPs and Indels per megabase (Mb) of DNA included in the exome. cTMB was the same calculation as used for TMB but only clonal variants were included in the total. cNEO was calculated as the number of predicted MHC-1 binding peptides that met the above filtering criteria which spanned any clonal non-synonymous variant per Mb of exome sequence; all peptides that met the filtering criteria were counted for each associated variant. Only variants present in the exome-cropped BAM files and subsequently called by Mutect2 were included in any calculation. The total exome size was 36.46 Mb which was based on hg38 genomic coordinates covered by any and all of the exome regions included in the Twist Exome 2.0 bed file.

### Study Design

The VITAL study was a randomized, Phase 2b, double-blind, placebo-controlled trial. Patient population and study design were previously published [27, 29]. Patients received 1×10e7 cells/injection of gemogenovatucel-T or placebo once per month. Gemogenovatucel-T plasmid construction, cGMP manufacturing, tissue processing and transfection were carried out as previously described [28, 34, 49, 50]. A minimum of 4 and maximum of 12 doses were given and treatment continued until product exhaustion or disease progression. All 45 HRP patients entered into the VITAL trial were followed for RFS, OS and late adverse events including possible development of secondary hematologic malignancy. Continued independent third party assessment by radio-imaging was monitored by World Care Clinical (Boston, MA, USA) using response evaluation criteria in solid tumors version 1.1 (RECIST 1.1) [27, 28]. Reports of late adverse events were provided by patient caretaker.

BRCA-1/2 mutation, HRD and HRP status were determined as previously described in 91 patients [27, 28]. Twenty-five patients were identified as HRP in the gemogenovatucel-T arm and 20 were HRP in the placebo arm. Per assay guidelines using MyChoice® CDx (Myriad, Inc., Salt Lake City, UT) a score of ≥42 was used to identify patients who were HRD, and <42 threshold was used to define HRP. Demographics of the 45 HRP patients which have previously been published revealed a higher number of Stage IV presenting disease and poor performance status (ECOG 1) patients in the gemogenovatucel-T arm [28].

### Results

### Demographics

Demographics of all patients have been previously published [27]. Table 10 shows demographic profile of the HRP/cTMB-H patients. No statistical difference between gemogenovatucel-T and placebo was observed in the HRP/cTMB-H subset. Good performance status (ECOG-0) was numerically higher in the placebo group, but not statistically significant (two-sided p-value of 0.22 per Fisher's exact test).

TABLE 10
Demographics and Baseline Characteristics
cTMB High Subjects in the HRP Population

| Characteristic | Gemogenovatucel-T | | Placebo | |
|---|---|---|---|---|
| No. of patients | 11 | | 12 | |
| Age, years | | | | |
| Median (IQR) | 66.0 | (62.0-71.5) | 64.5 | (61.5-67.3) |
| Range | 51.0-84.0 | | 49.0-79.0 | |
| <65 | 3 | (27.3%) | 6 | (50.0%) |
| ≥65 | 8 | (72.7%) | 6 | (50.0%) |
| Race | | | | |
| Asian | 0 | (0.0%) | 0 | (0.0%) |
| Black or African American | 0 | (0.0%) | 1 | (8.3%) |
| White | 11 | (100.0%) | 11 | (91.7%) |
| Not Reported | 0 | (0.0%) | 0 | (0.0%) |
| Ethnicity | | | | |
| No. of patients | 11 | | 12 | |
| Hispanic or Latino | 0 | (0.0%) | 0 | (0.0%) |
| Not Hispanic or Latino | 11 | (100.0%) | 11 | (91.7%) |
| Not Reported | 0 | (0.0%) | 1 | (8.3%) |
| ECOG | | | | |
| 0 | 4 | (36.4%) | 8 | (66.7%) |
| 1 | 7 | (63.6%) | 4 | (33.3%) |
| FIGO Stage | | | | |
| III | 8 | (72.7%) | 11 | (91.7%) |
| IV | 3 | (27.3%) | 1 | (8.3%) |
| Frontline Chemotherapy | | | | |
| Neoadjuvant | 3 | (27.3%) | 1 | (8.3%) |
| Adjuvant | 8 | (72.7%) | 11 | (91.7%) |
| No. of Chemotherapy cycles | | | | |
| Mean (SD) | 5.9 | (0.30) | 6.1 | (0.29) |
| Median (IQR) | 6.0 | (6.0-6.0) | 6.0 | (6.0-6.0) |
| Range | 5.0-6.0 | | 6.0-7.0 | |
| Frontline surgery | | | | |
| residual disease status | | | | |
| Macroscopic | 3 | (27.3%) | 3 | (25.0%) |
| Microscopic/NED | 8 | (72.7%) | 9 | (75.0%) |
| Histology | | | | |
| Endometrioid carcinoma | 1 | (9.1%) | 0 | (0.0%) |
| Mixed serous/clear cell | 0 | (0.0%) | 0 | (0.0%) |
| High grade serous carcinoma | 10 | (90.9%) | 12 | (100.0%) |
| No. of patients | 11 | | 12 | |
| Days from last chemo | | | | |
| given to first dose of | | | | |
| gemogenovatucel-T/placebo | | | | |
| Mean (SD) | 44.3 | (8.43) | 46.4 | (20.41) |
| Median (IQR) | 42.0 | (40.0-50.0) | 44.5 | (35.8-49.0) |

| | | |
|---|---|---|
| Range | 29.0-57.0 | 23.0-102.0 |

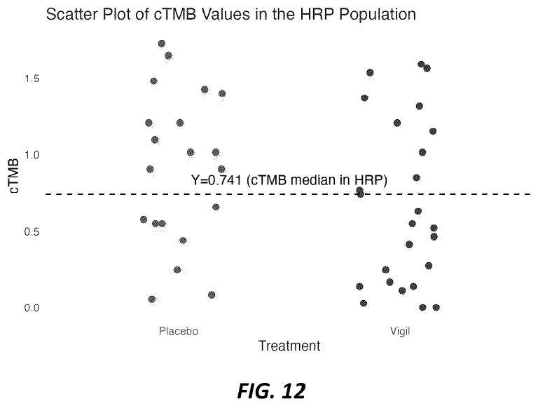

Subsequent anticancer therapy following completion of gemogenovatucel-T or placebo as shown in Table 11 was not different in the HRP/high cTMB sub population between gemogenovatucel-T and placebo. Display of cTMB by scatter plot is shown in FIG. 12 comparing gemogenovatucel-T to Placebo within the HRP subset. These results show no difference in distribution of cTMB signal between gemogenovatucel-T or placebo (two-sided p-value=0.133 based on the Welch two sample t-test) thus supporting effect related to gemogenovatucel-T over placebo and not related to difference in cTMB threshold between groups. Optimization assessment was also performed to explore alternative threshold. Interestingly, analysis of all HRP patients revealed similar optimal threshold as predicted by median value (0.658 vs. 0.741, respectively; mOS 68 vs. 22 months HR=0.22, 0.06-0.81, p=0.007).

TABLE 11
Subsequent Anti-cancer Therapy cTMB
High Subjects in the HRP Population

| | Gemogenovatucel-T | Placebo |
|---|---|---|
| No. of Patients Who Received | 8 | 11 |
| Subsequent Anti-cancer Therapy | | |
| Types of Subsequent Anti-cancer Therapy | | |
| Chemotherapy | 6 | 10 |
| Immunotherapy | 0 | 3 |
| PARP Inhibitor | 5 | 6 |
| VEGF Inhibitor | 3 | 4 |
| Other | 6 | 5 |

### Safety

Similar safety profile was achieved with gemogenovatucel-T administered to HRP/cTMB-H subpopulation as with all patients previously published (Table 12) [27]. One patient received gemogenovatucel-T dose interruption for Grade 3 non treatment related toxic effect (abdominal infection) per investigator decision. No product related ≥Grade 3 adverse events were observed. Dose reduction, discontinuation and treatment death was not observed. Long term follow up revealed no evidence of treatment related AML or MDS.

TABLE 12
Treatment related adverse events for gemogenovatucel-T versus
placebo in the VITAL trial HRP high cTMB population.

| System Organ Class | Gemogenovatucel-T (n = 11) | | | | | | Placebo (n = 12) | | | | | |
|---|---|---|---|---|---|---|---|---|---|---|---|---|
| Adverse Event | Grade 1 | | Grade 2 | | Grade 3 | | Grade 1 | | Grade 2 | | Grade 3 | |
| Gastrointestinal | 2 | (18.2%) | 0 | (0%) | 0 | (0%) | 2 | (16.7%) | 0 | (0%) | 0 | (0%) |
| disorders | | | | | | | | | | | | |
| Abdominal pain | 1 | (9.1%) | 0 | (0%) | 0 | (0%) | 0 | (0%) | 0 | (0%) | 0 | (0%) |
| Diarrhea | 1 | (9.1%) | 0 | (0%) | 0 | (0%) | 0 | (0%) | 0 | (0%) | 0 | (0%) |
| Nausea | 0 | (0%) | 0 | (0%) | 0 | (0%) | 2 | (16.7%) | 0 | (0%) | 0 | (0%) |
| General disorders and | 5 | (45.5%) | 0 | (0%) | 0 | (0%) | 4 | (33.3%) | 1 | (8.3%) | 0 | (0%) |
| administration site | | | | | | | | | | | | |
| conditions | | | | | | | | | | | | |
| Edema extremities | 1 | (9.1%) | 0 | (0%) | 0 | (0%) | 0 | (0%) | 0 | (0%) | 0 | (0%) |
| Fatigue | 0 | (0%) | 0 | (0%) | 0 | (0%) | 1 | (8.3%) | 1 | (8.3%) | 0 | (0%) |
| Injection site reaction | 4 | (36.4%) | 0 | (0%) | 0 | (0%) | 3 | (25%) | 0 | (0%) | 0 | (0%) |
| Musculoskeletal and | 1 | (9.1%) | 1 | (9.1%) | 0 | (0%) | 3 | (25%) | 0 | (0%) | 2 | (16.7%) |
| connective tissue | | | | | | | | | | | | |
| disorders | | | | | | | | | | | | |
| Arthralgia | 0 | (0%) | 1 | (9.1%) | 0 | (0%) | 1 | (8.3%) | 0 | (0%) | 0 | (0%) |
| Back Pain | 0 | (0%) | 0 | (0%) | 0 | (0%) | 1 | (8.3%) | 0 | (0%) | 0 | (0%) |
| Bone Pain | 0 | (0%) | 0 | (0%) | 0 | (0%) | 0 | (0%) | 0 | (0%) | 1 | (8.3%) |
| Flank Pain | 0 | (0%) | 0 | (0%) | 0 | (0%) | 1 | (8.3%) | 0 | (0%) | 0 | (0%) |
| Generalized Muscle | 0 | (0%) | 0 | (0%) | 0 | (0%) | 0 | (0%) | 0 | (0%) | 1 | (8.3%) |
| Weakness | | | | | | | | | | | | |
| Muscle pain | 1 | (9.1%) | 0 | (0%) | 0 | (0%) | 0 | (0%) | 0 | (0%) | 0 | (0%) |
| Nervous System | 0 | (0%) | 0 | (0%) | 0 | (0%) | 0 | (0%) | 0 | (0%) | 1 | (8.3%) |
| Disorders | | | | | | | | | | | | |
| Syncope | 0 | (0%) | 0 | (0%) | 0 | (0%) | 0 | (0%) | 0 | (0%) | 1 | (8.3%) |
| Renal and urinary | 1 | (9.1%) | 0 | (0%) | 0 | (0%) | 0 | (0%) | 0 | (0%) | 0 | (0%) |
| disorders | | | | | | | | | | | | |
| Urinary incontinence | 1 | (9.1%) | 0 | (0%) | 0 | (0%) | 0 | (0%) | 0 | (0%) | 0 | (0%) |
| Respiratory, thoracic, | 0 | (0.0%) | 0 | (0%) | 0 | (0%) | 0 | (0%) | 0 | (0%) | 1 | (8.3%) |
| and mediastinal | | | | | | | | | | | | |
| disorders | | | | | | | | | | | | |
| Dyspnea | 0 | (0%) | 0 | (0%) | 0 | (0%) | 0 | (0%) | 0 | (0%) | 1 | (8.3%) |
| Skin and | 0 | (0.0%) | 1 | (1.1%) | 0 | (0%) | 0 | (0%) | 0 | (0%) | 0 | (0%) |
| subcutaneous tissue | | | | | | | | | | | | |
| disorders | | | | | | | | | | | | |
| Rash maculo-papular | 0 | (0%) | 1 | (9.1%) | 0 | (0%) | 0 | (0%) | 0 | (0%) | 0 | (0%) |

No Grade 4 or 5 treatment related events were observed.

Primary Endpoint High cTMB Relationship to OS in HRP Population

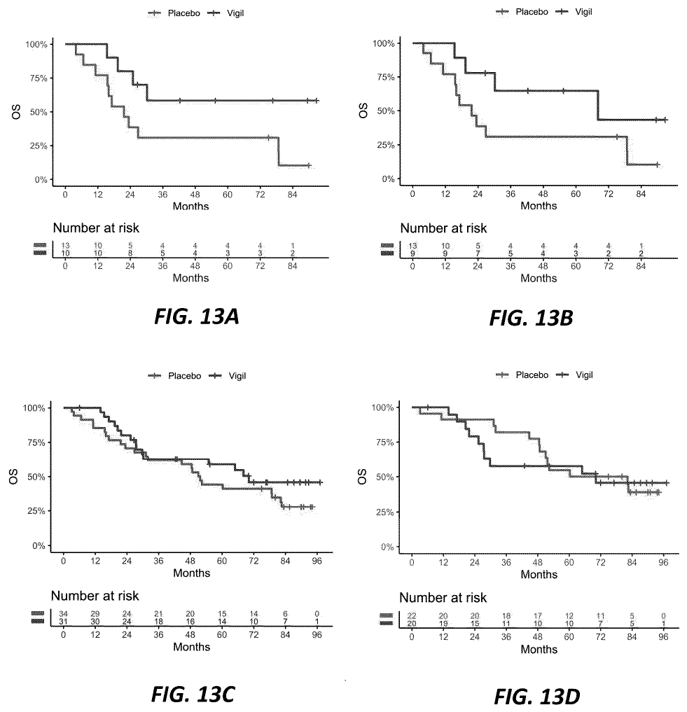

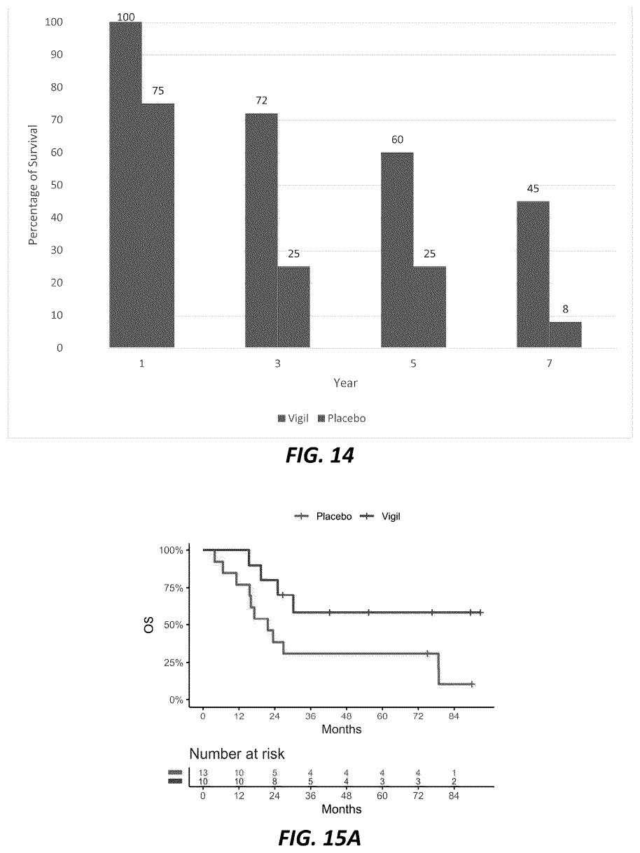

Patients entered into VITAL trial (CL-PTL-119) with high cTMB and HRP signal profile as defined in the Statistical Analysis Plan achieved robust OS advantage to gemogenovatucel-T vs. placebo (FIGS. 13A-13D) with demonstration of HR=0.23 and p value of 0.008. Kaplan Meier estimated median OS was 68 (30.2, NA, 95% CI) months for gemogenovatucel-T treated patients vs. 19 (15.6, NA) months for placebo treated patients. Breakdown of 1 year, 3 years, 5 years and 7 years OS percent of gemogenovatucel-T (left bar) vs. placebo (right bar) was 100% (1.0, 1.0, 95% CI) to 75% (0.5, 1.0, 95% CI), 72% (0.5, 1.0, 95% CI) to 25% (0.1, 0.7, 95% CI), 60% (0.4 to 1.0, 95%) to 25% (0.1, 0.7, 95% CI) and 45% (0.2 to 1.0, 95% CI) to 8% (0.01 to 0.5, 95% CI), respectively (FIG. 14). Advantage of mean OS is also confirmed with SAP preplanned RMST analysis [63 (48-78, 90% CI) vs. 33 (18-47, 90% CI) | months between gemogenovatucel-T and placebo, p=0.007]. No significant relationship of demographics (Table 10) or follow up therapy (Table 11) was demonstrated to influence OS between gemogenovatucel-T or placebo.

Key preplanned observations related to efficacy in the HRP/cTMB-H subpopulation are shown in Table 13.

### Ad-Hoc Analysis of Additional Patient Subpopulations and Genomic Metrics

Results of ad hoc analysis of a subpopulation of the 91 patients in the VITAL trial having a wild-type BRCA genotype (BRCA+) and higher than median levels of cTMB (n=42) is shown in Table 14. The benefit of gemogenovatucel-T treatment was confirmed in the BRCA+/high cTMB population as evidenced by significant improvements in OS and RFS both from randomization via Kaplan Meier analysis and with OS via RMST analysis (Table 14).

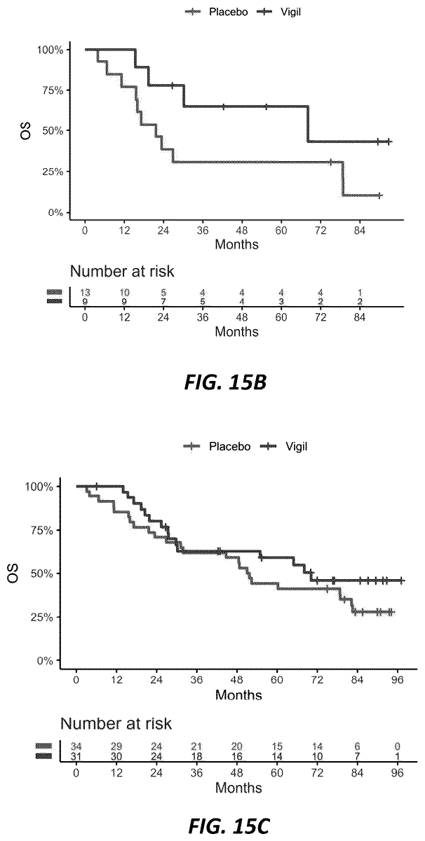

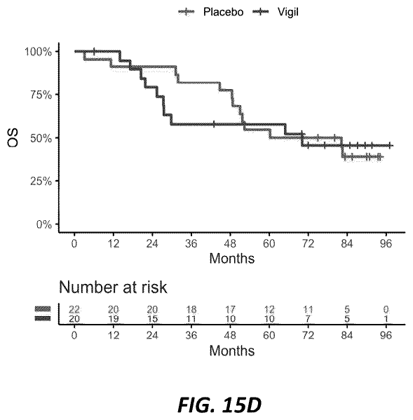

Consistent with high cTMB in HRP positive patients receiving gemogenovatucel-T, we also demonstrated that the high cTMB patients also included 9 of 10 high cNEO patients and all 9 of 9 of the low ITH patients. Separate correlation to OS of gemogenovatucel-T vs. placebo for high cNEO (FIG. 15A) and low ITH (FIG. 15B) demonstrated significance as expected HR of 0.39 (0.12, 1.26, 95% CI) and 0.30 (0.8, 1.10, 95% CI), respectively. The high cTMB group of all 91 ITT patients however as expected did not demonstrate advantage in OS (FIG. 15C) or RFS (data not shown) to gemogenovatucel-T over placebo [HR=0.69 (0.35, 1.38, 95% CI); HR=0.72 (0.39, 1.36, 95% CI)], respectively. Additionally, the nonHRP (BRCA mutant/HRD) patients did not demonstrate an advantage in the cTMB high group in OS [HR 0.99 (0.39, 2.47, 95% CI) p=0.488], or RFS [HR 0.94 (0.42, 2.10, 95% CI), p=0.442] (FIG. 15D). Collectively all of these results support beneficial relationship of gemogenovatucel-T to clonal signals within the HRP subpopulation.

TABLE 13
Key Planned Observations Related to Efficacy
in HRP High cTMB Population as outlined

| | | |
|---|---|---|
| Gemogenovatucel-T | Placebo | Significance |

Kaplan Meier Estimate of Median OS/RFS (95% CI), Hazard Ratio (95% CI)

| | | | |
|---|---|---|---|
| OS from Randomization | 68 (30.2, NA, | 19 (15.6, | HR = 0.23, (0.06, |
| Primary Objective | 95% CI) months | NA) months | 0.83) p = 0.008 |
| RFS from Randomization | 10 (5.8, | 6 (3.5, NA) | HR = 0.41, (0.13, |
| Secondary Objective | NA) months | months | 1.35) p = 0.066 |

RMST Estimate (90% CI)

| | | | |
|---|---|---|---|
| OS from Randomization | 63 (48-78) | 33 (18-47) | p = 0.007 |
| Prospective | months | months | |
| RFS from Randomization | 10 (8-11) | 6 (5-7) | p = 0.004 |
| Prospective | months | months | |

Kaplan Meier Estimate of Median OS/RFS (95% CI), Hazard Ratio (95% CI)

| | | | |
|---|---|---|---|
| OS from Procurement | 75 (39.6, | 26 (21.3, | HR = 0.23 (0.06, |
| Primary Objective | NA) months | NA) months | 0.83), p = 0.008 |
| RFS from Procurement | 17 (14, | 12 (11.3, | HR = 0.35 (0.11, |
| Secondary Objective | NA) months | NA) months | 1.14), p = 0.033 |

RMST Estimate from Procurement (90% CI)

| | | | |
|---|---|---|---|
| OS from Procurement | 70 (55-85) | 39 (25-53) | p = 0.006 |
| Pre-planned | months | months | |
| RFS from Procurement | 16 (15-18) | 6 (11-14) | p = 0.001 |
| Pre-planned | months | months | |

TABLE 14
Supportive ad hoc related analysis in BRCA-wt subpopulation
demonstrates OS benefit in relationship to high cTMB

| | | |
|---|---|---|
| Gemogenovatucel-T | Placebo | Significance |

Kaplan Meier Estimate of Median OS/RFS (95% CI), Hazard Ratio (95% CI)

| | | | |
|---|---|---|---|
| OS from Randomization | NA (30.2, | 36 (17.1, | HR = 0.37 (0.15, |
| Primary Objective | NA) months | NA) months | 0.91), p = 0.013 |
| RFS from Randomization | 11 (6.5, | 8 (5.6, 10.2) | HR = 0.49 (0.22, |
| Secondary Objective | NA) months | months | 1.12), p = 0.044 |

RMST Estimate (90% CI)

| | | | |
|---|---|---|---|
| OS | 64 (53-74) | 45 (32-58) | p = 0.030 |
| Ad hoc | months | months | |
| RFS | 16 (12-20) | 10 (6-13) | p = 0.033 |
| Ad hoc | months | months | |

### Discussion

Results demonstrate that gemogenovatucel-T provides substantial OS benefit without toxic effect when used as maintenance therapy in newly diagnosed HRP/cTMB-H Stage IIIb-IV ovarian cancer patients. Consistent with our hypothesis, we are the first to demonstrate prospective use of cTMB determination as a “mechanism based biomarker” to define an optimal immunotherapy treatment sensitive subset of patients with unmet medical need. Although several retrospective studies have shown consistent cancer benefit to immunotherapy involving cTMB subpopulations [12-15, 18, 22]. Our prospective study of the role of cTMB in identifying this high response population to gemogenovatucel-T provides justification for use of gemogenovatucel-T as treatment recommendation for maintenance use in Stage IIIb-IV newly diagnosed HRP/cTMB-H ovarian cancer.

Gemogenovatucel-T product educates and expands clonal neoantigen targeting effector cells, which are the natural effector mediators of anticancer activity generated by gemogenovatucel-T [19, 51]. Based on our knowledge, we developed and successfully qualified a molecular based bioinformatics pipeline process to determine clinically relevant cTMB expression from autologous cancer tissue of individual patients entered into VITAL trial.

Use of whole exome sequencing to characterize clonal signals from preserved and fresh tissue have been well developed [12-14, 18, 22], but not applied prospectively. Retrospective data by others demonstrate marked OS advantage in correlation with high cTMB when undergoing immunotherapy [12-14, 18, 22]. Prior to determining cTMB (and clonal neoantigen) of patients entered into CL-PTL-119 we prospectively predicted and identified high cTMB as a primary determinant of OS in response to gemogenovatucel-T against standard of care within the HRP population. Raw data utilized in this blinded analysis was generated using Twist Biosciences Exome 2.0 platform for library preparation and enrichment and the Illumina NovaSeq X instrument for sequencing. The raw data was processed through our novel bioinformatic pipeline. Both wet lab and dry lab procedures were carried out in coordination with our service partner, Frontage Laboratories (Deerfield Beach, FL). The suitability of the wet lab technology platform, Frontage Laboratories wet lab and dry lab services, and the bioinformatic pipeline was pre-qualified prior to generation of data from the full 91 patients included in the VITAL trial as described [39]. Specifically, the functionality of the bioinformatics pipeline was first assessed using exome sequencing data from three previous published studies. Using simulated tumor sequencing data from a benchmarking study of bioinformatic tools for subclonal deconvolution [40], a strong correlation between cancer cell fraction (CCF) generated from our pipeline and the benchmark CCF values was observed and positive predictive value and positive percent agreement of >97.5% and >96.5% was demonstrated for the variant calls generated by our pipeline as compared to the benchmark data. MHC-I and MHC-II haplotype calls generated from our bioinformatics pipeline using 1000 genomes project exome sequencing data from 99 patients [43], showed a 91.5% match to published benchmark data [52]. Furthermore, whole exome NGS data from a subset of melanoma patients included in another trial was also used to verify the integrity of the pipeline by comparing cTMB values generated from our bioinformatics pipeline to those calculated from the published data and a strong correlation (R2-0.9802) was observed [39]. Finally, we demonstrated the suitability of the entire wet lab and dry lab process by analyzing genomic DNA extracted from both the autologous cancer tissue used to construct gemogenovatucel-T and gemogenovatucel-T product, and matching PBMC samples for 9 patients and observed a robust correlation (R2-0.98) in cTMB levels between the tumor and its corresponding product across the set of patients (LNS 981558, LNS 964942) [39].

Others have shown that BRCA-wt and more specifically HRP tumors have elevated immunogenic signals including increased abundance of tumor infiltrating lymphocytes (TILs) demonstrating high IFN gamma-1 signaling [53, 54]. They also have stable DNA repair mechanisms. In comparison, BRCA-m, HRD tumors have poor DNA repair mechanisms which leads to increased mutational load including increased subclonal mutations and increased display of subclonal neoantigens [18, 30-32]. Gemogenovatucel-T anticancer activity was not shown in HRD/BRCA mutant patients involved in VITAL trial for RFS (HR=1.199, p=0.674) or OS (HR=1.787, p=0.827) [27]. Increased subclonal neoantigen heterogeneity in BRCA-m, HRD tumors potentially support less effective immunoediting and/or lower levels of active neoantigen specific T cells [18, 19, 26]. Moreover, ovarian cancer expresses higher immune suppressive cytokines (i.e., TGFβ-1/2) compared to other malignancies [55]. Systemic immune targeting is optimized by gemogenovatucel-T with autologous malignant cells containing high clonal neoantigen display as is observed with HRP vs. HRD malignant cell populations. Gemogenovatucel-T increases antitumor activity by simultaneously and synergistically upregulating activated CD8 T cells against cancer neoantigens via MHC presentation on DCs and macrophages, increasing the abundance of mature DCs via GMCSF, and downregulating TGFβ via bi-shRNA^{furin}. It is unclear if CD47-SIRPα regulatory signal pathway is involved with gemogenovatucel-T induced enhanced macrophage function [56, 57]. However, specifically targeting patients with high cTMB within the HRP subset clearly provides identification of an optimal subpopulation to gemogenovatucel-T. We have shown that the same cTMB and cNEO signaling indeed is maintained between original tumor used to construct gemogenovatucel-T and gemogenovatucel-T product [39]. This supports clinical initiative to define cTMB and given the robust OS advantage to consider cTMB-H/HRP patient for treatment with gemogenovatucel-T as maintenance therapy.

In conclusion, results demonstrate significant OS advantage to gemogenovatucel-T immunotherapy related to cTMB targeting involving HRP patients who are of unmet medical need. As we have demonstrated, Stage IIIb-IV/HRP/cTMB-H ovarian cancer patients have optimal clinical advantage to maintenance therapy with gemogenovatucel-T. Targeting of cTMB as shown with gemogenovatucel-T is unique. Other neoantigen based immunotherapy including CAR-Ts [58-61] may gain insight into targeting of clonal signals demonstrating clonal neoantigen peptide expression. There may be limits to immune response against all cancer cells within a patient when attempting to use tumor associated analysis on subclonal neoantigens as targets of immunotherapy in solid tumor directed therapy [62, 63]. Future development of gemogenovatucel-T will extend to high cTMB disease of HRP profile involving other solid tumor malignancy.

### References for Example 4

1. Jelovac, D. and D. K. Armstrong, Recent progress in the diagnosis and treatment of ovarian cancer. CA Cancer J Clin, 2011. 61 (3): p. 183-203.
2. Gogineni, V., et al., Current Ovarian Cancer Maintenance Strategies and Promising New Developments. J Cancer, 2021. 12 (1): p. 38-53.
3. Green, A. E., A. A. Garcia, and S. Ahmed. What is the prognosis of ovarian cancer? 2020 [cited 2021 Mar. 11]; Available from: https://www.medscape.com/answers/255771-194612/what-is-the-prognosis-of-ovarian-cancer.
4. Moore, K., et al., Maintenance Olaparib in Patients with Newly Diagnosed Advanced Ovarian Cancer. N Engl J Med, 2018. 379 (26): p. 2495-2505.
5. Gonzalez-Martin, A., et al., Niraparib in Patients with Newly Diagnosed Advanced Ovarian Cancer. N Engl J Med, 2019. 381 (25): p. 2391-2402.
6. Gonzalez-Martin, A., et al., Progression-free survival and safety at 3.5 years of follow-up: results from the randomised phase 3 PRIMA ENGOT-OV26 GOG-3012 trial of niraparib maintenance treatment in patients with newly diagnosed ovarian cancer. Eur J Cancer, 2023. 189: p. 112908.
7. Tewari, K. S., et al., Final Overall Survival of a Randomized Trial of Bevacizumab for Primary Treatment of Ovarian Cancer. J Clin Oncol, 2019. 37 (26): p. 2317-2328.
8. Monk, B. J., et al., LBA30 ATHENA-COMBO, a phase III, randomized trial comparing rucaparib (RUCA)+nivolumab (NIVO) combination therapy vs RUCA monotherapy as maintenance treatment in patients (pts) with newly diagnosed ovarian cancer (OC). Annals of Oncology, 2024. 35: p. S1223-S1224.
9. Kim, S. I., et al., Effect of BRCA mutational status on survival outcome in advanced-stage high-grade serous ovarian cancer. J Ovarian Res, 2019. 12 (1): p. 40.
10. Burger, R. A., et al., Incorporation of bevacizumab in the primary treatment of ovarian cancer. N Engl J Med, 2011. 365 (26): p. 2473-83.
11. Landen, C. N., et al., Influence of Genomic Landscape on Cancer Immunotherapy for Newly Diagnosed Ovarian Cancer: Biomarker Analyses from the IMagyn050 Randomized Clinical Trial. Clin Cancer Res, 2023. 29 (9): p. 1698-1707.
12. Riaz, N., et al., Tumor and Microenvironment Evolution during Immunotherapy with Nivolumab. Cell, 2017. 171 (4): p. 934-949 e16.
13. Boll, L. M., et al., The impact of mutational clonality in predicting the response to immune checkpoint inhibitors in advanced urothelial cancer. Sci Rep, 2023. 13 (1): p. 15287.
14. Rizvi, N. A., et al., Cancer immunology. Mutational landscape determines sensitivity to PD-1 blockade in non-small cell lung cancer. Science, 2015. 348 (6230): p. 124-8.
15. Snyder, A., et al., Genetic basis for clinical response to CTLA-4 blockade in melanoma. N Engl J Med, 2014. 371 (23): p. 2189-2199.
16. Johnson, B. E., et al., Mutational analysis reveals the origin and therapy-driven evolution of recurrent glioma. Science, 2014. 343 (6167): p. 189-193.
17. Hanada, K. I., et al., A phenotypic signature that identifies neoantigen-reactive T cells in fresh human lung cancers. Cancer Cell, 2022. 40 (5): p. 479-493.e6.
18. McGranahan, N., et al., Clonal neoantigens elicit T cell immunoreactivity and sensitivity to immune checkpoint blockade. Science, 2016. 351 (6280): p. 1463-9.
19. Nemunaitis, J., et al., Clonal Neoantigen: Emerging “Mechanism-based” Biomarker of Immunotherapy Response. Cancers (Basel), 2023. 15 (23): p. 5616.
20. Kristensen, N. P., et al., Neoantigen-reactive CD8+ Tcells affect clinical outcome of adoptive cell therapy with tumor-infiltrating lymphocytes in melanoma. The Journal of Clinical Investigation, 2022. 132 (2).
21. Peri, A., et al., The landscape of T cell antigens for cancer immunotherapy. Nature Cancer, 2023. 4 (7): p. 937-954.
22. Litchfield, K., et al., Meta-analysis of tumor-and T cell-intrinsic mechanisms of sensitization to checkpoint inhibition. Cell, 2021. 184 (3): p. 596-614 e14.
23. Al Bakir, M., et al., The evolution of non-small cell lung cancer metastases in TRACERx. Nature, 2023. 616 (7957): p. 534-542.
24. Rosenthal, R., et al., Neoantigen-directed immune escape in lung cancer evolution. Nature, 2019. 567 (7749): p. 479-485.
25. Wolf, Y., et al., UVB-Induced Tumor Heterogeneity Diminishes Immune Response in Melanoma. Cell, 2019. 179 (1): p. 219-235.e21.
26. Wu, D., et al., Identification of Clonal Neoantigens Derived From Driver Mutations in an EGFR-Mutated Lung Cancer Patient Benefitting From Anti-PD-1. Front Immunol, 2020. 11: p. 1366.
27. Rocconi, R. P., et al., Gemogenovatucel-T (Vigil) immunotherapy as maintenance in frontline stage III IV ovarian cancer (VITAL): a randomised, double-blind, placebo-controlled, phase 2b trial. Lancet Oncol, 2020. 21 (12): p. 1661-1672.
28. Rocconi, R. P., et al., Gemogenovatucel-T (Vigil) immunotherapy demonstrates clinical benefit in homologous recombination proficient (HRP) ovarian cancer. Gynecol Oncol, 2021. 161 (3): p. 676-680.
29. Walter, A., et al., Gemogenovatucel-T (Vigil) maintenance immunotherapy: 3-year survival benefit in homologous recombination proficient (HRP) ovarian cancer. Gynecol Oncol, 2021. 163 (3): p. 459-464.
30. Strickland, K. C., et al., Association and prognostic significance of BRCA1/2-mutation status with neoantigen load, number of tumor-infiltrating lymphocytes and expression of PD-1/PD-L1 in high grade serous ovarian cancer. Oncotarget, 2016. 7 (12): p. 13587-98.
31. Birkbak, N. J., et al., Tumor mutation burden forecasts outcome in ovarian cancer with BRCA1 or BRCA2 mutations. PLOS One, 2013. 8 (11): p. e80023.
32. Germano, G., et al., Inactivation of DNA repair triggers neoantigen generation and impairs tumour growth. Nature, 2017. 552 (7683): p. 116-120.
33. Senzer, N., et al., Long Term Follow Up: Phase I Trial of “bi-shRNA furin GMCSF DNA Autologous Tumor Cell” Immunotherapy (FANG™) in Advanced Cancer. Journal of Vaccines & Vaccination, 2013. 4 (8): p. 209.
34. Senzer, N., et al., Phase I trial of “bi-shRNAi (furin) GMCSF DNA autologous tumor cell” vaccine (FANG) in advanced cancer. Mol Ther, 2012. 20 (3): p. 679-86.
35. Ghisoli, M., et al., Pilot Trial of FANG Immunotherapy in Ewing's Sarcoma. Mol Ther, 2015. 23 (6): p. 1103-1109.
36. Maples P B, K. P., Oxendine I, Jay C, Yu Y, Kuhn J, Nemunaitis J, TAG Vaccine: Autologous Tumor Vaccine Genetically Modified to Express GM-CSF and Block Production of TGFβ2. BioProcessing Journal, 2009. 8 (2): p. 38-45.
37. Nemunaitis, J., Multifunctional vaccines in cancer: the ‘triad’ approach. Expert Rev Vaccines, 2011. 10 (6): p. 713-5.
38. Van Allen, E. M., et al., Genomic correlates of response to CTLA-4 blockade in metastatic melanoma. Science, 2015. 350 (6257): p. 207-211.
39. Willoughby, D., et al., Exome Sequencing Shows Same Pattern of Clonal Tumor Mutational Burden, Intratumor Heterogenicity and Clonal Neoantigen between Autologous Tumor and Vigil Product. Scientific Reports, (submitted).
40. Tanner, G., et al., Benchmarking pipelines for subclonal deconvolution of bulk tumour sequencing data. Nat Commun, 2021. 12 (1): p. 6396.
41. Hundal, J., et al., pVACtools: A Computational Toolkit to Identify and Visualize Cancer Neoantigens. Cancer Immunol Res, 2020. 8 (3): p. 409-420.
42. O'Donnell, T. J., A. Rubinsteyn, and U. Laserson, MHCflurry 2.0: Improved Pan-Allele Prediction of MHC Class I-Presented Peptides by Incorporating Antigen Processing. Cell Syst, 2020. 11 (4): p. 418-419.
43. Thuesen, N. H., et al., Benchmarking freely available HLA typing algorithms across varying genes, coverages and typing resolutions. Front Immunol, 2022. 13: p. 987655.
44. Shen, R. and V. E. Seshan, FACETS: allele-specific copy number and clonal heterogeneity analysis tool for high-throughput DNA sequencing. Nucleic Acids Res, 2016. 44 (16): p. e131.
45. Dilthey, A. T., et al., HLA*LA-HLA typing from linearly projected graph alignments. Bioinformatics, 2019. 35 (21): p. 4394-4396.
46. Gillis, S. and A. Roth, PyClone-VI: scalable inference of clonal population structures using whole genome data. BMC Bioinformatics, 2020. 21 (1): p. 571.
47. McLaren, W., et al., The Ensembl Variant Effect Predictor. Genome Biol, 2016. 17 (1): p. 122.
48. Hundal, J., et al., pVAC-Seq: A genome-guided in silico approach to identifying tumor neoantigens. Genome Med, 2016. 8 (1): p. 11.
49. Oh, J., et al., Phase II study of Vigil® DNA engineered immunotherapy as maintenance in advanced stage ovarian cancer. Gynecol Oncol, 2016. 143 (3): p. 504-510.
50. Maples, P., et al., FANG Vaccine: Autologous Tumor Cell Vaccine Genetically Modified to Express GM-CSF and Block Production of Furin. BioProcessing Journal, 2010. 8: p. 4-14.
51. Nemunaitis, J., et al., Gemogenovatucel-T (Vigil): bi-shRNA plasmid-based targeted immunotherapy. Future Oncol, 2024: p. 1-16.
52. Gourraud, P. A., et al., HLA diversity in the 1000 genomes dataset. PLOS One, 2014. 9 (7): p. e97282.
53. Kraya, A. A., et al., Genomic Signatures Predict the Immunogenicity of BRCA-Deficient Breast Cancer. Clin Cancer Res, 2019. 25 (14): p. 4363-4374.
54. Banerjee, A. and S. Gerondakis, Coordinating TLR-activated signaling pathways in cells of the immune system. Immunol Cell Biol, 2007. 85 (6): p. 420-4.
55. Bierie, B. and H. L. Moses, TGF-beta and cancer. Cytokine Growth Factor Rev, 2006. 17 (1-2): p. 29-40.
56. Zhang, F., et al., Juglanin Inhibits Osteoclastogenesis in Ovariectomized Mice via the Suppression of NF-kappaB Signaling Pathways. Front Pharmacol, 2020. 11: p. 596230.
57. Bian, Z., et al., Intratumoral SIRPalpha-deficient macrophages activate tumor antigen-specific cytotoxic T cells under radiotherapy. Nat Commun, 2021. 12 (1): p. 3229. Metzinger, M. N., et al., Chimeric Antigen Receptor T-Cell Therapy: Reach to Solid 58. Tumor Experience. Oncology, 2019. 97 (2): p. 59-74.
59. Majumder, A., Evolving CAR-T-Cell Therapy for Cancer Treatment: From Scientific Discovery to Cures. Cancers (Basel), 2023. 16 (1).
60. Daei Sorkhabi, A., et al., The current landscape of CAR T-cell therapy for solid tumors: Mechanisms, research progress, challenges, and counterstrategies. Front Immunol, 2023. 14: p. 1113882.
61. Drougkas, K., et al., Comprehensive clinical evaluation of CAR-T cell immunotherapy for solid tumors: a path moving forward or a dead end? J Cancer Res Clin Oncol, 2023. 149 (6): p. 2709-2734.
62. Gerlinger, M. and C. Swanton, How Darwinian models inform therapeutic failure initiated by clonal heterogeneity in cancer medicine. Br J Cancer, 2010. 103 (8): p. 1139-43.
63. White, L. G., et al., Controlling Cell Trafficking: Addressing Failures in CAR T and NK Cell Therapy of Solid Tumours. Cancers (Basel), 2022. 14 (4).

### Example 5. Appendix

Per the description in Example 3, there are three markers (cTMB, ITH, and cNEO) that have been identified for differentiating patients that will be responders vs non-responders to Vigil. ITH is determined as TMB-cTMB, and thus cTMB and ITH are related. The markers can be used in combination as well.

cTMB: FIG. 9 shows the separation of cTMB between responders and non-responders to Vigil for cTMB values. A cutoff of any value between 2 and 3 only provides one false negative, where patients with a cTMB higher than the cutoff are identified as responders. FIG. 10 shows the separation of cNEO between responders and non-responders to Vigil for cNEO values. A cutoff of 5 only provides one false negative and no false positives.

CTMB corresponds to the amount of non-synonymous mutations that are clonal, namely the mutations that occur in a vast majority of the cells and thus are original to the first tumor clone. CTMB can be expressed as the number of clonal mutations that occur per megabase (MB). To determine whether a mutation is clonal, a cancer cell fraction (CCF) is determined for the mutation. CCF is a measure of a percentage of the sampled cells that have the mutation. A CCF greater than a threshold (e.g., 0.9 or 90%) can indicate a mutation is clonal.

To determine CCF, a sample(s) of a tumor are sequenced, e.g., by subjecting the cells to sonication to fragment the DNA, amplifying the DNA fragments (e.g., by PCR) to obtain a sequencing library, and then sequencing the sequencing library to obtain sequence reads. The sequence reads are then aligned to a reference exome, as only mutations in the exome are used. Normal cells (e.g., white blood cells) are also sequenced. The two sets of sequence reads are then aligned to a reference exome and differences between the two sets of reads are identified. For example, at a given genomic position, the sequence reads of normal cells might all have an A, and thus the normal cells are homozygous A/A. The tumor reads might have A and G, and so a possible variant (mutation) is identified. Each possible mutation is then analyzed via multiple stages to determine if it is a true mutation and if it is a true mutation whether it is clonal.

As a first filtering step, the possible variant is analyzed to filter out sequencing errors as opposed to true variants. For variant calling, the software pipeline the combination of GATK Mutect2 and FilterMutectCalls. Such software is commercially available and looks at the allelic fraction to identify true mutations.

Once a true mutation is identified, PyClone 6.1 determines the CCF using allelic frequency and allele-specific copy number of tumor-specific variants. The CCF values for all the true mutations are clustered. Each cluster has a similar CCF. A cluster is identified as clonal if the CCF is greater than 0.9, although other thresholds can be used.

The total number of mutations in all of the clusters that are identified as clonal are used to determine the total number of clonal mutations, which can be normalized based on the amount of the exome that is analyzed.

The metric for clonal neoantigens (cNEO) quantifies the number of peptides expressed by the clonal mutations that will likely appear on the cell surface (i.e., bind with the MHC haplotype) and that satisfy certain criteria, e.g., (1) 50% of the peptide is expected to bind at 500 nano molar and (2) binding affinity is higher for the peptide of the mutant variant as compared to the peptide for the wild type.

The tumor sequence reads are analyzed to determine the MHC-1 haplotype calls, specifically HLA*LA software is used. For each clonal mutation, the software pVACSeq is used to generate the sequences for all non-synonymous variants associated peptides of length 8-11 amino acids and predicts peptide-MHC-1 binding affinity. PVACSeq estimated MHC-1 molecule binding affinity and fold-difference in binding affinity between mutant and wild-type peptides for each non-synonymous tumor-associated variant identified by Mutect2 and annotated with VEP.

The pVacSeq filter command was used to filter the peptide list to select peptides with IC50<500 nM, a fold-change difference in IC50 for reference versus variant peptide of >1, and finally to select peptides falling among the lowest 5% of total peptides in Kb value (selected experiments only).

### V. Example Techniques Using CTMB and CNEO to Identify Responders

As described above, cTMB and cNEO can be used to identify responders to an immunotherapy treatment, e.g., which includes genetically modified tumor cells of the subject and/or that induces the immune system to attack cells carrying the neoantigens. This section provides further details on example techniques to identify and treat such responders.

### A. Example Software Pipeline

Exome sequencing can be performed to detect mutations, e.g., non-synonymous mutations that alters the amino acid sequence of a protein. All or part of the exome can be sequenced. The mutations can be detected in various ways, e.g., as described below. Raw sequence read files can be received at a computer and analyzed to detect the mutations. The mutations can be limited to those that are clonal mutations such that they are assessed to be from a primary clone. After identifying mutations and whether that those are clonal or non-clonal, embodiments can calculate ITH, cTMB, and/or cNEO.

### 1. Unique Molecular Identifiers (UMIs)

In some embodiments, the developed bioinformatics pipeline can use unique molecular identifiers (UMIs) while generating BAM files, which can improve the accuracy of variant calling because reads derived from the same source DNA library molecule can be combined into a consensus sequence.

A UMI can be attached to a template nucleic acid molecule as a barcode that uniquely identifies that template molecule. Then, that template nucleic acid molecule can be amplified (e.g., using PCR) along with other template nucleic acid molecule in the sample. A UMI can be used to determine whether two reads are from the same template nucleic acid molecule and thus duplicates, so that the reads are only counted once, e.g., as a consensus read. An advantage of the UMI Processing is that reads that are derived from the same original molecule can be combined in a consensus read with higher base quality at each position where there is consistency between the input duplicate reads. The use of UMIs can provide accurate representation of the abundance of each mutation/variant.

If the exome sequence reads contain unique molecular identifiers (UMIs), the sequences can be processed (e.g., using fgbio software) to generate consensus reads, remove duplicates, and align the consensus reads to the genome. The process steps can include (1) creating BAM files containing unaligned reads with the UMI sequences removed and stored in tags, (2) aligning reads to the genome (e.g., with bwa-mem) and then recovering headers and tags for unmapped reads (e.g., using ZipperBams software), (3) generation of initial coverage metrics and grouping of reads based on identical UMIs sequences, (4) calling consensus reads based on UMI sequence and mapped position (e.g., with fgbio CallConsensusReads), alignment of these consensus reads to the genome with bwa-mem, recovery of headers and UMI tags (e.g., using ZipperBams), quality filtering of the mapped consensus reads, generation of coverage metrics for the mapped consensus reads, recalibration of base quality scores with GATK BaseRecalibrator, and generation of final BAM files by cropping the base-recalibrated mapped consensus reads to include only the exonic regions of the genome, and finally (5) taking the mapped reads for just the normal samples generated from step 2 above through base quality score recalibration, cropping, and merging with unaligned reads from step 1 above in order to create BAM files suitable for use with modules described herein, e.g., a module for calling MHC-I and MHC-II haplotypes using HLA*LA [19], a module for calling and annotating SNVs and InDels using GATK Mutect2 and Funcotator, and a module for determining allelic copy number using FACETS [20], which can be used to determine cTMB, ITH, and cNEO.

### 2. Detecting Mutations

Once a final set of reads is determined (e.g., using UMIs), the reads that map to a given position for the tumor sample and the normal sample can be compared to detect sequence variants as possible mutations. Certain criteria can be used to determine if a true mutation exists or if the variant is just a sequencing error (e.g., when the variant occurs in a small number of reads). For example, the reads can first be mapped to the reference genome to find the most likely origin in the genome for that read, and multiple reads (e.g., 100-600) can be identified at a given position for both samples. Software can then identify the highly significant differences between the nucleotide sequence between the tumor cell and the normal cell. A frequency of a variant allele can be used (e.g., compared to a threshold) along with other criteria, such as allelic copy number, quality of the basecall for the wildtype allele and variant allele, and whether the variant is in a list (database) of artifact variants or common variants that are not pathogenic. A threshold for a frequency and/or an absolute number of reads with the mutation can be used.

As an example, a mutation detection module (e.g., GATK mutect 2) can be used to call and annotate single nucleotide variants (SNVs) and small insertions/deletions (Indels). The mutation detection module can use the normal sample as a reference for the tumor sample. The genotype and coverage depth for each allele can be recorded in the output file (e.g., VCF file) for each called variant for both the tumor and normal sample. Importantly, a panel of normal and germline resource databases can be used to exclude variants that occur with some frequency in the healthy human population The files can be further processed (e.g., through GATK FilterMutect2Calls) to assign codes to each variant in the filter field indicating a specific failure mode or PASS if the variant is considered reliable. The filtered files can be annotated to called variants based on a set of data sources, each with its own matching criteria, e.g., using Funcotator version 4.3.0.0.

Allele specific copy number can be estimated for mutations, e.g., within chromosomal segments that appear to comprise the majority of the non-telomeric and -centromeric long arm and short arm of each chromosome. The allelic copy number can be determined using FACETs v0.5.14 (Shen & Seshan, 2016). FACETS can post-process the BAM files and call the single nucleotide variants within the tumor sample in a process that also utilizes the BAM file from the normal sample. The allelic depth at both heterozygous and homozygous variant sites can be used as the basis for estimating the depth for two alleles at each position and then uses the combination of the total estimated copy number for each region and the depths at each allele to derive the allelic copy number. Such a module can estimate and report the start and stop position, the total copy number, and the minor allele copy number for each of those segments and estimates the overall tumor purity for the tumor sample (i.e. determines the level of contamination from normal tissue).

The mutations can be of various types, e.g., point mutations, SNPs, copy number, tandem repeats for microsatellites, and the like.

B. Identifying Responders Using cTMB

A tumor mutation burden can be determined by counting a total number of mutations, e.g., the number of non-synonymous mutations within the exome region, per MB of exome. CTMB can be calculated as the tumor burden for mutations that are identified as clonal mutations from the primary clone. CTMB can be determined based on a clonal cancer cell fraction, described in more detail below. For example, a clonal cell fraction of greater than 90% can be used to identify clonal mutations. Various thresholds for the clonal cell fraction can be used, e.g., at least 20%, 30%, 40%, 50%, 60%, 70%, 80%, or 90%. The cTMB score described herein can be determined for mutations having a CCF score above the specified threshold, as those are mutations determined to be clonal.

In various embodiments, to identify clonal variants we utilize information from the variant calling software (e.g., GATK Mutect2 and FilterMutect2Calls) including one or more of: the chromosomal position, the sequencing read depth for the reads that match the reference sequence, and the sequencing read depth for the reads that match the variant sequence. The above information from the variant caller can be combined with the allelic copy number for the major and minor allele and the estimated percentage tumor DNA in the tumor genomic DNA sample that is obtained from the copy number analysis software (typically FACETS) and used as input for the clonal analysis, which can be performed using Pyclone 6.1 The output from the clonal analysis can generate a Cancer Cell Fraction (CCF) for each variant, which is the estimated percentage of cells in the sample that carry the variant. We then count the number of non-synonymous variants with a CCF above or equal to a specified threshold (typically 0.9 but could vary) and normalize this value to the length of the exome sequence interrogated during the sequencing to arrive at the cTMB value.

As shown in FIG. 9, there is a clear separation between responders (CR/PR) and non-responders (PD) using two different techniques for determining cTMB. For one technique, a threshold anywhere between 2-3 provides good discrimination. For another technique, a threshold anywhere between about 2.2-4 provides good discrimination. Depending on the immunotherapy being used, different thresholds can be used, e.g., different thresholds for ovarian cancer and lung cancer.

### 1. Cancer Cell Fraction

Cancer cell fraction (CCF) can be determined for each mutation and can be used to determine whether a mutation is clonal. The CCF can be determined as the percentage of the tumor cells that are likely to have that mutation. Such a value can be determined based on the fraction of reads that have the variant sequence and a local copy number. These clonal mutations can then be used to determine the clonal TMB (cTMB), e.g., as a number of clonal mutations per megabase of DNA that is covered by the exome.

As examples, the CCF can be determined from the fraction of reads that have the variant sequence alone or in combination with one or both of the allelic copy number and an estimate of the tumor purity.

Clusters of mutations can be identified, with each cluster having an associated CCF. For example, the PyClone software can identify cluster clones. Each mutation is only assigned to one of the clones. Thus, a clonal group has unique mutations to that clonal group.

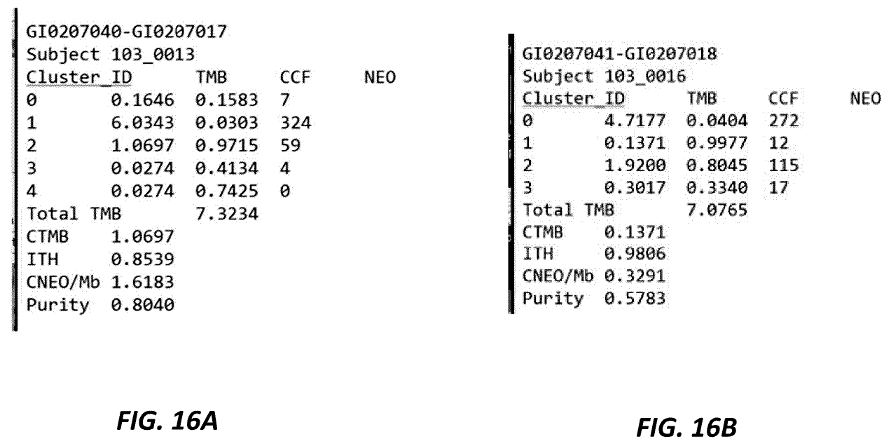

FIG. 16A shows an example report for a first subject that includes cTMB. Five clusters are shown (numbered zero to four), with a corresponding TMB in a next column, followed by CCF and number of neoantigens, which will be discussed later. We see that clone number 2 has cancer cell fraction above 0.9, which above the 0.9 (90%) threshold used in this example. That cluster can be considered clonal variants and that's why the number under cTMB is the same as the number for clone 2. The TMB, CCF, and NEO can be determined as averages for the mutations in that cluster. The cTMB is the TMB for the variants that are considered to be clonal based on their CCF. In the example of FIG. 16A, clone #2 has a sufficiently high CCF, and so the cTMB for that clone is used and would be 1.0697. If multiple clones have a sufficiently high CCF, their TMB values can be summed to provide the cTMB. In other examples, just the cluster with the maximum CCF can be used.

FIG. 16B shows an example report for a second subject that includes cTMB. In this example, clone #1 has a CCF greater than 0.9 (example threshold) and thus are determined to be clonal, and the TMB 0.1371 for clone #2 can contribute to the cTMb, as is shown.

The local copy number (e.g., as determine by software Facets) can be determined by independently analyzing all the mutations and their local abundances in both the tumor and the blood cell. Regions of the chromosome that may be amplified (duplicated or higher) or deleted can be identified. The minor and major allele copy number can be used to fine tune the predictions, e.g., correct undercount or overcount.

As an example, assume the normal blood sample is AA at a position and the tumor is AG, then if the G is present as 50% of the total reads at that position, then there would be 100% cancer cell fraction, assuming there is a normal copy number in that region. Because the tumor is made-up of a mixture of clones that started with one clone, the additional errors that are introduced into the DNA (sub-clones) are only present as a smaller percentage of the cells in the tumor.

Copy number can be used to accurately determine the amount (e.g., fraction) of cancer cells that have the mutation. For example, if a cancer cell has a particular haplotype with a 3× amplification for the wildtype (e.g., A) then there will be 3 copies with A and one copy with the mutation (e.g., G). The raw fraction is 25%, falsely indicating 50% of the cancer cells have the mutation G (e.g., one cell homozygous for A and one cell with the mutation G). However, the true cancer cell fraction is 100% because each cell would have the mutation G, but just extra copies of the wildtype A.

As an example of using tumor purity, if we know the tumor sample is 20% pure (20% tumor/80% normal cells) and the genotype is G/G in the normal cells and A/G in the tumor cells, the determined allelic fraction of G would only be 10%. The CCF would be 100% after correcting for the tumor purity (because in the tumor cells the true allelic fraction is 50%).

The threshold for determining whether a mutation is clonal (e.g., using the cancer cell fraction, CCF) can take various forms to distinguish clonal from non-clonal mutations. For example, the comparison of the CCF to a threshold can include statistical measures, such as using the CCF to determine a likelihood of clonality based on a probability distribution. The likelihood can be compared to a threshold, which still encompasses a comparison if a CCF to a threshold. As another example, a confidence interval can be used along with the CCF to determine whether the likelihood is above a threshold for distinguishing clonal from non-clonal mutations. The threshold can be determining from a training set with known statuses of clonality so as to maximize sensitivity and specificity.

### 2. Determination of HRP

As described above, an HRP status can be used as a further criterion for whether a patient will respond to an immunotherapy, such as Vigil®. When the HR pathway is working properly, DNA can be repaired effectively and is error free, maintaining genomic stability. When the HR pathway is disrupted by gene mutations, promoter methylation or unknown causes, the HR pathway stops working leading to genomic instability or homologous recombination deficiency. HRD is defined as the inability to repair double stranded DNA breaks. In some embodiments, a Myriad myChoice cutoff score of ≥42=HRD°; ≤41=HRP°

HRD results in genomic aberrations including telomeric allelic imbalance, loss of heterozygosity and large-scale transitions which are used to calculate an HRD score. Patients who have a defective pathway are more likely to respond to drugs that impact DNA stability such as platinum drugs and PARPi's. HRD high scores are associated with higher mutation rate and higher subclonal neoantigen content.

The results in FIGS. 13A-13B show the increased response of HRP patients with a high cTMB relative to non-HRP patients (FIGS. 13C-13D), illustrating the benefit of using HRP status.

Some further possible details on HR are as follows. A mutation of the BRCA gene leads to HR deficiency, therefore BRCA-m patients are HRD. BRCA-wt patients can be HRP or HRD. HRP prevalence is 48.5% of OvC, e.g., based on next generation sequencing and promoter methylation. HRD, BRCA-m achieve better OS, PFS to platinum-based chemotherapy over HRP3-9. HRD, BRCA-m achieve better OS, PFS to bevacizumab over HRP10.

### 3. Method for Identifying Responders

Using cTMB with or without HRP status can provide a higher hazard ratio. A higher drug effect can be achieved due to increased precision in identifying responders.

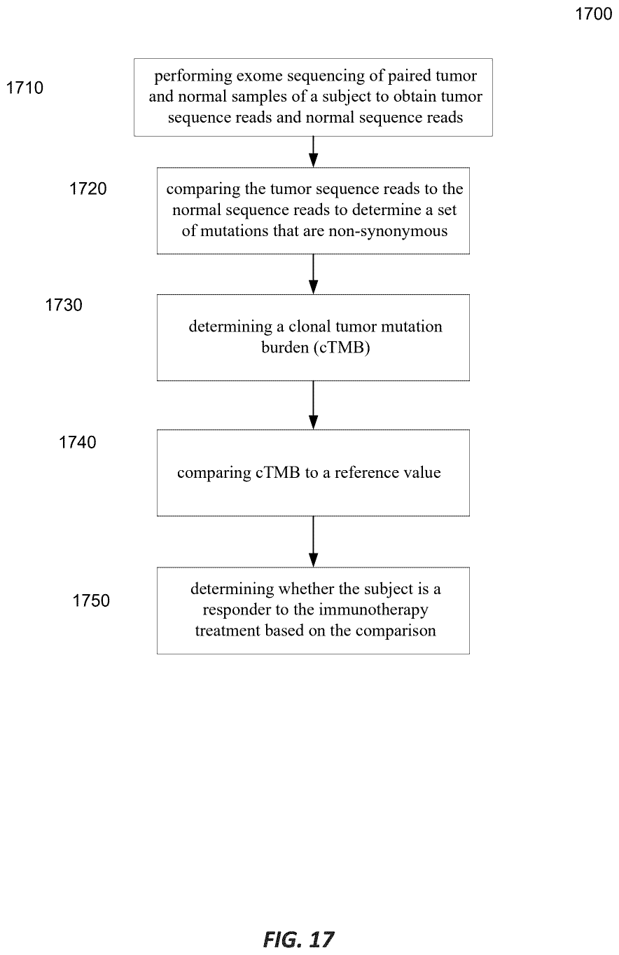

FIG. 17 is a flowchart of a method 1700 for using cTMB to identify responders to an immunotherapy treatment according to embodiments of the present disclosure. Method 1700 can be used to determine a treatment for a subject having a particular type of cancer. At least a portion of the steps of method 1700 can be performed by a computer system.

At block 1710, exome sequencing is performed on nucleic acids of paired tumor and normal samples of a subject to obtain tumor sequence reads and normal sequence reads. In some embodiments, the exome sequencing can use unique molecular identifiers. For example, unique molecular identifiers can be attached to template nucleic acids of the paired tumor and normal samples, and the template nucleic acids can be amplified prior to sequencing to generate reads. A consensus read can then be determined from one or more reads for each of the unique molecular identifiers, thereby generating consensus reads. The consensus reads can then be used for comparing the tumor sequence reads to the normal sequence reads to determine the set of mutations.

At block 1720, the tumor sequence reads are compared to the normal sequence reads to determine a set of mutations that are present in the tumor cells but not in the normal cells. As part of the exome sequencing, unique molecular identifiers (UMIs) can be attached to template nucleic acids of the paired tumor and normal samples. The template nucleic acids can then be amplified prior to sequencing to generate reads. The UMIs can be used to de-duplicate reads from the same template. For example, a consensus read can be determined from one or more reads for each of the unique molecular identifiers, thereby generating consensus reads. The consensus reads can then be used to compare the tumor sequence reads to the normal sequence reads in order to identify the that are in the tumor sample and not in the normal sample.

The mutations can be selected to be non-synonymous, which can be determined by checking to see whether the variant impacts an amino acid sequence spanning the genomic position. If the variant changes the amino acid sequence of a protein it is considered non-synonymous. This property, synonymous versus non-synonymous, can be determined using the variant genomic position and sequence change and comparing it to a database. It can be performed subsequently to identification of the true tumor-associated variants but before calculation of cTMB and cNEO scores. In some implementations, Funcotator is used to determine which variants mutations are non-synonymous, which can be done at an annotation step.

At block 1730, a clonal tumor mutation burden (cTMB) is determined. In some embodiments, the cTMB can be determined by, for each of the set of mutations, determining a respective amount of the tumor sequence reads that have the mutation and determining whether the mutation is a clonal mutation based on the respective amount, thereby determining clonal mutations. The cTMB can then be determined using the clonal mutations.

The cTMB can be normalized using a size of the exome sequenced. The respective amount of tumor sequence reads that have the mutation can be an allelic fraction. Determining whether the mutation is a clonal mutation can be further based on an allelic copy number and a tumor purity. In some embodiments, only the non-synonymous mutations are counted in the total tumor mutations used to calculate the cTMB.

At block 1740, the cTMB is compared to a reference value. The reference value can be determined using training samples of training subjects that have the particular type of cancer and that have a known responder classification to an immunotherapy treatment. The immunotherapy treatment can include genetically modified tumor cells of the subject, e.g., Vigil®.

At block 1750, it is determined whether the subject is a responder to the immunotherapy treatment based on the comparison.

Additionally to determine whether the subject is a responder, it can be determined whether the subject is homologous recombination proficient (HRP). It can be determined whether the subject is the responder to the immunotherapy treatment further based on the subject being homologous recombination proficient (HRP), as described herein. If the subject is not homologous recombination proficient (HRP), it can be determined to not respond to the immunotherapy treatment. The training samples can be partially or only of training subjects that are homologous recombination proficient (HRP).

### C. Identifying Responders Using CNEO

An amount of clonal neoantigens (cNEO) on a cell surface can be used to determine whether a patient is a responder to an immunotherapy, such as Nivolumab or Vigil®. The higher amount of clonal neoantigens, the more likely the immunotherapy will work. To determine cNEO, embodiments can use MHC/HLA competencies to determine an amount of clonal neoantigens on the cell surface. For example, it can be determined whether a mutation results in peptides that bind to the patient's HLA, thereby providing an amount of clonal neoantigens on the cell surface.

As shown in FIG. 10, the cNEO values are well separated between the responders and the non-responders. A threshold value of 5 provides 100% specificity and 85% (6 out of 7) sensitivity.

### 1. Amount of Clonal Neoantigens

Some embodiments can start with VCF files (or other type of files) that contain a list of clonal mutations (e.g., all clonal mutations identified) and information about the mutations, e.g., the number of reads representing each allele. A cNEO module can annotate the files with protein coding information for the mutations, and then generate peptides sequences having a specified length (e.g., 8-11 amino acids). Another part of the pipeline can identify the most likely MHC haplotype for each patient.

The generated peptides can be compared to the MHC haplotype of the patient to identify peptides that have sufficient likelihood (e.g., greater than a threshold) of the peptide being presented on the cell surface in complex with the MHC molecules (haplotype) encoded by the patient. A given peptide can be in more than one clonal neoantigen, and a clonal neoantigen can include multiple peptides with sufficient likelihood. If so, each instance can be used to increase the likelihood. In some implementations, the likelihood can be determined based on whether there is sufficient binding (e.g., greater than a threshold) such that the peptide would likely appear on the cell surface. A binding affinity score can be determined for each peptide; and if the score satisfies the criteria for inclusion (e.g., a threshold), then that peptide can be used to determine the amount of clonal neoantigens. Such criteria can include one or more of: (1) 50% of the peptide is expected to bind at 500 nano molar concentration and (2) the binding affinity is higher in the tumor in the mutant variant as compared to the wild type sequence in the normal sample, e.g., a blood sample.

FIGS. 16A and 16B also show values for cNEO according to an embodiment. For FIG. 16A, clonal group #2, which has CCF greater than 0.9, has 59 peptides that satisfy the above criteria. To get the value normalized by the size of the exome analyzed, we can divide the 59 by 36.458 to get the 1.6183 value for cNEO/Mb. Accordingly, a cNEO score can be determined for each cluster by summing the filtered peptide count (e.g., all qualifying peptides or mutations with a qualifying peptide) for all non-synonymous variants associated with the cluster. A cNEO value for the tumor/normal sample pair can then determined by dividing NEO for the primary clone by the size of the exome in Mb; a tumor with no primary clone will have a cNEO of zero

In some embodiments, a cNEO score can be determined as follows. For every non-synonymous clonal variant, the module can generate a list of all possible peptides within a specified length range (e.g., 8-11 amino acids) that contain one or more non-consensus amino acids derived from the variant. The module can further filter the raw list to select peptides that have a predicted high binding affinity (typically IC50 of 500 nM or below) for the patient's MHC-1 molecules and/or also higher binding affinity for the patient's MHC-I molecules than a matching peptide derived from the consensus sequence. The module can then count the peptides that meet the filtering criteria for each clonal non-synonymous variant, sum all of these peptides for the patient, and optionally normalize this value to the exome length to arrive at a cNEO value.

In other embodiments, instead of counting every peptide that meets the filtering criteria, the module can count the number of clonal mutational sites that have at least one peptide satisfying the criteria. For example, the module can count the first peptide that meets the filtering criteria (binding affinity and difference in binding affinity from consensus peptide) for each clonal non-synonymous variant. In this example, the total number of peptides counted for a patient would never be higher than the number of clonal non-synonymous variants. This revised total can be normalized to the exome length to arrive at the alternative cNEO value.

As to specific software tools that can be used, the outputs of Mutect2 and HLA*LA can used as input for pVAC-Seq to predict the sequence of peptide neoantigens, and the output of Mutect2 and FACETS can be used as input to PyClone 6.1 to perform a clonality analysis and assign each SNV and Indel in a patient tumor sample to a primary clone or one of several subclones. Custom scripts are used to add information about predicted peptide neoantigens to the PyClone report, to filter the report to include only non-synonymous variants, and to summarize and calculate the key metrics for each patient tumor sample.

As a further example, a neoantigens script can annotate Mutect2 VCF files using VEP software and then initiate a run of pVAC-Seq (Hundal et al., 2020) for each tumor sample using the VEP-annotated VCF and MHC-1 A, B, and C haplotypes from HLA*LA output as source information. pVAC-Seq generates all possible peptide sequences of parameterized length (e.g., 8-11 amino acids) that overlap with at least one variant amino acid at each SNP/Indel for non-synonymous variant of appropriate mutation type (missense, frameshift, etc.), and also generate the corresponding peptides from the same wild-type sequence. The software then uses one or more predictive models to calculate the binding affinity in nM for the possible peptides for each patient's MHC-I haplotypes derived from HLA*LA. The software reports the binding affinity, as determined by each algorithm run, for each pair of possible mutant and wild-type peptides for each variant in a table for each sample. In addition, the tables can include a best binding affinity, fold-change of best binding affinity for the mutant versus wild-type peptide, and the percentage rank of each peptide relative to all other peptides in the table. One mode for running the software is to select only MHCflurry 2.0 algorithm (O'Donnell et al., 2020) such that the best binding affinity is always derived from that algorithm.

A neoantigens filter script can filter the pVAC-Seq output based on user-provided criteria in a config file, which can include predicted MHC-1 binding affinity (default 500 nM or lower), difference in binding affinity between mutant and wild-type peptides (default 1-fold or more), and the overall rank of the mutant peptide's binding affinity relative to all peptides considered for the tumor sample. The script can create an updated version of the PyClone report for each tumor sample that includes the number of peptides for each mutation ID that meet each of the filtering criteria and the sequence of the best mutant peptide for each mutation ID, which is based on the lowest binding affinity score among peptides which also meet the mutant versus wild-type fold-change criteria. The updated PyClone report can include only nonsynonymous variants as compared to the original report which included all variants. The report can include a cNEO score, which can, for example, be determined by counting peptides associated with clonal mutations and binding with MHC or by the number of clonal mutations having at least one of such peptides.

### 2. Method for Identifying Responders

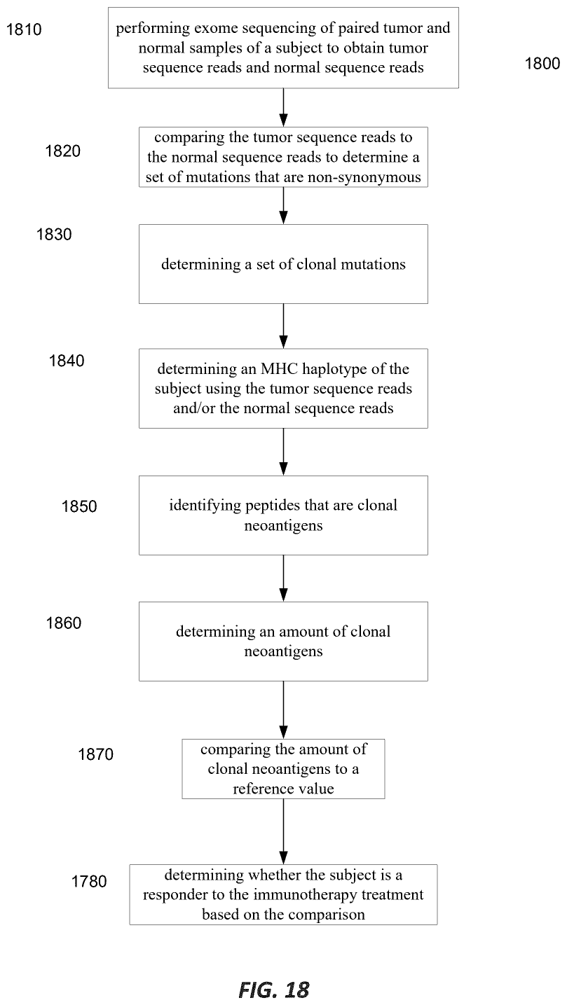

FIG. 18 is a flowchart of a method 1800 for using cNEO to identify responders to an immunotherapy treatment according to embodiments of the present disclosure. Method 1800 can be used to determine a treatment for a subject having a particular type of cancer. At least a portion of the steps of method 1800 can be performed by a computer system. Aspects of method 1800 can be performed in a similar manner as method 1700 and other methods performing similar steps.

At block 1810, exome sequencing is performed on nucleic acids of paired tumor and normal samples of a subject to obtain tumor sequence reads and normal sequence reads. Such example sequencing can use UMIs, as described for method 1700.

At block 1820, the tumor sequence reads are compared to the normal sequence reads to determine a set of mutations that are present in the tumor cells but absent from normal cells. As part of the exome sequencing, unique molecular identifiers (UMIs) can be attached to template nucleic acids of the paired tumor and normal samples. The template nucleic acids can then be amplified prior to sequencing to generate reads. The UMIs can be used to de-duplicate reads from the same template. For example, a consensus read can be determined from one or more reads for each of the unique molecular identifiers, thereby generating consensus reads. The consensus reads can then be used to compare the tumor sequence reads to the normal sequence reads in order to identify the mutations that that are in the tumor sample and not in the normal sample.

At block 1830, a set of clonal mutations that are non-synonymous is determined. Clonal mutations can be determined using techniques described in method 1700. As examples, determining a clonal mutation of the set of clonal mutations can use one or more of: a chromosomal position of the clonal mutation, a sequencing read depth for reads that match the reference sequence at the chromosomal position, a sequencing read depth for reads that match the variant sequence, allelic copy number for a major allele and a minor allele at the chromosomal position, and an estimated percentage tumor DNA in the tumor genomic DNA sample.

At block 1840, an MHC haplotype of the subject is determined using the tumor sequence reads and/or the normal sequence reads. Example techniques for determine the MHC haplotype are described herein.

At block 1850, certain peptides are identified as clonal neoantigens. For example, for each clonal mutation, a peptide corresponding to a non-synonymous clonal mutation at a genomic position can be generated on the computer. An MHC-1 binding affinity for the peptide can be determined, e.g., using software techniques and tools described herein.

A likelihood of the peptide being presented on the cell surface in complex with the MHC haplotype encoded by the patient can be determined. Then, whether the peptide is a clonal neoantigen can be determined based on the likelihood e.g., when the likelihood is greater than a threshold. The likelihood can be determined based on the MHC-1 binding affinity. The likelihood of the peptide being greater than a threshold can be determined using various criteria. For example, a peptide can be identified as a clonal neoantigen when the MHC-1 binding affinity is greater than a binding threshold. In addition or alternatively, the peptide can be identified as a clonal neoantigen when the MHC-1 binding affinity is greater than a reference binding affinity of a wildtype sequence at the genomic position. In such examples, the binding affinity can be a proxy for the likelihood. Thus, a likelihood of the peptide being presented on the cell surface can include comparing the MHC-1 binding affinity to the binding threshold and the reference binding affinity. Further criteria are provided herein, e.g., for the binding threshold.

At block 1860, an amount of clonal neoantigens is determined. The amount of clonal neoantigens can be a clonal neoantigen load (cNEO). The amount can be normalized by a length of an exome sequenced.

In one example, determining the amount of clonal neoantigens can include determining a count of all peptides having a specified length range and being identified as a clonal neoantigen. As examples, the specified length range can be 8-11 amino acids. Determining the count can includes generating of possible peptides within the specified length range that contain one or more non-consensus amino acids derived from the clonal mutation and determining a count of the possible peptides that are identified as clonal neoantigens. Accordingly, determining the determining the amount of clonal neoantigens can include counting a number of peptides identified as a clonal neoantigen

In another example, determining the amount of clonal neoantigens can include determining a number of the set of clonal mutations that have a peptide as being a clonal neoantigen. Accordingly, determining the amount of clonal neoantigens can include counting a number of clonal mutations resulting in at least one clonal neoantigen.

At block 1870, the amount of clonal neoantigens is compared to a reference value. The reference value can be determined using training samples having a known responder classification to an immunotherapy treatment. The immunotherapy treatment can induce the immune system to attack cells carrying the neoantigens. The immunotherapy treatment can include genetically modified tumor cells of the subject, e.g., Vigil®.

At block 1880, it is determined whether the subject is a responder to the immunotherapy treatment based on the comparison. When the subject is a responder to the immunotherapy treatment, the method can further comprise administering the immunotherapy treatment to the subject.

### D. ITH

Some embodiments can also use ITH to detect markers. Any one or more of cTMB, ITH, and cNEO can be used. For example, a machine learning model (e.g., decision tree or support vector machine, SVM) can use all three values to discriminate between responders and non-responders.

The higher the clonal TMB, the lower the ITH can be. However, not necessarily, because the clonal TMB is determined based on the total number of clonal mutations not the percentage of mutations that are clonal. If there is a lower ITH, then there are not many sub-clonal tumors. The ITH can be determined as 1 minus the TMB for the clonal cluster divided by the total TMB for all clusters. As an example, in FIG. 16A, one minus the clonal TMB (1.0697) divided by the total TMB (7.3234) provides an ITH of 0.8539.

### VI. Example Systems

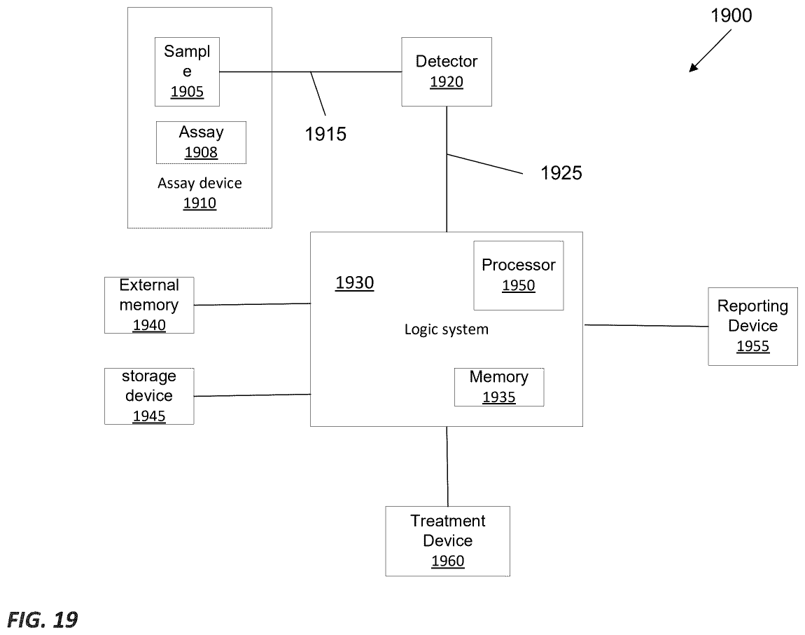

FIG. 19 illustrates a measurement system 1900 according to an embodiment of the present disclosure. The system as shown includes a sample 1905, such as cell-free nucleic acid molecules (e.g., DNA and/or RNA) within an assay device 1910, where an assay 1908 can be performed on sample 1905. For example, sample 1905 can be contacted with reagents of assay 1908 to provide a signal (e.g., an intensity signal) of a physical characteristic 1915 (e.g., sequence information of a cell-free nucleic acid molecule). An example of an assay device can be a flow cell that includes probes and/or primers of an assay or a tube through which a droplet moves (with the droplet including the assay). Physical characteristic 1915 (e.g., a fluorescence intensity, a voltage, or a current), from the sample is detected by detector 1920. Detector 1920 can take a measurement at intervals (e.g., periodic intervals) to obtain data points that make up a data signal. In one embodiment, an analog-to-digital converter converts an analog signal from the detector into digital form at a plurality of times.

Assay device 1910 and detector 1920 can form an assay system, e.g., a PCR system or a sequencing system that performs sequencing according to embodiments described herein. A data signal 1925 is sent from detector 1920 to logic system 1930. As an example, data signal 1925 can be used to determine sequences and/or locations in a reference genome of nucleic acid molecules (e.g., DNA and/or RNA). Data signal 1925 can include various measurements made at a same time, e.g., different colors of fluorescent dyes or different electrical signals for different molecule of sample 1905, and thus data signal 1925 can correspond to multiple signals. Data signal 1925 may be stored in a local memory 1935, an external memory 1940, or a storage device 1945. The assay system can be comprised of multiple assay devices and detectors.

Logic system 1930 may be, or may include, a computer system, ASIC, microprocessor, graphics processing unit (GPU), etc. It may also include or be coupled with a display (e.g., monitor, LED display, etc.) and a user input device (e.g., mouse, keyboard, buttons, etc.). Logic system 1930 and the other components may be part of a stand-alone or network connected computer system, or they may be directly attached to or incorporated in a device (e.g., a sequencing device) that includes detector 1920 and/or assay device 1910. Logic system 1930 may also include software that executes in a processor 1950. Logic system 1930 may include a computer readable medium storing instructions for controlling measurement system 1900 to perform any of the methods described herein. For example, logic system 1930 can provide commands to a system that includes assay device 1910 such that sequencing or other physical operations are performed. Such physical operations can be performed in a particular order, e.g., with reagents being added and removed in a particular order. Such physical operations may be performed by a robotics system, e.g., including a robotic arm, as may be used to obtain a sample and perform an assay. Logic system 1930 can perform any steps of methods described herein that perform computer processing.

Measurement system 1900 may also include a treatment device 1960, which can provide a treatment to the subject. Treatment device 1960 can determine a treatment and/or be used to perform a treatment. Examples of such treatment can include surgery, radiation therapy, chemotherapy, immunotherapy, targeted therapy, hormone therapy, and stem cell transplant. Logic system 1930 may be connected to treatment device 1960, e.g., to provide results of a method described herein. The treatment device may receive inputs from other devices, such as an imaging device and user inputs (e.g., to control the treatment, such as controls over a robotic system).

Measurement system 1900 may also include a reporting device 1955, which can present results of any of the methods describe herein, e.g., as determined using the measurement system. Reporting device 1955 can be in communication with a reporting module within logic system 1930 that can aggregate, format, and send a report to reporting device 1955. The reporting module can present information determined using any of the method described herein. The information can be presented by reporting device 1955 in any format that can be recognized and interpreted by a user of the measurement system 1900. For example, the information can be presented by reporting device 1955 in a displayed, printed, or transmitted format, or any combination thereof.

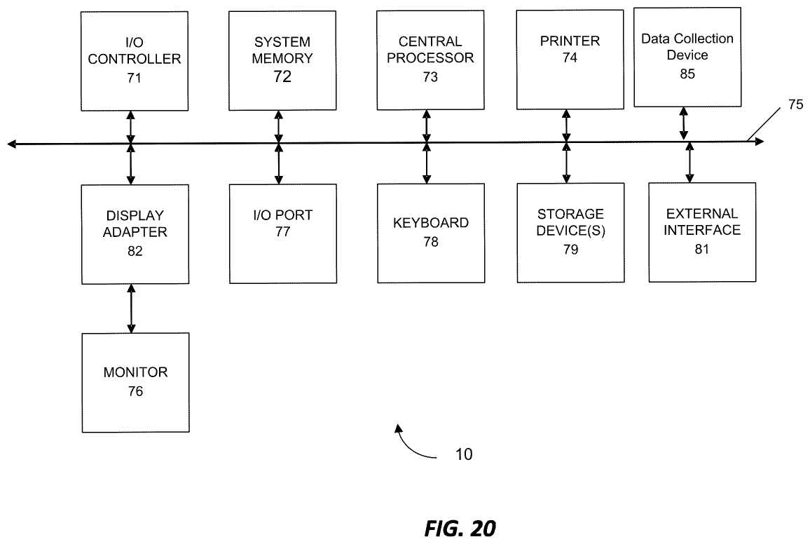

Any of the computer systems mentioned herein may utilize any suitable number of subsystems. Examples of such subsystems are shown in FIG. 20 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 20 are interconnected via a system bus 75. Additional subsystems such as a printer 74, keyboard 78, storage device(s) 79, monitor 76 (e.g., a display screen, such as an LED), which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire®). For example, I/O port 77 or external interface 81 (e.g., Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components. In various embodiments, methods may involve various numbers of clients and/or servers, including at least 10, 20, 50, 100, 200, 500, 1,000, or 10,000 devices. Methods can include various numbers of communication messages between devices, including at least 100, 200, 500, 1,000, 10,000, 50,000, 100,000, 500,00, or one million communication messages. Such communications can involve at least 1 MB, 10 MB, 100 MB, 1 GB, 10 GB, or 100 GB of data.

Aspects of embodiments can be implemented in the form of control logic using hardware circuitry (e.g., an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed but could have additional steps not included in a figure. A process may correspond to a method, a function, a procedure, a subroutine, a subprogram, etc. When a process corresponds to a function, its termination may correspond to a return of the function to the calling function or the main function.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device (e.g., as firmware) or provided separately from other devices (e.g., via Internet download). Any such computer readable medium may reside on or within a single computer product (e.g., a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Any operations performed with a processor (e.g., aligning, determining, comparing, computing, calculating) may be performed in real-time. The term “real-time” may refer to computing operations or processes that are completed within a certain time constraint. As examples, a time constraint may be 30 seconds, 1 minute, 10 minutes, 30 minutes, 1 hour, 4 hours, 1 day, or 7 days. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The claims may be drafted to exclude any element which may be optional. As such, this statement is intended to serve as antecedent basis for use of such exclusive terminology as “solely”, “only”, and the like in connection with the recitation of claim elements, or the use of a “negative” limitation.

All patents, patent applications, publications, and descriptions mentioned herein are incorporated by reference in their entirety for all purposes. None is admitted as prior art. Where a conflict exists between the instant application and a reference provided herein, the instant application shall dominate.

### Exemplary Embodiments

1. A method for treating cancer in a patient in need thereof, the method comprising: determining that the patient is homologous recombination proficient (HRP); and administering an immunotherapy to the patient.

2. The method of embodiment 1, wherein the immunotherapy comprises a therapeutically effective population of tumor infiltrating lymphocytes (TILs).

3. The method of embodiment 1, wherein the patient has a clonal neoantigen proportion greater than a threshold.

4. The method of embodiment 3, wherein the method further comprises administering to the patient:
(i) a neoantigen that is predicted to be presented by an HLA molecule encoded by an HLA allele which has been determined not to have been lost in a tumor;
(ii) an immune cell which recognizes a neoantigen that is predicted to be presented by an HLA molecule encoded by an HLA allele which has been determined not to have been lost in a tumor; or
(iii) an antibody which recognizes a neoantigen that is predicted to be presented by an HLA molecule encoded by an HLA allele which has been determined not to have been lost in a tumor.

5. The method of embodiment 4, wherein determination of whether said HLA allele has been lost comprises the steps of:
aligning HLA allele sequence information from a tumor sample from said subject with an HLA allele reference sequence which is based on said subject's HLA type; and
determining the specific copy number of said HLA allele in said tumor.

6. The method of embodiment 3, wherein the TILs specifically target the clonal neoantigen.

7. The method according to embodiment 1, wherein the patient has a clonal tumor mutation burden (cTMB) greater than a threshold.

8. The method according to embodiment 1, wherein the cancer is selected from the group consisting of a solid tumor cancer, ovarian cancer, adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, prostate cancer, sarcoma, stomach cancer, uterine cancer, thyroid cancer, and a hematological cancer.

9. The method of embodiment 1, wherein the method further comprises administering to the individual at least one dose of an additional therapeutic agent.

10. The method of embodiment 9, wherein the additional therapeutic agent is a checkpoint inhibitor or an angiogenesis inhibitor.

11. The method of embodiment 10, wherein the additional therapeutic agent is a checkpoint inhibitor.

12. The method of embodiment 1, wherein the immunotherapy comprises a therapeutically effective population of engineered cells selected from the group consisting of tumor infiltrating lymphocytes (TILs), T cell receptor (TCR) cells, chimeric antigen receptor (CAR) T cells, and natural killer (NK) cells.

13. The method of embodiment 12, wherein the therapeutically effective population of engineered cells is tumor infiltrating lymphocytes (TILs).

14. The method of embodiment 12, wherein the therapeutically effective population of engineered cells is chimeric antigen receptor (CAR) T cells.

15. The method of embodiment 12, wherein the therapeutically effective population of engineered cells is natural killer (NK) cells.

16. A method for identifying a patient being a responder to a tumor infiltrating lymphocytes (TIL) therapy for treating cancer, the method comprising:
administering an immunotherapy;
assessing the effectiveness of the immunotherapy for treating cancer; and
determining that the patient is homologous recombination proficient (HRP) if the immunotherapy is effective.

17. The method of embodiment 16, wherein the immunotherapy comprises a therapeutically effective population of TILs.

18. An engineered tumor cell construct comprising an exogenous gene to express an exogenous protein.

19. A method of treating cancer in a patient in need thereof, the method comprising: selecting a responder by first selecting a patient who is HRP; and administering the engineered tumor cell construct of embodiment 18 to the patient who is HRP.

20. A method of determining a treatment for a subject having a particular type of cancer, the method comprising:
performing exome sequencing of paired tumor and normal samples of a subject to obtain tumor sequence reads and normal sequence reads;
comparing the tumor sequence reads to the normal sequence reads to identify a set of mutations that are non-synonymous;
determining a clonal tumor mutation burden (cTMB) by:
for each of the set of mutations:
   determining a respective amount of the tumor sequence reads that have the mutation; and
   determining whether the mutation is a clonal mutation based on the respective amount, thereby determining clonal mutations;
   determining the cTMB from using the clonal mutations;
comparing cTMB to a reference value, wherein the reference value is determined using training samples of training subjects that have the particular type of cancer and that have a known responder classification to an immunotherapy treatment, wherein the immunotherapy treatment that includes genetically modified tumor cells of the subject; and determining whether the subject is a responder to the immunotherapy treatment based on the comparison.

21. A method of determining a treatment for a patient having cancer, the method comprising:
performing exome sequencing of paired tumor and normal samples of a subject to obtain tumor sequence reads and normal sequence reads;
comparing the tumor sequence reads to the normal sequence reads to identify mutations that are non-synonymous;
determining a set of the mutations that are clonal mutations;
determining an MHC haplotype of the patient using the tumor sequence reads and/or the normal sequence reads;
for each clonal mutation:
   generating a peptide corresponding to the clonal mutation at a genomic position;
   determining an MHC-1 binding affinity for the peptide; and
   identifying the peptide as a clonal neoantigen when the MHC-1 binding affinity is greater than a binding threshold and is greater than a reference binding affinity of a wildtype sequence at the genomic position;
determining an amount of clonal neoantigens;
comparing the amount of clonal neoantigens to a reference value, wherein the reference value is determined using training samples having a known responder classification to an immunotherapy treatment, wherein the immunotherapy treatment induces the immune system to attack the neoantigens; and
determining whether the subject is a responder to the immunotherapy treatment based on the comparison.

22. The method of embodiment 20 or 21, wherein the subject is a responder to the immunotherapy treatment, the method further comprising:
administering the immunotherapy treatment to the subject.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure.

### V. References Below are Incorporated by Reference

1. Litchfield K, Reading J L, Puttick C, et al. Meta-analysis of tumor- and T cell-intrinsic mechanisms of sensitization to checkpoint inhibition. Cell. 2021; 184:596-614 e514.
2. Turke A B, Zejnullahu K, Wu Y L, et al. Preexistence and clonal selection of MET amplification in EGFR mutant NSCLC. Cancer Cell. 2010; 17:77-88.
3. Diaz L A, Jr., Williams R T, Wu J, et al. The molecular evolution of acquired resistance to targeted EGFR blockade in colorectal cancers. Nature. 2012; 486:537-540.
4. Wilmott J S, Tembe V, Howle J R, et al. Intratumoral molecular heterogeneity in a BRAF-mutant, BRAF inhibitor-resistant melanoma: a case illustrating the challenges for personalized medicine. Mol Cancer Ther. 2012; 11:2704-2708.
5. Chen Z Y, Zhong W Z, Zhang X C, et al. EGFR mutation heterogeneity and the mixed response to EGFR tyrosine kinase inhibitors of lung adenocarcinomas. Oncologist. 2012; 17:978-985.
6. Engelman J A, Zejnullahu K, Mitsudomi T, et al. MET amplification leads to gefitinib resistance in lung cancer by activating ERBB3 signaling. Science. 2007; 316:1039-1043.
7. Katayama R, Shaw A T, Khan™, et al. Mechanisms of acquired crizotinib resistance in ALK-rearranged lung Cancers. Sci Transl Med. 2012; 4: 120ra117.
8. Hata A N, Niederst M J, Archibald H L, et al. Tumor cells can follow distinct evolutionary paths to become resistant to epidermal growth factor receptor inhibition. Nat Med. 2016; 22:262-269.
9. Russo M, Crisafulli G, Sogari A, et al. Adaptive mutability of colorectal cancers in response to targeted therapies. Science. 2019; 366:1473-1480.
10. Larkin J, Chiarion-Sileni V, Gonzalez R, et al. Combined Nivolumab and Ipilimumab or Monotherapy in Untreated Melanoma. N Engl J Med. 2015; 373:23-34.
11. Motzer R J, Escudier B, McDermott D F, et al. Nivolumab versus Everolimus in Advanced Renal-Cell Carcinoma. N Engl J Med. 2015; 373:1803-1813.
12. Borghaei H, Paz-Ares L, Horn L, et al. Nivolumab versus Docetaxel in Advanced Nonsquamous Non-Small-Cell Lung Cancer. N Engl J Med. 2015; 373:1627-1639.
13. Lennerz V, Fatho M, Gentilini C, et al. The response of autologous T cells to a human melanoma is dominated by mutated neoantigens. Proc Natl Acad Sci USA. 2005; 102:16013-16018.
14. Matsushita H, Vesely M D, Koboldt D C, et al. Cancer exome analysis reveals a T-cell-dependent mechanism of cancer immunoediting. Nature. 2012; 482:400-404.
15. DuPage M, Mazumdar C, Schmidt L M, Cheung A F, Jacks T. Expression of tumour-specific antigens underlies cancer immunoediting. Nature. 2012; 482:405-409.
16. Robbins P F, Lu Y C, El-Gamil M, et al. Mining exomic sequencing data to identify mutated antigens recognized by adoptively transferred tumor-reactive T cells. Nat Med. 2013; 19:747-752.
17. Gubin M M, Zhang X, Schuster H, et al. Checkpoint blockade cancer immunotherapy targets tumour-specific mutant antigens. Nature. 2014; 515:577-581.
18. Mellman I, Coukos G, Dranoff G. Cancer immunotherapy comes of age. Nature. 2011; 480:480-489.
19. Alspach E, Lussier D M, Miceli A P, et al. MHC-I I neoantigens shape tumour immunity and response to immunotherapy. Nature. 2019; 574:696-701.
20. Ceglia V, Kelley E J, Boyle A S, et al. A Framework to Identify Antigen-Expanded T Cell Receptor Clusters Within Complex Repertoires. Front Immunol. 2021; 12:735584.
21. Joshi K, Chain B M, Peggs K S, Quezada S A. The “Achilles' Heel” of Cancer and Its Implications for the Development of Novel Immunotherapeutic Strategies. Cold Spring Harb Perspect Med. 2018; 8.
22. McGranahan N, Furness A J, Rosenthal R, et al. Clonal neoantigens elicit T cell immunoreactivity and sensitivity to immune checkpoint blockade. Science. 2016; 351:1463-1469.
23. Riaz N, Morris L, Havel J J, Makarov V, Desrichard A, Chan T A. The role of neoantigens in response to immune checkpoint blockade. Int Immunol. 2016; 28:411-419.
24. Hanahan D. Hallmarks of Cancer: New Dimensions. Cancer Discov. 2022; 12:31-46.
25. Dupont C A, Riegel K, Pompaiah M, Juhl H, Rajalingam K. Druggable genome and precision medicine in cancer: current challenges. FEBS J. 2021; 288:6142-6158.
26. Al Bakir M, Huebner A, Martinez-Ruiz C, et al. The evolution of non-small cell lung cancer metastases in TRACERx. Nature. 2023; 616:534-542.
27. Gerlinger M, Rowan A J, Horswell S, et al. Intratumor heterogeneity and branched evolution revealed by multiregion sequencing. N Engl J Med. 2012; 366:883-892.
28. Wu Y, Biswas D, Swanton C. Impact of cancer evolution on immune surveillance and checkpoint inhibitor response. Semin Cancer Biol. 2022; 84:89-102.
29. Gonzalez P A, Carreno L J, Coombs D, et al. T cell receptor binding kinetics required for T cell activation depend on the density of cognate ligand on the antigen-presenting cell. Proc Natl Acad Sci USA. 2005; 102:4824-4829.
30. Valitutti S, Muller S, Cella M, Padovan E, Lanzavecchia A. Serial triggering of many T-cell receptors by a few peptide-MHC complexes. Nature. 1995; 375:148-151.
31. Davis M M, Boniface J J, Reich Z, et al. Ligand recognition by alpha beta T cell receptors. Annu Rev Immunol. 1998; 16:523-544.
32. Gejman R S, Chang A Y, Jones H F, et al. Rejection of immunogenic tumor clones is limited by clonal fraction. Elife. 2018; 7.
33. Wolf Y, Bartok O, Patkar S, et al. UVB-Induced Tumor Heterogeneity Diminishes Immune Response in Melanoma. Cell. 2019; 179:219-235 e221.
34. Tran E, Robbins P F, Lu Y C, et al. T-Cell Transfer Therapy Targeting Mutant KRAS in Cancer. N Engl J Med. 2016; 375:2255-2262.
35. Andersen M H, Fensterle J, Ugurel S, et al. Immunogenicity of constitutively active V599EBRaf. Cancer Res. 2004; 64:5456-5460.
36. Hoeijmakers J H. Genome maintenance mechanisms for preventing cancer. Nature. 2001; 411:366-374.
37. Bowman R L, Hennessey R C, Weiss T J, et al. UVB mutagenesis differs in Nras- and Braf-mutant mouse models of melanoma. Life Sci Alliance. 2021; 4.
38. Govindan R, Ding L, Griffith M, et al. Genomic landscape of non-small cell lung cancer in smokers and never-smokers. Cell. 2012; 150:1121-1134.
39. Cancer Genome Atlas Research N. Comprehensive molecular profiling of lung adenocarcinoma. Nature. 2014; 511:543-550.
40. Le D T, Uram J N, Wang H, et al. PD-1 Blockade in Tumors with Mismatch-Repair Deficiency. N Engl J Med. 2015; 372:2509-2520.
41. Rizvi N A, Hellmann M D, Snyder A, et al. Cancer immunology. Mutational landscape determines sensitivity to PD-1 blockade in non-small cell lung cancer. Science. 2015; 348:124-128.
42. Nemunaitis J, Stanbery L, Walter A, Rocconi R P, Stephens P. Rationale of Homologous Recombination Proficient Molecular Profile as Biomarker for Therapeutic Targeting in Ovarian Cancer. Oncology Reviews.
43. Rocconi R P, Grosen E A, Ghamande S A, et al. Gemogenovatucel-T (Vigil) immunotherapy as maintenance in frontline stage III/I V ovarian cancer (VITAL): a randomised, double-blind, placebo-controlled, phase 2b trial. Lancet Oncol. 2020; 21:1661-1672.
44. Rocconi R P, Monk B J, Walter A, et al. Gemogenovatucel-T (Vigil) immunotherapy demonstrates clinical benefit in homologous recombination proficient (HRP) ovarian cancer. Gynecol Oncol. 2021; 161:676-680.
45. Walter A, Rocconi R P, Monk B J, et al. Gemogenovatucel-T (Vigil) maintenance immunotherapy: 3-year survival benefit in homologous recombination proficient (HRP) ovarian cancer. Gynecol Oncol. 2021; 163:459-464.
46. Lythe G, Callard R E, Hoare R L, Molina-Paris C. How many TCR clonotypes does a body maintain? J Theor Biol. 2016; 389:214-224.
47. Wosen J E, Mukhopadhyay D, Macaubas C, Mellins E D. Epithelial MHC Class I I Expression and Its Role in Antigen Presentation in the Gastrointestinal and Respiratory Tracts. Front Immunol. 2018; 9:2144.
48. Coulie P G, Van den Eynde B J, van der Bruggen P, Boon T. Tumour antigens recognized by T lymphocytes: at the core of cancer immunotherapy. Nat Rev Cancer. 2014; 14:135-146.
49. Tran E, Turcotte S, Gros A, et al. Cancer immunotherapy based on mutation-specific CD4+ T cells in a patient with epithelial cancer. Science. 2014; 344:641-645.
50. Sarnaik A A, Hamid O, Khushalani N I, et al. Lifileucel, a Tumor-Infiltrating Lymphocyte Therapy, in Metastatic Melanoma. J Clin Oncol. 2021; 39:2656-2666.
51. Vogelstein B, Papadopoulos N, Velculescu V E, Zhou S, Diaz L A, Jr., Kinzler K W. Cancer genome landscapes. Science. 2013; 339:1546-1558.
52. Peri A, Greenstein E, Alon M, et al. Combined presentation and immunogenicity analysis reveals a recurrent RAS.Q61K neoantigen in melanoma. J Clin Invest. 2021; 131.
53. Duan F, Duitama J, Al Seesi S, et al. Genomic and bioinformatic profiling of mutational neoepitopes reveals new rules to predict anticancer immunogenicity. J Exp Med. 2014; 211:2231-2248.
54. Spierings E, Brickner A G, Caldwell J A, et al. The minor histocompatibility antigen HA-3 arises from differential proteasome-mediated cleavage of the lymphoid blast crisis (Lbc) oncoprotein. Blood. 2003; 102:621-629.
55. McKeithan T W. Kinetic proofreading in T-cell receptor signal transduction. Proc Natl Acad Sci USA. 1995; 92:5042-5046.
56. Grakoui A, Bromley S K, Sumen C, et al. The immunological synapse: a molecular machine controlling T cell activation. Science. 1999; 285:221-227.
57. Savage P A, Boniface J J, Davis M M. A kinetic basis for T cell receptor repertoire selection during an immune response. Immunity. 1999; 10:485-492.
58. Kalergis A M, Boucheron N, Doucey M A, et al. Efficient T cell activation requires an optimal dwell-time of interaction between the TCR and the pMHC complex. Nat Immunol. 2001; 2:229-234.
59. Snyder A, Makarov V, Merghoub T, et al. Genetic basis for clinical response to CTLA-4 blockade in melanoma. N Engl J Med. 2014; 371:2189-2199.
60. Rosenthal R, Cadieux E L, Salgado R, et al. Neoantigen-directed immune escape in lung cancer evolution. Nature. 2019; 567:479-485.
61. Leko V, Rosenberg S A. Identifying and Targeting Human Tumor Antigens for T Cell-Based Immunotherapy of Solid Tumors. Cancer Cell. 2020; 38:454-472.
62. Clark R E, Dodi I A, Hill S C, et al. Direct evidence that leukemic cells present HLA-associated immunogenic peptides derived from the BCR-ABL b3a2 fusion protein. Blood. 2001; 98:2887-2893.
63. van der Lee D I, Reijmers R M, Honders M W, et al. Mutated nucleophosmin 1 as immunotherapy target in acute myeloid leukemia. J Clin Invest. 2019; 129:774-785.
64. Segal N H, Parsons D W, Peggs K S, et al. Epitope landscape in breast and colorectal cancer. Cancer Res. 2008; 68:889-892.
65. Garcia-Garijo A, Fajardo C A, Gros A. Determinants for Neoantigen Identification. Front Immunol. 2019; 10:1392.
66. Kalaora S, Samuels Y. Cancer Exome-Based Identification of Tumor Neo-Antigens Using Mass Spectrometry. In: López-Soto A, Folgueras A R, editors. Cancer Immunosurveillance: Methods and Protocols. New York, N Y: Springer New York, 2019:203-214.
67. Castle J C, Kreiter S, Diekmann J, et al. Exploiting the Mutanome for Tumor Vaccination. Cancer Research. 2012; 72:1081-1091.
68. Bassani-Sternberg M, Coukos G. Mass spectrometry-based antigen discovery for cancer immunotherapy. Curr Opin Immunol. 2016; 41:9-17.
69. Schumacher T N, Scheper W, Kvistborg P. Cancer Neoantigens. Annu Rev Immunol. 2019; 37:173-200.
70. Roth A, Khattra J, Yap D, et al. PyClone: statistical inference of clonal population structure in cancer. Nat Methods. 2014; 11:396-398.
71. Kote S, Pirog A, Bedran G, Alfaro J, Dapic I. Mass spectrometry-based identification of MHC-associated peptides. Cancers. 2020; 12:535.
72. Haslem D S, Van Norman S B, Fulde G, et al. A Retrospective Analysis of Precision Medicine Outcomes in Patients With Advanced Cancer Reveals Improved Progression-Free Survival Without Increased Health Care Costs. J Oncol Pract. 2017; 13: e108-e119.
73. Radovich M, Kiel P J, Nance S M, et al. Clinical benefit of a precision medicine based approach for guiding treatment of refractory cancers. Oncotarget. 2016; 7:56491-56500.
74. Jardim D L, Schwaederle M, Wei C, et al. Impact of a Biomarker-Based Strategy on Oncology Drug Development: A Meta-analysis of Clinical Trials Leading to FDA Approval. J Natl Cancer Inst. 2015; 107.
75. Schwaederle M, Zhao M, Lee J J, et al. Association of Biomarker-Based Treatment Strategies With Response Rates and Progression-Free Survival in Refractory Malignant Neoplasms: A Meta-analysis. JAMA Oncol. 2016; 2:1452-1459.
76. Schwaederle M, Zhao M, Lee J J, et al. Impact of Precision Medicine in Diverse Cancers: A Meta-Analysis of Phase II Clinical Trials. J Clin Oncol. 2015; 33:3817-3825.
77. Dhir M, Choudry H A, Holtzman M P, et al. Impact of genomic profiling on the treatment and outcomes of patients with advanced gastrointestinal malignancies. Cancer Med. 2017; 6:195-206.
78. Chawla A, Janku F, Wheler J J, et al. Estimated Cost of Anticancer Therapy Directed by Comprehensive Genomic Profiling in a Single-Center Study. JCO Precis Oncol. 2018; 2.
79. Handorf E A, McElligott S, Vachani A, et al. Cost effectiveness of personalized therapy for first-line treatment of stage I V and recurrent incurable adenocarcinoma of the lung. J Oncol Pract. 2012; 8:267-274.
80. Pennell N A, Mutebi A, Zhou Z Y, et al. Economic Impact of Next-Generation Sequencing Versus Single-Gene Testing to Detect Genomic Alterations in Metastatic Non-Small-Cell Lung Cancer Using a Decision Analytic Model. JCO Precis Oncol. 2019; 3:1-9.
81. Barlesi F, Mazieres J, Merlio J P, et al. Routine molecular profiling of patients with advanced non-small-cell lung cancer: results of a 1-year nationwide programme of the French Cooperative Thoracic Intergroup (IFCT). Lancet. 2016; 387:1415-1426.
82. Signorovitch J, Zhou Z, Ryan J, Chawla A. Comprehensive genomic profiling (CGP) versus conventional molecular diagnostic testing of patients with advanced non-small cell lung cancer (NSCLC): Overall survival (OS) and cost in a U.S. health plan population. Journal of Clinical Oncology. 2017; 35:6599-6599.
83. Sicklick J K, Kato S, Okamura R, et al. Molecular profiling of cancer patients enables personalized combination therapy: the I-PREDICT study. Nat Med. 2019; 25:744-750.
84. Eggermont A M, Chiarion-Sileni V, Grob J J, et al. Adjuvant ipilimumab versus placebo after complete resection of high-risk stage III melanoma (EORTC 18071): a randomised, double-blind, phase 3 trial. Lancet Oncol. 2015; 16:522-530.
85. Freeman G J, Long A J, Iwai Y, et al. Engagement of the PD-1 immunoinhibitory receptor by a novel B7 family member leads to negative regulation of lymphocyte activation. J Exp Med. 2000; 192:1027-1034.
86. Dong H, Strome S E, Salomao D R, et al. Tumor-associated B7-H1 promotes T-cell apoptosis: a potential mechanism of immune evasion. Nat Med. 2002; 8:793-800.
87. Zou W, Chen L. Inhibitory B7-family molecules in the tumour microenvironment. Nat Rev Immunol. 2008; 8:467-477.
88. Hodi F S, O'Day S J, McDermott D F, et al. Improved survival with ipilimumab in patients with metastatic melanoma. N Engl J Med. 2010; 363:711-723.
89. Postow M A, Chesney J, Pavlick A C, et al. Nivolumab and ipilimumab versus ipilimumab in untreated melanoma. N Engl J Med. 2015; 372:2006-2017.
90. Wolchok J D, Chiarion-Sileni V, Gonzalez R, et al. Overall Survival with Combined Nivolumab and Ipilimumab in Advanced Melanoma. N Engl J Med. 2017; 377:1345-1356.
91. Motzer R J, Tannir N M, McDermott D F, et al. Nivolumab plus Ipilimumab versus Sunitinib in Advanced Renal-Cell Carcinoma. N Engl J Med. 2018; 378:1277-1290.
92. Lenz H J, Van Cutsem E, Luisa Limon M, et al. First-Line Nivolumab Plus Low-Dose Ipilimumab for Microsatellite Instability-High/Mismatch Repair-Deficient Metastatic Colorectal Cancer: The Phase II CheckMate 142 Study. J Clin Oncol. 2022; 40:161-170.
93. Weber J S, D'Angelo S P, Minor D, et al. Nivolumab versus chemotherapy in patients with advanced melanoma who progressed after anti-CTLA-4 treatment (CheckMate 037): a randomised, controlled, open-label, phase 3 trial. Lancet Oncol. 2015; 16:375-384.
94. Robert C, Long G V, Brady B, et al. Nivolumab in previously untreated melanoma without BRAF mutation. N Engl J Med. 2015; 372:320-330.
95. Weber J, Mandala M, Del Vecchio M, et al. Adjuvant Nivolumab versus Ipilimumab in Resected Stage III or IV Melanoma. N Engl J Med. 2017; 377:1824-1835.
96. Brahmer J, Reckamp K L, Baas P, et al. Nivolumab versus Docetaxel in Advanced Squamous-Cell Non-Small-Cell Lung Cancer. N Engl J Med. 2015; 373:123-135.
97. Ready N E, Ott P A, Hellmann M D, et al. Nivolumab Monotherapy and Nivolumab Plus Ipilimumab in Recurrent Small Cell Lung Cancer: Results From the CheckMate 032 Randomized Cohort. J Thorac Oncol. 2020; 15:426-435.
98. Ferris R L, Blumenschein G, Jr., Fayette J, et al. Nivolumab for Recurrent Squamous-Cell Carcinoma of the Head and Neck. N Engl J Med. 2016; 375:1856-1867.
99. Robert C, Ribas A, Wolchok J D, et al. Anti-programmed-death-receptor-1 treatment with pembrolizumab in ipilimumab-refractory advanced melanoma: a randomised dose-comparison cohort of a phase 1 trial. Lancet. 2014; 384:1109-1117.
100. Robert C, Schachter J, Long G V, et al. Pembrolizumab versus Ipilimumab in Advanced Melanoma. N Engl J Med. 2015; 372:2521-2532.
101. Eggermont A M M, Blank C U, Mandala M, et al. Adjuvant pembrolizumab versus placebo in resected stage III melanoma (EORTC 1325-M G/KEYNOTE-054): distant metastasis-free survival results from a double-blind, randomised, controlled, phase 3 trial. Lancet Oncol. 2021; 22:643-654.
102. Herbst R S, Baas P, Kim D W, et al. Pembrolizumab versus docetaxel for previously treated, PD-L1-positive, advanced non-small-cell lung cancer (KEYNOTE-010): a randomised controlled trial. Lancet. 2016; 387:1540-1550.
103. Reck M, Rodriguez-Abreu D, Robinson A G, et al. Pembrolizumab versus Chemotherapy for PD-L1-Positive Non-Small-Cell Lung Cancer. N Engl J Med. 2016; 375:1823-1833.
104. Borghaei H, Langer C, Gadgeel S, et al. O A 17.01 Pemetrexed-Carboplatin Plus Pembrolizumab as First-Line Therapy for Advanced Nonsquamous NSCLC: KEYNOTE-021 Cohort G Update. Journal of Thoracic Oncology. 2017; 12: S1791.
105. Gandhi L, Rodriguez-Abreu D, Gadgeel S, et al. Pembrolizumab plus Chemotherapy in Metastatic Non-Small-Cell Lung Cancer. N Engl J Med. 2018; 378:2078-2092.
106. Paz-Ares L, Luft A, Vicente D, et al. Pembrolizumab plus Chemotherapy for Squamous Non-Small-Cell Lung Cancer. N Engl J Med. 2018; 379:2040-2051.
107. Seiwert T Y, Burtness B, Mehra R, et al. Safety and clinical activity of pembrolizumab for treatment of recurrent or metastatic squamous cell carcinoma of the head and neck (KEYNOTE-012): an open-label, multicentre, phase 1b trial. Lancet Oncol. 2016; 17:956-965.
108. Burtness B, Harrington K J, Greil R, et al. Pembrolizumab alone or with chemotherapy versus cetuximab with chemotherapy for recurrent or metastatic squamous cell carcinoma of the head and neck (KEYNOTE-048): a randomised, open-label, phase 3 study. Lancet. 2019; 394:1915-1928.
109. Chen R, Zinzani P L, Fanale M A, et al. Phase II Study of the Efficacy and Safety of Pembrolizumab for Relapsed/Refractory Classic Hodgkin Lymphoma. J Clin Oncol. 2017; 35:2125-2132.
110. Armand P, Rodig S, Melnichenko V, et al. Pembrolizumab in Relapsed or Refractory Primary Mediastinal Large B-Cell Lymphoma. J Clin Oncol. 2019; 37:3291-3299.
111. Bellmunt J, de Wit R, Vaughn D J, et al. Pembrolizumab as Second-Line Therapy for Advanced Urothelial Carcinoma. N Engl J Med. 2017; 376:1015-1026.
112. Balar A V, Castellano D, O'Donnell P H, et al. First-line pembrolizumab in cisplatin-ineligible patients with locally advanced and unresectable or metastatic urothelial cancer (KEYNOTE-052): a multicentre, single-arm, phase 2 study. Lancet Oncol. 2017; 18:1483-1492.
113. Marabelle A, Fakih M, Lopez J, et al. Association of tumour mutational burden with outcomes in patients with advanced solid tumours treated with pembrolizumab: prospective biomarker analysis of the multicohort, open-label, phase 2 KEYNOTE-158 study. Lancet Oncol. 2020; 21:1353-1365.
114. Migden M R, Rischin D, Schmults C D, et al. PD-1 Blockade with Cemiplimab in Advanced Cutaneous Squamous-Cell Carcinoma. N Engl J Med. 2018; 379:341-351.
115. Pillai R N, Behera M, Owonikoko T K, et al. Comparison of the toxicity profile of PD-1 versus PD-L1 inhibitors in non-small cell lung cancer: A systematic analysis of the literature. Cancer. 2018; 124:271-277.
116. Kaufman H L, Russell J, Hamid O, et al. Avelumab in patients with chemotherapy-refractory metastatic Merkel cell carcinoma: a multicentre, single-group, open-label, phase 2 trial. Lancet Oncol. 2016; 17:1374-1385.
117. Keilholz U, Mehnert J M, Bauer S, et al. Avelumab in patients with previously treated metastatic melanoma: phase 1b results from the JAVELIN Solid Tumor trial. J Immunother Cancer. 2019; 7:12.
118. Motzer R J, Penkov K, Haanen J, et al. Avelumab plus Axitinib versus Sunitinib for Advanced Renal-Cell Carcinoma. N Engl J Med. 2019; 380:1103-1115.
119. Massard C, Gordon M S, Sharma S, et al. Safety and Efficacy of Durvalumab (MEDI4736), an Anti-Programmed Cell Death Ligand-1 Immune Checkpoint Inhibitor, in Patients With Advanced Urothelial Bladder Cancer. J Clin Oncol. 2016; 34:3119-3125.
120. Antonia S J, Villegas A, Daniel D, et al. Durvalumab after Chemoradiotherapy in Stage III Non-Small-Cell Lung Cancer. N Engl J Med. 2017; 377:1919-1929.
121. Balar A V, Galsky M D, Rosenberg J E, et al. Atezolizumab as first-line treatment in cisplatin-ineligible patients with locally advanced and metastatic urothelial carcinoma: a single-arm, multicentre, phase 2 trial. Lancet. 2017; 389:67-76.
122. Rittmeyer A, Barlesi F, Waterkamp D, et al. Atezolizumab versus docetaxel in patients with previously treated non-small-cell lung cancer (OAK): a phase 3, open-label, multicentre randomised controlled trial. Lancet. 2017; 389:255-265.
123. Fehrenbacher L, Spira A, Ballinger M, et al. Atezolizumab versus docetaxel for patients with previously treated non-small-cell lung cancer (POPLAR): a multicentre, open-label, phase 2 randomised controlled trial. Lancet. 2016; 387:1837-1846.
124. Horn L, Mansfield A S, Szczesna A, et al. First-Line Atezolizumab plus Chemotherapy in Extensive-Stage Small-Cell Lung Cancer. N Engl J Med. 2018; 379:2220-2229.
125. Johnson D B, Taylor K B, Cohen J V, et al. Anti-PD-1-Induced Pneumonitis Is Associated with Persistent Imaging Abnormalities in Melanoma Patients. Cancer Immunol Res. 2019; 7:1755-1759.
126. Ricciuti B, Wang X, Alessi J V, et al. Association of High Tumor Mutation Burden in Non-Small Cell Lung Cancers With Increased Immune Infiltration and Improved Clinical Outcomes of PD-L1 Blockade Across PD-L1 Expression Levels. JAMA Oncol. 2022; 8:1160-1168.
127. Rousseau B, Foote M B, Maron S B, et al. The Spectrum of Benefit from Checkpoint Blockade in Hypermutated Tumors. N Engl J Med. 2021; 384:1168-1170.
128. De Marchi P, Leal L F, Duval da Silva V, da Silva E C A, Cordeiro de Lima V C, Reis R M. PD-L1 expression by Tumor Proportion Score (TPS) and Combined Positive Score (CPS) are similar in non-small cell lung cancer (NSCLC). J Clin Pathol. 2021; 74:735-740.
129. Ito T, Okamoto I, Tokashiki K, et al. PD-L1 Expression and Survival Rates Using TPS and CPS for Nivolumab-treated Head-and-Neck Cancer. Anticancer Res. 2022; 42:1547-1554.
130. Planchard D, Popat S, Kerr K, et al. Metastatic non-small cell lung cancer: ESMO Clinical Practice Guidelines for diagnosis, treatment and follow-up. Ann Oncol. 2018; 29: iv192-iv237.
131. Schmid P, Adams S, Rugo H S, et al. Atezolizumab and Nab-Paclitaxel in Advanced Triple-Negative Breast Cancer. N Engl J Med. 2018; 379:2108-2121.
132. Garon E B, Rizvi N A, Hui R, et al. Pembrolizumab for the treatment of non-small-cell lung cancer. N Engl J Med. 2015; 372:2018-2028.
133. Saito Y, Horiuchi S, Morooka H, et al. Inter-tumor heterogeneity of PD-L1 expression in non-small cell lung cancer. J Thorac Dis. 2019; 11:4982-4991.
134. Li M, Li A, Zhou S, et al. Heterogeneity of PD-L1 expression in primary tumors and paired lymph node metastases of triple negative breast cancer. BMC Cancer. 2018; 18:4.
135. Hirsch F R, McElhinny A, Stanforth D, et al. PD-L1 Immunohistochemistry Assays for Lung Cancer: Results from Phase 1 of the Blueprint PD-L1 IHC Assay Comparison Project. J Thorac Oncol. 2017; 12:208-222.
136. Le D T, Durham J N, Smith K N, et al. Mismatch repair deficiency predicts response of solid tumors to PD-1 blockade. Science. 2017; 357:409-413.
137. Overman M J, Lonardi S, Wong K Y M, et al. Durable Clinical Benefit With Nivolumab Plus Ipilimumab in DNA Mismatch Repair-Deficient/Microsatellite Instability-High Metastatic Colorectal Cancer. J Clin Oncol. 2018; 36:773-779.
138. Marabelle A, Le D T, Ascierto P A, et al. Efficacy of Pembrolizumab in Patients With Noncolorectal High Microsatellite Instability/Mismatch Repair-Deficient Cancer: Results From the Phase II KEYNOTE-158 Study. J Clin Oncol. 2020; 38:1-10.
139. Hugo W, Zaretsky J M, Sun L, et al. Genomic and Transcriptomic Features of Response to Anti-PD-1 Therapy in Metastatic Melanoma. Cell. 2016; 165:35-44.
140. Choucair K, Morand S, Stanbery L, Edelman G, Dworkin L, Nemunaitis J. TMB: a promising immune-response biomarker, and potential spearhead in advancing targeted therapy trials. Cancer Gene Ther. 2020; 27:841-853.
141. Herbst R S, Soria J C, Kowanetz M, et al. Predictive correlates of response to the anti-PD-L1 antibody MPDL3280A in cancer patients. Nature. 2014; 515:563-567.
142. Van Allen E M, Miao D, Schilling B, et al. Genomic correlates of response to CTLA-4 blockade in metastatic melanoma. Science. 2015; 350:207-211.
143. Vilar E, Gruber S B. Microsatellite instability in colorectal cancer-the stable evidence. Nat Rev Clin Oncol. 2010; 7:153-162.
144. Yamamoto H, Imai K. An updated review of microsatellite instability in the era of next-generation sequencing and precision medicine. Semin Oncol. 2019; 46:261-270.
145. Germano G, Lamba S, Rospo G, et al. Inactivation of DNA repair triggers neoantigen generation and impairs tumour growth. Nature. 2017; 552:116-120.
146. Mardis E R. Neoantigens and genome instability: impact on immunogenomic phenotypes and immunotherapy response. Genome Med. 2019; 11:71.
147. Alexandrov L B, Kim J, Haradhvala N J, et al. The repertoire of mutational signatures in human cancer. Nature. 2020; 578:94-101.
148. Lawrence M S, Stojanov P, Polak P, et al. Mutational heterogeneity in cancer and the search for new cancer-associated genes. Nature. 2013; 499:214-218.
149. Riaz N, Havel J J, Makarov V, et al. Tumor and Microenvironment Evolution during Immunotherapy with Nivolumab. Cell. 2017; 171:934-949 e916.
150. Miao D, Margolis C A, Vokes N I, et al. Genomic correlates of response to immune checkpoint blockade in microsatellite-stable solid tumors. Nat Genet. 2018; 50:1271-1281.
151. Johnson B E, Mazor T, Hong C, et al. Mutational analysis reveals the origin and therapy-driven evolution of recurrent glioma. Science. 2014; 343:189-193.
152. Anagnostopoulos I, Herbst H, Niedobitek G, Stein H. Demonstration of monoclonal EBV genomes in Hodgkin's disease and Ki-1-positive anaplastic large cell lymphoma by combined Southern blot and in situ hybridization. Blood. 1989; 74:810-816.
153. Sastre-Garau X, Peter M, Avril M F, et al. Merkel cell carcinoma of the skin: pathological and molecular evidence for a causative role of MCV in oncogenesis. J Pathol. 2009; 218:48-56.
154. Ansell S M, Lesokhin A M, Borrello I, et al. PD-1 blockade with nivolumab in relapsed or refractory Hodgkin's lymphoma. N Engl J Med. 2015; 372:311-319.
155. Nghiem P T, Bhatia S, Lipson E J, et al. PD-1 Blockade with Pembrolizumab in Advanced Merkel-Cell Carcinoma. N Engl J Med. 2016; 374:2542-2552.
156. D'Angelo S P, Russell J, Lebbe C, et al. Efficacy and Safety of First-line Avelumab Treatment in Patients With Stage IV Metastatic Merkel Cell Carcinoma: A Preplanned Interim Analysis of a Clinical Trial. JAMA Oncol. 2018; 4: e180077.
157. AbdulJabbar K, Raza S E A, Rosenthal R, et al. Geospatial immune variability illuminates differential evolution of lung adenocarcinoma. Nat Med. 2020; 26:1054-1062.
158. Li J, Wang J, Chen R, Bai Y, Lu X. The prognostic value of tumor-infiltrating T lymphocytes in ovarian cancer. Oncotarget. 2017; 8:15621-15631.
159. Hamanishi J, Mandai M, Iwasaki M, et al. Programmed cell death 1 ligand 1 and tumor-infiltrating CD8+ T lymphocytes are prognostic factors of human ovarian cancer. Proc Natl Acad Sci USA. 2007; 104:3360-3365.
160. Hwang W T, Adams S F, Tahirovic E, Hagemann I S, Coukos G. Prognostic significance of tumor-infiltrating T cells in ovarian cancer: a meta-analysis. Gynecol Oncol. 2012; 124:192-198.
161. Rocconi R P, Stevens E E, Bottsford-Miller J N, et al. Proof of principle study of sequential combination atezolizumab and Vigil in relapsed ovarian cancer. Cancer Gene Ther. 2022; 29:369-382.
162. Barve M, Aaron P, Manning L, et al. Pilot Study of Combination Gemogenovatucel-T (Vigil) and Durvalumab in Women With Relapsed BRCA-wt Triple-Negative Breast or Ovarian Cancer. Clin Med Insights Oncol. 2022; 16:11795549221110501.
163. Moore K N, Bookman M, Sehouli J, et al. Atezolizumab, Bevacizumab, and Chemotherapy for Newly Diagnosed Stage III or IV Ovarian Cancer: Placebo-Controlled Randomized Phase III Trial (Imagyn050/GOG 3015/ENGOT-OV39). J Clin Oncol. 2021; 39:1842-1855.
164. Ledermann J A, Colombo N, Oza A M, et al. LBA 25-Scientific Plenary: Avelumab in combination with and/or following chemotherapy vs chemotherapy alone in patients with previously untreated epithelial ovarian cancer: Results from the phase 3 javelin ovarian 100 trial. Gynecologic Oncology. 2020; 159:13-14.
165. Monk B J, Colombo N, Oza A M, et al. Chemotherapy with or without avelumab followed by avelumab maintenance versus chemotherapy alone in patients with previously untreated epithelial ovarian cancer (JAVELIN Ovarian 100): an open-label, randomised, phase 3 trial. The Lancet Oncology. 2021; 22:1275-1289.
166. Ledermann J, Colombo N, Oza A, et al. 1 Avelumab in combination with and/or following chemotherapy vs chemotherapy in treatment-® patients with ovarian cancer: biomarker analyses from the phase 3 JAVELIN Ovarian 100 trial. International Journal of Gynecologic Cancer. 2020; 30: A1-A1.
167. Moore K N, Okamoto A, Wu F, Lin Y G, Pignata S. IMagyn050/GOG3015/ENGOT-ov39: A randomized, double-blind, phase III study of atezolizumab vs placebo combined with chemotherapy+bevacizumab in stage III-IV ovarian, fallopian tube & peritoneal cancers (O C). Annals of Oncology. 2017; 28: V350-V351.
168. Yu H, Boyle T A, Zhou C, Rimm D L, Hirsch F R. PD-L1 Expression in Lung Cancer. J Thorac Oncol. 2016; 11:964-975.
169. Goodman A M, Kato S, Bazhenova L, et al. Tumor Mutational Burden as an Independent Predictor of Response to Immunotherapy in Diverse Cancers. Mol Cancer Ther. 2017; 16:2598-2608.
170. Riviere P, Goodman A M, Okamura R, et al. High Tumor Mutational Burden Correlates with Longer Survival in Immunothe®Naive Patients with Diverse Cancers. Mol Cancer Ther. 2020; 19:2139-2145.
171. Strickler J H, Hanks B A, Khasraw M. Tumor Mutational Burden as a Predictor of Immunotherapy Response: Is More Always Better? Clin Cancer Res. 2021; 27:1236-1241.
172. Martin S D, Brown S D, Wick D A, et al. Low Mutation Burden in Ovarian Cancer May Limit the Utility of Neoantigen-Targeted Vaccines. PLOS One. 2016; 11: e0155189.
173. Maleki Vareki S. High and low mutational burden tumors versus immunologically hot and cold tumors and response to immune checkpoint inhibitors. J Immunother Cancer. 2018; 6:157.
174. Fan S, Gao X, Qin Q, Li H, Yuan Z, Zhao S. Association between tumor mutation burden and immune infiltration in ovarian cancer. Int Immunopharmacol. 2020; 89:107126.
175. Heeke A L, Pishvaian M J, Lynce F, et al. Prevalence of Homologous Recombination-Related Gene Mutations Across Multiple Cancer Types. JCO Precis Oncol. 2018; 2018.
176. Senzer N, Barve M, Kuhn J, et al. Phase I trial of “bi-shRNAi (furin)/GMCSF DNA/autologous tumor cell” vaccine (FANG) in advanced cancer. Mol Ther. 2012; 20:679-686.
177. Senzer N, Barve M, Nemunaitis J, et al. Long Term Follow Up: Phase I Trial of “bi-shRNA furin/GMCSF DNA/Autologous Tumor Cell” Immunotherapy (FANG™) in Advanced Cancer. Journal of Vaccines & Vaccination. 2013; 4:209.
178. Oh J, Barve M, Matthews C M, et al. Phase II study of Vigil® DNA engineered immunotherapy as maintenance in advanced stage ovarian cancer. Gynecol Oncol. 2016; 143:504-510.
179. Oh J, Barve M, Senzer N, et al. Long-term follow-up of Phase 2A trial results involving advanced ovarian cancer patients treated with Vigil® in frontline maintenance. Gynecol Oncol Rep. 2020; 34:100648.
180. Strickland K C, Howitt B E, Shukla S A, et al. Association and prognostic significance of BRCA1/2-mutation status with neoantigen load, number of tumor-infiltrating lymphocytes and expression of PD-1/PD-L1 in high grade serous ovarian cancer. Oncotarget. 2016; 7:13587-13598.
181. Birkbak N J, Kochupurakkal B, Izarzugaza J M, et al. Tumor mutation burden forecasts outcome in ovarian cancer with BRCA1 or BRCA2 mutations. PLOS One. 2013; 8: e80023.
182. Sliheet E, Robinson M, Morand S, et al. Network based analysis identifies TP53m-BRCA1/2 wt-homologous recombination proficient (HRP) population with enhanced susceptibility to Vigil immunotherapy. Cancer Gene Ther. 2022; 29:993-1000.
183. Rocconi R P, Stanbery L, Tang M, et al. ENTPD1/CD39 as a predictive marker of treatment response to gemogenovatucel-T as maintenance therapy in newly diagnosed ovarian cancer. Commun Med (Lond). 2022; 2:106.

### Embodiments

Embodiment 1. A method for treating a patient having a solid tumor cancer, the method comprising:
determining that the patient is homologous recombination proficient (HRP);
determining that the patient has a clonal tumor mutation burden (cTMB) greater than a threshold; and administering an immunotherapy to the patient.

Embodiment 2. The method of claim 1, wherein the patient has a clonal neoantigen load (cNEO) greater than a threshold.

Embodiment 3. The method of embodiment 1 or 2, wherein the patient has a wild-type BRCA1 gene and a wild-type BRCA2 gene.

Embodiment 4. The method of any preceding embodiments, wherein the clonal tumor mutation burden (cTMB) is determined by:
for each of a set of mutations:
determining a respective amount of tumor sequence reads that have the mutation; and
determining whether the mutation is a clonal mutation based on the respective amount, thereby determining clonal mutations; and
determining the cTMB from using the clonal mutations.

Embodiment 5. The method of embodiment 4, wherein the set of mutations is determined by:
performing exome sequencing of paired tumor and normal samples of a subject to obtain the tumor sequence reads and normal sequence reads; and
comparing the tumor sequence reads to the normal sequence reads to determine the set of mutations that are in the tumor sample and not in the normal sample.

Embodiment 6. The method of embodiment 5, further comprising normalizing the cTMB using a size of the exome sequenced.

Embodiment 7. The method of embodiment 5, further comprising:
attaching unique molecular identifiers to template nucleic acids of the paired tumor and normal samples;
amplifying the template nucleic acids prior to sequencing to generate reads; and
determining a consensus read from one or more reads for each of the unique molecular identifiers, thereby generating consensus reads, wherein the consensus reads are used for comparing the tumor sequence reads to the normal sequence reads to determine the set of mutations.

Embodiment 8. The method of embodiment 4, wherein the respective amount of tumor sequence reads that have the mutation is an allelic fraction.

Embodiment 9. The method of embodiment 4, wherein determining whether the mutation is a clonal mutation is further based on an allelic copy number and a tumor purity.

Embodiment 10. A method for treating a patient having a solid tumor cancer, the method comprising:
determining that the patient is homologous recombination proficient (HRP);
determining that the patient has a clonal neoantigen load (cNEO) greater than a threshold; and
administering an immunotherapy to the patient.

Embodiment 11. The method of embodiment 10, wherein the clonal neoantigen load (cNEO) is determined by:
determining a set of clonal mutations that are non-synonymous;
determining an MHC haplotype of the patient using tumor sequence reads and/or normal sequence reads;
for each clonal mutation:
generating a peptide corresponding to the clonal mutation at a genomic position;
determining a likelihood of the peptide being presented on a cell surface in complex with the MHC haplotype encoded by the patient;
identifying whether the peptide is a clonal neoantigen based on the likelihood; and
determining an amount of clonal neoantigens.

Embodiment 12. The method of embodiment 11, wherein identifying whether the peptide is a clonal neoantigen based on the likelihood includes:
determining an MHC-1 binding affinity for the peptide; and
identifying the peptide as a clonal neoantigen when the MHC-1 binding affinity is greater than a binding threshold and is greater than a reference binding affinity of a wildtype sequence at the genomic position.

Embodiment 13. The method of embodiment 11, wherein the set of clonal mutations is determined by:
performing exome sequencing of paired tumor and normal samples of a subject to obtain the tumor sequence reads and normal sequence reads; and
comparing the tumor sequence reads to the normal sequence reads to determine the set of clonal mutations that are present in the tumor samples and not in the normal samples.

Embodiment 14. The method of embodiment 13, further comprising:
attaching unique molecular identifiers to template nucleic acids of the paired tumor and normal samples;
amplifying the template nucleic acids prior to sequencing to generate reads; and
determining a consensus read from one or more reads for each of the unique molecular identifiers, thereby generating consensus reads, wherein the consensus reads are used for comparing the tumor sequence reads to the normal sequence reads to determine the set of clonal mutations.

Embodiment 15. The method of any one of embodiments 11-14, wherein determining the amount of clonal neoantigens includes counting a number of peptides identified as a clonal neoantigen.

Embodiment 16. The method of any one of embodiments 11-14, wherein determining the amount of clonal neoantigens includes counting a number of clonal mutations resulting in at least one clonal neoantigen.

Embodiment 17. The method of any one of embodiments 11-16, wherein the set of clonal mutations is determined by:
for each of a set of mutations:
determining a respective amount of tumor sequence reads that have the mutation; and
determining whether the mutation is a clonal mutation based on the respective amount, thereby determining clonal mutations.

Embodiment 18. The method of embodiment 17, wherein the respective amount of tumor sequence reads that have the mutation is an allelic fraction.

Embodiment 19. The method of embodiment 17, wherein determining whether the mutation is a clonal mutation is further based on an allelic copy number and a tumor purity.

Embodiment 20. The method of any one of embodiments 11-19, further comprising normalizing the amount of clonal neoantigens using a size of the exome sequenced.

Embodiment 21. The method of any one of embodiments 10-20, wherein the threshold is determined using training samples having a known responder classification to an immunotherapy treatment, wherein the immunotherapy treatment induces the immune system to attack cells carrying clonal neoantigens.

Embodiment 22. The method of embodiment 10, wherein the patient has a clonal tumor mutation burden (cTMB) greater than a threshold.

Embodiment 23. The method of embodiment 10 or 22, wherein the patient has a wild-type BRCA1 gene and a wild-type BRCA2 gene.

Embodiment 24. The method of any one of embodiments 1 to 23, wherein the immunotherapy comprises a therapeutically effective population of tumor infiltrating lymphocytes (TILs).

Embodiment 25. The method of embodiment 24, wherein the TILs specifically target the clonal neoantigen.

Embodiment 26. A method for treating cancer in a patient in need thereof, the method comprising:
determining that the patient is homologous recombination proficient (HRP); and
administering an immunotherapy to the patient.

Embodiment 27. The method of embodiment 26, wherein the patient has a clonal tumor mutation burden (cTMB) greater than a threshold.

Embodiment 28. The method of embodiment 26 or 27, wherein the patient has a clonal neoantigen load (cNEO) greater than a threshold.

Embodiment 29. The method of any one of embodiments 26 to 28, wherein the patient has a wild-type BRCA1 gene and a wild-type BRCA2 gene.

30. The method of any one of embodiments 1 to 29, wherein the method further comprises administering to the patient:
(i) a neoantigen that is predicted to be presented by an HLA molecule encoded by an HLA allele which has been determined not to have been lost in a tumor;
(ii) an immune cell which recognizes a neoantigen that is predicted to be presented by an HLA molecule encoded by an HLA allele which has been determined not to have been lost in a tumor; or
(iii) an antibody which recognizes a neoantigen that is predicted to be presented by an HLA molecule encoded by an HLA allele which has been determined not to have been lost in a tumor.

Embodiment 31. The method of embodiment 30, wherein determination of whether said HLA allele has been lost comprises the steps of:
aligning HLA allele sequence information from a tumor sample from said patient with an HLA allele reference sequence which is based on said patient's HLA type; and
determining a specific copy number of said HLA allele in said tumor.

Embodiment 32. The method of any one of embodiments 26 to 31, wherein the cancer is selected from the group consisting of a solid tumor cancer, ovarian cancer, adrenocortical carcinoma, bladder cancer, breast cancer, cervical cancer, cholangiocarcinoma, colorectal cancers, esophageal cancer, glioblastoma, glioma, hepatocellular carcinoma, head and neck cancer, kidney cancer, leukemia, lymphoma, lung cancer, melanoma, mesothelioma, multiple myeloma, pancreatic cancer, pheochromocytoma, plasmacytoma, neuroblastoma, prostate cancer, sarcoma, stomach cancer, uterine cancer, thyroid cancer, and a hematological cancer.

Embodiment 33. The method of any one of embodiments 1 to 32, wherein the method further comprises administering to the individual at least one dose of an additional therapeutic agent.

Embodiment 34. The method of embodiment 33, wherein the additional therapeutic agent is a checkpoint inhibitor or an angiogenesis inhibitor.

Embodiment 35. The method of embodiment 34, wherein the additional therapeutic agent is a checkpoint inhibitor.

Embodiment 36. The method of any one of embodiments 1 to 35, wherein the immunotherapy comprises a therapeutically effective population of engineered cells selected from the group consisting of tumor infiltrating lymphocytes (TILs), T cell receptor (TCR) cells, chimeric antigen receptor (CAR) T cells, and natural killer (NK) cells.

Embodiment 37. The method of embodiment 36, wherein the therapeutically effective population of engineered cells is tumor infiltrating lymphocytes (TILs).

Embodiment 38. The method of embodiment 36, wherein the therapeutically effective population of engineered cells is chimeric antigen receptor (CAR) T cells.

Embodiment 39. The method of embodiment 36, wherein the therapeutically effective population of engineered cells is natural killer (NK) cells.

Embodiment 40. A method for identifying a patient being a responder to a tumor infiltrating lymphocytes (TIL) therapy for treating cancer, the method comprising:
administering an immunotherapy;
assessing the effectiveness of the immunotherapy for treating cancer; and
determining that the patient is homologous recombination proficient (HRP) if the immunotherapy is effective.

Embodiment 41. The method of embodiment 40, further comprising, before the administering step, determining that the patient has a clonal tumor mutation burden (cTMB) greater than a threshold.

Embodiment 42. The method of embodiment 40 or 41, further comprising, before the administering step, determining that the patient has a clonal neoantigen (cNEO) proportion greater than a threshold.

Embodiment 43. The method of any one of embodiments 40 to 42, further comprising, before the administering step, determining that the patient has a wild-type BRCA1 gene and a wild-type BRCA2 gene.

Embodiment 44. The method of any one of embodiments 40 to 43, wherein the immunotherapy comprises a therapeutically effective population of TILs.

Embodiment 45. An engineered tumor cell construct comprising an exogenous gene to express an exogenous protein.

Embodiment 46. A method of treating cancer in a patient in need thereof, the method comprising:
selecting a responder by first selecting a patient who is HRP; and
administering the engineered tumor cell construct of embodiment 45 to the patient who is HRP.

Embodiment 47. The method of embodiment 46, further comprising, before the administering step, determining that the responder has a clonal tumor mutation burden (cTMB) greater than a threshold.

Embodiment 48. The method of embodiment 46 or 41, further comprising, before the administering step, determining that the responder has a clonal neoantigen (cNEO) proportion greater than a threshold.

Embodiment 49. The method of any one of embodiments 46 to 48, further comprising, before the administering step, determining that the responder has a wild-type BRCA1 gene and a wild-type BRCA2 gene.

Embodiment 50. A method of determining a treatment for a subject having a particular type of cancer, the method comprising:
performing exome sequencing of paired tumor and normal samples of a subject to obtain tumor sequence reads and normal sequence reads;
comparing the tumor sequence reads to the normal sequence reads to determine a set of mutations that are in the tumor sample and not in the normal sample;
determining a clonal tumor mutation burden (cTMB) by:
for each of the set of mutations:
determining a respective amount of the tumor sequence reads that have the mutation; and
determining whether the mutation is a clonal mutation based on the respective amount, thereby determining clonal mutations; and
determining the cTMB from using the clonal mutations;
comparing cTMB to a reference value, wherein the reference value is determined using training samples of training subjects that have the particular type of cancer and that have a known responder classification to an immunotherapy treatment, wherein the immunotherapy treatment that includes genetically modified tumor cells of the subject; and
determining whether the subject is a responder to the immunotherapy treatment based on the comparison.

Embodiment 51. The method of embodiment 50, further comprising:
determining that the subject is homologous recombination proficient (HRP); and
determining whether the subject is the responder to the immunotherapy treatment further based on the subject being homologous recombination proficient (HRP).

Embodiment 52. The method of embodiment 51, wherein the subject not being homologous recombination proficient (HRP) is determined to not respond to the immunotherapy treatment.

Embodiment 53. The method of embodiment 50, wherein the training samples are of training subjects that are homologous recombination proficient (HRP).

Embodiment 54. The method of embodiment 50, where the cTMB is determined from only the non-synonymous clonal mutations.

Embodiment 55. A method of determining a treatment for a subject having cancer, the method comprising:
performing exome sequencing of paired tumor and normal samples of the subject to obtain tumor sequence reads and normal sequence reads;
comparing the tumor sequence reads to the normal sequence reads to identify mutations that are present in only the tumor samples but not the normal samples;
determining a set of non-synonymous clonal mutations;
determining an MHC haplotype of the subject using the tumor sequence reads and/or the normal sequence reads;
for each non-synonymous clonal mutation:
generating a peptide corresponding to the mutation at a genomic position;
determining a likelihood of the peptide being presented on a cell surface in complex with the MHC haplotype encoded by the subject; and
identifying whether the peptide is a clonal neoantigen based on the likelihood;
determining an amount of clonal neoantigens;
comparing the amount of clonal neoantigens to a reference value, wherein the reference value is determined using training samples having a known responder classification to an immunotherapy treatment, wherein the immunotherapy treatment induces the immune system to attack cells carrying the clonal neoantigens; and
determining whether the subject is a responder to the immunotherapy treatment based on the comparison.

Embodiment 56. The method of embodiment 50 or 55, wherein the subject is a responder to the immunotherapy treatment, the method further comprising:
administering the immunotherapy treatment to the subject.

Embodiment 57. The method of embodiment 50 or 55, wherein determining a clonal mutation of the set of clonal mutations uses a chromosomal position of the clonal mutation, a sequencing read depth for reads that match a normal sequence at the chromosomal position, a sequencing read depth for reads that match a variant sequence, allelic copy number for a major allele and a minor allele at the chromosomal position, and an estimated percentage tumor DNA in the tumor genomic DNA sample.

Embodiment 58. The method of embodiment 55, wherein identifying whether the peptide is a clonal neoantigen based on the likelihood includes:
determining an MHC-1 binding affinity for the peptide; and
identifying the peptide as a clonal neoantigen when the MHC-1 binding affinity is greater than a binding threshold and is greater than a reference binding affinity of a wildtype sequence at the genomic position.

Embodiment 59. The method of embodiment 55, wherein determining the amount of clonal neoantigens includes determining a count of all peptides having a specified length range and being identified as a clonal neoantigen.

Embodiment 60. The method of embodiment 59, wherein the specified length range is 8-11 amino acids.

Embodiment 61. The method of embodiment 59, wherein determining the count includes:
generating of possible peptides within the specified length range that contain one or more non-consensus amino acids derived from the clonal mutation; and
determining a count of the possible peptides that are identified as clonal neoantigens.

Embodiment 62. The method of embodiment 40, wherein the amount is normalized by a length of an exome sequenced.

Embodiment 63. The method of embodiment 55, wherein determining the amount of clonal neoantigens includes:
determining a number of the set of non-synonymous clonal mutations that have a peptide as being a clonal neoantigen.

Embodiment 64. A computer product comprising a non-transitory computer readable medium storing a plurality of instructions that, when executed, cause a computer system to perform the method of any one of the preceding embodiments.

Embodiment 65. A system comprising:
the computer product of embodiment 64; and
one or more processors configured to execute instructions stored on the computer readable medium.

The patents and references mentioned herein incorporated by reference. It is intended that the following claims define the scope of the disclosure and that methods and structures within the scope of these claims and their equivalents be covered thereby.

What is claimed is:

1. A method for treating a patient having a solid tumor cancer, the method comprising:
determining that the patient is homologous recombination proficient (HRP);
determining that the patient has a clonal tumor mutation burden (cTMB) greater than a threshold; and
administering an immunotherapy to the patient.

2. The method of claim 1, wherein the patient has a clonal neoantigen load (cNEO) greater than a threshold.

3. The method of claim 1, wherein the patient has a wild-type BRCA1 gene and a wild-type BRCA2 gene.

4. The method of claim 1, wherein the clonal tumor mutation burden (cTMB) is determined by:
for each of a set of mutations:
determining a respective amount of tumor sequence reads that have the mutation; and
determining whether the mutation is a clonal mutation based on the respective amount, thereby determining clonal mutations; and
determining the cTMB from using the clonal mutations.

5. The method of claim 4, wherein the set of mutations is determined by:
performing exome sequencing of paired tumor and normal samples of a subject to obtain the tumor sequence reads and normal sequence reads; and
comparing the tumor sequence reads to the normal sequence reads to determine the set of mutations that are in the tumor sample and not in the normal sample.

6. The method of claim 5, further comprising normalizing the cTMB using a size of the exome sequenced.

7. The method of claim 5, further comprising:
attaching unique molecular identifiers to template nucleic acids of the paired tumor and normal samples;
amplifying the template nucleic acids prior to sequencing to generate reads; and
determining a consensus read from one or more reads for each of the unique molecular identifiers, thereby generating consensus reads, wherein the consensus reads are used for comparing the tumor sequence reads to the normal sequence reads to determine the set of mutations.

8. The method of claim 4, wherein the respective amount of tumor sequence reads that have the mutation is an allelic fraction.

9. The method of claim 4, wherein determining whether the mutation is a clonal mutation is further based on an allelic copy number and a tumor purity.

10. A method for treating a patient having a solid tumor cancer, the method comprising:
determining that the patient is homologous recombination proficient (HRP);
determining that the patient has a clonal neoantigen load (cNEO) greater than a threshold; and
administering an immunotherapy to the patient.

11. A method for treating cancer in a patient in need thereof, the method comprising:
determining that the patient is homologous recombination proficient (HRP); and
administering an immunotherapy to the patient.

12. The method of claim 11, wherein the patient has a clonal tumor mutation burden (cTMB) greater than a threshold.

13. The method of claim 11, wherein the patient has a clonal neoantigen load (cNEO) greater than a threshold.

14. A method for identifying a patient being a responder to a tumor infiltrating lymphocytes (TIL) therapy for treating cancer, the method comprising:
administering an immunotherapy;
assessing the effectiveness of the immunotherapy for treating cancer; and
determining that the patient is homologous recombination proficient (HRP) if the immunotherapy is effective.

15. The method of claim 14, further comprising, before the administering step, determining that the patient has a clonal tumor mutation burden (cTMB) greater than a threshold.

16. The method of claim 14, further comprising, before the administering step, determining that the patient has a clonal neoantigen (cNEO) proportion greater than a threshold.

17. The method of claim 14, further comprising, before the administering step, determining that the patient has a wild-type BRCA1 gene and a wild-type BRCA2 gene.

18. A method of treating cancer in a patient in need thereof, the method comprising:
selecting a responder by first selecting a patient who is HRP; and
administering an engineered tumor cell construct to the patient who is HRP.

19. A method of determining a treatment for a subject having a particular type of cancer, the method comprising:
performing exome sequencing of paired tumor and normal samples of a subject to obtain tumor sequence reads and normal sequence reads;
comparing the tumor sequence reads to the normal sequence reads to determine a set of mutations that are in the tumor sample and not in the normal sample;
determining a clonal tumor mutation burden (cTMB) by:
for each of the set of mutations:
determining a respective amount of the tumor sequence reads that have the mutation; and
determining whether the mutation is a clonal mutation based on the respective amount, thereby determining clonal mutations; and
determining the cTMB from using the clonal mutations;
comparing cTMB to a reference value, wherein the reference value is determined using training samples of training subjects that have the particular type of cancer and that have a known responder classification to an immunotherapy treatment, wherein the immunotherapy treatment that includes genetically modified tumor cells of the subject; and
determining whether the subject is a responder to the immunotherapy treatment based on the comparison.

20. A method of determining a treatment for a subject having cancer, the method comprising:
performing exome sequencing of paired tumor and normal samples of the subject to obtain tumor sequence reads and normal sequence reads;
comparing the tumor sequence reads to the normal sequence reads to identify mutations that are present in only the tumor samples but not the normal samples;
determining a set of non-synonymous clonal mutations;
determining an MHC haplotype of the subject using the tumor sequence reads and/or the normal sequence reads;
for each non-synonymous clonal mutation:
generating a peptide corresponding to the mutation at a genomic position;
determining a likelihood of the peptide being presented on a cell surface in complex with the MHC haplotype encoded by the subject; and
identifying whether the peptide is a clonal neoantigen based on the likelihood;
determining an amount of clonal neoantigens;
comparing the amount of clonal neoantigens to a reference value, wherein the reference value is determined using training samples having a known responder classification to an immunotherapy treatment, wherein the immunotherapy treatment induces the immune system to attack cells carrying the clonal neoantigens; and
determining whether the subject is a responder to the immunotherapy treatment based on the comparison.
